SEQUENCE LISTING (38 sequences)

SEQ ID NO: 1 - protein, 147 aa, Homo sapiens
ARALCRLPRRGLWLLLAHHLFMTTACQEANYGALLRELCLTQFQVDMEAVGETLWCDWGRTIRSYRELADCTWHMAEKLGCFWPNAEVDRFFLAVHGRYFRSCPISGRAVRDPPGSILYPFIVVPITVTLLVTALVVWQSKRTEGIV

SEQ ID NO: 2 - protein, 174 aa, Homo sapiens
ASLRVERAGGPRLPRTRVGRPAALRLLLLLGAVLNPHEALAQPLPTTGTPGSEGGTVKNYETAVQFCWNHYKDQMDPIEKDWCDWAMISRPYSTLRDCLEHFAELFDLGFPNPLAERIIFETHQIHFANCSLVQPTFSDPPEDVLLAMIIAPICLIPFLITLVVWRSKDSEAQA

SEQ ID NO: 3 - protein, 147 aa, Homo sapiens
ETGALRRPQLLPLLLLLCGGCPRAGGCNETGMLERLPLCGKAFADMMGKVDVWKWCNLSEFIVYYESFTNCTEMEANVVGCYWPNPLAQGFITGIHRQFFSNCTVDRVHLEDPPDEVLIPLIVIPVVLTVAMAGLVVWRSKRTDTLL

SEQ ID NO: 4 - protein, 628 aa, Homo sapiens
GCKVLLNIGQQMLRRKVVDCSREETRLSRCLNTFDLVALGVGSTLGAGVYVLAGAVARENAGPAIVISFLIAALASVLAGLCYGEFGARVPKTGSAYLYSYVTVGELWAFITGWNLILSYIIGTSSVARAWSATFDELIGRPIGEFSRTHMTLNAPGVLAENPDIFAVIIILILTGLLTLGVKESAMVNKIFTCINVLVLGFIMVSGFVKGSVKNWQLTEEDFGNTSGRLCLNNDTKEGKPGVGGFMPFGFSGVLSGAATCFYAFVGFDCIATTGEEVKNPQKAIPVGIVASLLICFIAYFGVSAALTLMMPYFCLDNNSPLPDAFKHVGWEGAKYAVAVGSLCALSASLLGSMFPMPRVIYAMAEDGLLFKFLANVNDRTKTPIIATLASGAVAAVMAFLFDLKDLVDLMSIGTLLAYSLVAACVLVLRYQPEQPNLVYQMASTSDELDPADQNELASTNDSQLGFLPEAEMFSLKTILSPKNMEPSKISGLIVNISTSLIAVLIITFCIVTVLGREALTKGALWAVFLLAGSALLCAVVTGVIWRQPESKTKLSFKVPFLPVLPILSIFVNVYLMMQLDQGTWVRFAVWMLIGFIIYFGYGLWHSEEASLDADQARTPDGNLDQCK

SEQ ID NO: 5 - protein, 37 aa, synthetic construct
  SITE 37: note = amide C-terminus
  DISULFID 2..7: note = disulfid bond
KCNTATCATQRLANFLVHSSNNFGPILPPTNVGSNTY

SEQ ID NO: 6 - protein, 37 aa, synthetic construct
  DISULFID 2..7: note = disulfide bond
  SITE 1: note = yE-C18diacid
  SITE 23: note = (S)-2-amino-2-methyl-3-phenylpropanoic acid
  SITE 37: note = amide C-terminus
XCNTATCATQRLAEFLRHSSNNFGPILPPTNVGSNTY

SEQ ID NO: 7 - protein, 37 aa, synthetic construct
  DISULFID 2..7: note = disulfid bond
  SITE 1: note = side chain of Lys is connected to yE-yE-C18diacid
  SITE 37: note = amide C-terminus
  SITE 23: note = (S)-2-amino-2-methyl-3-phenylpropanoic acid
KCNTATCATQRLAEFLRHSSNNFGPILPPTNVGSNTY

SEQ ID NO: 8 - protein, 38 aa, synthetic construct
  DISULFID 3..8: note = disulfid bond
  SITE 1: note = side chain of Lys is connected to yE-C18diacid
  SITE 38: note = amide C-terminus
  SITE 24: note = (S)-2-amino-2-methyl-3-phenylpropanoic acid
KKCNTATCATQRLAEFLRHSSNNFGPILPPTNVGSNTY

SEQ ID NO: 9 - protein, 38 aa, synthetic construct
  DISULFID 3..8: note = disulfid bond
  SITE 1: note = side chain of Lys is connected to yE-C18diacid
  SITE 38: note = amide C-terminus
  SITE 24: note = (S)-2-amino-2-methyl-3-phenylpropanoic acid
KKCNTATCATQRLANFLRHSSNNFGPILPPTEVGSNTY

SEQ ID NO: 10 - protein, 37 aa, synthetic construct
  DISULFID 2..7: note = disulfid bond
  SITE 1: note = side chain of Lys is connected to O2Oc-O2Oc-yE-C18diacid
  SITE 23: note = (S)-2-amino-2-methyl-3-phenylpropanoic acid
  SITE 37: note = amide C-terminus
KCNTATCATQRLANFLRHSSNNFGPILPPTEVGSNTY

SEQ ID NO: 11 - protein, 37 aa, synthetic construct; identical to SEQ ID NO: 10
  DISULFID 2..7: note = disulfide bond
  SITE 1: note = side chain of Lys is connected to yE-yE-C18diacid
  SITE 23: note = (S)-2-amino-2-methyl-3-phenylpropanoic acid
  SITE 37: note = amide C-terminus

SEQ ID NO: 12 - protein, 37 aa, synthetic construct
  DISULFID 2..7: note = disulfid bond
  SITE 1: note = side chain of Lys is connected to yE-C18diacid
  SITE 23: note = (S)-2-amino-2-methyl-3-phenylpropanoic acid
  SITE 37: note = amide C-terminus
KCNTATCATQRLANFLRHSSNNFGPILPPTNVGSNTY

SEQ ID NO: 13 - protein, 37 aa, synthetic construct; identical to SEQ ID NO: 12
  DISULFID 2..7: note = disulfid bond
  SITE 1: note = side chain of Lys is connected to yE-yE-C18diacid
  SITE 23: note = (S)-2-amino-2-methyl-3-phenylpropanoic acid
  SITE 37: note = amide C-terminus

SEQ ID NO: 14 - protein, 37 aa, synthetic construct
  DISULFID 2..7: note = disulfid bond
  SITE 1: note = side chain of Lys is connected to yE-yE-C18diacid
  SITE 23: note = (S)-2-amino-2-methyl-3-phenylpropanoic acid
  SITE 37: note = amide C-terminus
KCNTATCATQRLANFLVHSSNNFGPILPPTNVGSRTY

SEQ ID NO: 15 - protein, 37 aa, synthetic construct
  DISULFID 2..7: note = disulfid bond
  SITE 1: note = side chain of Lys is connected to yE-yE-C18diacid
  SITE 23: note = (S)-2-amino-2-methyl-3-phenylpropanoic acid
  SITE 37: note = amide C-terminus
KCNTATCATQRLANFLVHSSNNFGPILPPTRVGSNTY

SEQ ID NO: 16 - protein, 37 aa, synthetic construct; identical to SEQ ID NO: 15
  DISULFID 2..7: note = disulfid bond
  SITE 1: note = side chain of Lys is connected to yE-C18diacid
  SITE 23: note = (S)-2-amino-2-methyl-3-phenylpropanoic acid
  SITE 37: note = amide C-terminus

SEQ ID NO: 17 - protein, 38 aa, synthetic construct
  DISULFID 3..8: note = disulfid bond
  SITE 1: note = side chain of Lys is connected to yE-C18diacid
  SITE 15: note = Dbu
  SITE 38: note = amide C-terminus
KKCNTATCATQRLAXFLVHSSNNFGPILPPTNVGSNTY

SEQ ID NO: 18 - protein, 38 aa, synthetic construct
  DISULFID 3..8: note = disulfid bond
  SITE 1: note = side chain of Lys is connected to yE-C18diacid
  SITE 22: note = Aib
  SITE 38: note = amide C-terminus
KKCNTATCATQRLANFLVHSSXNFGPILPPTHVGSNTY

SEQ ID NO: 19 - protein, 37 aa, synthetic construct
  DISULFID 2..7: note = disulfid bond
  SITE 1: note = side chain of Lys is connected to yE-C18diacid
  SITE 21: note = Aib
  SITE 37: note = amide C-terminus
KCNTATCATQRLANFLRHSSXNFGPILPPTEVGSNTY

SEQ ID NO: 20 - protein, 37 aa, synthetic construct
  DISULFID 2..7: note = disulfid bond
  SITE 1: note = side chain of Lys is connected to yE-C18diacid
  SITE 21: note = Aib
  SITE 37: note = amide C-terminus
KCNTATCATQRLANFLRHSSXNFGPILPPTNVGSNTY

SEQ ID NO: 21 - protein, 38 aa, synthetic construct
  DISULFID 3..8: note = disulfid bond
  SITE 1: note = side chain of Lys is connected to yE-yE-C18diacid
  SITE 22: note = Aib
  SITE 38: note = amide C-terminus
KKCNTATCATQRLANFLRHSSXNFGPILPPTNVGSNTY

SEQ ID NO: 22 - protein, 38 aa, synthetic construct
  DISULFID 3..8: note = disulfid bond
  SITE 1: note = side chain of Lys is connected to yE-yE-C18diacid
  SITE 22: note = Aib
  SITE 38: note = amide C-terminus
KKCNTATCATQRLANFLVHSSXNFGPILPPTRVGSNTY

SEQ ID NO: 23 - protein, 38 aa, synthetic construct
  DISULFID 3..8: note = disulfid bond
  SITE 1: note = side chain of Lys is connected to yE-C18diacid
  SITE 22: note = Aib
  SITE 32: note = Aib
  SITE 38: note = amide C-terminus
KKCNTATCATQRLANFLVHSSXNFGPILPPTXVGSNTY

SEQ ID NO: 24 - protein, 38 aa, synthetic construct
  DISULFID 3..8: note = disulfid bond
  SITE 1: note = side chain of Lys is connected to yE-C18diacid
  SITE 18: note = Aib
  SITE 38: note = amide C-terminus
KKCNTATCATQRLANFLXHSSNNFGPILPPTNVGSNTY

SEQ ID NO: 25 - protein, 38 aa, synthetic construct
  DISULFID 3..8: note = disulfid bond
  SITE 1: note = side chain of Lys is connected to yE-C18diacid
  SITE 15: note = Dbu
  SITE 18: note = Aib
  SITE 38: note = amide C-terminus
KKCNTATCATQRLAXFLXHSSNNFGPILPPTEVGSNTY

SEQ ID NO: 26 - protein, 37 aa, synthetic construct; identical to SEQ ID NO: 10
  DISULFID 2..7: note = disulfid bond
  SITE 23: note = (S)-2-amino-2-methyl-3-phenylpropanoic acid
  SITE 37: note = amide C-terminus

SEQ ID NO: 27 - protein, 30 aa, Homo sapiens
  SITE 30: note = amide C-terminus
HAEGTFTSDVSSYLEGQAAKEFIAWLVKGR

SEQ ID NO: 28 - protein, 31 aa, Homo sapiens
HAEGTFTSDVSSYLEGQAAKEFIAWLVKGRG

SEQ ID NO: 29 - protein, 462 aa, Homo sapiens
AGAPGLLRLALLLLGMVGRAGPRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLYIIYTVGYALSFSALVIASAILLGFRHLHCTRNYIHLNLFASFILRALSVFIKDAALKWMYSTAAQQHQWDGLLSYQDSLSCRLVFLLMQYCVAANYYWLLVEGVYLYTLLAFSVLSEQWIFRLYVSIGWGVPLLFVVPWGIVKYLYEDEGCWTRNSNMNYWLIIRLPILFAIGVNFLIFVRVICIVVSKLKANLMCKTDIKCRLAKSTLTLIPLLGTHEVIFAFVMDEHARGTLRFIKLFTELSFTSFQGLMVAILYCFVNNEVQLEFRKSWERWRLEHLHIQRDSSMKPLKCPTSSLSSGATAGSSMYTATCQASCS

SEQ ID NO: 30 - protein, 179 aa, Homo sapiens
KSIYFVAGLFVMLVQGSWQRSLQDTEEKSRSFSASQADPLSDPDQMNEDKRHSQGTFTSDYSKYLDSRRAQDFVQWLMNTKRNRNNIAKRHDEFERHAEGTFTSDVSSYLEGQAAKEFIAWLVKGRGRRDFPEEVAIVEELGRRHADGSFSDEMNTILDNLAARDFINWLIQTKITDRK

SEQ ID NO: 31 - protein, 476 aa, Homo sapiens
PPCQPQRPLLLLLLLLACQPQVPSAQVMDFLFEKWKLYGDQCHHNLSLLPPPTELVCNRTFDKYSCWPDTPANTTANISCPWYLPWHHKVQHRFVFKRCGPDGQWVRGPRGQPWRDASQCQMDGEEIEVQKEVAKMYSSFQVMYTVGYSLSLGALLLALAILGGLSKLHCTRNAIHANLFASFVLKASSVLVIDGLLRTRYSQKIGDDLSVSTWLSDGAVAGCRVAAVFMQYGIVANYCWLLVEGLYLHNLLGLATLPERSFFSLYLGIGWGAPMLFVVPWAVVKCLFENVQCWTSNDNMGFWWILRFPVFLAILINFFIFVRIVQLLVAKLRARQMHHTDYKFRLAKSTLTLIPLLGVHEVVFAFVTDEHAQGTLRSAKLFFDLFLSSFQGLLVAVLYCFLNKEVQSELRRRWHRWRLGKVLWEERNTSNHRASSSPGHGPPSKELQFGRGGGSQDSSAETPLAGGLPRLAESPF

SEQ ID NO: 32 - protein, 30 aa, synthetic construct
  SITE 10: note = side chain of Lys is connected to yE-Palmitoyl
HSQGTFTSDKSEYLDSERARDFVAWLEAGG

SEQ ID NO: 33 - protein, 30 aa, synthetic construct
  SITE 2: note = Aib
  SITE 13: note = (S)-2-amino-2-methyl-3-phenylpropanoic acid
  SITE 29: note = Aib
HXQGTFTSDVSKFLDTKRARDFVQWLLEXG

SEQ ID NO: 34 - protein, 29 aa, synthetic construct
  SITE 2: note = Aib
  SITE 6: note = (S)-2-amino-2-methyl-3-phenylpropanoic acid
  SITE 13: note = (S)-2-amino-2-methyl-3-phenylpropanoic acid
  SITE 22: note = (S)-2-amino-2-methyl-3-phenylpropanoic acid
  SITE 25: note = Aib
  SITE 29: note = amide C-terminus
HXHGSFTSDVSKFLDSRAAKDFVQXIANT

SEQ ID NO: 35 - protein, 30 aa, synthetic construct; identical to SEQ ID NO: 33
  SITE 2: note = Aib
  SITE 29: note = Aib
  SITE 13: note = (S)-2-amino-2-methyl-3-phenylpropanoic acid
  SITE 17: note = side chain of Lys is connected to O2Oc-O2Oc-yE-C18diacid

SEQ ID NO: 36 - protein, 29 aa, synthetic construct
  SITE 2: note = Aib
  SITE 6: note = (S)-2-amino-2-methyl-3-phenylpropanoic acid
  SITE 13: note = (S)-2-amino-2-methyl-3-phenylpropanoic acid
  SITE 20: note = side chain of Lys is connected to O2Oc-O2Oc-yE-C18diacid
  SITE 22: note = (S)-2-amino-2-methyl-3-phenylpropanoic acid
  SITE 25: note = Aib
  SITE 29: note = amide C-terminus
HXHGSFTSDVSKFLDSRAAKDFVQXAINT

SEQ ID NO: 37 - protein, 30 aa, synthetic construct; identical to SEQ ID NO: 33
  SITE 2: note = Aib
  SITE 13: note = (S)-2-amino-2-methyl-3-phenylpropanoic acid
  SITE 17: note = side chain of Lys is connected to O2Oc-O2Oc-yE-C20diacid
  SITE 29: note = Aib

SEQ ID NO: 38 - protein, 29 aa, synthetic construct
  SITE 2: note = Aib
  SITE 6: note = (S)-2-amino-2-methyl-3-phenylpropanoic acid
  SITE 13: note = (S)-2-amino-2-methyl-3-phenylpropanoic acid
  SITE 20: note = side chain of Lys is connected to O2Oc-O2Oc-yE-yE-C20diacid
  SITE 22: note = (S)-2-amino-2-methyl-3-phenylpropanoic acid
  SITE 25: note = Aib
  SITE 29: note = amide C-terminus
HXHGSFTSDVSKFLDSRAAKDFVQXIANT

(19) United States
(12) Patent Application Publication
(10) Pub. No.: US 2026/0284148 A1
(43) Pub. Date: Sep. 24, 2026

(54) POLYPEPTIDE COMBINATION DOSAGE REGIMENS

(71) Applicant: Medlmmune Limited, Cambridge (GB)

(72) Inventor: Lars HANSEN, Wilmington, DE (US)

(21) Appl. No.: 19/374,372

(22) Filed: Oct. 30, 2025

Related U.S. Application Data
(60) Provisional application No. 63/715,254, filed on Nov. 1, 2024.

(51) Int. Cl. A61K 38/26; A61K 9/00; A61K 38/17; A61K 45/06; A61P 3/04

(52) U.S. Cl. CPC A61K 38/26; A61K 9/0019; A61K 38/1716; A61K 45/06; A61P 3/04

(57) ABSTRACT

The present invention relates to a method of treating and/or preventing a disease or disorder in a subject, the method comprising administering to the subject about 1 mg to about 15 mg of an amylin receptor (AMYR) agonist and about 0.1 mg to about 10 mg of a GLP-1 receptor (GLP-1R) agonist (e.g. a GLP-1R/glucagon receptor dual agonist). Also provided are corresponding compositions and kits.

### CROSS-REFERENCE TO RELATED APPLICATIONS

This application claims priority from U.S. provisional application No. 63/715,254, filed Nov. 1, 2024, which is incorporated herein by reference.

### REFERENCE TO SEQUENCE LISTING

The content of the electronically submitted sequence listing (Name: 2025-10-30-USPSP-SEQ-LISTING.xml, Size: 82,254 bytes, and Date of Creation: Oct. 28, 2025) submitted in this application is incorporated herein by reference in its entirety.

### FIELD OF THE INVENTION

The present invention relates to dosage regimens for treating and/or preventing a disease or disorder in a subject by administering a combination of an amylin receptor agonist and a GLP-1 receptor agonist.

### BACKGROUND

The incidence of obesity and diabetes have been rising in epidemic proportions.

Obesity is the result of complex relationships between genetic, socioeconomic, and cultural influences, and is defined as a BMI of 30 kg/m². It is a risk factor for the development of many comorbid conditions, including type 2 diabetes mellitus, CV disease, and chronic kidney disease, and it is estimated that obesity affects nearly a third of the world's population (Chooi et al. 2019 Metabolism. 2019; 92:6-10). Weight loss has been associated with improvements in comorbidities, however, achieving and maintaining weight loss through lifestyle interventions alone is difficult. Bariatric surgery is currently the most effective treatment for sustained body weight loss (O'Brien et al. 2019 Obes Surg. 2019; 29(1):3-1); however, such surgical intervention is only appropriate for a minority of the patient population.

Diabetes is characterized by high levels of blood glucose resulting from defects in insulin production, insulin action, or both. Type 2 diabetes mellitus (T2DM) accounts for some 90 to 95 percent of all diagnosed cases of diabetes, and the risk of type 2 diabetes rises with increasing body weight. The prevalence of type 2 diabetes is three to seven times higher in those who are affected by obesity than in normal weight adults, and is 20 times more likely in those with a body mass index (BMI) greater than 35 kg/m². However, weight-loss can improve control or cure type 2 diabetes.

Despite the development of several new treatments for overweight and obesity, concerns regarding tolerability, suboptimal response and the need for additional weight loss persist among a significant number of patients, highlighting an unmet need for additional treatment modalities.

Glucagon and glucagon-like peptide-1 (GLP-1) derive from pre-proglucagon, a 158 amino acid precursor polypeptide that is processed in different tissues to form a number of different proglucagon-derived peptides, including glucagon, glucagon-like peptide-1 (GLP-1), glucagon-like peptide-2 (GLP-2) and oxyntomodulin (OXM), that are involved in a wide variety of physiological functions, including glucose homeostasis, insulin secretion, gastric emptying, and intestinal growth, as well as the regulation of food intake. Glucagon is a 29-amino acid peptide that corresponds to amino acids 33 through 61 of proglucagon (53 to 81 of preproglucagon), while GLP-1 is produced as a 37-amino acid peptide that corresponds to amino acids 72 through 108 of proglucagon (92 to 128 of preproglucagon). GLP-1(7-36) amide or GLP-1(7-37) acid are biologically active forms of GLP-1, that demonstrate essentially equivalent activity at the GLP-1 receptor (GLP-1R).

Glucagon is produced by the pancreas and interacts with the glucagon receptor (“GCGR”). Glucagon acts in the liver to raise blood glucose via gluconeogenesis and glycogenolysis. When blood glucose begins to fall, glucagon signals the liver to break down glycogen and release glucose, causing blood glucose levels to rise toward a normal level.

GLP-1 has different biological activities compared to glucagon. It is secreted from gut L− cells and binds to the GLP-1R. Its activities include stimulation of insulin secretion, inhibition of glucagon secretion, and inhibition of food intake.

Both glucagon and GLP-1, acting as agonists at their respective receptors, have been shown to be effective in weight loss. Certain GLP-1 analogues are being sold or are in development for treatment of obesity including, e.g., Liraglutide (Saxenda® from Novo Nordisk) and Semaglutide (Wegovy® from Novo Nordisk). Glucagon/GLP-1 dual agonist peptides such as cotadutide are also known and are in clinical development for treatment of diabetes, obesity, and metabolic dysfunction associated steatohepatitis (MASH).

Amylin analogues are also being considered for the treatment of obesity, excess food intake, and diabetes (see e.g., WO 2018/046719). Pramlintide, a synthetic analogue of human amylin, is clinically used in amylin replacement therapies and simulates the important glucoregulatory actions of amylin. These glucoregulatory actions complement those of insulin by regulating the rate of appearance of glucose in the circulation, and are achieved through three primary mechanisms: slowing the rate of gastric emptying, suppression of post-meal glucagon secretion and suppression of food intake (Roth J D et. al. GLP-1R and amylin agonism in metabolic disease: complementary mechanisms and future opportunities. Br J Pharmacol. 2012; 166(1):121-136). Pramlintide has been used as an adjunct to insulin in patients with diabetes who have failed to reach desired glucose control despite optimal insulin therapy (Pullman J, et. al. Pramlintide is used in the management of insulin-using patients with type 2 and type 1 diabetes. Vasc Health Risk Manag. 2006; 2(3):203-212). Pramlintide analogues conjugated to lipids to extend their half-life are also known.

Existing agents are not without their problems. GLP-1 agonists, glucagon/GLP-1 agonists and some amylin analogues are reported to be associated with poor tolerability, as nausea and vomiting are common side effects. In addition, GLP-1 agonists and glucagon/GLP-1 agonists are known to result in the loss of lean muscle mass, which is clinically undesirable. Moreover, GLP-1 agonists and glucagon/GLP-1 dual agonists alone may not achieve weight loss to a sufficiently desirable level.

Accordingly, there remains a need for treatments that can treat and/or prevent a disease or disorder, improve glycemic control, reduce weight, treat type 2 diabetes mellitus (T2DM), and/or treat NASH in a subject, while minimizing burdens associated with a reduction in lean muscle mass. Further, there remains a need for treatments that can achieve greater levels of weight loss and the concomitant improvements in health outcomes. It is an object of the present invention to address one or more of these issues.

### SUMMARY OF THE INVENTION

AZD6234 is a potent agonist of human, cynomolgus monkey, and rat amylin receptors (AMYR), displaying in vitro selectivity over calcitonin receptor activation. AZD9550 is a synthetic 30-mer peptide with balanced dual activity for GLP-1 and GCG receptors. Both peptides have been acylated (i.e., lipidated) to confer binding to serum albumin, and therefore have a long half-life enabling once-weekly administration in humans. In vitro studies and in vivo toxicology studies have demonstrated acceptable safety profiles in rats and cynomolgus monkey for both peptides.

The present inventors sought to determine, for the first time, how a combination of AZD6234 and AZD9550 behaves when administered to humans at various doses. The inventors have previously established through Phase I human clinical trials that AZD6234 monotherapies are generally safe and well tolerated when administered to humans. The inventors have now further established through Phase I human clinical trials that AZD9550 monotherapies are generally safe and well tolerated when administered to humans. The inventors have combined in vivo pharmacokinetic and safety data obtained from the in-human trial with preclinical efficacy data obtained from animal models to devise dosages for a combination therapy of AZD6234 and AZD9550 with the potential to achieve an efficacious weight reducing effect in humans without compromising patient safety.

Further, the inventors have established that combining a particular class of AMYR agonists (e.g. AMYR agonists which have selectivity for AMYR as compared with calcitonin receptor (CTR)) and GLP-1R agonists (e.g., GLP-1R/GCGR dual agonists) result in improved tolerability (e.g., reduction in nausea and/or vomiting), e.g., as compared to other anti-obesity drugs, such as a therapeutically effective dose of a dual amylin-calcitonin receptor agonist (DACRA e.g., davalintide); a GLP-1R agonist (e.g., semaglutide); a GLP-1R/GCGR dual agonist (e.g. survodutide); a GLP1/GCG/GIP triple agonist (e.g. retatrutide); and/or a DACRA-GLP-1Ra combination (e.g., semaglutide-cagrilintide, “cagri-sema”). By way of further benefit, the combination treatments with an AMYR agonist and a GLP1-R agonist provided herein can promote fat mass loss and preserve lean mass without compromising tolerability, making this a clinically attractive treatment paradigm. As further demonstrated herein, this combination treatment has the potential to provide synergistic effects beyond those observed using AMYR agonists and GLP1-R agonists as monotherapies.

Hence, administering a combination of an AMYR agonist, such as AZD6234, and a GLP-1R agonist (e.g., a GLP-1R/GCGR dual agonist), such as AZD9550, at the doses described herein has the potential to achieve clinical benefit when administered to humans for treating and/or preventing a disease. For example, the combination therapy may be useful in reducing body weight, including the treatment of obesity and obesity-related conditions.

Such treatments may be particularly suitable for achieving additional reduction in body weight over and above treatment with other weight loss actives, such as GLP-1 receptor (GLP-1R) agonists (e.g. semaglutide) and/or GLP-1R/glucagon receptor (GCGR) dual-agonists (e.g. tirzepatide), and/or AMYR or DACRA agonists, and particularly monotherapies using such actives.

Accordingly, the present invention provides a method for treating and/or preventing a disease or disorder in a subject, the method comprising administering to the subject (a) about 1 mg to about 15 mg of an amylin receptor (AMYR) agonist; and (b) about 0.1 mg to about 10 mg of a GLP-1 receptor (GLP-1R) agonist.

The GLP-1R agonist may activate both a GLP-1R and a glucagon receptor (GCGR).

The method may further comprise administering to the subject a glucagon receptor (GCGR) agonist.

The amount of GLP-1R agonist may be from about 0.1 mg to about 7 mg.

The amount of GLP-1R agonist may be from about 0.1 mg to about 5 mg.

The amount of GLP-1R agonist may be from about 0.1 mg to about 1.5 mg.

The amount of GLP-1R agonist may be from about 0.1 mg to about 1 mg.

The amount of GLP-1R agonist may be from about 0.1 mg to about 0.5 mg

The amount of GLP-1R agonist may be from about 0.1 mg to about 0.2 mg.

The amount of GLP-1R agonist may be from about 0.2 mg to about 10 mg.

The amount of GLP-1R agonist may be from about 0.2 mg to about 7 mg.

The amount of GLP-1R agonist may be from about 0.2 mg to about 5 mg.

The amount of GLP-1R agonist may be from about 0.2 mg to about 1.5 mg.

The amount of GLP-1R agonist may be from about 0.2 mg to about 1 mg.

The amount of GLP-1R agonist may be from about 0.2 mg to about 0.5 mg.

The amount of GLP-1R agonist may be from about 0.5 mg to about 10 mg.

The amount of GLP-1R agonist may be from about 0.5 mg to about 7 mg.

The amount of GLP-1R agonist may be from about 0.5 mg to about 5 mg.

The amount of GLP-1R agonist may be from about 0.5 mg to about 1.5 mg.

The amount of GLP-1R agonist may be from about 0.5 mg to about 1 mg.

The amount of GLP-1R agonist may be from about 1 mg to about 10 mg.

The amount of GLP-1R agonist may be from about 1 mg to about 7 mg.

The amount of GLP-1R agonist may be from about 1 mg to about 5 mg.

The amount of GLP-1R agonist may be from about 1 mg to about 1.5 mg.

The amount of GLP-1R agonist may be from about 1.5 mg to about 5 mg.

The amount of GLP-1R agonist may be from about 2 mg to about 10 mg.

The amount of GLP-1R agonist may be from about 2 mg to about 7 mg.

The amount of GLP-1R agonist may be from about 2 mg to about 5 mg.

The amount of GLP-1R agonist may be from about 4 mg to about 10 mg.

The amount of GLP-1R agonist may be from about 4 mg to about 7 mg.

The amount of GLP-1R agonist may be from about 4 mg to about 5 mg.

The amount of GLP-1R agonist may be from about 5 mg to about 10 mg.

The amount of GLP-1R agonist may be from about 5 mg to about 7 mg.

The amount of GLP-1R agonist may be from about 8 mg to about 10 mg.

The amount of GLP-1R agonist may be about 0.15 mg, about 0.3 mg, about 0.6 mg, about 1.2 mg, about 2.3 mg, about 4.6 mg, about 7.0 mg, or about 9.4 mg, preferably about 1.2 mg, about 4.6 mg, about 7 mg, or about 9.4 mg.

The amount of AMYR agonist may be from about 1 mg to about 10 mg.

The amount of AMYR agonist may be from about 1 mg to about 5 mg.

The amount of AMYR agonist may be from about 4 mg to about 10 mg.

The amount of AMYR agonist may be from about 1 mg to about 3 mg.

The amount of AMYR agonist may be from about 2.5 mg to about 5 mg.

The amount of AMYR agonist may be from about 7 mg to about 10 mg.

The amount of AMYR agonist may be about 1.5 mg (e.g. 1.6 mg), about 4.5 mg, about 6 mg, or about 9 mg.

The amount of AMYR agonist may be from about 7 mg to about 10 mg, optionally about 9 mg, and the amount of GLP-1R agonist may be from about 8 mg to about 10 mg, optionally about 9.4 mg.

The amount of AMYR agonist may be from about 7 mg to about 10 mg, optionally about 7 mg to about 10 mg, optionally about 9 mg, and the amount of GLP-1R agonist may be from about 5 mg to about 10 mg, optionally about 7 mg.

The amount of AMYR agonist may be from about 7 mg to about 10 mg, optionally about 7 mg to about 10 mg, optionally about 9 mg, and the amount of GLP-1R agonist may be from about 3 mg to about 5 mg, optionally about 4.6 mg.

The amount of AMYR agonist may be from about 7 mg to about 10 mg, optionally about 7 mg to about 10 mg, optionally about 9 mg, and the amount of GLP-1R agonist may be from about 2 mg to about 4 mg, optionally about 2.3 mg.

The amount of AMYR agonist may be from about 7 mg to about 10 mg, optionally about 7 mg to about 10 mg, optionally about 9 mg, and the amount of GLP-1R agonist may be from about 1 mg to about 2 mg, optionally about 1.2 mg.

The amount of AMYR agonist may be from about 7 mg to about 10 mg, optionally about 4 mg to about 7 mg, optionally about 6 mg, and the amount of GLP-1R agonist may be from about 8 mg to about 10 mg, optionally about 9.4 mg.

The amount of AMYR agonist may be from about 7 mg to about 10 mg, optionally about 4 mg to about 7 mg, optionally about 6 mg, and the amount of GLP-1R agonist may be from about 5 mg to about 10 mg, optionally about 7 mg.

The amount of AMYR agonist may be from about 7 mg to about 10 mg, optionally about 4 mg to about 7 mg, optionally about 6 mg, and the amount of GLP-1R agonist may be from about 3 mg to about 5 mg, optionally about 4.6 mg.

The amount of AMYR agonist may be from about 7 mg to about 10 mg, optionally about 4 mg to about 7 mg, optionally about 6 mg, and the amount of GLP-1R agonist may be from about 2 mg to about 4 mg, optionally about 2.3 mg.

The amount of AMYR agonist may be from about 7 mg to about 10 mg, optionally about 4 mg to about 7 mg, optionally about 6 mg, and the amount of GLP-1R agonist may be from about 1 mg to about 2 mg, optionally about 1.2 mg.

The amount of AMYR agonist may be from about 7 mg to about 10 mg, optionally about 4 mg to about 5 mg, optionally about 4.5 mg, and the amount of GLP-1R agonist may be from about 8 mg to about 10 mg, optionally about 9.4 mg.

The amount of AMYR agonist may be from about 7 mg to about 10 mg, optionally about 4 mg to about 5 mg, optionally about 4.5 mg, and the amount of GLP-1R agonist may be from about 5 mg to about 10 mg, optionally about 7 mg.

The amount of AMYR agonist may be from about 7 mg to about 10 mg, optionally about 4 mg to about 5 mg, optionally about 4.5 mg, and the amount of GLP-1R agonist may be from about 3 mg to about 5 mg, optionally about 4.6 mg.

The amount of AMYR agonist may be from about 7 mg to about 10 mg, optionally about 4 mg to about 5 mg, optionally about 4.5 mg, and the amount of GLP-1R agonist may be from about 2 mg to about 4 mg, optionally about 2.3 mg.

The amount of AMYR agonist may be from about 7 mg to about 10 mg, optionally about 4 mg to about 5 mg, optionally about 4.5 mg, and the amount of GLP-1R agonist may be from about 1 mg to about 2 mg, optionally about 1.2 mg.

The amount of AMYR agonist may be from about 7 mg to about 10 mg, optionally about 1 mg to about 2 mg, optionally about 1.5 mg (e.g. 1.6 mg), and the amount of GLP-1R agonist may be from about 8 mg to about 10 mg, optionally about 9.4 mg.

The amount of AMYR agonist may be from about 7 mg to about 10 mg, optionally about 1 mg to about 2 mg, optionally about 1.5 mg (e.g. 1.6 mg), and the amount of GLP-1R agonist may be from about 5 mg to about 10 mg, optionally about 7 mg.

The amount of AMYR agonist may be from about 7 mg to about 10 mg, optionally about 1 mg to about 2 mg, optionally about 1.5 mg (e.g. 1.6 mg), and the amount of GLP-1R agonist may be from about 3 mg to about 5 mg, optionally about 4.6 mg.

The amount of AMYR agonist may be from about 7 mg to about 10 mg, optionally about 1 mg to about 2 mg, optionally about 1.5 mg (e.g. 1.6 mg), and the amount of GLP-1R agonist may be from about 2 mg to about 4 mg, optionally about 2.3 mg.

The amount of AMYR agonist may be from about 7 mg to about 10 mg, optionally about 1 mg to about 2 mg, optionally about 1.5 mg (e.g. 1.6 mg), and the amount of GLP-1R agonist may be from about 1 mg to about 2 mg, optionally about 1.2 mg.

In any method of the invention, the AMYR agonist, GLP-1R agonist, and/or GCGR agonist may be administered by subcutaneous injection.

In any method of the invention the AMYR agonist, GLP-1R agonist, and/or GCGR agonist is administered to the subject by self-administration.

In any method of the invention the AMYR agonist, GLP-1R agonist, and/or GCGR agonist may be administered about once every 4, 5, 6, 7, 8, 9, 10, 11, 12, 13, or 14 days. The AMYR agonist, GLP-1R agonist, and/or GCGR agonist may be administered about once a week.

In any method of the invention the AMYR agonist, GLP-1R agonist, and/or GCGR agonist may be administered to the subject multiple times over a period of at least 3 months, 6 months, 9 months, 1 year, 2 years, or 5 years.

In any method of the invention the AMYR agonist, GLP-1R agonist, and/or GCGR agonist may be titrated to a final dose over a period between about 4 weeks and about 36 weeks, optionally over a period of about 5 weeks, about 17 weeks or about 21 weeks.

The dose of the AMYR agonist, GLP-1R agonist, and/or GCGR agonist may be increased every one week, every two weeks or every four weeks until the final dose is reached; optionally wherein: (a) a first dose of the AMYR agonist, GLP-1R agonist, and/or GCGR agonist is administered for the first two weeks; (b) following (a) the dose of the AMYR agonist, GLP-1R agonist, and/or GCGR agonist is increased for the next two weeks; and (c) following (b) the dose of the AMYR agonist, GLP-1R agonist, and/or GCGR agonist is increased every four weeks for the next 16 weeks.

In any method of the invention the subject's body weight may be reduced by ≥5%, optionally, the subject's body weight may be reduced by ≥10%, ≥15%, or ≥20%.

The disease or disorder may be obesity.

In any method of the invention, the disease or disorder may be an obesity-related condition; optionally wherein the obesity-related condition is overweight, morbid obesity, obesity prior to surgery, obesity-linked inflammation, obesity-linked gallbladder disease, sleep apnoea and respiratory problems, hyperlipidaemia, degeneration of cartilage, osteoarthritis, or reproductive health complications of obesity or overweight such as infertility.

In any method of the invention, the disease or disorder may be a metabolic disease, optionally the metabolic disease may include diabetes, type 1 diabetes, type 2 diabetes, gestational diabetes, pre-diabetes, insulin resistance, impaired glucose tolerance (IGI), disease states associated with elevated blood glucose levels, metabolic syndrome, or hyperglycaemia (e.g. abnormal postprandial hyperglycaemia).

In any method of the invention, the disease or disorder may be hepatic steatosis (“fatty liver”), e.g., Metabolic dysfunction associated steatohepatitis (MASH).

In any method of the invention, the method may further comprise treating, improving, and/or protecting liver and/or kidney function in the subject.

The method of treating, improving, and/or protecting liver function may comprise reducing steatohepatitis and/or fibrosis in the liver.

In any method of the invention the method of treatment may reduce the body weight of a subject, optionally wherein the subject's reduction in body weight is fat-specific weight loss.

In any method of the invention the reduction in the body weight of the subject may be greater than the reduction in body weight with a therapeutically effective dose of a dual amylin-calcitonin receptor agonist (DACRA), a GLP-1R agonist, a GLP-1R/GCGR dual agonist, a GLP1/GCG/GIP triple agonist, and/or a DACRA-GLP-1Ra combination.

In any method of the invention the subject may experience reduced nausea as compared to a therapeutically effective dose of a dual amylin-calcitonin receptor agonist (DACRA), a GLP-1R agonist, a GLP-1R/GCGR dual agonist; a GLP1/GCG/GIP triple agonist; and/or a DACRA-GLP-1Ra combination.

In any method of the invention the AMYR agonist, GLP-1R agonist, and/or GCGR agonist may be a polypeptide, small molecule drug, antibody, antibody-drug conjugate, or aptamer; or a pharmaceutically acceptable salt thereof.

The AMYR agonist, GLP-1R agonist, and/or GCGR agonist may be a polypeptide, or a pharmaceutically acceptable salt thereof.

In any method of the invention: the AMYR may be a human AMYR and/or the AMYR may be AMY1R, AMY2R and/or AMY3R; the GLP-1R may be a human GLP-1R; and/or the GCGR may be a human GCGR.

In any method of the invention the AMYR agonist may have selectivity to AMYR as compared to a calcitonin receptor (CTR).

The AMYR agonist may have at least a 2-fold, at least 3-fold, at least 4-fold, at least 5-fold, at least 6-fold, at least 7-fold, at least 8-fold, at least 9-fold, at least 10-fold, at least 12-fold, at least 15-fold, at least 17-fold, at least 20-fold, or at least 25-fold selectivity to AMYR as compared to CTR; optionally wherein the AMYR agonist has at least a 10-fold selectivity to AMYR as compared to CTR.

In any method of the invention, the AMYR agonist may be an AMYR agonist polypeptide, or a pharmaceutically acceptable salt thereof, which comprises an amino acid sequence having at least 90% identity to pramlintide (KCNTATCATQRLANFLVHSSNNFGPILPPTNVGSNTY-amide, SEQ ID NO: 5).

The AMYR agonist polypeptide may be lipidated and/or the lipid may be attached to an amino acid residue in the AMYR agonist polypeptide by a linker.

The AMYR agonist polypeptide may comprise an amino acid sequence having at least 90%, at least 95%, at least 97%, at least 99%, or 100% identity to the amino acid sequence K[CNTATC]ATQRLANFLRHSSNN(αMePhe)GPILPPTEVGSNTY-amide (SEQ ID NO: 26).

The AMYR agonist polypeptide, or pharmaceutically acceptable salt thereof, may comprise the amino acid sequence K(γE-γE-C18diacid)[CNTATC]ATQRLANFLRHSSNN(αMePhe)GPILPPTEVGSNTY-amide (SEQ ID NO: 11).

In any method of the invention, the GLP-1R agonist may activate both GLP-1R and GCGR, and optionally the GLP-1R agonist may be selective to GLP-1R as compared to GCGR.

The GLP-1R agonist may activate GLP-1R and GCGR equally, or the GLP-1R agonist may have at least a 1.5-fold, at least 2-fold, at least 3-fold, at least 4-fold, at least 5-fold, at least 6-fold, at least 7-fold, at least 8-fold, at least 9-fold, at least 10-fold, at least 12-fold, at least 15-fold, at least 17-fold, at least 20-fold, or at least 25-fold selectivity to GLP-1R as compared to GCGR; optionally wherein the GLP-1R agonist has at least a 1.5-fold selectivity to GLP-1R as compared to GCGR.

In any method of the invention the GLP-1R agonist polypeptide, or pharmaceutically acceptable salt thereof, may comprise an amino acid sequence having at least 90%, at least 95%, at least 97%, at least 99%, or 100% identity to the amino acid sequence H(Aib)QGTFTSDVSK(αMePhe)LDTKRARDFVQWLLE(Aib)G-acid (SEQ ID NO: 33).

The GLP-1R agonist polypeptide may be lipidated and/or the lipid may be attached to an amino acid residue in the GLP-1R agonist polypeptide by a linker.

The GLP-1R agonist polypeptide, or pharmaceutically acceptable salt thereof, may comprise the amino acid sequence H(Aib)QGTFTSDVSK(αMePhe)LDTK(O2Oc-O2Oc-γE-C18diacid)RARDFVQWLLE(Aib)G-acid (SEQ ID NO: 35).

In any method of the invention, the AMYR agonist may be a polypeptide, or a pharmaceutically acceptable salt thereof, comprising the amino acid sequence K(γE-γE-C18diacid)[CNTATC]ATQRLANFLRHSSNN(αMePhe)GPILPPTEVGSNTY-amide (SEQ ID NO: 11) in an amount of about 1.5 mg (e.g. 1.6 mg), about 4.5 mg, about 6 mg, or about 9 mg; and the GLP-1R agonist may be a polypeptide, or a pharmaceutically acceptable salt thereof, comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35) in an amount of about 0.15 mg, about 0.3 mg, about 0.6 mg, about 1.2 mg, about 2.3 mg, about 4.6 mg, about 7 mg, or about 9.4 mg.

The subject may have obesity or an obesity-related condition.

The invention also provides a method of inhibiting or reducing weight gain, promoting weight loss, reducing food intake, increasing satiety, and/or reducing excess body weight in a subject, the method comprising administering to the subject about 1 mg to about 15 mg of an AMYR agonist and about 0.1 to about 10 mg of a GLP-1R agonist.

The invention also provides a cosmetic method of inhibiting or reducing weight gain, promoting weight loss, reducing food intake, increasing satiety, and/or reducing excess body weight in a subject, the method comprising administering to the subject about 1 mg to about 15 mg of an AMYR agonist and about 0.1 to about 10 mg of a GLP-1R agonist.

The invention also provides a method of reducing fat-mass specific body weight in a subject, the method comprising administering to the subject about 1 mg to about 15 mg of an AMYR agonist and about 0.1 to about 10 mg of a GLP-1R agonist.

The invention also provides a cosmetic method of reducing fat-mass specific body weight in a subject, the method comprising administering to the subject about 1 mg to about 15 mg of an AMYR agonist and about 0.1 to about 10 mg of a GLP-1R agonist.

The invention also provides a method of improving glycemic and/or metabolic control in a subject, the method comprising administering to the subject about 1 mg to about 15 mg of an AMYR agonist and about 0.1 to about 10 mg of a GLP-1R agonist.

The method of improving glycemic and/or metabolic control in a subject may comprise increasing insulin secretion, delaying gastric emptying, increasing mitochondria function, inhibiting de novo lipogenesis, decreasing HbAlc, enhancing fatty oxidation, decreasing hepatic mitochondrial oxidative stress, decreasing steatosis, decreasing fibrosis, decreasing glycogen synthesis, increasing gluconeogenesis, reducing or reversing fibrosis (e.g., liver fibrosis), reducing steatohepatitis, and/or reducing risk of death due to cirrhosis, hepatocellular carcinoma, and/or cardiorenal disease in the subject.

In any method of the invention, the AMYR agonist and GLP-1R agonist, or pharmaceutically acceptable salts thereof, may be administered simultaneously.

In any method of the invention, the AMYR agonist and GLP-1R agonist, or pharmaceutically acceptable salts thereof, may be administered sequentially.

In any method of the invention, the AMYR agonist and GLP-1R agonist, or pharmaceutically acceptable salts thereof, may be formulated in a dual-chamber device.

The invention also provides a method of treating and/or preventing obesity or an obesity-related condition in a subject in need thereof, the method comprising administering to the subject about 1 mg to about 15 mg of an AMYR agonist and about 0.1 to about 10 mg of a GLP-1R agonist.

The invention also provides a method of treating and/or preventing a metabolic disease in a subject in need thereof, the method comprising administering to the subject about 1 mg to about 15 mg of an AMYR agonist and about 0.1 to about 10 mg of a GLP-1R agonist.

The invention also provides a method of reducing the body weight of a subject, in a subject in need thereof, the method comprising administering to the subject about 1 mg to about 15 mg of an AMYR agonist and about 0.1 to about 10 mg of a GLP-1R agonist.

The invention also provides a cosmetic method of reducing the body weight of a subject, the method comprising administering to the subject about 1 mg to about 15 mg of an AMYR agonist and about 0.1 to about 10 mg of a GLP-1R agonist.

The invention also provides an AMYR agonist and a GLP-1R agonist for use in a method of treating and/or preventing obesity or an obesity-related condition in a subject in need thereof, the method comprising administering to the subject about 1 mg to about 15 mg of the AMYR agonist and about 0.1 to about 10 mg of a GLP-1R agonist.

The invention also provides an AMYR agonist and a GLP-1R agonist for use in a method of treating and/or preventing a metabolic disease in a subject in need thereof, the method comprising administering to the subject about 1 mg to about 15 mg of the AMYR agonist and about 0.1 to about 10 mg of a GLP-1R agonist.

The invention also provides an AMYR agonist and a GLP-1R agonist for use in a method of reducing body weight in a subject, the method comprising administering to the subject about 1 mg to about 15 mg of the AMYR agonist and about 0.1 to about 10 mg of a GLP-1R agonist.

The subject may have a disease or disorder selected from obesity, an obesity-related condition, and metabolic disease.

The invention also provides an article of manufacture comprising (a) an AMYR agonist at a dose of about 1 mg to about 15 mg; and (b) a GLP-1R agonist at a dose of about 0.1 to about 10 mg.

The invention also provides a kit comprising (a) an AMYR agonist, (b) a GLP-1R agonist, and (c) instructions for use of the same for treating or preventing obesity or an obesity-related condition at a dose of about 1 mg to about 15 mg of the AMYR agonist and about 0.1 to about 10 mg of the GLP-1R agonist.

The kit may comprise a dual-chamber device.

### BRIEF DESCRIPTION OF THE DRAWINGS

FIG. 1 shows that an AMYR agonist demonstrated dose dependent weight loss following a single dose of either 0.3 mg, 0.9 mg, 1.5 mg, 2.7 mg, or 4.2 mg AMYR agonist in a Phase I trial of healthy subjects who are overweight or obese.

FIG. 2 shows that administration of the AMYR agonist demonstrated a good tolerability profile in the Phase I trial, in which no safety concerns were identified at single doses up to 4.2 mg.

FIG. 3 shows that sustained weight loss was achieved following (FIG. 3A) six weeks of repeat dosing (once weekly) of 2.7 mg AMYR agonist (s.c.) and (FIG. 3B) twelve weeks of repeat dosing (once weekly) gradually titrated from 1.5 mg to 9 mg AMYR agonist (s.c.) in preliminary data from a Phase Ib trial of healthy subjects who are overweight or obese.

FIG. 4 shows that good tolerability was achieved (FIG. 4A) on repeat dosing of 2.7 mg AMYR agonist (s.c.) and (FIG. 4B) on repeat dosing of AMYR agonist (s.c.) gradually titrated from 1.5 mg to 9 mg in preliminary data from the Phase Ib trial.

FIG. 5 shows that a GLP-1R/GCGR dual agonist treatment led to a dose-dependent decrease in body weight with improved hepatic lipid and circulating insulin levels in a pre-clinical mouse model.

FIG. 6 shows a Phase I single ascending dose (SAD) study design for a GLP-1R/GCGR dual agonist treatment in humans.

FIG. 7 shows a plasma concentration-time profile following single subcutaneous (SC) or intravenous (IV) administration of a GLP-1R/GCGR dual agonist in a Phase I SAD study.

FIG. 8 shows an exposure response for decreased appetite, nausea, and vomiting following administration of a GLP-1R/GCGR dual agonist in a Phase I SAD study.

FIG. 9 shows a Phase I/II repeat dose study design for a GLP-1R/GCGR dual agonist treatment in humans.

FIG. 10 shows that sustained weight loss was achieved following repeat dosing (once weekly) of a GLP-1R/GCGR dual agonist (s.c.) in preliminary data from a Phase I/II trial of healthy subjects who are overweight or obese.

FIG. 11 shows that sustained weight loss (approximately 6.8%; 6.6% placebo-corrected) was achieved following 16 weeks of multiple-once weekly ascending doses of GLP-1R/GCGR dual agonist (sc) for 16 weeks treatment titrated up to a (non-final) dose of 7 mg in preliminary data from a Phase I/II trial of healthy subjects who are overweight or obese.

FIG. 12 shows: A Graph showing Day 20% body weight change for DIO rats treated SC once daily with Vehicle, long-acting AMYR agonist (LAA) (10 nmol/kg), GLP-1/GCG agonist (GLP-1/GCG) (5.4 nmol/kg) or combination of both (10 and 5.4 nmol/kg respectively), with an n=8/group. Data reported as mean±standard error of the mean (SEM); data analysis performed using GraphPad Prism—statistical analysis performed using One-way ANOVA with Tukey ad hoc analysis. (*p<0.05, **p<0.01, ***0<0.001, ****p<0.0001). B Graph showing Day 26 change in lean mass for DIO rats treated with LAA, GLP-1/GCG or combination of both. C Graph showing Day 26 change in fat mass for DIO rats treated with LAA, GLP-1/GCG or combination of both.

FIG. 13 shows Day 26 body composition data for DIO rats treated with long-acting LAA, GLP-1/GCG or combination of both, with an n=8/group. A Body composition measured by nuclear magnetic resonance (NMR)—Mean % fat and lean mass reflected as percentage of overall body weight and B Day 26 body weight reflected as % of non-obese control as also shown with data from study day 26.

FIG. 14 shows percent change in body weight normalized to Vehicle. A Rats were dosed once daily SC for 28 days with Vehicle, GLP-1/GCG (1.5 nmol/kg), LAA (7.5 nmol/kg) or a combination of GLP-1/GCG and LAA (1.5 and 7.5 nmol/kg, respectively) with an n=6/group. B Rats were dosed once daily SC for 28 days with Vehicle, GLP-1/GCG (5 nmol/kg), LAA (7.5 nmol/kg) or a combination of GLP-1/GCG and LAA (5 nmol/kg and 7.5 nmol/kg, respectively) with an n=6/group. Data reported as mean±standard error of the mean (SEM); data analysis performed using GraphPad Prism—statistical analysis performed using One-way ANOVA with Tukey ad hoc analysis (*p<0.05, **p<0.01, ****p<0.0001)

FIG. 15 shows percent change in body weight normalized to Vehicle. Rats were dosed every other day (Q2D) SC for 19 days with Vehicle, GLP1-Fc (0.1 or 1 mg/kg), Fc-LAA (0.1 or 2 mg/kg) or a combination of GLP1-Fc and Fc-LAA (0.1+0.1 mg/kg, 0.1+2 mg/kg, 1+0.1 mg/kg or 1+2 mg/kg, respectively) with an n=7/group. Data reported as mean±standard error of the mean (SEM); data analysis performed using GraphPad Prism.

FIG. 16 shows an AMYR agonist adjunct therapy while maintaining an existing GLP-1 therapy. Rats were dosed SC for 15 days with Vehicle (n=16) or GLP-1 RA agonist (GLP-1RA) (n=32) (5 nmol/kg) once-daily (QD). Beginning on day 16, rats initially treated with Vehicle either continued with Vehicle (n=8) or were switched to LAA (7.5 nmol/kg QD, n=8) administration. Rats initially receiving GLP-1RA treatment either continued with GLP-1RA, were switched to LAA (7.5 nmol/kg QD), were switched to Vehicle, or continued with GLP-1RA with addition of LAA treatment (7.5 nmol/kg QD) for duration of study (day 30), with n=8/group. A Graph showing % Day 0 body weight change over time. B Graph showing % body weight change normalised to Vehicle over time. C Change in body weight at the end of the study. D Food intake at the end of the study.

FIG. 17 shows a combination of an AMYR agonist with a GLP/GIP agonist. DIO rats were dosed once daily SC for 28 days (n=8/group) with either Vehicle, LAA (10 nmol/kg), GLP/GIP dual agonist (2, 10 or 30 nmol/kg), or a combination of LAA (10 nmol/kg for all groups) and GLP/GIP dual agonist (2, 10 or 30 nmol/kg). Control rats were dosed SC with Vehicle. Graphs showing: A Body weight during study over time. B Baseline-corrected body weight change (%). C Body composition data represented as change in fat mass (g). D Body composition data represented as change in lean mass (g). E Body composition data represented as percentage fat mass (%) of total body weight at both week −1 (baseline) or week 4 (end of study). F Body composition data represented as percentage lean mass (%) of total body weight at both week −1 (baseline) or week 4 (end of study). Statistical analysis performed using two-way RM ANOVA with Dunnett analysis (***p<0.001, ****p<0.0001).

FIG. 18 shows that a SARA has improved body weight reduction as compared to a DACRA. DIO rats were dosed QD for 4 weeks with either Vehicle, AMYR agonist (2.5 or 10 nmol/kg), or DACRA (1, 3, 10 or 20 nmol/kg). FIG. 18A. Graph showing body weight change (%) normalized to vehicle over time. FIG. 18B. Body composition change (fat mass (L) and fat-free mass (R), g) at end of study. FIG. 18C. Total body weight change (%) normalized to vehicle and corresponding proportion of fat mass loss and fat-free mass-loss at end of study. FIG. 18D. Emax model showing relationship between calcitonin receptor engagement with lean-mass change (g, corrected for control) (L) and amylin receptor engagement with fat-mass change (g, corrected for control) (R).

FIG. 19 shows aversion data for a SARA as compared to a DACRA. FIG. 19A Graph showing result of saccharin conditioned tasted aversion dose response experiment for AZD6234 and a DACRA. Data reported as mean±standard error of the mean (SEM); data analysis performed using GraphPad Prism—statistical analysis performed using One-way ANOVA with Tukey ad hoc analysis. (*p<0.05, **p<0.01, ***0<0.001, ****p<0.0001 vs. Vehicle). FIG. 19B Emax model showing relationship between calcitonin receptor engagement (L) or amylin receptor engagement (R) (fold in vitro potency normalised for fraction unbound) with saccharine aversion in lean rats.

FIGS. 20-23 show exemplary titration schedules for a Phase IIb study evaluating the co-administration of an AMYR agonist and a GLP-1/GCG dual agonist in subjects living with obesity or overweight with co-morbidity.

### DETAILED DESCRIPTION

### Definitions

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. The terminology used herein is for the purpose of describing particular embodiments only and is not intended to limit the scope of the present invention, which is defined solely by the claims.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognise. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

The headings provided herein are not limitations of the various aspects or embodiments of this disclosure.

As used herein, the term “capable of” when used with a verb, encompasses, or means the action of the corresponding verb. For example, “capable of activating” also means activates, “capable of agonising” also means agonises, “capable of binding” also means binds and “capable of specifically activating . . . ” also means specifically activates.

Numeric ranges are inclusive of the numbers defining the range. Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

As used herein, the articles “a” and “an” may refer to one or to more than one (e.g., to at least one) of the grammatical object of the article. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting.

“About” may generally mean an acceptable degree of error for the quantity measured given the nature or precision of the measurements. Exemplary degrees of error are within 20 percent (%), typically, within 10%, and more typically, within 5% of a given value or range of values. Preferably, the term “about” shall be understood herein as plus or minus (±) 5%, preferably ±4%, ±3%, ±2%, ±1%, ±0.5%, ±0.1%, of the numerical value of the number with which it is being used.

The term “consisting of” refers to compositions, methods, and respective components thereof as described herein, which are exclusive of any element not recited in that description of the invention.

As used herein the term “consisting essentially of” refers to those elements required for a given invention. The term permits the presence of elements that do not materially affect the basic and novel or functional characteristic(s) of that invention (i.e., inactive, or non-immunogenic ingredients).

Embodiments described herein as “comprising” one or more features may also be considered as disclosure of the corresponding embodiments “consisting of” and/or “consisting essentially of” such features. of the corresponding embodiments “consisting of” such features.

Amino acids are referred to herein using the name of the amino acid, the three-letter abbreviation, or the single letter abbreviation. Unless otherwise indicated, amino acid sequences are written left to right in amino to carboxy orientation.

The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

A “fragment” of a polypeptide typically comprises at least 50%, at least 60%, at least 70%, at least 80%, at least 90%, at least 95%, at least 97% or more of the original polypeptide.

A “variant” amino acid sequence has substantial homology or substantial similarity to a reference amino acid sequence (or a fragment thereof). A amino acid sequence or fragment thereof is “substantially homologous” (or “substantially identical”) to a reference sequence if, when optimally aligned (with appropriate amino acid insertions or deletions) with the other amino acid (or its complementary strand), there is nucleotide sequence identity in at least about 70%, 75%, 80%, 85%, 90%, 91%, 92%, 93%, 94%, 95%, 96%, 97%, 98%, 99% or more % of the amino acids. Methods for homology determination of amino acid sequences are known in the art. Typically, a variant polypeptide of the invention retains the function or activity of the full-length polypeptide.

A variant polypeptide may be one in which amino acid residues from one species are substituted for the corresponding residue in another species, either at the conserved or non-conserved positions. Variants of amylin receptor agonists and/or GLP-1R agonists disclosed herein may be produced and used in the present invention. Following the lead of computational chemistry in applying multivariate data analysis techniques to the structure/property-activity relationships [see for example, Wold, et al. Multivariate data analysis in chemistry. Chemometrics-Mathematics and Statistics in Chemistry (Ed.: B. Kowalski); D. Reidel Publishing Company, Dordrecht, Holland, 1984 (ISBN 90-277-1846-6] quantitative activity-property relationships of DNMTs can be derived using well-known mathematical techniques, such as statistical regression, pattern recognition and classification [see for example Norman et al. Applied Regression Analysis. Wiley-Interscience; 3rd edition (April 1998) ISBN: 0471170828; Kandel, Abraham et al. Computer-Assisted Reasoning in Cluster Analysis. Prentice Hall PTR, (May 11, 1995), ISBN: 0133418847; Krzanowski, Wojtek. Principles of Multivariate Analysis: A User's Perspective (Oxford Statistical Science Series, No 22 (Paper)). Oxford University Press; (December 2000), ISBN: 0198507089; Witten, Ian H. et al Data Mining: Practical Machine Learning Tools and Techniques with Java Implementations. Morgan Kaufmann; (Oct. 11, 1999), ISBN:1558605525; Denison David G. T. (Editor) et al Bayesian Methods for Nonlinear Classification and Regression (Wiley Series in Probability and Statistics). John Wiley & Sons; (July 2002), ISBN: 0471490369; Ghose, Arup K. et al. Combinatorial Library Design and Evaluation Principles, Software, Tools, and Applications in Drug Discovery. ISBN: 0-8247-0487-8]. The properties of a DNMT can be derived from empirical and theoretical models (for example, analysis of likely contact residues or calculated physicochemical property) of the DNMT sequence, functional and three-dimensional structures and these properties can be considered individually and in combination.

Amino acid residues at non-conserved positions may be substituted with conservative or non-conservative residues. In particular, conservative amino acid replacements are contemplated. A “conservative amino acid substitution” is one in which the amino acid residue is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative. The inclusion of conservatively modified variants in a DNMT of the invention does not exclude other forms of variant, for example polymorphic variants, interspecies homologs, and alleles.

“Non-conservative amino acid substitutions” include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, lie, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly).

As used herein, the terms “polynucleotides”, “nucleic acid” and “nucleic acid sequence” refers to any molecule, preferably a polymeric molecule, incorporating units of ribonucleic acid, deoxyribonucleic acid, or an analogue thereof. The nucleic acid can be either single-stranded or double-stranded. A single-stranded nucleic acid can be one nucleic acid strand of a denatured double-stranded DNA Alternatively, it can be a single-stranded nucleic acid not derived from any double-stranded DNA. In one aspect, the nucleic acid can be DNA. In another aspect, the nucleic acid can be RNA Suitable nucleic acid molecules are DNA, including genomic DNA or cDNA. Other examples of nucleic acid molecules are RNA, including siRNA, shRNA, and antisense oligonucleotides. Typically, the methods of the invention relate to the production of oligonucleotides (short DNA or RNA sequences typically less than about 300 bases in length).

Unless otherwise indicated, any nucleic acid sequences are written left to right in 5′ to 3′ orientation.

The polynucleotides of the present invention may be prepared by any means known in the art. For example, large amounts of the polynucleotides may be produced by replication in a suitable host cell. The natural or synthetic DNA fragments coding for a desired fragment will be incorporated into recombinant nucleic acid constructs, typically DNA constructs, capable of introduction into and replication in a prokaryotic or eukaryotic cell. Usually, the DNA constructs will be suitable for autonomous replication in a unicellular host, such as yeast or bacteria, but may also be intended for introduction to and integration within the genome of a cultured insect, mammalian, plant, or other eukaryotic cell lines.

The polynucleotides of the present invention may also be produced by chemical synthesis, e.g., by the phosphoramidite method or the tri-ester method and may be performed on commercial automated oligonucleotide synthesisers. A double-stranded fragment may be obtained from the single stranded product of chemical synthesis either by synthesising the complementary strand and annealing the strand together under appropriate conditions or by adding the complementary strand using DNA polymerase with an appropriate primer sequence.

In view of the degeneracy of the genetic code, considerable sequence variation is possible among the polynucleotides of the present invention. One of ordinary skill in the art will appreciate that flexibility exists when determining a degenerate codon, representative of all possible codons encoding each amino acid. For example, some polynucleotides encompassed by the degenerate sequence may encode variant amino acid sequences, but one of ordinary skill in the art can easily identify such variant sequences by reference to the amino acid sequences of the present invention.

A “variant” nucleic acid sequence has substantial homology or substantial similarity to a reference nucleic acid sequence (or a fragment thereof). A nucleic acid sequence or fragment thereof is “substantially homologous” (or “substantially identical”) to a reference sequence if, when optimally aligned (with appropriate nucleotide insertions or deletions) with the other nucleic acid (or its complementary strand), there is nucleotide sequence identity in at least about 70%, 75%, 80%, 85%, 90%, 91%, 92%, 93%, 94%, 95%, 96%, 97%, 98%, 99% or more % of the nucleotide bases. Methods for homology determination of nucleic acid sequences are known in the art.

Alternatively, a “variant” nucleic acid sequence is substantially homologous with (or substantially identical to) a reference sequence (or a fragment thereof) if the “variant” and the reference sequence they are capable of hybridising under stringent (e.g., highly stringent) hybridisation conditions. Nucleic acid sequence hybridisation will be affected by such conditions as salt concentration (e.g. NaCl), temperature, or organic solvents, in addition to the base composition, length of the complementary strands, and the number of nucleotide base mismatches between the hybridising nucleic acids, as will be readily appreciated by those skilled in the art. Stringent temperature conditions are preferably employed, and generally include temperatures in excess of 30° C., typically in excess of 37° C. and preferably in excess of 45° C. Stringent salt conditions will ordinarily be less than 1000 mM, typically less than 500 mM, and preferably less than 200 mM. The pH is typically between 7.0 and 8.3. The combination of parameters is much more important than any single parameter.

Methods of determining nucleic acid percentage sequence identity are known in the art. By way of example, when assessing nucleic acid sequence identity, a sequence having a defined number of contiguous nucleotides may be aligned with a nucleic acid sequence (having the same number of contiguous nucleotides) from the corresponding portion of a nucleic acid sequence of the present invention. Tools known in the art for determining nucleic acid percentage sequence identity include Nucleotide BLAST (as described below).

One of ordinary skill in the art appreciates that different species exhibit “preferential codon usage”. As used herein, the term “preferential codon usage” refers to codons that are most frequently used in cells of a certain species, thus favouring one or a few representatives of the possible codons encoding each amino acid. For example, the amino acid threonine (Thr) may be encoded by ACA, ACC, ACG, or ACT, but in mammalian host cells ACC is the most commonly used codon; in other species, different codons may be preferential. Preferential codons for a particular host cell species can be introduced into the polynucleotides of the present invention by a variety of methods known in the art. Introduction of preferential codon sequences into recombinant DNA can, for example, enhance production of the protein by making protein translation more efficient within a particular cell type or species.

A “fragment” of a polynucleotide of interest comprises a series of consecutive nucleotides from the sequence of said full-length polynucleotide. By way of example, a “fragment” of a polynucleotide of interest may comprise (or consist of) at least 30 consecutive nucleotides from the sequence of said polynucleotide (e.g., at least 35, 50, 75, 100, 150, 200, 250, 300, 350, 400, 450, 500, 550, 600, 650, 700, 750, 800 850, 900, 950 or 1000 consecutive nucleic acid residues of said polynucleotide). Typically, a fragment as defined herein retains the same function as the full-length polynucleotide.

When applied to a nucleic acid sequence, the term “isolated” denotes that the polynucleotide sequence has been removed from its natural genetic milieu and is thus free of other extraneous or unwanted coding sequences (but may include naturally occurring 5′ and 3′ untranslated regions such as promoters and terminators) and is in a form suitable for use within genetically engineered protein production systems. When applied to a protein, such as an amylin receptor agonist or GLP-1R agonist amino acid sequence, the term “isolated” denotes that the protein has been removed from its natural cellular milieu and is thus free of other extraneous or unwanted coding proteins and/or genetic material and is in a form suitable for use within genetically engineered protein production systems. Such isolated molecules are those that are separated from their natural environment. In the context of the invention, an isolated protein nucleic acid is one which has been separated from one or more of the reagents used in its production according to methods of the invention; one which has been separated from the other proteins and/or nucleic acid sequences synthesised in the same iteration of the method and/or one which has been separated from the one or more units on which it was synthesised.

The terms “decrease”, “reduce”, “reduction”, or “inhibit” are all used herein to mean a decrease by a statistically significant amount. The terms “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” encompasses a complete inhibition or reduction as compared to a reference level. “Complete inhibition” is a 100% inhibition (i.e., abrogation) as compared to a reference level.

The terms “increased”, “increase”, “enhance”, or “activate” are all used herein to mean an increase by a statically significant amount. The terms “increased”, “increase”, “enhance”, or “activate” can mean an increase of at least 25%, at least 50% as compared to a reference level, for example an increase of at least about 50%, or at least about 75%, or at least about 80%, or at least about 90%, at least about 95%, or at least about 98%, or at least about 99%, or at least about 100%, or at least about 250% or more compared with a reference level, or at least about a 1.5-fold, or at least about a 2-fold, or at least about a 2.5-fold, or at least about a 3-fold, or at least about a 4-fold, or at least about a 5-fold or at least about a 10-fold increase, or any increase between 1.5-fold and 10-fold or greater as compared to a reference level.

As used herein, the term “sample” refers to a sample of biological materials (cells, tissue, fluid, etc.) obtained from an individual. The sample may be any suitable biological material, for example blood, plasma, saliva, serum, sputum, urine, cerebral spinal fluid, cells, a cellular extract, a tissue sample, a tissue biopsy, a stool sample and the like. Typically, the sample is blood sample. The precise biological sample that is taken from the individual may vary, but the sampling preferably is minimally invasive and is easily performed by conventional techniques. The sample may be a whole blood sample, a purified peripheral blood leukocyte sample or a cell type sorted leukocyte sample, such as a sample of the individual's neutrophils.

The term “pharmaceutically acceptable” as used herein means approved by a regulatory agency of the Federal or a state government, or listed in the U.S. Pharmacopeia, European Pharmacopeia, or other generally recognised pharmacopeia.

The publications discussed herein are provided solely for their disclosure prior to the filing date of the present application. Nothing herein is to be construed as an admission that such publications constitute prior art to the claims appended hereto. All documents cited herein are each entirely incorporated by reference herein, including all data, tables, figures, and text presented in the cited documents.

### Treatment and Dosage Regimens

The invention relates to therapies comprising an amylin receptor (AMYR) agonist and a GLP-1 receptor (GLP-1R) agonist. An AMYR agonist is a molecule that is capable of binding to, and inducing signalling by, one or more receptors or receptor complexes regarded as physiological receptors for human amylin. A GLP-1R agonist is a molecule that is capable of binding to, and inducing signalling by, one or more receptors or receptor complexes regarded as physiological receptors for human GLP-1R. Suitable AMYR agonists and GLP-1R agonists are described in more detail below.

### Treatment Methods

The present invention encompasses therapies which involve administering (a) between about 1 mg to about 15 mg of an AMYR agonist and (b) about 0.1 mg to about 10 mg of a GLP-1R agonist (e.g. a GLP-1R/GCGR dual-agonist) to a subject for preventing, treating, or ameliorating symptoms associated with a disease, disorder, or infection. In any method, therapeutic indication, use or other disclosure herein, the subject may be any animal, typically a mammal, preferably a human.

Accordingly, the invention provides a method of treating and/or preventing a disease or disorder in a subject, the method comprising administering to the subject (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof.

The invention also provides an AMYR agonist and GLP-1R agonist (e.g. a GLP-1R/GCGR dual-agonist) or pharmaceutically acceptable salts thereof of the invention for use in therapy, for example in a method of treating and/or preventing a disease or disorder, the method comprising administering to the subject (a) between about 1 mg to about 15 mg of the AMYR agonist, and (b) between about 0.1 mg to about 10 mg of a GLP-1R agonist (e.g. a GLP-1R/GCGR dual-agonist), or pharmaceutically acceptable salts thereof.

Also provided is the use of an AMYR agonist and a GLP-1R agonist (e.g. a GLP-1R/GCGR dual-agonist) in the manufacture of a medicament for treating and/or preventing a disease or disorder in a subject, wherein the medicament comprises about 1 mg to about 15 mg of the AMYR agonist and about 0.1 mg to about 10 mg of the GLP-1R agonist (e.g. a GLP-1R/GCGR dual-agonist).

The use or method may comprise administering a therapeutically effective schedule that has less frequent doses of the AMYR agonist and the GLP-1R agonist (e.g. a GLP-1R/GCGR dual-agonist) than the therapeutically effective dosing schedule of a different anti-obesity drug, such as pramlintide, cotadutide, or a dual amylin-calcitonin receptor agonist (DACRA, e.g., davalintide); an incretin mimetic drug, such as a GLP-1R agonist (e.g., semaglutide); a GLP-1R/GCGR dual agonist (e.g. survodutide); a GLP1/GCG/GIP triple agonist (e.g. retatrutide); and/or a DACRA-GLP-1Ra combination (e.g., semaglutide-cagrilintide, “cagri-sema”).

The invention further provides (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt for use in the treatment and/or prevention of obesity, metabolic diseases (such as diabetes, e.g. type 1 or type 2 diabetes), and/or obesity-related conditions. The invention also provides a method of treating and/or preventing obesity, metabolic diseases (such as diabetes, e.g. type 1 or type 2 diabetes), and/or obesity-related conditions in a subject, the method comprises administering (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof, in the subject.

In preferred embodiments, the AMYR agonist and the GLP-1R agonist (e.g. a GLP-1R/GCGR dual-agonist) may be used in a method of treating and/or preventing obesity. Said method may comprise administering (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof, to the subject. The invention also provides an AMYR agonist and a GLP-1R agonist (e.g. a GLP-1R/GCGR dual-agonist) for use in such a method, the method comprising administering (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof, to the subject.

In some embodiments, the AMYR agonist and the GLP-1R agonist (e.g. a GLP-1R/GCGR dual-agonist) may be used in a method of treating and/or preventing an obesity-related condition in a subject. Said method may comprise administering (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof, to the subject. The invention also provides an AMYR agonist and a GLP-1R agonist (e.g. a GLP-1R/GCGR dual-agonist) for use in such a method, the method comprising administering (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof, to the subject. In some embodiments, the obesity-related condition is overweight, morbid obesity, obesity prior to surgery, obesity-linked inflammation, obesity-linked gallbladder disease, sleep apnoea and respiratory problems, hyperlipidaemia, degeneration of cartilage, osteoarthritis, or reproductive health complications of obesity or overweight such as infertility in a subject.

In some embodiments, the AMYR agonist and the GLP-1R agonist (e.g. a GLP-1R/GCGR dual-agonist) may be used in a method of treating and/or preventing a metabolic disease. Said method may comprise administering (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof, to the subject. The invention also provides an AMYR agonist and a GLP-1R agonist (e.g. a GLP-1R/GCGR dual-agonist) for use in such a method, the method comprising administering (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof, to the subject. Said metabolic disease may be diabetes, type 1 diabetes, type 2 diabetes, gestational diabetes, pre-diabetes, insulin resistance, impaired glucose tolerance (IGI), disease states associated with elevated blood glucose levels, metabolic disease including metabolic syndrome, or hyperglycaemia, e.g. abnormal postprandial hyperglycaemia. In preferred embodiments, the AMYR agonist and the GLP-1R agonist (e.g. a GLP-1R/GCGR dual-agonist) of the invention are used for the treatment of type 1 diabetes or type 2 diabetes. In particularly preferred embodiments, the AMYR agonist and the GLP-1R agonist (e.g. a GLP-1R/GCGR dual-agonist) of the invention are used for the treatment of type 2 diabetes.

In some embodiments, the AMYR agonist and the GLP-1R agonist (e.g. a GLP-1R/GCGR dual-agonist) may be used in a method of treating and/or preventing metabolic dysfunction associated steatohepatitis (MASH). Said method may comprise administering (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof, to the subject. The invention also provides an AMYR agonist and a GLP-1R agonist (e.g. a GLP-1R/GCGR dual-agonist) for use in such a method, the method comprising administering (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof, to the subject.

The treatment and/or prevention of a disease or disorder using (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof, may further comprise treating, improving, and/or protecting liver and/or kidney function in the subject. Treating, improving, and/or protecting liver function may comprise reducing steatohepatitis and/or fibrosis in the liver.

Also provided is a method of inhibiting or reducing weight gain, promoting weight loss, reducing food intake, increasing satiety, and/or reducing excess body weight, the method comprising administering (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof, to the subject. The invention also provides an AMYR agonist and a GLP-1R agonist (e.g. a GLP-1R/GCGR dual-agonist) for use in such a method, the method comprising administering (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof, to the subject. Said method may be therapeutic, e.g. when the subject is obese, suffers from an obesity-related condition and/or a metabolic disease. Alternatively, said method may be cosmetic (non-therapeutic), e.g. when the patient wishes to lose weight for aesthetic reasons.

Thus, the invention also provides a cosmetic use of an AMYR agonist and a GLP-1R agonist to inhibit or reduce weight gain, promote weight loss, reduce food intake, increase satiety, and/or reduce excess body weight in a subject, comprising administering (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof.

In particular, the invention provides a method of reducing the body weight of a subject, the method comprising administering (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof, to the subject. The invention also provides an AMYR agonist and a GLP-1R agonist (e.g. a GLP-1R/GCGR dual-agonist) for use in such a method, the method comprising administering (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof, to the subject.

In the context of the invention, “reducing the body weight of a subject” can be measured by any suitable means, and may be determined by a reduction in the absolute amount of body weight in a subject (e.g., in grams) from baseline, and/or a percent change in body weight from baseline. Treatment according to the present invention may reduce the body weight of a subject by ≥5% from baseline, ≥6% from baseline, ≥7% from baseline, ≥8% from baseline, ≥9% from baseline, ≥10% from baseline, ≥15% from baseline, ≥20% from baseline, ≥25% from baseline, ≥30% from baseline, or more. Thus, the subject may have a weight reduction of ≥5% from baseline following treatment. The subject may have a weight reduction of ≥10% from baseline following treatment. The subject may have a weight reduction of ≥15% from baseline following treatment. The subject may have a weight reduction of ≥20% from baseline following treatment.

The reduction in body weight may be cosmetic (non-therapeutic), e.g. when the patient wishes to lose weight for aesthetic reasons. Thus, the invention provides a cosmetic method of reducing the body weight of a subject, the method comprising administering (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof, to the subject. The invention also provides a cosmetic use of an AMYR agonist and a GLP-1R agonist, or pharmaceutically acceptable salts thereof, of the invention for reducing the body weight of a subject, wherein said use comprises administering (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof, to the subject.

The reduction in body weight may be therapeutic, e.g. when the subject is obese, suffers from an obesity-related condition and/or suffers from a metabolic disease.

In a method of the invention, the reduction in the body weight of the subject is typically greater than the reduction in body weight as compared to a therapeutically effective dose of a different anti-obesity drug, such a dual amylin-calcitonin receptor agonist (DACRA, e.g., davalintide); an incretin mimetic drug, such as a GLP-1R agonist (e.g., semaglutide); a GLP-1R/GCGR dual agonist (e.g. survodutide); a GLP1/GCG/GIP triple agonist (e.g. retatrutide); and/or a DACRA-GLP-1Ra combination (e.g., semaglutide-cagrilintide, “cagri-sema”). Typically, said reduction is compared with an equivalent or corresponding dose of the different anti-obesity drug, such as common clinically used doses thereof. Treatment according to the present invention may reduce the body weight of a subject by ≥5%, ≥6%, ≥7%, ≥8%, ≥9%, ≥10%, ≥15%, ≥20%, ≥25%, ≥30%, or more, as compared to a therapeutically effective dose of a different anti-obesity drug, such a dual amylin-calcitonin receptor agonist (DACRA, e.g., davalintide); an incretin mimetic drug, such as a GLP-1R agonist (e.g., semaglutide); a GLP-1R/GCGR dual agonist (e.g. survodutide); a GLP1/GCG/GIP triple agonist (e.g. retatrutide); and/or a DACRA-GLP-1Ra combination (e.g., semaglutide-cagrilintide, “cagri-sema”). Thus, the subject may have a weight reduction greater than ≥5% compared to a different anti-obesity drug following treatment. The subject may have a weight reduction of ≥10% compared to a different anti-obesity drug following treatment. The subject may have a weight reduction of ≥15% compared to a different anti-obesity drug following treatment. The subject may have a weight reduction of 20% compared to a different anti-obesity drug following treatment.

There is also provided a method of reducing fat mass-specific body weight in a subject, the method comprising administering (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof, to the subject. In the context of the invention, “reducing fat mass-specific body weight” (also referred to as “reducing fat-specific weight) may comprise reducing the absolute amount of fat-specific body weight in a subject (e.g., in grams), and/or reducing the proportion of the fat-specific body weight component of total body weight (e.g., as a percentage of total body weight). A method of the invention may preferentially reduce fat-mass specific body weight as compared to lean mass-specific (e.g. muscle) body weight (e.g., preserving lean-mass specific body weight while reducing overall body weight). For the avoidance of doubt, any and all reference herein to “reducing the body weight of a subject” applies equally and without reservation to “reducing fat mass-specific body weight of a subject”, unless expressly stated to the contrary.

In a method of the invention, the reduction in the fat-mass specific body weight of the subject is typically greater than the reduction in fat-mass specific body weight as compared to a therapeutically effective dose of a different anti-obesity drug, such a dual amylin-calcitonin receptor agonist (DACRA, e.g., davalintide); an incretin mimetic drug, such as a GLP-1R agonist (e.g., semaglutide); a GLP-1R/GCGR dual agonist (e.g. survodutide); a GLP1/GCG/GIP triple agonist (e.g. retatrutide); and/or a DACRA-GLP-1Ra combination (e.g., semaglutide-cagrilintide, “cagri-sema”). Typically, said reduction is compared with an equivalent or corresponding dose of the different anti-obesity drug, such as common clinically used doses thereof. Treatment according to the present invention may reduce the fat-mass specific body weight of a subject by ≥5%, ≥6%, ≥7%, ≥8%, ≥9%, ≥10%, ≥15%, ≥20%, ≥25%, ≥30%, or more, as compared to a therapeutically effective dose of a different anti-obesity drug, such a dual amylin-calcitonin receptor agonist (DACRA, e.g., davalintide); an incretin mimetic drug, such as a GLP-1R agonist (e.g., semaglutide); a GLP-1R/GCGR dual agonist (e.g. survodutide); a GLP1/GCG/GIP triple agonist (e.g. retatrutide); and/or a DACRA-GLP-1Ra combination (e.g., semaglutide-cagrilintide, “cagri-sema”). Thus, the subject may have a fat-mass specific weight reduction greater than ≥5% compared to a different anti-obesity drug following treatment. The subject may have a fat-mass specific weight reduction of ≥10% compared to a different anti-obesity drug following treatment. The subject may have a fat-mass specific weight reduction of ≥15% compared to a different anti-obesity drug following treatment. The subject may have a fat-mass specific weight reduction of ≥20% compared to a different anti-obesity drug following treatment.

The invention also provides an AMYR agonist and a GLP-1R agonist (e.g. a GLP-1R/GCGR dual-agonist) for use in a method of reducing fat mass-specific body weight in a subject, the method comprising administering (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof. Said method may be therapeutic, e.g. when the subject is obese, suffers from an obesity-related condition and/or a metabolic disease. Alternatively, said method may be cosmetic (non-therapeutic), e.g. when the patient wishes to lose weight for aesthetic reasons.

Thus, the invention also provides a cosmetic use of (a) about 1 mg to about 15 mg of an AMYR agonist, or pharmaceutically acceptable salt thereof, and (b) about 0.1 mg to about 10 mg of a GLP-1R agonist (e.g. a GLP-1R/GCGR dual-agonist), or pharmaceutically acceptable salt thereof to reduce fat-mass specific body weight in a subject.

Also provided herein is a method of treating, improving, or protecting liver and/or kidney function in a subject, the method comprising administering (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof, to the subject. The invention also provides an AMYR agonist and a GLP-1R agonist (e.g. a GLP-1R/GCGR dual-agonist) for use in such a method, the method comprising administering (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof, to the subject.

Also provided herein is a method of improving glycemic and/or metabolic control in a subject, the method comprising administering (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof, to the subject. The invention also provides an AMYR agonist and a GLP-1R agonist (e.g. a GLP-1R/GCGR dual-agonist) for use in such a method, the method comprising administering (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof, to the subject. Improving glycemic and/or metabolic control may comprise one or more of: increasing insulin secretion, delaying gastric emptying, increasing mitochondria function, inhibiting de novo lipogenesis, decreasing HbAlc, enhancing fatty oxidation, decreasing hepatic mitochondrial oxidative stress, decreasing steatosis, decreases fibrosis, decreasing glycogen synthesis, increasing gluconeogenesis, improving glycemic control, reducing or reversing fibrosis (e.g., liver fibrosis), reducing steatohepatitis, and/or reducing risk of death due to cirrhosis, hepatocellular carcinoma, and/or cardiorenal disease in a subject in need thereof.

There is also provided a method of improving glucose homeostasis, β-cell function, and/or insulin sensitivity in a subject, the method comprising administering (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof, to the subject. The invention also provides an AMYR agonist and a GLP-1R agonist (e.g. a GLP-1R/GCGR dual-agonist) for use in such a method, the method comprising administering (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof, to the subject. An improvement in glucose homeostasis, β-cell function, and/or insulin sensitivity may be measured by change from baseline in the updated homeostatic model assessment (HOMA) scores (HOMA-IR, HOMA-B, and HOMA-S) over time. HOMA is well-known in the art, such as described in Levy et al. (Diabetes Care (1998) 21(12):2191-2192), which is herein incorporated by reference in its entirety. An improvement in glucose homeostasis, β-cell function, and/or insulin sensitivity may be measured by change from baseline in HbA1c, fasting serum glucose, fasting serum insulin, fasting serum c-peptide, plasma proinsulin levels over time, proinsulin/c-peptide ratio over time and/or adiponectin levels over time.

Also provided is a method of promoting end-organ protection (e.g., cardiovascular, renal and/or liver) in a subject, the method comprising administering (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof, to the subject. The invention also provides an AMYR agonist and a GLP-1R agonist (e.g. a GLP-1R/GCGR dual-agonist) for use in such a method, the method comprising administering (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof, to the subject. End-organ protection may be measured by assessing change from baseline on cardiovascular endpoints, such as ApoB/ApoA1 and/or high sensitivity Troponin T (hs Troponin T) or Tropinin I (hs Troponin I). Alternatively or in addition, end-organ protection can be measured by assessing change on renal endpoints, such as estimated glomerular filtration rate (eGFR, calculated using creatinine and cystatin C), UACR (urine albumin and creatinine ratio), and/or renin. Further alternatively or in addition, end-organ protection can be measured by: assessing change from baseline on liver endpoints, such as pro-peptide of type Ill collagen (ProC3), alanine transaminase (ALT), aspartate aminotransferase (AST), FIB-4 (which is a standard clinical index for measuring liver fibrosis, calculated using AST, ALT, age, and/or platelet), and/or an enhanced liver fibrosis (ELF) test (which is a standard clinical index for measuring cirrhosis, calculated using hyaluronic acid (HA), procollagen Ill amino-terminal peptide (PIIINP), and tissue inhibitor of matrix metalloproteinase 1 (TIMP-1)); biomarkers of chronic inflammation (e.g. hs CRP, hs IL-6), the sarcopenia index (based on creatinine/cystatin C); and/or thyroid function (e.g. based on TSH, total T3 or fT4). Said method may be therapeutic, e.g. when the subject is obese, suffers from an obesity-related condition and/or a metabolic disease. Alternatively, said method may be cosmetic (non-therapeutic), e.g. when the patient wishes to lose weight for aesthetic reasons.

Also provided is a method of improving weight-loss quality in a subject, the method comprising administering (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof, to the subject. The invention also provides an AMYR agonist and a GLP-1R agonist (e.g. a GLP-1R/GCGR dual-agonist) for use in such a method, the method comprising administering (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof, to the subject. Weight loss quality may be measured by change from baseline in waist circumference, hip circumference and/or waist:hip ratio. Alternatively, or in addition, weight loss quality may be measured by change from baseline to on metabolic/adipokine health, as assessed by levels of adiponectin and/or leptin, lipid profile (such as levels of triglycerides (TG), total cholesterol, non-esterified fatty acids (NEFA), high-density lipoprotein (HDL), low-density lipoprotein (LDL), and/or very low-density lipoprotein (VLDL)), and/or change from baseline in systemic inflammation (such as levels of C-reactive protein (CRP), e.g. a high-sensitivity CRP test (hsCRP), and/or interleukin-6 (IL-6), e.g. a high-sensitivity IL-6 test (hsIL6)). Said method may be therapeutic, e.g. when the subject is obese, suffers from an obesity-related condition and/or a metabolic disease. Alternatively, said method may be cosmetic (non-therapeutic), e.g. when the patient wishes to lose weight for aesthetic reasons.

Also provided is a method of improving the lipid profile of a subject, the method comprising administering (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof, to the subject. The invention also provides an AMYR agonist and a GLP-1R agonist (e.g. a GLP-1R/GCGR dual-agonist) for use in such a method, the method comprising administering (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof, to the subject. Lipid profile may be measured by a change in based line in total cholesterol, HDL-cholesterol, LDL-cholesterol, non-HDL-cholesterol, VLDL-cholesterol, triglycerides and/or non-esterified fatty acids. Said method may be therapeutic, e.g. when the subject is obese, suffers from an obesity-related condition and/or a metabolic disease. Alternatively, said method may be cosmetic (non-therapeutic), e.g. when the patient wishes to lose weight for aesthetic reasons.

Also provided is a method of improving physical function and/or mood or cognitive function in a subject, the method comprising administering (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof, to the subject. The invention also provides an AMYR agonist and a GLP-1R agonist (e.g. a GLP-1R/GCGR dual-agonist) for use in such a method, the method comprising administering (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof, to the subject. Improving physical function in a subject may be assessed by change from baseline in IWQoL-Lite CT Physical Function score, SF-36 v2 Physical functioning score, IWQoL-Lite CT Total Score, SF-36 v2 physical component, mental component scores, Patient Global Impression (PGI) of Limitation in Physical Function, and/or FACIT-GP5 scores. Mood and/or cognitive function may be assessed by change in the Columbia-suicide severity rating scale (C-SSRS) and/or Patient Health Questionnaire (PHQ-9). Treatment according to the invention may also result in a reduction in alcohol consumption (e.g. as measured by change in phophatidylethanol value over time).

Treatment according to the present invention may result in a good immunogenicity profile. Immunogenicity of the AMYR agonist and GLP-1R agonist (e.g. a GLP-1R/GCGR dual-agonist) may be assessed using any appropriate means, which are within the routine practice of one of ordinary skill in the art, e.g. using a bioanalytical assay on a blood sample from a subject. Non-limiting examples of parameters which may be used to assess tolerance include measuring the prevalence, incidence and/or titres of anti-drug antibodies (ADAs). Prevalence, incidence and/or titres in ADAs may be assessed using any suitable means. Treatment according to the present invention may increase the prevalence, incidence and/or titres of ADAs of a subject by ≤5%, ≤6%, ≤7%, ≤8%, ≤9%, ≤10%, ≤15%, ≤20%, ≤25%, ≤50%, ≤75%, ≤100%, ≤200%, or ≤400% from baseline.

Also provided herein is (a) about 1 mg to about 15 mg of an AMYR agonist, or pharmaceutically acceptable salt thereof, and (b) about 0.1 mg to about 10 mg of a GLP-1R agonist (e.g. a GLP-1R/GCGR dual-agonist) for use in the manufacture of a medicament. Said medicament may be for use in the treatment and/or prevention of any disease or disorder, or for any clinical purpose (e.g. (e.g. improving glycemic and/or metabolic control, improving glucose homeostasis, β-cell function, and/or insulin sensitivity, or promoting end-organ protection) as described herein.

The subject may tolerate the combination of the AMYR agonist and GLP-1R agonist (e.g. a GLP-1R/GCGR dual-agonist) well. Tolerance of the AMYR agonist and GLP-1R agonist (e.g. a GLP-1R/GCGR dual-agonist) may be assessed using any appropriate means, which are within the routine practice of one of ordinary skill in the art. Non-limiting examples of parameters which may be used to assess tolerance include nausea and vomiting. A subject who tolerates the combination of the AMYR agonist and GLP-1R agonist (e.g. a GLP-1R/GCGR dual-agonist) well may exhibit reduced nausea and/or vomiting (e.g. reduced incidence of nausea and/or vomiting). Alternatively or in addition, tolerance to the AMYR agonist and GLP-1R agonist (e.g. a GLP-1R/GCGR dual-agonist) may be assessed by change in growth/differentiation factor 15 (GDF15) from baseline (i.e. reduced GDF15). Change in GDF15 may be assessed using any suitable sample from the subject, as described herein, e.g. a blood sample. The subject may have increased tolerability to the combination of the AMYR agonist and GLP-1R agonist (e.g. a GLP-1R/GCGR dual-agonist) (such as reduced nausea and/or vomiting) as compared with their tolerance to a therapeutically effective dose of a different anti-obesity drug, such a dual amylin-calcitonin receptor agonist (DACRA, e.g., davalintide); an incretin mimetic drug, such as a GLP-1R agonist (e.g., semaglutide); a GLP-1R/GCGR dual agonist (e.g. survodutide); a GLP1/GCG/GIP triple agonist (e.g. retatrutide); and/or a DACRA-GLP-1Ra combination (e.g., semaglutide-cagrilintide, “cagri-sema”). Typically, said tolerability is compared with an equivalent or corresponding dose of the different anti-obesity drug, such as common clinically used doses thereof. Treatment according to the present invention may reduce the incidence of nausea of a subject by ≥5%, ≥6%, ≥7%, ≥8%, ≥9%, ≥10%, ≥15%, ≥20%, ≥25%, ≥30%, or more, as compared to a therapeutically effective dose of a different anti-obesity drug, such a dual amylin-calcitonin receptor agonist (DACRA, e.g., davalintide); an incretin mimetic drug, such as a GLP-1R agonist (e.g., semaglutide); a GLP-1R/GCGR dual agonist (e.g. survodutide); a GLP1/GCG/GIP triple agonist (e.g. retatrutide); and/or a DACRA-GLP-1Ra combination (e.g., semaglutide-cagrilintide, “cagri-sema”). Treatment according to the present invention may reduce the incidence of vomiting of a subject by ≥5%, ≥6%, ≥7%, ≥8%, ≥9%, ≥10%, ≥15%, ≥20%, ≥25%, ≥30%, or more, as compared to a therapeutically effective dose of a different anti-obesity drug, such a dual amylin-calcitonin receptor agonist (DACRA, e.g., davalintide); an incretin mimetic drug, such as a GLP-1R agonist (e.g., semaglutide); a GLP-1R/GCGR dual agonist (e.g. survodutide); a GLP1/GCG/GIP triple agonist (e.g. retatrutide); and/or a DACRA-GLP-1Ra combination (e.g., semaglutide-cagrilintide, “cagri-sema”). Treatment according to the present invention may reduce the levels of GDF-15 of a subject by ≥5%, ≥6%, ≥7%, ≥8%, ≥9%, ≥10%, ≥15%, ≥20%, ≥25%, ≥30%, or more, from baseline.

Treatment according to the present invention may reduce the need for the subject to be administered an anti-emetic medication as compared to a therapeutically effective dose of a different anti-obesity drug, such a dual amylin-calcitonin receptor agonist (DACRA, e.g., davalintide); an incretin mimetic drug, such as a GLP-1R agonist (e.g., semaglutide); a GLP-1R/GCGR dual agonist (e.g. survodutide); a GLP1/GCG/GIP triple agonist (e.g. retatrutide); and/or a DACRA-GLP-1Ra combination (e.g., semaglutide-cagrilintide, “cagri-sema”). Suitable anti-emetic medications will be familiar to a person skilled in the art. Thus, treatment according to the present invention may allow the subject to be administered a reduced dose of an anti-emetic medication as compared to a therapeutically effective dose of a different anti-obesity drug. Alternatively or in addition, treatment according to the present invention may allow the subject to be administered an anti-emetic medication at a reduced dosing frequency as compared to a therapeutically effective dose of a different anti-obesity drug. Treatment according to the present invention may eliminate the need for a subject to be administered an anti-emetic medication, i.e., the subject is not administered an anti-emetic medication.

Treatment according to the present invention may result in an equivalent reduction in body weight as compared to a therapeutically effective dose of a different anti-obesity drug, such a dual amylin-calcitonin receptor agonist (DACRA, e.g., davalintide); an incretin mimetic drug, such as a GLP-1R agonist (e.g., semaglutide); a GLP-1R/GCGR dual agonist (e.g. survodutide); a GLP1/GCG/GIP triple agonist (e.g. retatrutide); and/or a DACRA-GLP-1Ra combination (e.g., semaglutide-cagrilintide, “cagri-sema”), but with a reduction in side effects as compared to the different anti-obesity drug. Typically, said reduction is compared with an equivalent or corresponding dose of the different anti-obesity drug, such as common clinically used doses thereof. Side-effects (or adverse effects) may be assessed using any appropriate means, which are within the routine practice of one of ordinary skill in the art. Non-limiting examples of parameters which may be used to assess a reduction in side-effects include incidence and/or severity of nausea, vomiting, (reduction in) appetite, and/or headache, as discussed herein. Thus, treatment according to the present invention may result in a reduction in the incidence of nausea in a subject by ≥5%, ≥6%, ≥7%, ≥8%, ≥9%, ≥10%, ≥15%, ≥20%, ≥25%, ≥30%, or more, as compared to a therapeutically effective dose of a different anti-obesity drug. Thus, treatment according to the present invention may result in a reduction in the incidence of vomiting in a subject by ≥5%, ≥6%, ≥7%, ≥8%, ≥9%, ≥10%, ≥15%, ≥20%, ≥25%, ≥30%, or more, as compared to a therapeutically effective dose of a different anti-obesity drug, Treatment according to the present invention may result in a reduction in the incidence of reduced appetite of a subject by ≥5%, ≥6%, ≥7%, ≥8%, ≥9%, ≥10%, ≥15%, ≥20%, ≥25%, ≥30%, or more, as compared to a therapeutically effective dose of a different anti-obesity drug. Treatment according to the present invention may result in a reduction in the incidence of headache in a subject by ≥5%, ≥6%, ≥7%, ≥8%, ≥9%, ≥10%, ≥15%, ≥20%, ≥25%, ≥30%, or more, as compared to a therapeutically effective dose of a different anti-obesity drug.

The route of administration of an AMYR agonist and a GLP-1R agonist (e.g. a GLP-1R/GCGR dual-agonist), or pharmaceutical compositions thereof, can be, for example, oral, parenteral, by inhalation or topical. In preferred embodiments, the AMYR agonist and the GLP-1R agonist (e.g. the GLP-1R/GCGR dual-agonist) or pharmaceutical compositions thereof are administered by parenteral administration to a subject or patient. The term “parenteral” as used herein includes, e.g., intravenous, intraarterial, intraperitoneal, intramuscular, subcutaneous, rectal, or vaginal administration. In preferred embodiments, the AMYR agonist and the GLP-1R agonist (e.g. the GLP-1R/GCGR dual-agonist) or pharmaceutical compositions thereof are administered by injection, such as by intravenous, subcutaneous or intramuscular injection, to a subject or patient. In some embodiments, the AMYR agonist and the GLP-1R agonist (e.g. the GLP-1R/GCGR dual-agonist) or pharmaceutical compositions thereof are administered orally. In particularly preferred embodiments, the AMYR agonist and the GLP-1R agonist (e.g. the GLP-1R/GCGR dual-agonist) or pharmaceutical compositions thereof are administered by subcutaneous injection. Oral administration, or administration by injection, such as by subcutaneous injection, offers the advantage of better comfort for the subject or patient and the opportunity to administer to a subject or patient outside of a hospital setting. In some embodiments, the AMYR agonist and the GLP-1R agonist (e.g. the GLP-1R/GCGR dual-agonist) or pharmaceutical compositions thereof are administered by self-administration.

In some embodiments the subject or patient is a mammal, in particular a human.

The subject to be treated may have diabetes (e.g. type 1 or type 2 diabetes). Alternatively, the subject may not have type 1 or type 2 diabetes. Alternatively or in addition, the subject may not have obesity induced by other endocrine disorders, such as insulinoma, Cushing's syndrome or Prader-Willi syndrome. Further alternatively or in addition, the subject may not have previous or planned (within the treatment course) bariatric surgery or fitting of a weight loss device (e.g. a gastric balloon or duodenal barrier). Further alternatively or in addition, the subject may not have gastroparesis.

The subject to be treated may be a human having a BMI of at least 25 kg/m² at baseline. The subject to be treated may be a human having a BMI of at least 27 kg/m² at baseline. The subject to be treated may be a human having a BMI of at least 30 kg/m². The subject may be a human having a BMI of at least 25, 26, 27, 28, 29, 30, 31, 32, 33, 34, 35, 36, 37, 38, 39 or 40 kg/m² at baseline. The subject may be a human having a BMI of less than or equal to 50 kg/m² at baseline. The subject may be a human having a BMI of less than or equal to 50, 49, 48, 47, 46, 45, 44, 43, 42, 41, or 40 kg/m² at baseline. The subject may be a human having a BMI of 25-40 kg/m² at baseline. The subject may be a human having a BMI of 27-40 kg/m² at baseline. The subject may be a human having a BMI of 30-40 kg/m² at baseline. Preferably, the subject is a human having a BMI of at least 30 kg/m² at baseline. Preferably, the subject is a human having a BMI of at least 27 kg/m² at baseline with at least one weight-related comorbidity (e.g., hypertension, dyslipidaemia, and/or obstructive sleep apnoea).

The subject may have previously been administered an anti-obesity drug, such as a dual amylin and calcitonin receptor agonist (DACRA, e.g., davalintide); an incretin mimetic drug, such as a GLP-1R agonist (e.g., semaglutide); an incretin mimetic drug, such as a GLP-1R agonist (e.g., semaglutide); a GLP-1R/GCGR dual agonist (e.g. survodutide); a GLP1/GCG/GIP triple agonist (e.g. retatrutide); and/or a DACRA-GLP-1Ra combination (e.g., semaglutide-cagrilintide, “cagri-sema”). In some embodiments, the subject experiences increased body weight reduction as compared to an anti-obesity drug, such as a dual amylin and calcitonin receptor agonist (DACRA e.g., davalintide); an incretin mimetic drug, such as a GLP-1R agonist (e.g., semaglutide); a GLP-1R/GCGR dual agonist (e.g. survodutide); a GLP1/GCG/GIP triple agonist (e.g. retatrutide); and/or a DACRA-GLP-1Ra combination (e.g., semaglutide-cagrilintide, “cagri-sema”).

In some embodiments, the subject does not tolerate an anti-obesity drug, such as a DACRA (e.g., davalintide); an incretin mimetic drug, such as a GLP-1R agonist (e.g., semaglutide; a GLP-1R/GCGR dual agonist (e.g. survodutide); a GLP1/GCG/GIP triple agonist (e.g. retatrutide); and/or a DACRA-GLP-1Ra combination (e.g., semaglutide-cagrilintide, “cagri-sema”). As used herein, a subject that does not or cannot tolerate a drug may have had, or be at risk of, adverse events (such as nausea and/or vomiting) in response to the drug. Thus, the subject may have experienced adverse events following administration of an anti-obesity drug; discontinued administration of the anti-obesity drug due to the burden of adverse events of the drug; or been advised that they are at risk of adverse events if administered an anti-obesity drug.

In some embodiments, the AMYR agonist and the GLP-1R agonist (e.g. the GLP-1R/GCGR dual-agonist) or pharmaceutical compositions thereof are administered to the subject in combination with insulin.

In some aspects, the AMYR agonist and the GLP-1R agonist (e.g. the GLP-1R/GCGR dual-agonist) or pharmaceutical compositions thereof are administered once a week, every two weeks, every three weeks, or every four weeks. In some aspects, the AMYR agonist and the GLP-1R agonist (e.g. the GLP-1R/GCGR dual-agonist) or pharmaceutical compositions thereof are administered about once every 4, 5, 6, 7, 8, 9, 10, 11, 12, 13, or 14 days. In a preferred embodiment, the AMYR agonist and the GLP-1R agonist (e.g. the GLP-1R/GCGR dual-agonist) or pharmaceutical compositions thereof are administered about once a week.

The AMYR agonist and the GLP-1R agonist (e.g. the GLP-1R/GCGR dual-agonist) or pharmaceutical compositions thereof may be administered simultaneously. Alternatively, the AMYR agonist and the GLP-1R agonist (e.g. the GLP-1R/GCGR dual-agonist) or pharmaceutical compositions thereof may be administered sequentially.

The AMYR agonist and the GLP-1R agonist (e.g. the GLP-1R/GCGR dual-agonist) or pharmaceutical compositions thereof may be administered using a dual-chamber device, as described herein.

Any AMYR agonist which has selectivity for AMYR as compared with CTR, and any GLP-1R agonist as described herein may be used for therapy, in a method of treatment or in the manufacture of a medicament according to the present invention. Preferably, the AMYR agonist with selectivity for AMYR as compared with CTR is an AMYR agonist polypeptide and GLP-1R agonist is a GLP-1R agonist polypeptide, as described herein. In some preferred embodiments, the AMYR agonist with selectivity for AMYR as compared with CTR is combined with a GLP-1R/GCGR dual-agonist, as described herein. Thus, in some particularly preferred embodiments, the AMYR agonist with selectivity for AMYR as compared with CTR is an AMYR agonist polypeptide and GLP-1R agonist is a GLP-1R/GCGR dual-agonist polypeptide, as described herein. By way of non-limiting example, an AMYR agonist polypeptide, or pharmaceutically acceptable salt thereof, comprising the amino acid sequence K(γE-γE-C18diacid)[CNTATC]ATQRLANFLRHSSNN(αMePhe)GPILPPTEVGSNTY-amide (SEQ ID NO: 11); and a GLP-1R agonist polypeptide, or pharmaceutically acceptable salt thereof, comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35) may be used in a therapy as described herein. By way of further non-limiting example, an AMYR agonist polypeptide, consisting of the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof; and a GLP-1R agonist polypeptide consisting of the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof may be used in a therapy as described herein.

Alternatively, any AMYR agonist which has selectivity for AMYR as compared with CTR, and any GLP-1R agonist as described herein may be used for therapy, in a method of treatment or in the manufacture of a medicament according to the present invention in combination with a GCGR agonist. Thus, a method of the invention may comprise administering an AMYR agonist which has selectivity for AMYR as compared with CTR, and a GLP-1R agonist as described herein to a subject, and further comprise administering to the subject a GCGR agonist.

### Dosage Regimens

The invention provides a combination of an AMYR agonist and a GLP-1R agonist (e.g. a GLP-1R/GCGR dual-agonist) for the treatment and/or prevention of a disease or disorder, or for a clinical purpose (e.g. improving glycemic and/or metabolic control, improving glucose homeostasis, β-cell function, and/or insulin sensitivity, or promoting end-organ protection) as described herein. In particular, said AMYR agonist and GLP-1R agonist (e.g. a GLP-1R/GCGR dual-agonist) are used at: (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof.

Dosage regimens for said treatments are provided below. For the avoidance of doubt, any dosage regimen herein may be used in relation to the treatment and/or prevention of any disease or disorder, or for any clinical purpose as described herein.

In particular, the invention provides a method of treating and/or preventing a disease or disorder in a subject, the method comprising administering to the subject (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof. The invention also provides an AMYR agonist and GLP-1R agonist (e.g. a GLP-1R/GCGR dual-agonist) or pharmaceutically acceptable salts thereof of the invention for use in therapy, for example in a method of treating and/or preventing a disease or disorder, the method comprising administering to the subject (a) between about 1 mg to about 15 mg of the AMYR agonist, and (b) between about 0.1 mg to about 10 mg of a GLP-1R agonist (e.g. a GLP-1R/GCGR dual-agonist), or pharmaceutically acceptable salts thereof.

The invention also provides a method of inhibiting or reducing weight gain, promoting weight loss, reducing food intake, increasing satiety, and/or reducing excess body weight in a subject, the method comprising administering to the subject (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof. The invention also provides an AMYR agonist and GLP-1R agonist (e.g. a GLP-1R/GCGR dual-agonist) or pharmaceutically acceptable salts thereof of the invention for use in a method of inhibiting or reducing weight gain, promoting weight loss, reducing food intake, increasing satiety, and/or reducing excess body weight in a subject, the method comprising administering to the subject (a) between about 1 mg to about 15 mg of the AMYR agonist, and (b) between about 0.1 mg to about 10 mg of a GLP-1R agonist (e.g. a GLP-1R/GCGR dual-agonist), or pharmaceutically acceptable salts thereof.

The invention also provides a method of reducing fat-mass specific body weight in a subject, the method comprising administering to the subject (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof. The invention also provides an AMYR agonist and GLP-1R agonist (e.g. a GLP-1R/GCGR dual-agonist) or pharmaceutically acceptable salts thereof of the invention for use in a method of reducing fat-mass specific body weight in a subject, the method comprising administering to the subject (a) between about 1 mg to about 15 mg of the AMYR agonist, and (b) between about 0.1 mg to about 10 mg of a GLP-1R agonist (e.g. a GLP-1R/GCGR dual-agonist), or pharmaceutically acceptable salts thereof.

The invention also provides a method of improving glycemic and/or metabolic control in a subject, the method comprising administering to the subject (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof. The invention also provides an AMYR agonist and GLP-1R agonist (e.g. a GLP-1R/GCGR dual-agonist) or pharmaceutically acceptable salts thereof of the invention for use in a method of improving glycemic and/or metabolic control in a subject, the method comprising administering to the subject (a) between about 1 mg to about 15 mg of the AMYR agonist, and (b) between about 0.1 mg to about 10 mg of a GLP-1R agonist (e.g. a GLP-1R/GCGR dual-agonist), or pharmaceutically acceptable salts thereof.

The invention also provides a method of treating and/or preventing obesity or an obesity-related condition in a subject in need thereof, the method comprising administering to the subject (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof. The invention also provides an AMYR agonist and GLP-1R agonist (e.g. a GLP-1R/GCGR dual-agonist) or pharmaceutically acceptable salts thereof of the invention for use in a method of treating and/or preventing obesity or an obesity-related condition in a subject in need thereof, the method comprising administering to the subject (a) between about 1 mg to about 15 mg of the AMYR agonist, and (b) between about 0.1 mg to about 10 mg of a GLP-1R agonist (e.g. a GLP-1R/GCGR dual-agonist), or pharmaceutically acceptable salts thereof.

The invention also provides a method of treating and/or preventing a metabolic disease in a subject in need thereof, the method comprising administering to the subject (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof. The invention also provides an AMYR agonist and GLP-1R agonist (e.g. a GLP-1R/GCGR dual-agonist) or pharmaceutically acceptable salts thereof of the invention for use in a method of treating and/or preventing a metabolic disease in a subject in need thereof, the method comprising administering to the subject (a) between about 1 mg to about 15 mg of the AMYR agonist, and (b) between about 0.1 mg to about 10 mg of a GLP-1R agonist (e.g. a GLP-1R/GCGR dual-agonist), or pharmaceutically acceptable salts thereof.

The invention also provides a method of reducing the body weight of a subject, the method comprising administering to the subject (a) between about 1 mg to about 15 mg of an AMYR agonist, or pharmaceutically acceptable salt thereof, and (b) between about 0.1 mg to about 10 mg of a GLP-1R agonist (e.g. a GLP-1R/GCGR dual-agonist), or pharmaceutically acceptable salt thereof. The invention also provides an AMYR agonist and GLP-1R agonist (e.g. a GLP-1R/GCGR dual-agonist) or pharmaceutically acceptable salts thereof of the invention for use in a method of reducing the body weight of a subject, the method comprising administering to the subject (a) between about 1 mg to about 15 mg of the AMYR agonist, and (b) between about 0.1 mg to about 10 mg of a GLP-1R agonist (e.g. a GLP-1R/GCGR dual-agonist), or pharmaceutically acceptable salts thereof.

Also provided is a kit comprising (a) an AMYR agonist, (b) a GLP-1R agonist, and (c) instructions for use of the same for treating or preventing a disease or disorder as disclosed herein (such as obesity or an obesity-related condition, or a metabolic disease as described herein) at a dose of about 1 mg to about 15 mg of the AMYR agonist and about 0.1 to about 10 mg of the GLP-1R agonist. Also provided is an article of manufacture comprising (a) an AMYR agonist at a dose of about 1 mg to about 15 mg; and (b) a GLP-1R agonist at a dose of about 0.1 to about 10 mg.

The AMYR agonist and GLP-1R agonist (e.g. a GLP-1R/GCGR dual-agonist) of the invention may be any AMYR agonist and GLP-1R agonist (e.g. a GLP-1R/GCGR dual-agonist) as disclosed herein.

Preferably, the doses as disclosed herein are fixed doses. As used herein, the term “fixed dose” refers to a dose which is used for all subjects, e.g., the dose is an amount that does not vary based on the weight of the subject. A fixed dose may be a specific, unchanging amount, wherein the same dose is used for all subjects, or for all adult subjects, such as a unit dosage form as disclosed herein. A fixed dose is typically expressed in terms of an amount of the active agent, such as the amylin receptor agonist, GLP-1R agonist (e.g. GLP-1R/GCGR dual agonist), or pharmaceutically acceptable salts thereof, rather than an amount that is expressed relative to the weight or mass of the subject to be treated.

The methods, uses, compositions, and kits of the invention utilise a dose (such as a fixed dose) of about 1 mg to about 15 mg of an AMYR agonist, or pharmaceutically acceptable salt thereof, and about 0.1 mg to about 10 mg of a GLP-1R agonist (e.g. a GLP-1R/GCGR dual-agonist) or pharmaceutically acceptable salt thereof, for example comprise administering such a (fixed) dose to a subject.

### AMYR Agonist Dosages

The amylin receptor agonist in a method of the invention, an amylin receptor agonist for use in the invention, and the uses, compositions or kits of the invention may be any amylin receptor agonist as disclosed herein.

The (fixed) dose of amylin receptor agonist of about 1 mg to about 15 mg may be, for example, at least 1 mg, such as at least 2 mg, at least 3 mg, at least 4 mg, at least 5 mg, at least 6 mg, at least 7 mg, at least 8 mg, at least 9 mg, at least 10 mg, at least 11 mg, or at least 12 mg.

The (fixed) dose of about 1 mg to about 15 mg may be up to 15 mg, such as up to 12 mg, up to 11 mg, up to 10 mg, up to 9 mg, up to 8 mg, up to 7 mg, up to 6 mg, up to 5 mg, up to 4 mg, up to 3 mg, or up to 2 mg. Any of these upper and lower end points may be combined within the range of about 1 mg to about 15 mg. For example, the AMYR agonist, or pharmaceutically acceptable salt thereof, may be administered to the subject at a dose, such as a fixed dose, of about 1 mg to about 10 mg, about 1 mg to about 6 mg, about 1 mg to about 5 mg, about 4 mg to about 10 mg, about 1 mg to about 3 mg, about 2.5 mg to about 5 mg, or about 7 mg to about 10 mg.

The AMYR agonist, or pharmaceutically acceptable salt thereof, may be administered to the subject at a dose, such as a fixed dose, of about 1 mg to about 12 mg. The AMYR agonist, or pharmaceutically acceptable salt thereof, may be administered to the subject at a dose, such as a fixed dose, of about 1 mg to about 10 mg. The AMYR agonist, or pharmaceutically acceptable salt thereof, may be administered to the subject at a dose, such as a fixed dose, of about 1.5 mg to about 9 mg.

The AMYR agonist, or pharmaceutically acceptable salt thereof, may be administered to the subject at a dose, such as a fixed dose, of about 1 mg to about 8 mg, about 1 mg to about 7 mg, about 1 mg to about 6 mg, or about 1 mg to about 5 mg. The AMYR agonist, or pharmaceutically acceptable salt thereof, may be administered to the subject at a dose, such as a fixed dose, of about 1.5 mg to about 8 mg, about 1.5 mg to about 7 mg, about 1.5 mg to about 6 mg, about 1.5 mg to about 5 mg, or about 1.5 mg to about 4.5 mg. Preferably, the AMYR agonist, or pharmaceutically acceptable salt thereof, may be administered to the subject at a dose, such as a fixed dose, of about 1 mg to about 5 mg. Preferably, the AMYR agonist, or pharmaceutically acceptable salt thereof, may be administered to the subject at a dose, such as a fixed dose, of about 1.5 mg to about 4.5 mg.

The AMYR agonist, or pharmaceutically acceptable salt thereof, may be administered to the subject at a dose, such as a fixed dose, of about 1.6 mg to about 8 mg, about 1.6 mg to about 7 mg, about 1.6 mg to about 6 mg, about 1.6 mg to about 5 mg, or about 1.6 mg to about 4.5 mg.

The AMYR agonist, or pharmaceutically acceptable salt thereof, may be administered to the subject at a dose, such as a fixed dose of about 2 mg to about 15 mg, about 3 mg to about 15 mg, about 4 mg to about 15 mg, about 4.5 mg to about 15 mg, or about 6 mg to about 15 mg. The AMYR agonist, or pharmaceutically acceptable salt thereof, may be administered to the subject at a dose, such as a fixed dose of about 2 mg to about 12 mg, about 3 mg to about 12 mg, about 4 mg to about 12 mg, about 4.5 mg to about 12 mg, or about 6 mg to about 12 mg. The AMYR agonist, or pharmaceutically acceptable salt thereof, may be administered to the subject at a dose, such as a fixed dose of about 2 mg to about 10 mg, about 3 mg to about 10 mg, about 4 mg to about 10 mg, about 4.5 mg to about 10 mg, or about 6 mg to about 10 mg. The AMYR agonist, or pharmaceutically acceptable salt thereof, may be administered to the subject at a dose, such as a fixed dose of about 2 mg to about 9 mg, about 3 mg to about 9 mg, about 4 mg to about 9 mg, about 4.5 mg to about 9 mg, or about 6 mg to about 9 mg. The AMYR agonist, or pharmaceutically acceptable salt thereof, may be administered to the subject at a dose, such as a fixed dose, of about 4 mg to about 15 mg, about 4 mg to about 12 mg, about 4 mg to about 10 mg, about 4 mg to about 9 mg, or about 6 mg to about 9 mg. The AMYR agonist, or pharmaceutically acceptable salt thereof, may be administered to the subject at a dose, such as a fixed dose, of about 6 mg to about 15 mg, about 6 mg to about 12 mg, about 6 mg to about 10 mg, or about 6 mg to about 9 mg. The AMYR agonist, or pharmaceutically acceptable salt thereof, may be administered to the subject at a dose, such as a fixed dose, of about 4.5 mg to about 15 mg, about 4.5 mg to about 12 mg, about 4.5 mg to about 10 mg, or about 4.5 mg to about 9 mg. Preferably, the AMYR agonist, or pharmaceutically acceptable salt thereof, may be administered to the subject at a dose, such as a fixed dose, of about 4 mg to about 10 mg. Preferably, the AMYR agonist, or pharmaceutically acceptable salt thereof, may be administered to the subject at a dose, such as a fixed dose, of about 4 mg to about 9 mg. Preferably, the AMYR agonist, or pharmaceutically acceptable salt thereof, may be administered to the subject at a dose, such as a fixed dose, of about 4.5 mg to about 9 mg.

The AMYR agonist, or pharmaceutically acceptable salt thereof, may be administered to the subject at a dose, such as a fixed dose of about 1 mg to about 4 mg, about 1 mg to about 3 mg, or about 1 mg to about 2 mg. Preferably, the AMYR agonist, or pharmaceutically acceptable salt thereof, may be administered to the subject at a dose, such as a fixed dose, of about 1 mg to about 3 mg.

The AMYR agonist, or pharmaceutically acceptable salt thereof, may be administered to the subject at a dose, such as a fixed dose, of about 2 mg to about 8 mg, about 2 mg to about 7 mg, about 2 mg to about 6 mg, or about 2 mg to about 5 mg. The AMYR agonist, or pharmaceutically acceptable salt thereof, may be administered to the subject at a dose, such as a fixed dose, of about 3 mg to about 8 mg, about 3 mg to about 7 mg, about 3 mg to about 6 mg, or about 3 mg to about 5 mg. The AMYR agonist, or pharmaceutically acceptable salt thereof, may be administered to the subject at a dose, such as a fixed dose, of about 4 mg to about 8 mg, about 4 mg to about 7 mg, about 4 mg to about 6 mg, or about 4 mg to about 5 mg. The AMYR agonist, or pharmaceutically acceptable salt thereof, may be administered to the subject at a dose, such as a fixed dose, of about 2.5 mg to about 8 mg, about 3 mg to about 8 mg, or about 4 mg to about 8 mg. The AMYR agonist, or pharmaceutically acceptable salt thereof, may be administered to the subject at a dose, such as a fixed dose, of about 2.5 mg to about 7 mg, about 3 mg to about 7 mg, or about 4 mg to about 7 mg. The AMYR agonist, or pharmaceutically acceptable salt thereof, may be administered to the subject at a dose, such as a fixed dose, of about 2.5 mg to about 6 mg, about 3 mg to about 6 mg, or about 4 mg to about 6 mg. The AMYR agonist, or pharmaceutically acceptable salt thereof, may be administered to the subject at a dose, such as a fixed dose, of about 2.5 mg to about 5 mg, about 3 mg to about 5 mg, or about 4 mg to about 5 mg. Preferably, the AMYR agonist, or pharmaceutically acceptable salt thereof, may be administered to the subject at a dose, such as a fixed dose, of about 2.5 mg to about 5 mg.

The AMYR agonist, or pharmaceutically acceptable salt thereof, may be administered to the subject at a dose, such as a fixed dose, of about 5 mg to about 12 mg, about 5 mg to about 10 mg, or about 5 mg to about 9 mg. The AMYR agonist, or pharmaceutically acceptable salt thereof, may be administered to the subject at a dose, such as a fixed dose, of about 6 mg to about 12 mg, about 6 mg to about 10 mg, or about 6 mg to about 9 mg. The AMYR agonist, or pharmaceutically acceptable salt thereof, may be administered to the subject at a dose, such as a fixed dose, of about 7 mg to about 12 mg, about 7 mg to about 10 mg, or about 7 mg to about 9 mg. The AMYR agonist, or pharmaceutically acceptable salt thereof, may be administered to the subject at a dose, such as a fixed dose, of about 8 mg to about 9 mg. Preferably, the AMYR agonist, or pharmaceutically acceptable salt thereof, may be administered to the subject at a dose, such as a fixed dose, of about 7 mg to about 10 mg.

The AMYR agonist may be administered to the subject at a dose, such as a fixed dose, of up to 15 mg. For example, the AMYR agonist may be administered to the subject at a dose, such as a fixed dose, of about 1 mg to about 10 mg, about 1 mg to about 5 mg, about 4 mg to about 10 mg, about 1 mg to about 3 mg, about 2.5 mg to about 5 mg, or about 7 mg to about 10 mg.

The AMYR agonist may be administered to the subject at a dose, such as a fixed dose, of up to 12 mg. For example, the AMYR agonist may be administered to the subject at a dose, such as a fixed dose, of about 1 mg to about 10 mg, about 1 mg to about 5 mg, about 4 mg to about 10 mg, about 1 mg to about 3 mg, about 2.5 mg to about 5 mg, or about 7 mg to about 10 mg.

The AMYR agonist may be administered to the subject at a dose, such as a fixed dose, of up to 10 mg. For example, the AMYR agonist may be administered to the subject at a dose, such as a fixed dose, of about 1 mg to about 10 mg, about 1 mg to about 5 mg, about 4 mg to about 10 mg, about 1 mg to about 3 mg, about 2.5 mg to about 5 mg, or about 7 mg to about 10 mg.

The AMYR agonist may be administered to the subject at a dose, such as a fixed dose, of up to 9 mg. For example, the AMYR agonist may be administered to the subject at a dose, such as a fixed dose, of about 1 mg to about 9 mg, about 1 mg to about 5 mg, about 4 mg to about 9 mg, about 1 mg to about 3 mg, about 2.5 mg to about 5 mg, about 7 mg to about 9 mg, or about 9 mg.

The AMYR agonist may be administered to the subject at a dose, such as a fixed dose, of up to 8 mg. For example, the AMYR agonist may be administered to the subject at a dose, such as a fixed dose, of about 1 mg to about 8 mg, about 1 mg to about 5 mg, about 4 mg to about 8 mg, about 1 mg to about 3 mg, about 2.5 mg to about 5 mg, or about 7 mg to about 8 mg.

The AMYR agonist may be administered to the subject at a dose, such as a fixed dose, of up to 7 mg. For example, the AMYR agonist may be administered to the subject at a dose, such as a fixed dose, of about 1 mg to about 7 mg, about 1 mg to about 5 mg, about 4 mg to about 7 mg, about 1 mg to about 3 mg, or about 2.5 mg to about 5 mg.

The AMYR agonist may be administered to the subject at a dose, such as a fixed dose, of up to 6 mg. For example, the AMYR agonist may be administered to the subject at a dose, such as a fixed dose, of about 1 mg to about 6 mg, about 1 mg to about 5 mg, about 4 mg to about 6 mg, about 1 mg to about 3 mg, or about 2.5 mg to about 5 mg.

The AMYR agonist may be administered to the subject at a dose, such as a fixed dose, of up to 5 mg. For example, the AMYR agonist may be administered to the subject at a dose, such as a fixed dose, of about 1 mg to about 5 mg, about 4 mg to about 5 mg, about 1 mg to about 3 mg, about 2.5 mg to about 5 mg, or about 4.5 mg.

The AMYR agonist may be administered to the subject at a dose, such as a fixed dose, of up to 4 mg. For example, the AMYR agonist may be administered to the subject at a dose, such as a fixed dose, of about 1 mg to about 4 mg, about 1 mg to about 3 mg, or about 2.5 mg to about 4 mg.

The AMYR agonist may be administered to the subject at a dose, such as a fixed dose, of up to 3 mg. For example, the AMYR agonist may be administered to the subject at a dose, such as a fixed dose, of about 1 mg to about 3 mg.

The AMYR agonist may be administered to the subject at a dose, such as a fixed dose, of up to 2 mg. For example, the AMYR agonist may be administered to the subject at a dose, such as a fixed dose, of about 1 mg to about 2 mg, or about 1.5 mg (e.g. 1.6 mg).

Preferably, the AMYR agonist may be administered to the subject at a dose, such as a fixed 30 dose, of about 1.5 mg (e.g., about 1.6 mg), about 4.5 mg, about 6 mg, or about 9 mg.

Preferably, the AMYR agonist may be administered to the subject at a dose, such as a fixed dose, of about 1.5 mg (e.g. 1.6 mg), about 4.5 mg, or about 9 mg.

Preferably, the AMYR agonist may be administered to the subject at a dose, such as a fixed dose, of about 1.5 mg, such as a dose of 1.5 mg.

Preferably, the AMYR agonist may be administered to the subject at a dose, such as a fixed dose, of about 1.6 mg, such as a dose of 1.6 mg.

Preferably, the AMYR agonist may be administered to the subject at a dose, such as a fixed dose, of about 4.5 mg, such as a dose of 4.5 mg.

Preferably, the AMYR agonist may be administered to the subject at a dose, such as a fixed dose, of about 6 mg, such as a dose of 6 mg.

Preferably, the AMYR agonist may be administered to the subject at a dose, such as a fixed dose, of about 9 mg, such as a dose of 9 mg.

### GLP-1R Agonist Dosages

The GLP-1R receptor agonist in a method of the invention, an GLP-1R agonist for use in the invention, and the uses, compositions or kits of the invention may be any GLP-1R agonist as disclosed herein.

The (fixed) dose of GLP-1R agonist (e.g., GLP-1R/GCGR dual agonist), or pharmaceutically acceptable salt thereof of about 0.1 mg to about 10 mg may be, for example, at least 0.1 mg, such as at least 0.15 mg, at least 0.2 mg, at least 0.3 mg, at least 0.4 mg, at least 0.5 mg, at least 0.6 mg, at least 0.7 mg, at least 0.8 mg, at least 0.9 mg, at least 1 mg, at least 1.5 mg, at least 1.2 mg, at least 2 mg, at least 3 mg, at least 4 mg, at least 4.5 mg, at least 4.6 mg, at least 5 mg, at least 6 mg, at least 7 mg, at least 8 mg, at least 9 mg, at least 9.4 mg, or at least 10 mg. The (fixed) dose of about 0.1 mg to about 10 mg may be up to 10 mg, such as up to 9.4 mg, up to 9 mg, up to 8 mg, up to 7 mg, up to 6 mg, up to 5 mg, up to 4.6 mg, up to 4.5 mg, up to 4 mg, up to 3 mg, up to 2 mg, up to 1.5 mg, up to 1.2 mg, up to 1 mg, up to 0.9 mg, up to 0.8 mg, up to 0.7 mg, up to 0.6 mg, up to 0.5 mg, up to 0.4 mg, up to 0.3 mg, or up to 0.2 mg. Any of these upper and lower end points may be combined within the range of about 0.1 mg to about 10 mg.

The GLP-1R agonist (e.g., GLP-1R/GCGR dual agonist), or pharmaceutically acceptable salt thereof, may be administered to the subject at a dose, such as a fixed dose, of about 0.1 mg to about 9 mg, for example, from about 0.1 mg to about 8 mg, from about 0.1 mg to about 7 mg, from about 0.1 mg to about 6 mg, from about 0.1 mg to about 5 mg, from about 0.1 mg to about 4 mg, from about 0.1 mg to about 3 mg, from about 0.1 mg to about 2 mg, or from about 0.1 mg to about 1 mg.

The GLP-1R agonist (e.g., GLP-1R/GCGR dual agonist), or pharmaceutically acceptable salt thereof, may be administered to the subject at a dose, such as a fixed dose, of about 0.15 mg to about 10 mg, for example, from about 0.15 mg to about 9 mg, from about 0.15 mg to about 8 mg, from about 0.15 mg to about 7 mg, from about 0.15 mg to about 6 mg, from about 0.15 mg to about 5 mg, from about 0.15 mg to about 4 mg, from about 0.15 mg to about 3 mg, from about 0.15 mg to about 2 mg, or from about 0.15 mg to about 1 mg.

The GLP-1R agonist (e.g., GLP-1R/GCGR dual agonist), or pharmaceutically acceptable salt thereof, may be administered to the subject at a dose, such as a fixed dose, of about 0.30 mg to about 10 mg, for example, from about 0.30 mg to about 9 mg, from about 0.30 mg to about 8 mg, from about 0.30 mg to about 7 mg, from about 0.30 mg to about 6 mg, from about 0.30 mg to about 5 mg, from about 0.30 mg to about 4 mg, from about 0.30 mg to about 3 mg, from about 0.30 mg to about 2 mg, or from about 0.30 mg to about 1 mg.

The GLP-1R agonist (e.g., GLP-1R/GCGR dual agonist), or pharmaceutically acceptable salt thereof, may be administered to the subject at a dose, such as a fixed dose, of about 0.60 mg to about 10 mg, for example, from about 0.60 mg to about 9 mg, from about 0.60 mg to about 8 mg, from about 0.60 mg to about 7 mg, from about 0.60 mg to about 6 mg, from about 0.60 mg to about 5 mg, from about 0.60 mg to about 4 mg, from about 0.60 mg to about 3 mg, from about 0.60 mg to about 2 mg, or from about 0.60 mg to about 1 mg.

The GLP-1R agonist (e.g., GLP-1R/GCGR dual agonist), or pharmaceutically acceptable salt thereof, may be administered to the subject at a dose, such as a fixed dose, of about 1.20 mg to about 10 mg, for example, from about 1.20 mg to about 9 mg, from about 1.20 mg to about 8 mg, from about 1.20 mg to about 7 mg, from about 1.20 mg to about 6 mg, from about 1.20 mg to about 5 mg, from about 1.20 mg to about 4 mg, from about 1.20 mg to about 3 mg, or from about 1.20 mg to about 2 mg.

The GLP-1R agonist (e.g., GLP-1R/GCGR dual agonist), or pharmaceutically acceptable salt thereof, may be administered to the subject at a dose, such as a fixed dose, of about 2.30 mg to about 10 mg, for example, from about 2.30 mg to about 9 mg, from about 2.30 mg to about 8 mg, from about 2.30 mg to about 7 mg, from about 2.30 mg to about 6 mg, from about 2.30 mg to about 5 mg, from about 2.30 mg to about 4 mg, or from about 2.30 mg to about 3 mg.

The GLP-1R agonist (e.g., GLP-1R/GCGR dual agonist), or pharmaceutically acceptable salt thereof, may be administered to the subject at a dose, such as a fixed dose, of about 4.60 mg to about 10 mg, for example, from about 4.60 mg to about 9 mg, from about 4.60 mg to about 8 mg, from about 4.60 mg to about 7 mg, from about 4.60 mg to about 6 mg, or from about 4.60 mg to about 5 mg.

The GLP-1R agonist (e.g., GLP-1R/GCGR dual agonist), or pharmaceutically acceptable salt thereof, may be administered to the subject at a dose, such as a fixed dose, of about 8 mg to about 10 mg, for example, about 9 mg to about 10 mg, about 9.5 mg to about 10 mg, about 9.4 mg to about 10 mg, or about 9.4 mg.

The GLP-1R agonist (e.g., GLP-1R/GCGR dual agonist), or pharmaceutically acceptable salt thereof, may be administered to the subject at a dose, such as a fixed dose, of about 0.15 mg to about 10 mg, about 0.20 to about 10 mg, about 0.30 mg to about 10 mg, about 0.50 mg to about 10 mg, about 0.60 mg to about 10 mg, about 1.00 mg to about 10 mg, about 1.20 mg to about 10 mg, about 1.50 mg to about 10 mg, about 2 mg to about 10 mg, about 2.30 mg to about 10 mg, about 2.50 mg to about 10 mg, about 3 mg to about 10 mg, about 4 mg to about 10 mg, about 4.50 mg to about 10 mg, about 4.60 mg to about 10 mg, about 5 mg to about 10 mg, about 6 mg to about 10 mg, about 7 mg to about 10 mg, or about 9 mg to about 10 mg.

The GLP-1R agonist (e.g., GLP-1R/GCGR dual agonist), or pharmaceutically acceptable salt thereof, may be administered to the subject at a dose, such as a fixed dose, of about 0.1 mg to about 9.5 mg (e.g. about 9.4 mg), for example, about 0.15 mg to about 9.5 mg (e.g. about 9.4 mg), about 0.20 mg to about 9.5 mg (e.g. about 9.4 mg), about 0.30 mg to about 9.5 mg (e.g. about 9.4 mg), about 0.50 mg to about 9.5 mg (e.g. about 9.4 mg), about 0.60 mg to about 9.5 mg (e.g. about 9.4 mg), about 1.00 mg to about 9.5 mg (e.g. about 9.4 mg), about 1.20 mg to about 9.5 mg (e.g. about 9.4 mg), about 1.50 mg to about 9.5 mg (e.g. about 9.4 mg), about 2 mg to about 9.5 mg (e.g. about 9.4 mg), about 2.30 mg to about 9.5 mg (e.g. about 9.4 mg), about 2.50 mg to about 9.5 mg (e.g. about 9.4 mg), about 3 mg to about 9.5 mg (e.g. about 9.4 mg), about 4 mg to about 9.5 mg (e.g. about 9.4 mg), about 4.50 mg to about 9.5 mg (e.g. about 9.4 mg), about 4.60 mg to about 9.5 mg (e.g. about 9.4 mg), about 5 mg to about 9.5 mg (e.g. about 9.4 mg), or about 7 mg to about 9.5 mg (e.g. about 9.4 mg).

The GLP-1R agonist (e.g., GLP-1R/GCGR dual agonist), or pharmaceutically acceptable salt thereof, may be administered to the subject at a dose, such as a fixed dose, of about 0.1 mg to about 7 mg, for example, about 0.15 mg to about 7 mg, about 0.20 mg to about 7 mg, about 0.30 mg to about 7 mg, about 0.50 mg to about 7 mg, about 0.60 mg to about 7 mg, about 1.00 mg to about 7 mg, about 1.20 mg to about 7 mg, about 1.50 mg to about 7 mg, about 2 mg to about 7 mg, about 2.30 mg to about 7 mg, about 2.50 mg to about 7 mg, about 3 mg to about 7 mg, about 4 mg to about 7 mg, about 4.50 mg to about 7 mg, about 4.60 mg to about 7 mg, or about 5 mg to about 7 mg.

The GLP-1R agonist (e.g., GLP-1R/GCGR dual agonist), or pharmaceutically acceptable salt thereof, may be administered to the subject at a dose, such as a fixed dose, of about 0.1 mg to about 5 mg, for example, about 0.15 mg to about 5 mg, about 0.2 mg to about 5 mg, about 0.30 mg to about 5 mg, about 0.50 mg to about 5 mg, about 0.60 mg to about 5 mg, about 1.00 mg to about 5 mg, about 1.20 mg to about 5 mg, about 1.50 mg to about 5 mg, about 2 mg to about 5 mg, about 2.30 mg to about 5 mg, about 2.50 mg to about 5 mg, about 3 mg to about 5 mg, about 4 mg to about 5 mg, or about 4.6 mg to about 5 mg.

The GLP-1R agonist (e.g., GLP-1R/GCGR dual agonist), or pharmaceutically acceptable salt thereof, may be administered to the subject at a dose, such as a fixed dose, of about 0.1 mg to about 4.60 mg, for example, about 0.15 mg to about 4.60 mg, about 0.30 mg to about 4.60 mg, about 0.60 mg to about 4.60 mg, about 1.00 mg to about 4.60 mg, about 1.20 mg to about 4.60 mg, about 1.50 mg to about 4.60 mg, about 2 mg to about 4.60 mg, about 2.30 mg to about 4.60 mg, about 2.50 mg to about 4.60 mg, about 3 mg to about 4.60 mg, or about 4 mg to about 4.60 mg.

The GLP-1R agonist (e.g., GLP-1R/GCGR dual agonist), or pharmaceutically acceptable salt thereof, may be administered to the subject at a dose, such as a fixed dose, of about 0.1 mg to about 2.30 mg, for example, about 0.15 mg to about 2.30 mg, about 0.30 mg to about 2.30 mg, about 0.60 mg to about 2.30 mg, about 1.00 mg to about 2.30 mg, about 1.20 mg to about 2.30 mg, about 1.50 mg to about 2.30 mg, or about 2 mg to about 2.30.

The GLP-1R agonist (e.g., GLP-1R/GCGR dual agonist), or pharmaceutically acceptable salt thereof, may be administered to the subject at a dose, such as a fixed dose, of about 0.1 mg to about 1.50 mg, for example, about 0.15 mg to about 1.50 mg, about 0.20 mg to about 1.50 mg, about 0.30 mg to about 1.50 mg, about 0.50 mg to about 1.50 mg, about 0.60 mg to about 1.50 mg, or about 1 mg to about 1.50 mg.

The GLP-1R agonist (e.g., GLP-1R/GCGR dual agonist), or pharmaceutically acceptable salt thereof, may be administered to the subject at a dose, such as a fixed dose, of about 0.1 mg to about 1.20 mg, for example, about 0.15 mg to about 1.20 mg, about 0.30 mg to about 1.20 mg, about 0.60 mg to about 1.20 mg, or about 1 mg to about 1.20 mg.

The GLP-1R agonist (e.g., GLP-1R/GCGR dual agonist), or pharmaceutically acceptable salt thereof, may be administered to the subject at a dose, such as a fixed dose, of about 0.1 mg to about 1 mg, for example, about 0.15 mg to about 1 mg, about 0.20 mg to about 1 mg, about 0.30 mg to about 1 mg, about 0.50 mg to about 1 mg, about 0.60 mg to about 1 mg, or about 1 mg.

The GLP-1R agonist (e.g., GLP-1R/GCGR dual agonist), or pharmaceutically acceptable salt thereof, may be administered to the subject at a dose, such as a fixed dose, of about 0.1 mg to about 0.60 mg, for example, about 0.15 mg to about 0.60 mg, or about 0.30 mg to about 0.60 mg.

The GLP-1R agonist (e.g., GLP-1R/GCGR dual agonist), or pharmaceutically acceptable salt thereof, may be administered to the subject at a dose, such as a fixed dose, of about 0.1 mg to about 0.50 mg, for example, about 0.15 mg to about 0.50 mg, about 0.20 mg to about 0.50 mg, or about 0.30 mg to about 0.50 mg.

The GLP-1R agonist (e.g., GLP-1R/GCGR dual agonist), or pharmaceutically acceptable salt thereof, may be administered to the subject at a dose, such as a fixed dose, of about 0.1 mg to about 0.30 mg, for example, or about 0.15 mg to about 0.30 mg.

The GLP-1R agonist (e.g., GLP-1R/GCGR dual agonist), or pharmaceutically acceptable salt thereof, may be administered to the subject at a dose, such as a fixed dose, of about 0.1 mg to about 0.20 mg, for example, or about 0.15 mg to about 0.20 mg.

The GLP-1R agonist (e.g., GLP-1R/GCGR dual agonist), or pharmaceutically acceptable salt thereof, may be administered to the subject at a dose, such as a fixed dose, of about 1 mg to about 10 mg, for example, about 1 mg to about 9.5 mg (for example, about 1.20 mg to about 9.4 mg).

The GLP-1R agonist (e.g., GLP-1R/GCGR dual agonist), or pharmaceutically acceptable salt thereof, may be administered to the subject at a dose, such as a fixed dose, of about 1 mg to about 10 mg, for example, about 1 mg to about 7 mg (for example, about 1.20 mg to about 7 mg).

The GLP-1R agonist (e.g., GLP-1R/GCGR dual agonist), or pharmaceutically acceptable salt thereof, may be administered to the subject at a dose, such as a fixed dose, of about 0.1 mg to about 5 mg, for example, about 1 mg to about 5 mg, about 1.2 mg to about 5 mg, or about 1.2 mg to about 4.6 mg. For example, the GLP-1R agonist (e.g., GLP-1R/GCGR dual agonist), or pharmaceutically acceptable salt thereof, may be administered to the subject at a dose, such as a fixed dose, about 1 mg to about 5 mg.

The GLP-1R agonist (e.g., GLP-1R/GCGR dual agonist), or pharmaceutically acceptable salt thereof, may be administered to the subject at a dose, such as a fixed dose, of about 2 mg to about 10 mg, for example, about 2 mg to about 9.5 mg, or about 2 mg to about 9.4 mg. The GLP-1R agonist (e.g., GLP-1R/GCGR dual agonist), or pharmaceutically acceptable salt thereof, may be administered to the subject at a dose, such as a fixed dose, of about 2.3 mg to about 10 mg, for example, about 2.3 mg to about 9.5 mg, or about 2.3 mg to about 9.4 mg.

The GLP-1R agonist (e.g., GLP-1R/GCGR dual agonist), or pharmaceutically acceptable salt thereof, may be administered to the subject at a dose, such as a fixed dose, of about 2 mg to about 7 mg. The GLP-1R agonist (e.g., GLP-1R/GCGR dual agonist), or pharmaceutically acceptable salt thereof, may be administered to the subject at a dose, such as a fixed dose, of about 2.3 mg to about 7 mg.

The GLP-1R agonist (e.g., GLP-1R/GCGR dual agonist), or pharmaceutically acceptable salt thereof, may be administered to the subject at a dose, such as a fixed dose, of about 0.1 mg to about 3 mg, for example, about 1 mg to about 3 mg, about 1.2 mg to about 3 mg, or about 1.2 mg to about 2.3 mg. For example, the GLP-1R agonist (e.g., GLP-1R/GCGR dual agonist), or pharmaceutically acceptable salt thereof, may be administered to the subject at a dose, such as a fixed dose, about 1 mg to about 3 mg.

The GLP-1R agonist (e.g., GLP-1R/GCGR dual agonist), or pharmaceutically acceptable salt thereof, may be administered to the subject at a dose, such as a fixed dose, of about 2 mg to about 5 mg, for example, about 2 mg to about 4.6 mg. The GLP-1R agonist (e.g., GLP-1R/GCGR dual agonist), or pharmaceutically acceptable salt thereof, may be administered to the subject at a dose, such as a fixed dose, of about 2.3 mg to about 5 mg, for example, about 2.3 mg to about 4.6 mg.

The GLP-1R agonist (e.g., GLP-1R/GCGR dual agonist), or pharmaceutically acceptable salt thereof, may be administered to the subject at a dose, such as a fixed dose, of about 4 mg to about 10 mg, for example, about 4 mg to about 7 mg. The GLP-1R agonist (e.g., GLP-1R/GCGR dual agonist), or pharmaceutically acceptable salt thereof, may be administered to the subject at a dose, such as a fixed dose, of about 4.5 mg to about 10 mg, for example, about 4.5 mg to about 7 mg. The GLP-1R agonist (e.g., GLP-1R/GCGR dual agonist), or pharmaceutically acceptable salt thereof, may be administered to the subject at a dose, such as a fixed dose, of about 4.6 mg to about 10 mg, for example, about 4.6 mg to about 7 mg.

The GLP-1R agonist (e.g., GLP-1R/GCGR dual agonist), or pharmaceutically acceptable salt thereof, may be administered to the subject at a dose, such as a fixed dose, of about 4 mg to about 10 mg, for example, about 4 mg to about 9.5 mg (e.g. about 9.4 mg). The GLP-1R agonist (e.g., GLP-1R/GCGR dual agonist), or pharmaceutically acceptable salt thereof, may be administered to the subject at a dose, such as a fixed dose, of about 4.5 mg to about 10 mg, for example, about 4.5 mg to about 9.5 mg (e.g. about 9.4 mg). The GLP-1R agonist (e.g., GLP-1R/GCGR dual agonist), or pharmaceutically acceptable salt thereof, may be administered to the subject at a dose, such as a fixed dose, of about 4.6 mg to about 10 mg, for example, about 4.6 mg to about 9.5 mg (e.g. about 9.4 mg).

The GLP-1R agonist (e.g., GLP-1R/GCGR dual agonist), or pharmaceutically acceptable salt thereof, may be administered to the subject at a dose, such as a fixed dose, of about 0.1 mg to about 2 mg, for example, about 1 mg to about 2 mg, about 1 mg to about 1.5 mg, or about 1.2 mg.

The GLP-1R agonist (e.g., GLP-1R/GCGR dual agonist), or pharmaceutically acceptable salt thereof, may be administered to the subject at a dose, such as a fixed dose, of about 2 mg to about 3 mg, for example, about 2 mg to about 2.5 mg, or about 2.3 mg.

The GLP-1R agonist (e.g., GLP-1R/GCGR dual agonist), or pharmaceutically acceptable salt thereof, may be administered to the subject at a dose, such as a fixed dose, of about 3 mg to about 6 mg, for example, about 4 mg to about 5 mg, about 4.5 mg to about 5 mg, or about 4.6 mg.

The GLP-1R agonist (e.g., GLP-1R/GCGR dual agonist), or pharmaceutically acceptable salt thereof, may be administered to the subject at a dose, such as a fixed dose, of about 5 mg to about 10 mg, for example, about 5 mg to about 8 mg, about 6 mg to about 8 mg, about 6 mg to about 7.5 mg, about 6.5 mg to about 7.5 mg, or about 7 mg.

The GLP-1R agonist (e.g., GLP-1R/GCGR dual agonist), or pharmaceutically acceptable salt thereof, may be administered to the subject at a dose, such as a fixed dose, of about 8 mg to about 10 mg, for example, about 9 mg to about 10 mg, about 9 mg to about 9.5 mg, or about 9.4 mg.

The GLP-1R agonist (e.g., GLP-1R/GCGR dual agonist), or pharmaceutically acceptable salt thereof, may be administered to the subject at a dose, such as a fixed dose, of about 0.15 mg. The GLP-1R agonist (e.g., GLP-1R/GCGR dual agonist), or pharmaceutically acceptable salt thereof, may be administered to the subject at a dose, such as a fixed dose, of about 0.30 mg. The GLP-1R agonist (e.g., GLP-1R/GCGR dual agonist), or pharmaceutically acceptable salt thereof, may be administered to the subject at a dose, such as a fixed dose, of about 0.60 mg. The GLP-1R agonist (e.g., GLP-1R/GCGR dual agonist), or pharmaceutically acceptable salt thereof, may be administered to the subject at a dose, such as a fixed dose, of about 1.20 mg. The GLP-1R agonist (e.g., GLP-1R/GCGR dual agonist), or pharmaceutically acceptable salt thereof, may be administered to the subject at a dose, such as a fixed dose, of about 2.30 mg. The GLP-1R agonist (e.g., GLP-1R/GCGR dual agonist), or pharmaceutically acceptable salt thereof, may be administered to the subject at a dose, such as a fixed dose, of about 4.60 mg. The GLP-1R agonist (e.g., GLP-1R/GCGR dual agonist), or pharmaceutically acceptable salt thereof, may be administered to the subject at a dose, such as a fixed dose, of about 7 mg. The GLP-1R agonist (e.g., GLP-1R/GCGR dual agonist), or pharmaceutically acceptable salt thereof, may be administered to the subject at a dose, such as a fixed dose, of about 9.4 mg.

### AMYR Agonist and GLP-1R Agonist Combination Dosages

The present invention relates to a combination therapy of an AMYR agonist and a GLP-1R agonist (e.g., GLP-1R/GCGR dual agonist), or pharmaceutically acceptable salts thereof.

The present invention encompasses any combination of a fixed dose of AMYR agonist as disclosed herein and a fixed dose of GLP-1R agonist (e.g., GLP-1R/GCGR dual agonist) as disclosed herein (or pharmaceutically acceptable salts thereof).

The AMYR agonist, or pharmaceutically acceptable salt thereof, may be administered to the subject at a dose, such as a fixed dose, of about 1 mg to about 15 mg, and the GLP-1R agonist (e.g., GLP-1R/GCGR dual agonist), or pharmaceutically acceptable salt thereof, may be administered to the subject at a dose, such as a fixed dose, of about 0.1 mg to about 10 mg.

The AMYR agonist, or pharmaceutically acceptable salt thereof, may be administered to the subject at a dose, such as a fixed dose, of about 1 mg to about 15 mg, and the GLP-1R agonist (e.g., GLP-1R/GCGR dual agonist), or pharmaceutically acceptable salt thereof, may be administered to the subject at a dose, such as a fixed dose, of about 0.1 mg to about 9.5 mg (for example, about 0.1 mg to about 9.4 mg).

The AMYR agonist, or pharmaceutically acceptable salt thereof, may be administered to the subject at a dose, such as a fixed dose, of about 1 mg to about 15 mg, and the GLP-1R agonist (e.g., GLP-1R/GCGR dual agonist), or pharmaceutically acceptable salt thereof, may be administered to the subject at a dose, such as a fixed dose, of about 0.1 mg to about 7 mg.

The AMYR agonist, or pharmaceutically acceptable salt thereof, may be administered to the subject at a dose, such as a fixed dose, of about 1 mg to about 15 mg, and the GLP-1R agonist (e.g., GLP-1R/GCGR dual agonist), or pharmaceutically acceptable salt thereof, may be administered to the subject at a dose, such as a fixed dose, of about 0.1 mg to about 5 mg (for example, about 0.1 mg to about 4.60 mg).

The AMYR agonist, or pharmaceutically acceptable salt thereof, may be administered to the subject at a dose, such as a fixed dose, of about 1 mg to about 15 mg, and the GLP-1R agonist (e.g., GLP-1R/GCGR dual agonist), or pharmaceutically acceptable salt thereof, may be administered to the subject at a dose, such as a fixed dose, of about 0.1 mg to about 2.50 mg (for example, about 0.1 mg to about 2.30 mg).

The AMYR agonist, or pharmaceutically acceptable salt thereof, may be administered to the subject at a dose, such as a fixed dose, of about 1 mg to about 15 mg, and the GLP-1R agonist (e.g., GLP-1R/GCGR dual agonist), or pharmaceutically acceptable salt thereof, may be administered to the subject at a dose, such as a fixed dose, of about 0.1 mg to about 1.50 mg (for example, about 0.1 mg to about 1.20 mg).

The AMYR agonist, or pharmaceutically acceptable salt thereof, may be administered to the subject at a dose, such as a fixed dose, of about 1 mg to about 15 mg, and the GLP-1R agonist (e.g., GLP-1R/GCGR dual agonist), or pharmaceutically acceptable salt thereof, may be administered to the subject at a dose, such as a fixed dose, of about 0.25 mg to about 9.5 mg (for example, about 0.30 mg to about 9.4 mg).

The AMYR agonist, or pharmaceutically acceptable salt thereof, may be administered to the subject at a dose, such as a fixed dose, of about 1 mg to about 15 mg, and the GLP-1R agonist (e.g., GLP-1R/GCGR dual agonist), or pharmaceutically acceptable salt thereof, may be administered to the subject at a dose, such as a fixed dose, of about 0.25 mg to about 7 mg (for example, about 0.30 mg to about 7 mg).

The AMYR agonist, or pharmaceutically acceptable salt thereof, may be administered to the subject at a dose, such as a fixed dose, of about 1 mg to about 15 mg, and the GLP-1R agonist (e.g., GLP-1R/GCGR dual agonist), or pharmaceutically acceptable salt thereof, may be administered to the subject at a dose, such as a fixed dose, of about 0.25 mg to about 5 mg (for example, about 0.30 mg to about 4.60 mg).

The AMYR agonist, or pharmaceutically acceptable salt thereof, may be administered to the subject at a dose, such as a fixed dose, of about 1 mg to about 15 mg, and the GLP-1R agonist (e.g., GLP-1R/GCGR dual agonist), or pharmaceutically acceptable salt thereof, may be administered to the subject at a dose, such as a fixed dose, of about 0.25 mg to about 2.5 mg (for example, about 0.30 mg to about 2.30 mg).

The AMYR agonist, or pharmaceutically acceptable salt thereof, may be administered to the subject at a dose, such as a fixed dose, of about 1 mg to about 15 mg, and the GLP-1R agonist (e.g., GLP-1R/GCGR dual agonist), or pharmaceutically acceptable salt thereof, may be administered to the subject at a dose, such as a fixed dose, of about 0.25 mg to about 1.5 mg (for example, about 0.30 mg to about 1.20 mg).

The AMYR agonist, or pharmaceutically acceptable salt thereof, may be administered to the subject at a dose, such as a fixed dose, of about 1 mg to about 15 mg, and the GLP-1R agonist (e.g., GLP-1R/GCGR dual agonist), or pharmaceutically acceptable salt thereof, may be administered to the subject at a dose, such as a fixed dose, of about 0.50 mg to about 9.5 mg (for example, about 0.60 mg to about 9.4 mg).

The AMYR agonist, or pharmaceutically acceptable salt thereof, may be administered to the subject at a dose, such as a fixed dose, of about 1 mg to about 15 mg, and the GLP-1R agonist (e.g., GLP-1R/GCGR dual agonist), or pharmaceutically acceptable salt thereof, may be administered to the subject at a dose, such as a fixed dose, of about 0.50 mg to about 7 mg (for example, about 0.60 mg to about 7 mg).

The AMYR agonist, or pharmaceutically acceptable salt thereof, may be administered to the subject at a dose, such as a fixed dose, of about 1 mg to about 15 mg, and the GLP-1R agonist (e.g., GLP-1R/GCGR dual agonist), or pharmaceutically acceptable salt thereof, may be administered to the subject at a dose, such as a fixed dose, of about 0.50 mg to about 5 mg (for example, about 0.60 mg to about 4.60 mg).

The AMYR agonist, or pharmaceutically acceptable salt thereof, may be administered to the subject at a dose, such as a fixed dose, of about 1 mg to about 15 mg, and the GLP-1R agonist (e.g., GLP-1R/GCGR dual agonist), or pharmaceutically acceptable salt thereof, may be administered to the subject at a dose, such as a fixed dose, of about 0.50 mg to about 2.5 mg (for example, about 0.60 mg to about 2.30 mg).

The AMYR agonist, or pharmaceutically acceptable salt thereof, may be administered to the subject at a dose, such as a fixed dose, of about 1 mg to about 15 mg, and the GLP-1R agonist (e.g., GLP-1R/GCGR dual agonist), or pharmaceutically acceptable salt thereof, may be administered to the subject at a dose, such as a fixed dose, of about 0.50 mg to about 1.5 mg (for example, about 0.60 mg to about 1.20 mg).

The AMYR agonist, or pharmaceutically acceptable salt thereof, may be administered to the subject at a dose, such as a fixed dose, of about 1 mg to about 15 mg, and the GLP-1R agonist (e.g., GLP-1R/GCGR dual agonist), or pharmaceutically acceptable salt thereof, may be administered to the subject at a dose, such as a fixed dose, of about 1 mg to about 9.5 mg (for example, about 1.2 mg to about 9.4 mg).

The AMYR agonist, or pharmaceutically acceptable salt thereof, may be administered to the subject at a dose, such as a fixed dose, of about 1 mg to about 15 mg, and the GLP-1R agonist (e.g., GLP-1R/GCGR dual agonist), or pharmaceutically acceptable salt thereof, may be administered to the subject at a dose, such as a fixed dose, of about 1 mg to about 7 mg (for example, about 1.2 mg to about 7 mg).

The AMYR agonist, or pharmaceutically acceptable salt thereof, may be administered to the subject at a dose, such as a fixed dose, of about 1 mg to about 15 mg, and the GLP-1R agonist (e.g., GLP-1R/GCGR dual agonist), or pharmaceutically acceptable salt thereof, may be administered to the subject at a dose, such as a fixed dose, of about 1 mg to about 5 mg (for example, about 1.2 mg to about 4.60 mg).

The AMYR agonist, or pharmaceutically acceptable salt thereof, may be administered to the subject at a dose, such as a fixed dose, of about 1 mg to about 15 mg, and the GLP-1R agonist (e.g., GLP-1R/GCGR dual agonist), or pharmaceutically acceptable salt thereof, may be administered to the subject at a dose, such as a fixed dose, of about 1 mg to about 2.5 mg (for example, about 1.2 mg to about 2.30 mg).

The AMYR agonist, or pharmaceutically acceptable salt thereof, may be administered to the subject at a dose, such as a fixed dose, of about 1 mg to about 15 mg, and the GLP-1R agonist (e.g., GLP-1R/GCGR dual agonist), or pharmaceutically acceptable salt thereof, may be administered to the subject at a dose, such as a fixed dose, of about 2 mg to about 9.5 mg (for example, about 2.3 mg to about 9.4 mg).

The AMYR agonist, or pharmaceutically acceptable salt thereof, may be administered to the subject at a dose, such as a fixed dose, of about 1 mg to about 15 mg, and the GLP-1R agonist (e.g., GLP-1R/GCGR dual agonist), or pharmaceutically acceptable salt thereof, may be administered to the subject at a dose, such as a fixed dose, of about 2 mg to about 7 mg (for example, about 2.3 mg to about 7 mg).

The AMYR agonist, or pharmaceutically acceptable salt thereof, may be administered to the subject at a dose, such as a fixed dose, of about 1 mg to about 15 mg, and the GLP-1R agonist (e.g., GLP-1R/GCGR dual agonist), or pharmaceutically acceptable salt thereof, may be administered to the subject at a dose, such as a fixed dose, of about 2 mg to about 5 mg (for example, about 2.3 mg to about 4.60 mg).

The AMYR agonist, or pharmaceutically acceptable salt thereof, may be administered to the subject at a dose, such as a fixed dose, of about 1 mg to about 15 mg, and the GLP-1R agonist (e.g., GLP-1R/GCGR dual agonist), or pharmaceutically acceptable salt thereof, may be administered to the subject at a dose, such as a fixed dose, of about 4 mg to about 9.5 mg (for example, about 4.60 mg to about 9.4 mg).

The AMYR agonist, or pharmaceutically acceptable salt thereof, may be administered to the subject at a dose, such as a fixed dose, of about 1 mg to about 15 mg, and the GLP-1R agonist (e.g., GLP-1R/GCGR dual agonist), or pharmaceutically acceptable salt thereof, may be administered to the subject at a dose, such as a fixed dose, of about 4 mg to about 7 mg (for example, about 4.60 mg to about 7 mg).

The AMYR agonist, or pharmaceutically acceptable salt thereof, may be administered to the subject at a dose, such as a fixed dose, of about 1 mg to about 15 mg, and the GLP-1R agonist (e.g., GLP-1R/GCGR dual agonist), or pharmaceutically acceptable salt thereof, may be administered to the subject at a dose, such as a fixed dose, of about 7 mg to about 9.5 mg (for example, about 7 mg to about 9.4 mg).

The AMYR agonist, or pharmaceutically acceptable salt thereof, may be administered to the subject at a dose, such as a fixed dose, of about 1 mg to about 15 mg, and the GLP-1R agonist (e.g., GLP-1R/GCGR dual agonist), or pharmaceutically acceptable salt thereof, may be administered to the subject at a dose, such as a fixed dose, of about 1.2 mg.

The AMYR agonist, or pharmaceutically acceptable salt thereof, may be administered to the subject at a dose, such as a fixed dose, of about 1 mg to about 15 mg, and the GLP-1R agonist (e.g., GLP-1R/GCGR dual agonist), or pharmaceutically acceptable salt thereof, may be administered to the subject at a dose, such as a fixed dose, of about 2.3 mg.

The AMYR agonist, or pharmaceutically acceptable salt thereof, may be administered to the subject at a dose, such as a fixed dose, of about 1 mg to about 15 mg, and the GLP-1R agonist (e.g., GLP-1R/GCGR dual agonist), or pharmaceutically acceptable salt thereof, may be administered to the subject at a dose, such as a fixed dose, of about 4.6 mg.

The AMYR agonist, or pharmaceutically acceptable salt thereof, may be administered to the subject at a dose, such as a fixed dose, of about 1 mg to about 15 mg, and the GLP-1R agonist (e.g., GLP-1R/GCGR dual agonist), or pharmaceutically acceptable salt thereof, may be administered to the subject at a dose, such as a fixed dose, of about 7 mg.

The AMYR agonist, or pharmaceutically acceptable salt thereof, may be administered to the subject at a dose, such as a fixed dose, of about 1 mg to about 15 mg, and the GLP-1R agonist (e.g., GLP-1R/GCGR dual agonist), or pharmaceutically acceptable salt thereof, may be administered to the subject at a dose, such as a fixed dose, of about 9.5 mg (e.g. about 9.4 mg).

The AMYR agonist, or pharmaceutically acceptable salt thereof, may be administered to the subject at a dose, such as a fixed dose, of about 1 mg to about 12 mg, and the GLP-1R agonist (e.g., GLP-1R/GCGR dual agonist), or pharmaceutically acceptable salt thereof, may be administered to the subject at a dose, such as a fixed dose, of about 0.1 mg to about 9.5 mg (for example, about 0.1 mg to about 9.4 mg).

The AMYR agonist, or pharmaceutically acceptable salt thereof, may be administered to the subject at a dose, such as a fixed dose, of about 1 mg to about 12 mg, and the GLP-1R agonist (e.g., GLP-1R/GCGR dual agonist), or pharmaceutically acceptable salt thereof, may be administered to the subject at a dose, such as a fixed dose, of about 0.1 mg to about 7 mg.

The AMYR agonist, or pharmaceutically acceptable salt thereof, may be administered to the subject at a dose, such as a fixed dose, of about 1 mg to about 12 mg, and the GLP-1R agonist (e.g., GLP-1R/GCGR dual agonist), or pharmaceutically acceptable salt thereof, may be administered to the subject at a dose, such as a fixed dose, of about 0.1 mg to about 5 mg (for example, about 0.1 mg to about 4.60 mg).

The AMYR agonist, or pharmaceutically acceptable salt thereof, may be administered to the subject at a dose, such as a fixed dose, of about 1 mg to about 12 mg, and the GLP-1R agonist (e.g., GLP-1R/GCGR dual agonist), or pharmaceutically acceptable salt thereof, may be administered to the subject at a dose, such as a fixed dose, of about 0.1 mg to about 2.50 mg (for example, about 0.1 mg to about 2.30 mg).

The AMYR agonist, or pharmaceutically acceptable salt thereof, may be administered to the subject at a dose, such as a fixed dose, of about 1 mg to about 12 mg, and the GLP-1R agonist (e.g., GLP-1R/GCGR dual agonist), or pharmaceutically acceptable salt thereof, may be administered to the subject at a dose, such as a fixed dose, of about 0.1 mg to about 1.50 mg (for example, about 0.1 mg to about 1.20 mg).

The AMYR agonist, or pharmaceutically acceptable salt thereof, may be administered to the subject at a dose, such as a fixed dose, of about 1 mg to about 12 mg, and the GLP-1R agonist (e.g., GLP-1R/GCGR dual agonist), or pharmaceutically acceptable salt thereof, may be administered to the subject at a dose, such as a fixed dose, of about 0.25 mg to about 9.5 mg (for example, about 0.30 mg to about 9.4 mg).

The AMYR agonist, or pharmaceutically acceptable salt thereof, may be administered to the subject at a dose, such as a fixed dose, of about 1 mg to about 12 mg, and the GLP-1R agonist (e.g., GLP-1R/GCGR dual agonist), or pharmaceutically acceptable salt thereof, may be administered to the subject at a dose, such as a fixed dose, of about 0.25 mg to about 7 mg (for example, about 0.30 mg to about 7 mg).

The AMYR agonist, or pharmaceutically acceptable salt thereof, may be administered to the subject at a dose, such as a fixed dose, of about 1 mg to about 12 mg, and the GLP-1R agonist (e.g., GLP-1R/GCGR dual agonist), or pharmaceutically acceptable salt thereof, may be administered to the subject at a dose, such as a fixed dose, of about 0.25 mg to about 5 mg (for example, about 0.30 mg to about 4.60 mg).

The AMYR agonist, or pharmaceutically acceptable salt thereof, may be administered to the subject at a dose, such as a fixed dose, of about 1 mg to about 12 mg, and the GLP-1R agonist (e.g., GLP-1R/GCGR dual agonist), or pharmaceutically acceptable salt thereof, may be administered to the subject at a dose, such as a fixed dose, of about 0.25 mg to about 2.5 mg (for example, about 0.30 mg to about 2.30 mg).

The AMYR agonist, or pharmaceutically acceptable salt thereof, may be administered to the subject at a dose, such as a fixed dose, of about 1 mg to about 12 mg, and the GLP-1R agonist (e.g., GLP-1R/GCGR dual agonist), or pharmaceutically acceptable salt thereof, may be administered to the subject at a dose, such as a fixed dose, of about 0.25 mg to about 1.5 mg (for example, about 0.30 mg to about 1.20 mg).

The AMYR agonist, or pharmaceutically acceptable salt thereof, may be administered to the subject at a dose, such as a fixed dose, of about 1 mg to about 12 mg, and the GLP-1R agonist (e.g., GLP-1R/GCGR dual agonist), or pharmaceutically acceptable salt thereof, may be administered to the subject at a dose, such as a fixed dose, of about 0.50 mg to about 9.5 mg (for example, about 0.60 mg to about 9.4 mg).

The AMYR agonist, or pharmaceutically acceptable salt thereof, may be administered to the subject at a dose, such as a fixed dose, of about 1 mg to about 12 mg, and the GLP-1R agonist (e.g., GLP-1R/GCGR dual agonist), or pharmaceutically acceptable salt thereof, may be administered to the subject at a dose, such as a fixed dose, of about 0.50 mg to about 7 mg (for example, about 0.60 mg to about 7 mg).

The AMYR agonist, or pharmaceutically acceptable salt thereof, may be administered to the subject at a dose, such as a fixed dose, of about 1 mg to about 12 mg, and the GLP-1R agonist (e.g., GLP-1R/GCGR dual agonist), or pharmaceutically acceptable salt thereof, may be administered to the subject at a dose, such as a fixed dose, of about 0.50 mg to about 5 mg (for example, about 0.60 mg to about 4.60 mg).

The AMYR agonist, or pharmaceutically acceptable salt thereof, may be administered to the subject at a dose, such as a fixed dose, of about 1 mg to about 12 mg, and the GLP-1R agonist (e.g., GLP-1R/GCGR dual agonist), or pharmaceutically acceptable salt thereof, may be administered to the subject at a dose, such as a fixed dose, of about 0.50 mg to about 2.5 mg (for example, about 0.60 mg to about 2.30 mg).

The AMYR agonist, or pharmaceutically acceptable salt thereof, may be administered to the subject at a dose, such as a fixed dose, of about 1 mg to about 12 mg, and the GLP-1R agonist (e.g., GLP-1R/GCGR dual agonist), or pharmaceutically acceptable salt thereof, may be administered to the subject at a dose, such as a fixed dose, of about 0.50 mg to about 1.5 mg (for example, about 0.60 mg to about 1.20 mg).

The AMYR agonist, or pharmaceutically acceptable salt thereof, may be administered to the subject at a dose, such as a fixed dose, of about 1 mg to about 12 mg, and the GLP-1R agonist (e.g., GLP-1R/GCGR dual agonist), or pharmaceutically acceptable salt thereof, may be administered to the subject at a dose, such as a fixed dose, of about 1 mg to about 9.5 mg (for example, about 1.2 mg to about 9.4 mg).

The AMYR agonist, or pharmaceutically acceptable salt thereof, may be administered to the subject at a dose, such as a fixed dose, of about 1 mg to about 12 mg, and the GLP-1R agonist (e.g., GLP-1R/GCGR dual agonist), or pharmaceutically acceptable salt thereof, may be administered to the subject at a dose, such as a fixed dose, of about 1 mg to about 7 mg (for example, about 1.2 mg to about 7 mg).

The AMYR agonist, or pharmaceutically acceptable salt thereof, may be administered to the subject at a dose, such as a fixed dose, of about 1 mg to about 12 mg, and the GLP-1R agonist (e.g., GLP-1R/GCGR dual agonist), or pharmaceutically acceptable salt thereof, may be administered to the subject at a dose, such as a fixed dose, of about 1 mg to about 5 mg (for example, about 1.2 mg to about 4.60 mg).

The AMYR agonist, or pharmaceutically acceptable salt thereof, may be administered to the subject at a dose, such as a fixed dose, of about 1 mg to about 12 mg, and the GLP-1R agonist (e.g., GLP-1R/GCGR dual agonist), or pharmaceutically acceptable salt thereof, may be administered to the subject at a dose, such as a fixed dose, of about 1 mg to about 2.5 mg (for example, about 1.2 mg to about 2.30 mg).

The AMYR agonist, or pharmaceutically acceptable salt thereof, may be administered to the subject at a dose, such as a fixed dose, of about 1 mg to about 12 mg, and the GLP-1R agonist (e.g., GLP-1R/GCGR dual agonist), or pharmaceutically acceptable salt thereof, may be administered to the subject at a dose, such as a fixed dose, of about 2 mg to about 9.5 mg (for example, about 2.3 mg to about 9.4 mg).

The AMYR agonist, or pharmaceutically acceptable salt thereof, may be administered to the subject at a dose, such as a fixed dose, of about 1 mg to about 12 mg, and the GLP-1R agonist (e.g., GLP-1R/GCGR dual agonist), or pharmaceutically acceptable salt thereof, may be administered to the subject at a dose, such as a fixed dose, of about 2 mg to about 7 mg (for example, about 2.3 mg to about 7 mg).

The AMYR agonist, or pharmaceutically acceptable salt thereof, may be administered to the subject at a dose, such as a fixed dose, of about 1 mg to about 12 mg, and the GLP-1R agonist (e.g., GLP-1R/GCGR dual agonist), or pharmaceutically acceptable salt thereof, may be administered to the subject at a dose, such as a fixed dose, of about 2 mg to about 5 mg (for example, about 2.3 mg to about 4.60 mg).

The AMYR agonist, or pharmaceutically acceptable salt thereof, may be administered to the subject at a dose, such as a fixed dose, of about 1 mg to about 12 mg, and the GLP-1R agonist (e.g., GLP-1R/GCGR dual agonist), or pharmaceutically acceptable salt thereof, may be administered to the subject at a dose, such as a fixed dose, of about 4 mg to about 9.5 mg (for example, about 4.60 mg to about 9.4 mg).

The AMYR agonist, or pharmaceutically acceptable salt thereof, may be administered to the subject at a dose, such as a fixed dose, of about 1 mg to about 12 mg, and the GLP-1R agonist (e.g., GLP-1R/GCGR dual agonist), or pharmaceutically acceptable salt thereof, may be administered to the subject at a dose, such as a fixed dose, of about 4 mg to about 7 mg (for example, about 4.60 mg to about 7 mg).

The AMYR agonist, or pharmaceutically acceptable salt thereof, may be administered to the subject at a dose, such as a fixed dose, of about 1 mg to about 12 mg, and the GLP-1R agonist (e.g., GLP-1R/GCGR dual agonist), or pharmaceutically acceptable salt thereof, may be administered to the subject at a dose, such as a fixed dose, of about 7 mg to about 9.5 mg (for example, about 7 mg to about 9.4 mg).

The AMYR agonist, or pharmaceutically acceptable salt thereof, may be administered to the subject at a dose, such as a fixed dose, of about 1 mg to about 12 mg, and the GLP-1R agonist (e.g., GLP-1R/GCGR dual agonist), or pharmaceutically acceptable salt thereof, may be administered to the subject at a dose, such as a fixed dose, of about 1.2 mg.

The AMYR agonist, or pharmaceutically acceptable salt thereof, may be administered to the subject at a dose, such as a fixed dose, of about 1 mg to about 12 mg, and the GLP-1R agonist (e.g., GLP-1R/GCGR dual agonist), or pharmaceutically acceptable salt thereof, may be administered to the subject at a dose, such as a fixed dose, of about 2.3 mg.

The AMYR agonist, or pharmaceutically acceptable salt thereof, may be administered to the subject at a dose, such as a fixed dose, of about 1 mg to about 12 mg, and the GLP-1R agonist (e.g., GLP-1R/GCGR dual agonist), or pharmaceutically acceptable salt thereof, may be administered to the subject at a dose, such as a fixed dose, of about 4.6 mg.

The AMYR agonist, or pharmaceutically acceptable salt thereof, may be administered to the subject at a dose, such as a fixed dose, of about 1 mg to about 12 mg, and the GLP-1R agonist (e.g., GLP-1R/GCGR dual agonist), or pharmaceutically acceptable salt thereof, may be administered to the subject at a dose, such as a fixed dose, of about 7 mg.

The AMYR agonist, or pharmaceutically acceptable salt thereof, may be administered to the subject at a dose, such as a fixed dose, of about 1 mg to about 12 mg, and the GLP-1R agonist (e.g., GLP-1R/GCGR dual agonist), or pharmaceutically acceptable salt thereof, may be administered to the subject at a dose, such as a fixed dose, of about 9.5 mg (e.g. about 9.4 mg).

The AMYR agonist, or pharmaceutically acceptable salt thereof, may be administered to the subject at a dose, such as a fixed dose, of about 1 mg to about 10 mg, and the GLP-1R agonist (e.g., GLP-1R/GCGR dual agonist), or pharmaceutically acceptable salt thereof, may be administered to the subject at a dose, such as a fixed dose, of about 0.1 mg to about 9.5 mg (for example, about 0.1 mg to about 9.4 mg).

The AMYR agonist, or pharmaceutically acceptable salt thereof, may be administered to the subject at a dose, such as a fixed dose, of about 1 mg to about 10 mg (for example, about 1 mg to about 9 mg), and the GLP-1R agonist (e.g., GLP-1R/GCGR dual agonist), or pharmaceutically acceptable salt thereof, may be administered to the subject at a dose, such as a fixed dose, of about 0.1 mg to about 7 mg.

The AMYR agonist, or pharmaceutically acceptable salt thereof, may be administered to the subject at a dose, such as a fixed dose, of about 1 mg to about 10 mg (for example, about 1 mg to about 9 mg), and the GLP-1R agonist (e.g., GLP-1R/GCGR dual agonist), or pharmaceutically acceptable salt thereof, may be administered to the subject at a dose, such as a fixed dose, of about 0.1 mg to about 5 mg (for example, about 0.1 mg to about 4.60 mg).

The AMYR agonist, or pharmaceutically acceptable salt thereof, may be administered to the subject at a dose, such as a fixed dose, of about 1 mg to about 10 mg (for example, about 1 mg to about 9 mg), and the GLP-1R agonist (e.g., GLP-1R/GCGR dual agonist), or pharmaceutically acceptable salt thereof, may be administered to the subject at a dose, such as a fixed dose, of about 0.1 mg to about 2.50 mg (for example, about 0.1 mg to about 2.30 mg).

The AMYR agonist, or pharmaceutically acceptable salt thereof, may be administered to the subject at a dose, such as a fixed dose, of about 1 mg to about 10 mg (for example, about 1 mg to about 9 mg), and the GLP-1R agonist (e.g., GLP-1R/GCGR dual agonist), or pharmaceutically acceptable salt thereof, may be administered to the subject at a dose, such as a fixed dose, of about 0.1 mg to about 1.50 mg (for example, about 0.1 mg to about 1.20 mg).

The AMYR agonist, or pharmaceutically acceptable salt thereof, may be administered to the subject at a dose, such as a fixed dose, of about 1 mg to about 10 mg, and the GLP-1R agonist (e.g., GLP-1R/GCGR dual agonist), or pharmaceutically acceptable salt thereof, may be administered to the subject at a dose, such as a fixed dose, of about 0.25 mg to about 9.5 mg (for example, about 0.30 mg to about 9.4 mg).

The AMYR agonist, or pharmaceutically acceptable salt thereof, may be administered to the subject at a dose, such as a fixed dose, of about 1 mg to about 10 mg (for example, about 1 mg to about 9 mg), and the GLP-1R agonist (e.g., GLP-1R/GCGR dual agonist), or pharmaceutically acceptable salt thereof, may be administered to the subject at a dose, such as a fixed dose, of about 0.25 mg to about 7 mg (for example, about 0.30 mg to about 7 mg).

The AMYR agonist, or pharmaceutically acceptable salt thereof, may be administered to the subject at a dose, such as a fixed dose, of about 1 mg to about 10 mg, and the GLP-1R agonist (e.g., GLP-1R/GCGR dual agonist), or pharmaceutically acceptable salt thereof, may be administered to the subject at a dose, such as a fixed dose, of about 0.25 mg to about 5 mg (for example, about 0.30 mg to about 4.60 mg).

The AMYR agonist, or pharmaceutically acceptable salt thereof, may be administered to the subject at a dose, such as a fixed dose, of about 1 mg to about 10 mg, and the GLP-1R agonist (e.g., GLP-1R/GCGR dual agonist), or pharmaceutically acceptable salt thereof, may be administered to the subject at a dose, such as a fixed dose, of about 0.25 mg to about 2.5 mg (for example, about 0.30 mg to about 2.30 mg).

The AMYR agonist, or pharmaceutically acceptable salt thereof, may be administered to the subject at a dose, such as a fixed dose, of about 1 mg to about 10 mg, and the GLP-1R agonist (e.g., GLP-1R/GCGR dual agonist), or pharmaceutically acceptable salt thereof, may be administered to the subject at a dose, such as a fixed dose, of about 0.25 mg to about 1.5 mg (for example, about 0.30 mg to about 1.20 mg).

The AMYR agonist, or pharmaceutically acceptable salt thereof, may be administered to the subject at a dose, such as a fixed dose, of about 1 mg to about 10 mg, and the GLP-1R agonist (e.g., GLP-1R/GCGR dual agonist), or pharmaceutically acceptable salt thereof, may be administered to the subject at a dose, such as a fixed dose, of about 0.50 mg to about 9.5 mg (for example, about 0.60 mg to about 9.4 mg).

The AMYR agonist, or pharmaceutically acceptable salt thereof, may be administered to the subject at a dose, such as a fixed dose, of about 1 mg to about 10 mg (for example, about 1 mg to about 9 mg), and the GLP-1R agonist (e.g., GLP-1R/GCGR dual agonist), or pharmaceutically acceptable salt thereof, may be administered to the subject at a dose, such as a fixed dose, of about 0.50 mg to about 7 mg (for example, about 0.60 mg to about 7 mg).

The AMYR agonist, or pharmaceutically acceptable salt thereof, may be administered to the subject at a dose, such as a fixed dose, of about 1 mg to about 10 mg, and the GLP-1R agonist (e.g., GLP-1R/GCGR dual agonist), or pharmaceutically acceptable salt thereof, may be administered to the subject at a dose, such as a fixed dose, of about 0.50 mg to about 5 mg (for example, about 0.60 mg to about 4.60 mg).

The AMYR agonist, or pharmaceutically acceptable salt thereof, may be administered to the subject at a dose, such as a fixed dose, of about 1 mg to about 10 mg, and the GLP-1R agonist (e.g., GLP-1R/GCGR dual agonist), or pharmaceutically acceptable salt thereof, may be administered to the subject at a dose, such as a fixed dose, of about 0.50 mg to about 2.5 mg (for example, about 0.60 mg to about 2.30 mg).

The AMYR agonist, or pharmaceutically acceptable salt thereof, may be administered to the subject at a dose, such as a fixed dose, of about 1 mg to about 10 mg, and the GLP-1R agonist (e.g., GLP-1R/GCGR dual agonist), or pharmaceutically acceptable salt thereof, may be administered to the subject at a dose, such as a fixed dose, of about 0.50 mg to about 1.5 mg (for example, about 0.60 mg to about 1.20 mg).

The AMYR agonist, or pharmaceutically acceptable salt thereof, may be administered to the subject at a dose, such as a fixed dose, of about 1 mg to about 10 mg, and the GLP-1R agonist (e.g., GLP-1R/GCGR dual agonist), or pharmaceutically acceptable salt thereof, may be administered to the subject at a dose, such as a fixed dose, of about 1 mg to about 9.5 mg (for example, about 1.2 mg to about 9.4 mg).

The AMYR agonist, or pharmaceutically acceptable salt thereof, may be administered to the subject at a dose, such as a fixed dose, of about 1 mg to about 10 mg (for example, about 1 mg to about 9 mg), and the GLP-1R agonist (e.g., GLP-1R/GCGR dual agonist), or pharmaceutically acceptable salt thereof, may be administered to the subject at a dose, such as a fixed dose, of about 1 mg to about 7 mg (for example, about 1.2 mg to about 7 mg).

The AMYR agonist, or pharmaceutically acceptable salt thereof, may be administered to the subject at a dose, such as a fixed dose, of about 1 mg to about 10 mg (for example, about 1 mg to about 9 mg), and the GLP-1R agonist (e.g., GLP-1R/GCGR dual agonist), or pharmaceutically acceptable salt thereof, may be administered to the subject at a dose, such as a fixed dose, of about 1 mg to about 5 mg (for example, about 1.2 mg to about 4.60 mg).

The AMYR agonist, or pharmaceutically acceptable salt thereof, may be administered to the subject at a dose, such as a fixed dose, of about 1 mg to about 10 mg (for example, about 1 mg to about 9 mg), and the GLP-1R agonist (e.g., GLP-1R/GCGR dual agonist), or pharmaceutically acceptable salt thereof, may be administered to the subject at a dose, such as a fixed dose, of about 1 mg to about 2.5 mg (for example, about 1.2 mg to about 2.30 mg).

The AMYR agonist, or pharmaceutically acceptable salt thereof, may be administered to the subject at a dose, such as a fixed dose, of about 1 mg to about 10 mg, and the GLP-1R agonist (e.g., GLP-1R/GCGR dual agonist), or pharmaceutically acceptable salt thereof, may be administered to the subject at a dose, such as a fixed dose, of about 2 mg to about 9.5 mg (for example, about 2.3 mg to about 9.4 mg).

The AMYR agonist, or pharmaceutically acceptable salt thereof, may be administered to the subject at a dose, such as a fixed dose, of about 1 mg to about 10 mg (for example, about 1 mg to about 9 mg), and the GLP-1R agonist (e.g., GLP-1R/GCGR dual agonist), or pharmaceutically acceptable salt thereof, may be administered to the subject at a dose, such as a fixed dose, of about 2 mg to about 7 mg (for example, about 2.3 mg to about 7 mg).

The AMYR agonist, or pharmaceutically acceptable salt thereof, may be administered to the subject at a dose, such as a fixed dose, of about 1 mg to about 10 mg (for example, about 1 mg to about 9 mg), and the GLP-1R agonist (e.g., GLP-1R/GCGR dual agonist), or pharmaceutically acceptable salt thereof, may be administered to the subject at a dose, such as a fixed dose, of about 2 mg to about 5 mg (for example, about 2.3 mg to about 4.60 mg).

The AMYR agonist, or pharmaceutically acceptable salt thereof, may be administered to the subject at a dose, such as a fixed dose, of about 1 mg to about 10 mg, and the GLP-1R agonist (e.g., GLP-1R/GCGR dual agonist), or pharmaceutically acceptable salt thereof, may be administered to the subject at a dose, such as a fixed dose, of about 4 mg to about 9.5 mg (for example, about 4.60 mg to about 9.4 mg).

The AMYR agonist, or pharmaceutically acceptable salt thereof, may be administered to the subject at a dose, such as a fixed dose, of about 1 mg to about 10 mg (for example, about 1 mg to about 9 mg), and the GLP-1R agonist (e.g., GLP-1R/GCGR dual agonist), or pharmaceutically acceptable salt thereof, may be administered to the subject at a dose, such as a fixed dose, of about 4 mg to about 7 mg (for example, about 4.60 mg to about 7 mg).

The AMYR agonist, or pharmaceutically acceptable salt thereof, may be administered to the subject at a dose, such as a fixed dose, of about 1 mg to about 10 mg, and the GLP-1R agonist (e.g., GLP-1R/GCGR dual agonist), or pharmaceutically acceptable salt thereof, may be administered to the subject at a dose, such as a fixed dose, of about 7 mg to about 9.5 mg (for example, about 7 mg to about 9.4 mg).

The AMYR agonist, or pharmaceutically acceptable salt thereof, may be administered to the subject at a dose, such as a fixed dose, of about 1 mg to about 10 mg, and the GLP-1R agonist (e.g., GLP-1R/GCGR dual agonist), or pharmaceutically acceptable salt thereof, may be administered to the subject at a dose, such as a fixed dose, of about 1.2 mg.

The AMYR agonist, or pharmaceutically acceptable salt thereof, may be administered to the subject at a dose, such as a fixed dose, of about 1 mg to about 10 mg, and the GLP-1R agonist (e.g., GLP-1R/GCGR dual agonist), or pharmaceutically acceptable salt thereof, may be administered to the subject at a dose, such as a fixed dose, of about 2.3 mg.

The AMYR agonist, or pharmaceutically acceptable salt thereof, may be administered to the subject at a dose, such as a fixed dose, of about 1 mg to about 10 mg, and the GLP-1R agonist (e.g., GLP-1R/GCGR dual agonist), or pharmaceutically acceptable salt thereof, may be administered to the subject at a dose, such as a fixed dose, of about 4.6 mg.

The AMYR agonist, or pharmaceutically acceptable salt thereof, may be administered to the subject at a dose, such as a fixed dose, of about 1 mg to about 10 mg, and the GLP-1R agonist (e.g., GLP-1R/GCGR dual agonist), or pharmaceutically acceptable salt thereof, may be administered to the subject at a dose, such as a fixed dose, of about 7 mg.

The AMYR agonist, or pharmaceutically acceptable salt thereof, may be administered to the subject at a dose, such as a fixed dose, of about 1 mg to about 10 mg, and the GLP-1R agonist (e.g., GLP-1R/GCGR dual agonist), or pharmaceutically acceptable salt thereof, may be administered to the subject at a dose, such as a fixed dose, of about 9.5 mg (e.g. about 9.4 mg).

The AMYR agonist, or pharmaceutically acceptable salt thereof, may be administered to the subject at a dose, such as a fixed dose, of about 1 mg to about 15 mg, and the GLP-1R agonist (e.g., GLP-1R/GCGR dual agonist), or pharmaceutically acceptable salt thereof, may be administered to the subject at a dose, such as a fixed dose, of about 0.1 mg to about 10 mg.

The AMYR agonist, or pharmaceutically acceptable salt thereof, may be administered to the subject at a dose, such as a fixed dose, of about 1 mg to about 12 mg, and the GLP-1R agonist (e.g., GLP-1R/GCGR dual agonist), or pharmaceutically acceptable salt thereof, may be administered to the subject at a dose, such as a fixed dose, of about 0.1 mg to about 10 mg.

The AMYR agonist, or pharmaceutically acceptable salt thereof, may be administered to the subject at a dose, such as a fixed dose, of about 1 mg to about 10 mg (e.g. about 1.5 mg to about 10 mg), and the GLP-1R agonist (e.g., GLP-1R/GCGR dual agonist), or pharmaceutically acceptable salt thereof, may be administered to the subject at a dose, such as a fixed dose, of about 0.1 mg to about 10 mg.

The AMYR agonist, or pharmaceutically acceptable salt thereof, may be administered to the subject at a dose, such as a fixed dose, of about 1 mg to about 9 mg (for example, about 1.5 mg to about 9 mg), and the GLP-1R agonist (e.g., GLP-1R/GCGR dual agonist), or pharmaceutically acceptable salt thereof, may be administered to the subject at a dose, such as a fixed dose, of about 0.1 mg to about 10 mg.

The AMYR agonist, or pharmaceutically acceptable salt thereof, may be administered to the subject at a dose, such as a fixed dose, of about 1 mg to about 7 mg (for example, about 1.5 mg to about 7 mg), and the GLP-1R agonist (e.g., GLP-1R/GCGR dual agonist), or pharmaceutically acceptable salt thereof, may be administered to the subject at a dose, such as a fixed dose, of about 0.1 mg to about 10 mg.

The AMYR agonist, or pharmaceutically acceptable salt thereof, may be administered to the subject at a dose, such as a fixed dose, of about 1 mg to about 5 mg (for example, about 1.5 mg to about 5 mg, or about 1.5 mg to about 4.5 mg), and the GLP-1R agonist (e.g., GLP-1R/GCGR dual agonist), or pharmaceutically acceptable salt thereof, may be administered to the subject at a dose, such as a fixed dose, of about 0.1 mg to about 10 mg.

The AMYR agonist, or pharmaceutically acceptable salt thereof, may be administered to the subject at a dose, such as a fixed dose, of about 1 mg to about 3 mg (e.g., about 1.5 mg to about 3 mg, e.g. about 1.5 mg, or about 1.6 mg), and the GLP-1R agonist (e.g., GLP-1R/GCGR dual agonist), or pharmaceutically acceptable salt thereof, may be administered to the subject at a dose, such as a fixed dose, of about 0.1 mg to about 10 mg.

The AMYR agonist, or pharmaceutically acceptable salt thereof, may be administered to the subject at a dose, such as a fixed dose, of about 4 mg to about 10 mg (for example, about 4.5 mg to about 10 mg, about 4 mg to about 9 mg, or 4.5 mg to about 9 mg), and the GLP-1R agonist (e.g., GLP-1R/GCGR dual agonist), or pharmaceutically acceptable salt thereof, may be administered to the subject at a dose, such as a fixed dose, of about 0.1 mg to about 10 mg.

The AMYR agonist, or pharmaceutically acceptable salt thereof, may be administered to the subject at a dose, such as a fixed dose, of about 4 mg to about 7 mg (e.g., about 4.5 mg to about 7 mg, or about 6 mg), and the GLP-1R agonist (e.g., GLP-1R/GCGR dual agonist), or pharmaceutically acceptable salt thereof, may be administered to the subject at a dose, such as a fixed dose, of about 0.1 mg to about 10 mg.

The AMYR agonist, or pharmaceutically acceptable salt thereof, may be administered to the subject at a dose, such as a fixed dose, of about 4 mg to about 5 mg (e.g., about 4.5 mg), and the GLP-1R agonist (e.g., GLP-1R/GCGR dual agonist), or pharmaceutically acceptable salt thereof, may be administered to the subject at a dose, such as a fixed dose, of about 0.1 mg to about 10 mg.

The AMYR agonist, or pharmaceutically acceptable salt thereof, may be administered to the subject at a dose, such as a fixed dose, of about 7 mg to about 10 mg (e.g., about 7 mg to about 9 mg, or about 9 mg), and the GLP-1R agonist (e.g., GLP-1R/GCGR dual agonist), or pharmaceutically acceptable salt thereof, may be administered to the subject at a dose, such as a fixed dose, of about 0.1 mg to about 10 mg.

The AMYR agonist, or pharmaceutically acceptable salt thereof, may be administered to the subject at a dose, such as a fixed dose, of about 1.5 mg and the GLP-1R agonist (e.g., GLP-1R/GCGR dual agonist), or pharmaceutically acceptable salt thereof, may be administered to the subject at a dose, such as a fixed dose, of about 0.1 mg to about 10 mg.

The AMYR agonist, or pharmaceutically acceptable salt thereof, may be administered to the subject at a dose, such as a fixed dose, of about 1.6 mg and the GLP-1R agonist (e.g., GLP-1R/GCGR dual agonist), or pharmaceutically acceptable salt thereof, may be administered to the subject at a dose, such as a fixed dose, of about 0.1 mg to about 10 mg.

The AMYR agonist, or pharmaceutically acceptable salt thereof, may be administered to the subject at a dose, such as a fixed dose, of about 4.5 mg and the GLP-1R agonist (e.g., GLP-1R/GCGR dual agonist), or pharmaceutically acceptable salt thereof, may be administered to the subject at a dose, such as a fixed dose, of about 0.1 mg to about 10 mg.

The AMYR agonist, or pharmaceutically acceptable salt thereof, may be administered to the subject at a dose, such as a fixed dose, of about 6 mg, and the GLP-1R agonist (e.g., GLP-1R/GCGR dual agonist), or pharmaceutically acceptable salt thereof, may be administered to the subject at a dose, such as a fixed dose, of about 0.1 mg to about 10 mg.

The AMYR agonist, or pharmaceutically acceptable salt thereof, may be administered to the subject at a dose, such as a fixed dose, of about 9 mg, and the GLP-1R agonist (e.g., GLP-1R/GCGR dual agonist), or pharmaceutically acceptable salt thereof, may be administered to the subject at a dose, such as a fixed dose, of about 0.1 mg to about 10 mg.

The AMYR agonist, or pharmaceutically acceptable salt thereof, may be administered to the subject at a dose, such as a fixed dose, of about 1 mg to about 15 mg, and the GLP-1R agonist (e.g., GLP-1R/GCGR dual agonist), or pharmaceutically acceptable salt thereof, may be administered to the subject at a dose, such as a fixed dose, of about 0.1 mg to about 9.4 mg.

The AMYR agonist, or pharmaceutically acceptable salt thereof, may be administered to the subject at a dose, such as a fixed dose, of about 1 mg to about 12 mg, and the GLP-1R agonist (e.g., GLP-1R/GCGR dual agonist), or pharmaceutically acceptable salt thereof, may be administered to the subject at a dose, such as a fixed dose, of about 0.1 mg to about 9.4 mg.

The AMYR agonist, or pharmaceutically acceptable salt thereof, may be administered to the subject at a dose, such as a fixed dose, of about 1 mg to about 10 mg (for example, about 1.5 mg to about 10 mg), and the GLP-1R agonist (e.g., GLP-1R/GCGR dual agonist), or pharmaceutically acceptable salt thereof, may be administered to the subject at a dose, such as a fixed dose, of about 0.1 mg to about 9.4 mg.

The AMYR agonist, or pharmaceutically acceptable salt thereof, may be administered to the subject at a dose, such as a fixed dose, of about 1 mg to about 9 mg (for example, about 1.5 mg to about 9 mg), and the GLP-1R agonist (e.g., GLP-1R/GCGR dual agonist), or pharmaceutically acceptable salt thereof, may be administered to the subject at a dose, such as a fixed dose, of about 0.1 mg to about 9.4 mg.

The AMYR agonist, or pharmaceutically acceptable salt thereof, may be administered to the subject at a dose, such as a fixed dose, of about 1 mg to about 7 mg (for example, about 1.5 mg to about 7 mg), and the GLP-1R agonist (e.g., GLP-1R/GCGR dual agonist), or pharmaceutically acceptable salt thereof, may be administered to the subject at a dose, such as a fixed dose, of about 0.1 mg to about 9.4 mg.

The AMYR agonist, or pharmaceutically acceptable salt thereof, may be administered to the subject at a dose, such as a fixed dose, of about 1 mg to about 5 mg (for example, about 1.5 mg to about 5 mg, or about 1.5 mg to about 4.5 mg), and the GLP-1R agonist (e.g., GLP-1R/GCGR dual agonist), or pharmaceutically acceptable salt thereof, may be administered to the subject at a dose, such as a fixed dose, of about 0.1 mg to about 9.4 mg.

The AMYR agonist, or pharmaceutically acceptable salt thereof, may be administered to the subject at a dose, such as a fixed dose, of about 1 mg to about 3 mg (e.g., about 1.5 mg to about 3 mg, e.g. about 1.5 mg, or about 1.6 mg), and the GLP-1R agonist (e.g., GLP-1R/GCGR dual agonist), or pharmaceutically acceptable salt thereof, may be administered to the subject at a dose, such as a fixed dose, of about 0.1 mg to about 9.4 mg.

The AMYR agonist, or pharmaceutically acceptable salt thereof, may be administered to the subject at a dose, such as a fixed dose, of about 4 mg to about 10 mg (for example, about 4.5 mg to about 10 mg, about 4 mg to about 9 mg, or 4.5 mg to about 9 mg), and the GLP-1R agonist (e.g., GLP-1R/GCGR dual agonist), or pharmaceutically acceptable salt thereof, may be administered to the subject at a dose, such as a fixed dose, of about 0.1 mg to about 9.4 mg.

The AMYR agonist, or pharmaceutically acceptable salt thereof, may be administered to the subject at a dose, such as a fixed dose, of about 4 mg to about 7 mg (e.g., e.g., about 4.5 mg to about 7 mg, or about 6 mg), and the GLP-1R agonist (e.g., GLP-1R/GCGR dual agonist), or pharmaceutically acceptable salt thereof, may be administered to the subject at a dose, such as a fixed dose, of about 0.1 mg to about 9.4 mg.

The AMYR agonist, or pharmaceutically acceptable salt thereof, may be administered to the subject at a dose, such as a fixed dose, of about 4 mg to about 5 mg (e.g., about 4.5 mg), and the GLP-1R agonist (e.g., GLP-1R/GCGR dual agonist), or pharmaceutically acceptable salt thereof, may be administered to the subject at a dose, such as a fixed dose, of about 0.1 mg to about 9.4 mg.

The AMYR agonist, or pharmaceutically acceptable salt thereof, may be administered to the subject at a dose, such as a fixed dose, of about 7 mg to about 10 mg (e.g., about 7 mg to about 9 mg, or about 9 mg), and the GLP-1R agonist (e.g., GLP-1R/GCGR dual agonist), or pharmaceutically acceptable salt thereof, may be administered to the subject at a dose, such as a fixed dose, of about 0.1 mg to about 9.4 mg.

The AMYR agonist, or pharmaceutically acceptable salt thereof, may be administered to the subject at a dose, such as a fixed dose, of about 1.5 mg and the GLP-1R agonist (e.g., GLP-1R/GCGR dual agonist), or pharmaceutically acceptable salt thereof, may be administered to the subject at a dose, such as a fixed dose, of about 0.1 mg to about 9.4 mg.

The AMYR agonist, or pharmaceutically acceptable salt thereof, may be administered to the subject at a dose, such as a fixed dose, of about 1.6 mg and the GLP-1R agonist (e.g., GLP-1R/GCGR dual agonist), or pharmaceutically acceptable salt thereof, may be administered to the subject at a dose, such as a fixed dose, of about 0.1 mg to about 9.4 mg.

The AMYR agonist, or pharmaceutically acceptable salt thereof, may be administered to the subject at a dose, such as a fixed dose, of about 4.5 mg and the GLP-1R agonist (e.g., GLP-1R/GCGR dual agonist), or pharmaceutically acceptable salt thereof, may be administered to the subject at a dose, such as a fixed dose, of about 0.1 mg to about 9.4 mg.

The AMYR agonist, or pharmaceutically acceptable salt thereof, may be administered to the subject at a dose, such as a fixed dose, of about 6 mg, and the GLP-1R agonist (e.g., GLP-1R/GCGR dual agonist), or pharmaceutically acceptable salt thereof, may be administered to the subject at a dose, such as a fixed dose, of about 0.1 mg to about 9.4 mg.

The AMYR agonist, or pharmaceutically acceptable salt thereof, may be administered to the subject at a dose, such as a fixed dose, of about 9 mg, and the GLP-1R agonist (e.g., GLP-1R/GCGR dual agonist), or pharmaceutically acceptable salt thereof, may be administered to the subject at a dose, such as a fixed dose, of about 0.1 mg to about 9.4 mg.

The AMYR agonist, or pharmaceutically acceptable salt thereof, may be administered to the subject at a dose, such as a fixed dose, of about 1 mg to about 15 mg, and the GLP-1R agonist (e.g., GLP-1R/GCGR dual agonist), or pharmaceutically acceptable salt thereof, may be administered to the subject at a dose, such as a fixed dose, of about 0.1 mg to about 7 mg.

The AMYR agonist, or pharmaceutically acceptable salt thereof, may be administered to the subject at a dose, such as a fixed dose, of about 1 mg to about 12 mg, and the GLP-1R agonist (e.g., GLP-1R/GCGR dual agonist), or pharmaceutically acceptable salt thereof, may be administered to the subject at a dose, such as a fixed dose, of about 0.1 mg to about 7 mg.

The AMYR agonist, or pharmaceutically acceptable salt thereof, may be administered to the subject at a dose, such as a fixed dose, of about 1 mg to about 10 mg (e.g. about 1.5 mg to about 10 mg), and the GLP-1R agonist (e.g., GLP-1R/GCGR dual agonist), or pharmaceutically acceptable salt thereof, may be administered to the subject at a dose, such as a fixed dose, of about 0.1 mg to about 7 mg.

The AMYR agonist, or pharmaceutically acceptable salt thereof, may be administered to the subject at a dose, such as a fixed dose, of about 1 mg to about 9 mg (for example, about 1.5 mg to about 9 mg), and the GLP-1R agonist (e.g., GLP-1R/GCGR dual agonist), or pharmaceutically acceptable salt thereof, may be administered to the subject at a dose, such as a fixed dose, of about 0.1 mg to about 7 mg.

The AMYR agonist, or pharmaceutically acceptable salt thereof, may be administered to the subject at a dose, such as a fixed dose, of about 1 mg to about 7 mg (for example, about 1.5 mg to about 7 mg), and the GLP-1R agonist (e.g., GLP-1R/GCGR dual agonist), or pharmaceutically acceptable salt thereof, may be administered to the subject at a dose, such as a fixed dose, of about 0.1 mg to about 7 mg.

The AMYR agonist, or pharmaceutically acceptable salt thereof, may be administered to the subject at a dose, such as a fixed dose, of about 1 mg to about 5 mg (for example, about 1.5 mg to about 5 mg, or about 1.5 mg to about 4.5 mg), and the GLP-1R agonist (e.g., GLP-1R/GCGR dual agonist), or pharmaceutically acceptable salt thereof, may be administered to the subject at a dose, such as a fixed dose, of about 0.1 mg to about 7 mg.

The AMYR agonist, or pharmaceutically acceptable salt thereof, may be administered to the subject at a dose, such as a fixed dose, of about 1 mg to about 3 mg (e.g., about 1.5 mg to about 3 mg, e.g. about 1.5 mg, or about 1.6 mg), and the GLP-1R agonist (e.g., GLP-1R/GCGR dual agonist), or pharmaceutically acceptable salt thereof, may be administered to the subject at a dose, such as a fixed dose, of about 0.1 mg to about 7 mg.

The AMYR agonist, or pharmaceutically acceptable salt thereof, may be administered to the subject at a dose, such as a fixed dose, of about 4 mg to about 10 mg (for example, about 4.5 mg to about 10 mg, about 4 mg to about 9 mg, or 4.5 mg to about 9 mg), and the GLP-1R agonist (e.g., GLP-1R/GCGR dual agonist), or pharmaceutically acceptable salt thereof, may be administered to the subject at a dose, such as a fixed dose, of about 0.1 mg to about 7 mg.

The AMYR agonist, or pharmaceutically acceptable salt thereof, may be administered to the subject at a dose, such as a fixed dose, of about 4 mg to about 7 mg (e.g., about 4.5 mg to about 7 mg, or about 6 mg), and the GLP-1R agonist (e.g., GLP-1R/GCGR dual agonist), or pharmaceutically acceptable salt thereof, may be administered to the subject at a dose, such as a fixed dose, of about 0.1 mg to about 7 mg.

The AMYR agonist, or pharmaceutically acceptable salt thereof, may be administered to the subject at a dose, such as a fixed dose, of about 4 mg to about 5 mg (e.g., about 4.5 mg), and the GLP-1R agonist (e.g., GLP-1R/GCGR dual agonist), or pharmaceutically acceptable salt thereof, may be administered to the subject at a dose, such as a fixed dose, of about 0.1 mg to about 7 mg.

The AMYR agonist, or pharmaceutically acceptable salt thereof, may be administered to the subject at a dose, such as a fixed dose, of about 7 mg to about 10 mg (e.g., about 7 mg to about 9 mg, or about 9 mg), and the GLP-1R agonist (e.g., GLP-1R/GCGR dual agonist), or pharmaceutically acceptable salt thereof, may be administered to the subject at a dose, such as a fixed dose, of about 0.1 mg to about 7 mg.

The AMYR agonist, or pharmaceutically acceptable salt thereof, may be administered to the subject at a dose, such as a fixed dose, of about 1.5 mg and the GLP-1R agonist (e.g., GLP-1R/GCGR dual agonist), or pharmaceutically acceptable salt thereof, may be administered to the subject at a dose, such as a fixed dose, of about 0.1 mg to about 7 mg.

The AMYR agonist, or pharmaceutically acceptable salt thereof, may be administered to the subject at a dose, such as a fixed dose, of about 1.6 mg and the GLP-1R agonist (e.g., GLP-1R/GCGR dual agonist), or pharmaceutically acceptable salt thereof, may be administered to the subject at a dose, such as a fixed dose, of about 0.1 mg to about 7 mg.

The AMYR agonist, or pharmaceutically acceptable salt thereof, may be administered to the subject at a dose, such as a fixed dose, of about 4.5 mg and the GLP-1R agonist (e.g., GLP-1R/GCGR dual agonist), or pharmaceutically acceptable salt thereof, may be administered to the subject at a dose, such as a fixed dose, of about 0.1 mg to about 7 mg.

The AMYR agonist, or pharmaceutically acceptable salt thereof, may be administered to the subject at a dose, such as a fixed dose, of about 6 mg, and the GLP-1R agonist (e.g., GLP-1R/GCGR dual agonist), or pharmaceutically acceptable salt thereof, may be administered to the subject at a dose, such as a fixed dose, of about 0.1 mg to about 7 mg.

The AMYR agonist, or pharmaceutically acceptable salt thereof, may be administered to the subject at a dose, such as a fixed dose, of about 9 mg, and the GLP-1R agonist (e.g., GLP-1R/GCGR dual agonist), or pharmaceutically acceptable salt thereof, may be administered to the subject at a dose, such as a fixed dose, of about 0.1 mg to about 7 mg.

The AMYR agonist, or pharmaceutically acceptable salt thereof, may be administered to the subject at a dose, such as a fixed dose, of about 7 mg to about 10 mg (e.g., about 9 mg), and the GLP-1R agonist (e.g., GLP-1R/GCGR dual agonist), or pharmaceutically acceptable salt thereof, may be administered to the subject at a dose, such as a fixed dose, of about 8 mg to about 10 mg (e.g. about 9.4 mg).

The AMYR agonist, or pharmaceutically acceptable salt thereof, may be administered to the subject at a dose, such as a fixed dose, of about 7 mg to about 10 mg (e.g., about 9 mg), and the GLP-1R agonist (e.g., GLP-1R/GCGR dual agonist), or pharmaceutically acceptable salt thereof, may be administered to the subject at a dose, such as a fixed dose, of about 5 mg to about 10 mg (e.g. about 7 mg).

The AMYR agonist, or pharmaceutically acceptable salt thereof, may be administered to the subject at a dose, such as a fixed dose, of about 7 mg to about 10 mg (e.g., about 9 mg), and the GLP-1R agonist (e.g., GLP-1R/GCGR dual agonist), or pharmaceutically acceptable salt thereof, may be administered to the subject at a dose, such as a fixed dose, of about 3 mg to about 5 mg (e.g. about 4.6 mg).

The AMYR agonist, or pharmaceutically acceptable salt thereof, may be administered to the subject at a dose, such as a fixed dose, of about 7 mg to about 10 mg (e.g., about 9 mg), and the GLP-1R agonist (e.g., GLP-1R/GCGR dual agonist), or pharmaceutically acceptable salt thereof, may be administered to the subject at a dose, such as a fixed dose, of about 2 mg to about 4 mg (e.g. about 2.3 mg).

The AMYR agonist, or pharmaceutically acceptable salt thereof, may be administered to the subject at a dose, such as a fixed dose, of about 7 mg to about 10 mg (e.g., about 9 mg), and the GLP-1R agonist (e.g., GLP-1R/GCGR dual agonist), or pharmaceutically acceptable salt thereof, may be administered to the subject at a dose, such as a fixed dose, of about 1 mg to about 2 mg (e.g. about 1.2 mg).

The AMYR agonist, or pharmaceutically acceptable salt thereof, may be administered to the subject at a dose, such as a fixed dose, of about 4 mg to about 7 mg (e.g. about 6 mg), and the GLP-1R agonist (e.g., GLP-1R/GCGR dual agonist), or pharmaceutically acceptable salt thereof, may be administered to the subject at a dose, such as a fixed dose, of about 8 mg to about 10 mg (e.g. about 9.4 mg).

The AMYR agonist, or pharmaceutically acceptable salt thereof, may be administered to the subject at a dose, such as a fixed dose, of about 4 mg to about 7 mg (e.g. about 6 mg), and the GLP-1R agonist (e.g., GLP-1R/GCGR dual agonist), or pharmaceutically acceptable salt thereof, may be administered to the subject at a dose, such as a fixed dose, of about 5 mg to about 10 mg (e.g. about 7 mg).

The AMYR agonist, or pharmaceutically acceptable salt thereof, may be administered to the subject at a dose, such as a fixed dose, of about 4 mg to about 7 mg (e.g. about 6 mg), and the GLP-1R agonist (e.g., GLP-1R/GCGR dual agonist), or pharmaceutically acceptable salt thereof, may be administered to the subject at a dose, such as a fixed dose, of about 3 mg to about 5 mg (e.g. about 4.6 mg).

The AMYR agonist, or pharmaceutically acceptable salt thereof, may be administered to the subject at a dose, such as a fixed dose, of about 4 mg to about 7 mg (e.g. about 6 mg), and the GLP-1R agonist (e.g., GLP-1R/GCGR dual agonist), or pharmaceutically acceptable salt thereof, may be administered to the subject at a dose, such as a fixed dose, of about 2 mg to about 4 mg (e.g. about 2.3 mg).

The AMYR agonist, or pharmaceutically acceptable salt thereof, may be administered to the subject at a dose, such as a fixed dose, of about 4 mg to about 7 mg (e.g. about 6 mg), and the GLP-1R agonist (e.g., GLP-1R/GCGR dual agonist), or pharmaceutically acceptable salt thereof, may be administered to the subject at a dose, such as a fixed dose, of about 1 mg to about 2 mg (e.g. about 1.2 mg).

The AMYR agonist, or pharmaceutically acceptable salt thereof, may be administered to the subject at a dose, such as a fixed dose, of about 4 mg to about 5 mg (e.g. about 4.5 mg), and the GLP-1R agonist (e.g., GLP-1R/GCGR dual agonist), or pharmaceutically acceptable salt thereof, may be administered to the subject at a dose, such as a fixed dose, of about 8 mg to about 10 mg (e.g. about 9.4 mg).

The AMYR agonist, or pharmaceutically acceptable salt thereof, may be administered to the subject at a dose, such as a fixed dose, of about 4 mg to about 5 mg (e.g. about 4.5 mg), and the GLP-1R agonist (e.g., GLP-1R/GCGR dual agonist), or pharmaceutically acceptable salt thereof, may be administered to the subject at a dose, such as a fixed dose, of about 5 mg to about 10 mg (e.g. about 7 mg).

The AMYR agonist, or pharmaceutically acceptable salt thereof, may be administered to the subject at a dose, such as a fixed dose, of about 4 mg to about 5 mg (e.g. about 4.5 mg), and the GLP-1R agonist (e.g., GLP-1R/GCGR dual agonist), or pharmaceutically acceptable salt thereof, may be administered to the subject at a dose, such as a fixed dose, of about 3 mg to about 5 mg (e.g. about 4.6 mg).

The AMYR agonist, or pharmaceutically acceptable salt thereof, may be administered to the subject at a dose, such as a fixed dose, of about 4 mg to about 5 mg (e.g. about 4.5 mg), and the GLP-1R agonist (e.g., GLP-1R/GCGR dual agonist), or pharmaceutically acceptable salt thereof, may be administered to the subject at a dose, such as a fixed dose, of about 2 mg to about 4 mg (e.g. about 2.3 mg).

The AMYR agonist, or pharmaceutically acceptable salt thereof, may be administered to the subject at a dose, such as a fixed dose, of about 4 mg to about 5 mg (e.g. about 4.5 mg), and the GLP-1R agonist (e.g., GLP-1R/GCGR dual agonist), or pharmaceutically acceptable salt thereof, may be administered to the subject at a dose, such as a fixed dose, of about 1 mg to about 2 mg (e.g. about 1.2 mg).

The AMYR agonist, or pharmaceutically acceptable salt thereof, may be administered to the subject at a dose, such as a fixed dose, of about 1 mg to about 2 mg (for example, about 1.50 mg or about 1.60 mg), and the GLP-1R agonist (e.g., GLP-1R/GCGR dual agonist), or pharmaceutically acceptable salt thereof, may be administered to the subject at a dose, such as a fixed dose, of about 8 mg to about 10 mg (e.g. about 9.4 mg).

The AMYR agonist, or pharmaceutically acceptable salt thereof, may be administered to the subject at a dose, such as a fixed dose, of about 1 mg to about 2 mg (for example, about 1.50 mg or about 1.60 mg), and the GLP-1R agonist (e.g., GLP-1R/GCGR dual agonist), or pharmaceutically acceptable salt thereof, may be administered to the subject at a dose, such as a fixed dose, of about 5 mg to about 10 mg (e.g. about 7 mg).

The AMYR agonist, or pharmaceutically acceptable salt thereof, may be administered to the subject at a dose, such as a fixed dose, of about 1 mg to about 2 mg (for example, about 1.50 mg or about 1.60 mg), and the GLP-1R agonist (e.g., GLP-1R/GCGR dual agonist), or pharmaceutically acceptable salt thereof, may be administered to the subject at a dose, such as a fixed dose, of about 3 mg to about 5 mg (e.g. about 4.6 mg).

The AMYR agonist, or pharmaceutically acceptable salt thereof, may be administered to the subject at a dose, such as a fixed dose, of about 1 mg to about 2 mg (for example, about 1.50 mg or about 1.60 mg), and the GLP-1R agonist (e.g., GLP-1R/GCGR dual agonist), or pharmaceutically acceptable salt thereof, may be administered to the subject at a dose, such as a fixed dose, of about 2 mg to about 4 mg (e.g. about 2.3 mg).

The AMYR agonist, or pharmaceutically acceptable salt thereof, may be administered to the subject at a dose, such as a fixed dose, of about 1 mg to about 2 mg (for example, about 1.50 mg or about 1.60 mg), and the GLP-1R agonist (e.g., GLP-1R/GCGR dual agonist), or pharmaceutically acceptable salt thereof, may be administered to the subject at a dose, such as a fixed dose, of about 1 mg to about 2 mg (e.g. about 1.2 mg).

The AMYR agonist, or pharmaceutically acceptable salt thereof, may be administered to the subject at a dose, such as a fixed dose, of about 9 mg, and the GLP-1R agonist (e.g., GLP-1R/GCGR dual agonist), or pharmaceutically acceptable salt thereof, may be administered to the subject at a dose, such as a fixed dose, of about 9.4 mg.

The AMYR agonist, or pharmaceutically acceptable salt thereof, may be administered to the subject at a dose, such as a fixed dose, of about 9 mg, and the GLP-1R agonist (e.g., GLP-1R/GCGR dual agonist), or pharmaceutically acceptable salt thereof, may be administered to the subject at a dose, such as a fixed dose, of about 7 mg.

The AMYR agonist, or pharmaceutically acceptable salt thereof, may be administered to the subject at a dose, such as a fixed dose, of about 9 mg, and the GLP-1R agonist (e.g., GLP-1R/GCGR dual agonist), or pharmaceutically acceptable salt thereof, may be administered to the subject at a dose, such as a fixed dose, of about 4.6 mg.

The AMYR agonist, or pharmaceutically acceptable salt thereof, may be administered to the subject at a dose, such as a fixed dose, of about 9 mg, and the GLP-1R agonist (e.g., GLP-1R/GCGR dual agonist), or pharmaceutically acceptable salt thereof, may be administered to the subject at a dose, such as a fixed dose, of about 2.3 mg.

The AMYR agonist, or pharmaceutically acceptable salt thereof, may be administered to the subject at a dose, such as a fixed dose, of about 9 mg, and the GLP-1R agonist (e.g., GLP-1R/GCGR dual agonist), or pharmaceutically acceptable salt thereof, may be administered to the subject at a dose, such as a fixed dose, of about 1.2 mg.

The AMYR agonist, or pharmaceutically acceptable salt thereof, may be administered to the subject at a dose, such as a fixed dose, of about 6 mg, and the GLP-1R agonist (e.g., GLP-1R/GCGR dual agonist), or pharmaceutically acceptable salt thereof, may be administered to the subject at a dose, such as a fixed dose, of about 9.4 mg.

The AMYR agonist, or pharmaceutically acceptable salt thereof, may be administered to the subject at a dose, such as a fixed dose, of about 6 mg, and the GLP-1R agonist (e.g., GLP-1R/GCGR dual agonist), or pharmaceutically acceptable salt thereof, may be administered to the subject at a dose, such as a fixed dose, of about 7 mg.

The AMYR agonist, or pharmaceutically acceptable salt thereof, may be administered to the subject at a dose, such as a fixed dose, of about 6 mg, and the GLP-1R agonist (e.g., GLP-1R/GCGR dual agonist), or pharmaceutically acceptable salt thereof, may be administered to the subject at a dose, such as a fixed dose, of about 4.6 mg.

The AMYR agonist, or pharmaceutically acceptable salt thereof, may be administered to the subject at a dose, such as a fixed dose, of about 6 mg, and the GLP-1R agonist (e.g., GLP-1R/GCGR dual agonist), or pharmaceutically acceptable salt thereof, may be administered to the subject at a dose, such as a fixed dose, of about 2.3 mg.

The AMYR agonist, or pharmaceutically acceptable salt thereof, may be administered to the subject at a dose, such as a fixed dose, of about 6 mg, and the GLP-1R agonist (e.g., GLP-1R/GCGR dual agonist), or pharmaceutically acceptable salt thereof, may be administered to the subject at a dose, such as a fixed dose, of about 1.2 mg.

The AMYR agonist, or pharmaceutically acceptable salt thereof, may be administered to the subject at a dose, such as a fixed dose, of about 4.5 mg, and the GLP-1R agonist (e.g., GLP-1R/GCGR dual agonist), or pharmaceutically acceptable salt thereof, may be administered to the subject at a dose, such as a fixed dose, of about 9.4 mg.

The AMYR agonist, or pharmaceutically acceptable salt thereof, may be administered to the subject at a dose, such as a fixed dose, of about 4.5 mg, and the GLP-1R agonist (e.g., GLP-1R/GCGR dual agonist), or pharmaceutically acceptable salt thereof, may be administered to the subject at a dose, such as a fixed dose, of about 7 mg.

The AMYR agonist, or pharmaceutically acceptable salt thereof, may be administered to the subject at a dose, such as a fixed dose, of about 4.5 mg, and the GLP-1R agonist (e.g., GLP-1R/GCGR dual agonist), or pharmaceutically acceptable salt thereof, may be administered to the subject at a dose, such as a fixed dose, of about 4.6 mg.

The AMYR agonist, or pharmaceutically acceptable salt thereof, may be administered to the subject at a dose, such as a fixed dose, of about 4.5 mg, and the GLP-1R agonist (e.g., GLP-1R/GCGR dual agonist), or pharmaceutically acceptable salt thereof, may be administered to the subject at a dose, such as a fixed dose, of about 2.3 mg.

The AMYR agonist, or pharmaceutically acceptable salt thereof, may be administered to the subject at a dose, such as a fixed dose, of about 4.5 mg, and the GLP-1R agonist (e.g., GLP-1R/GCGR dual agonist), or pharmaceutically acceptable salt thereof, may be administered to the subject at a dose, such as a fixed dose, of about 1.2 mg.

The AMYR agonist, or pharmaceutically acceptable salt thereof, may be administered to the subject at a dose, such as a fixed dose, of about 1.5 mg (for example, about 1.60 mg), and the GLP-1R agonist (e.g., GLP-1R/GCGR dual agonist), or pharmaceutically acceptable salt thereof, may be administered to the subject at a dose, such as a fixed dose, of about 9.4 mg.

The AMYR agonist, or pharmaceutically acceptable salt thereof, may be administered to the subject at a dose, such as a fixed dose, of about 1.5 mg (for example, about 1.60 mg), and the GLP-1R agonist (e.g., GLP-1R/GCGR dual agonist), or pharmaceutically acceptable salt thereof, may be administered to the subject at a dose, such as a fixed dose, of about 7 mg.

The AMYR agonist, or pharmaceutically acceptable salt thereof, may be administered to the subject at a dose, such as a fixed dose, of about 1.5 mg (for example, about 1.60 mg), and the GLP-1R agonist (e.g., GLP-1R/GCGR dual agonist), or pharmaceutically acceptable salt thereof, may be administered to the subject at a dose, such as a fixed dose, of about 4.6 mg.

The AMYR agonist, or pharmaceutically acceptable salt thereof, may be administered to the subject at a dose, such as a fixed dose, of about 1.5 mg (for example, about 1.60 mg), and the GLP-1R agonist (e.g., GLP-1R/GCGR dual agonist), or pharmaceutically acceptable salt thereof, may be administered to the subject at a dose, such as a fixed dose, of about 2.3 mg.

The AMYR agonist, or pharmaceutically acceptable salt thereof, may be administered to the subject at a dose, such as a fixed dose, of about 1.5 mg (for example, about 1.60 mg), and the GLP-1R agonist (e.g., GLP-1R/GCGR dual agonist), or pharmaceutically acceptable salt thereof, may be administered to the subject at a dose, such as a fixed dose, of about 1.2 mg.

Treatment or prevention may be effected by a single administration or by multiple administrations of the combination therapy. For example, the treatment or prevention may comprise administering two doses, three doses, four doses, five doses, six doses, seven doses, eight doses, nine doses, ten doses, or more than ten doses of the combination therapy to the subject. Each dose may be a dose, such as a fixed dose, as disclosed herein. The treatment or prevention may comprise multiple administrations as described herein, such as repeated administrations, of the combination therapy over a course of treatment.

By a course of treatment is meant a treatment plan comprising several rounds of administration, as part of a method of treating and/or preventing a disease or disorder as described herein (e.g. obesity or an obesity-related condition and/or a metabolic disease) in accordance with the invention. A course of treatment may last for one or more weeks, one or more months, such as two, three, four, five, six, seven, eight, nine, ten or eleven months, or for one or more years, such as one year, two years, three years, four years, five years, or longer. A course of treatment may be continued with no fixed end-point, for example a course of treatment may be continued for the lifetime of the subject being treated. Multiple administrations, such as repeated administrations, may be continued at regular or irregular intervals during the course of the treatment as determined to be needed by a medical practitioner. A course of treatment of any duration as described herein may comprise a titration schedule as discussed below.

The combination therapy may be administered to a subject multiple times, at an interval of once a week, every two weeks, every three weeks, or every four weeks. The combination therapy may be administered about once every 4, 5, 6, 7, 8, 9, 10, 11, 12, 13, or 14 days. Preferably, the combination therapy is administered about once a week. Accordingly, the combination therapy may be administered to the subject at a dose, such as a fixed dose, of an AMYR agonist as disclosed herein and a fixed dose of a GLP-1R agonist (e.g., GLP-1R/GCGR dual agonist) as disclosed herein (or pharmaceutically acceptable salts thereof), about once a week. For the avoidance of doubt, these administration intervals apply equally and without reservation to each dosing regimen herein, unless expressly stated to the contrary.

Thus, the AMYR agonist, or pharmaceutically acceptable salt thereof, may be administered to the subject at a dose, such as a fixed dose, of about 1 mg to about 15 mg, and the GLP-1R agonist (e.g., GLP-1R/GCGR dual agonist), or pharmaceutically acceptable salt thereof, may be administered to the subject at a dose, such as a fixed dose, of about 0.1 mg to about 10 mg; wherein said administration is about once every 4, 5, 6, 7, 8, 9, 10, 11, 12, 13, or 14 days. Preferably, the combination therapy is administered about once a week.

By way of example, the AMYR agonist, or pharmaceutically acceptable salt thereof, may be administered to the subject at a dose, such as a fixed dose, of about 7 mg to about 10 mg (e.g., about 9 mg), and the GLP-1R agonist (e.g., GLP-1R/GCGR dual agonist), or pharmaceutically acceptable salt thereof, may be administered to the subject at a dose, such as a fixed dose, of about 8 mg to about 10 mg (e.g. about 9.4 mg); wherein said administration is about once every 4, 5, 6, 7, 8, 9, 10, 11, 12, 13, or 14 days. Preferably, the combination therapy is administered about once a week.

By way of further example, the AMYR agonist, or pharmaceutically acceptable salt thereof, may be administered to the subject at a dose, such as a fixed dose, of about 7 mg to about 10 mg (e.g., about 9 mg), and the GLP-1R agonist (e.g., GLP-1R/GCGR dual agonist), or pharmaceutically acceptable salt thereof, may be administered to the subject at a dose, such as a fixed dose, of about 5 mg to about 10 mg (e.g. about 7 mg); wherein said administration is about once every 4, 5, 6, 7, 8, 9, 10, 11, 12, 13, or 14 days. Preferably, the combination therapy is administered about once a week.

By way of further example, the AMYR agonist, or pharmaceutically acceptable salt thereof, may be administered to the subject at a dose, such as a fixed dose, of about 7 mg to about 10 mg (e.g., about 9 mg), and the GLP-1R agonist (e.g., GLP-1R/GCGR dual agonist), or pharmaceutically acceptable salt thereof, may be administered to the subject at a dose, such as a fixed dose, of about 3 mg to about 5 mg (e.g. about 4.6 mg); wherein said administration is about once every 4, 5, 6, 7, 8, 9, 10, 11, 12, 13, or 14 days. Preferably, the combination therapy is administered about once a week.

By way of further example, the AMYR agonist, or pharmaceutically acceptable salt thereof, may be administered to the subject at a dose, such as a fixed dose, of about 7 mg to about 10 mg (e.g., about 9 mg), and the GLP-1R agonist (e.g., GLP-1R/GCGR dual agonist), or pharmaceutically acceptable salt thereof, may be administered to the subject at a dose, such as a fixed dose, of about 2 mg to about 4 mg (e.g. about 2.3 mg); wherein said administration is about once every 4, 5, 6, 7, 8, 9, 10, 11, 12, 13, or 14 days. Preferably, the combination therapy is administered about once a week.

By way of further example, the AMYR agonist, or pharmaceutically acceptable salt thereof, may be administered to the subject at a dose, such as a fixed dose, of about 7 mg to about 10 mg (e.g., about 9 mg), and the GLP-1R agonist (e.g., GLP-1R/GCGR dual agonist), or pharmaceutically acceptable salt thereof, may be administered to the subject at a dose, such as a fixed dose, of about 1 mg to about 2 mg (e.g. about 1.2 mg); wherein said administration is about once every 4, 5, 6, 7, 8, 9, 10, 11, 12, 13, or 14 days. Preferably, the combination therapy is administered about once a week.

By way of further example, the AMYR agonist, or pharmaceutically acceptable salt thereof, may be administered to the subject at a dose, such as a fixed dose, of about 4 mg to about 7 mg (e.g. about 6 mg), and the GLP-1R agonist (e.g., GLP-1R/GCGR dual agonist), or pharmaceutically acceptable salt thereof, may be administered to the subject at a dose, such as a fixed dose, of about 8 mg to about 10 mg (e.g. about 9.4 mg); wherein said administration is about once every 4, 5, 6, 7, 8, 9, 10, 11, 12, 13, or 14 days. Preferably, the combination therapy is administered about once a week.

By way of further example, the AMYR agonist, or pharmaceutically acceptable salt thereof, may be administered to the subject at a dose, such as a fixed dose, of about 4 mg to about 7 mg (e.g. about 6 mg), and the GLP-1R agonist (e.g., GLP-1R/GCGR dual agonist), or pharmaceutically acceptable salt thereof, may be administered to the subject at a dose, such as a fixed dose, of about 5 mg to about 10 mg (e.g. about 7 mg); wherein said administration is about once every 4, 5, 6, 7, 8, 9, 10, 11, 12, 13, or 14 days. Preferably, the combination therapy is administered about once a week.

By way of further example, the AMYR agonist, or pharmaceutically acceptable salt thereof, may be administered to the subject at a dose, such as a fixed dose, of about 4 mg to about 7 mg (e.g. about 6 mg), and the GLP-1R agonist (e.g., GLP-1R/GCGR dual agonist), or pharmaceutically acceptable salt thereof, may be administered to the subject at a dose, such as a fixed dose, of about 3 mg to about 5 mg (e.g. about 4.6 mg); wherein said administration is about once every 4, 5, 6, 7, 8, 9, 10, 11, 12, 13, or 14 days. Preferably, the combination therapy is administered about once a week.

By way of further example, the AMYR agonist, or pharmaceutically acceptable salt thereof, may be administered to the subject at a dose, such as a fixed dose, of about 4 mg to about 7 mg (e.g. about 6 mg), and the GLP-1R agonist (e.g., GLP-1R/GCGR dual agonist), or pharmaceutically acceptable salt thereof, may be administered to the subject at a dose, such as a fixed dose, of about 2 mg to about 4 mg (e.g. about 2.3 mg); wherein said administration is about once every 4, 5, 6, 7, 8, 9, 10, 11, 12, 13, or 14 days. Preferably, the combination therapy is administered about once a week.

By way of further example, the AMYR agonist, or pharmaceutically acceptable salt thereof, may be administered to the subject at a dose, such as a fixed dose, of about 4 mg to about 7 mg (e.g. about 6 mg), and the GLP-1R agonist (e.g., GLP-1R/GCGR dual agonist), or pharmaceutically acceptable salt thereof, may be administered to the subject at a dose, such as a fixed dose, of about 1 mg to about 2 mg (e.g. about 1.2 mg); wherein said administration is about once every 4, 5, 6, 7, 8, 9, 10, 11, 12, 13, or 14 days. Preferably, the combination therapy is administered about once a week.

By way of further example, the AMYR agonist, or pharmaceutically acceptable salt thereof, may be administered to the subject at a dose, such as a fixed dose, of about 4 mg to about 5 mg (e.g. about 4.5 mg), and the GLP-1R agonist (e.g., GLP-1R/GCGR dual agonist), or pharmaceutically acceptable salt thereof, may be administered to the subject at a dose, such as a fixed dose, of about 8 mg to about 10 mg (e.g. about 9.4 mg); wherein said administration is about once every 4, 5, 6, 7, 8, 9, 10, 11, 12, 13, or 14 days. Preferably, the combination therapy is administered about once a week.

By way of further example, the AMYR agonist, or pharmaceutically acceptable salt thereof, may be administered to the subject at a dose, such as a fixed dose, of about 4 mg to about 5 mg (e.g. about 4.5 mg), and the GLP-1R agonist (e.g., GLP-1R/GCGR dual agonist), or pharmaceutically acceptable salt thereof, may be administered to the subject at a dose, such as a fixed dose, of about 5 mg to about 10 mg (e.g. about 7 mg); wherein said administration is about once every 4, 5, 6, 7, 8, 9, 10, 11, 12, 13, or 14 days. Preferably, the combination therapy is administered about once a week.

By way of further example, the AMYR agonist, or pharmaceutically acceptable salt thereof, may be administered to the subject at a dose, such as a fixed dose, of about 4 mg to about 5 mg (e.g. about 4.5 mg), and the GLP-1R agonist (e.g., GLP-1R/GCGR dual agonist), or pharmaceutically acceptable salt thereof, may be administered to the subject at a dose, such as a fixed dose, of about 3 mg to about 5 mg (e.g. about 4.6 mg); wherein said administration is about once every 4, 5, 6, 7, 8, 9, 10, 11, 12, 13, or 14 days. Preferably, the combination therapy is administered about once a week.

By way of further example, the AMYR agonist, or pharmaceutically acceptable salt thereof, may be administered to the subject at a dose, such as a fixed dose, of about 4 mg to about 5 mg (e.g. about 4.5 mg), and the GLP-1R agonist (e.g., GLP-1R/GCGR dual agonist), or pharmaceutically acceptable salt thereof, may be administered to the subject at a dose, such as a fixed dose, of about 2 mg to about 4 mg (e.g. about 2.3 mg); wherein said administration is about once every 4, 5, 6, 7, 8, 9, 10, 11, 12, 13, or 14 days. Preferably, the combination therapy is administered about once a week.

By way of further example, the AMYR agonist, or pharmaceutically acceptable salt thereof, may be administered to the subject at a dose, such as a fixed dose, of about 4 mg to about 5 mg (e.g. about 4.5 mg), and the GLP-1R agonist (e.g., GLP-1R/GCGR dual agonist), or pharmaceutically acceptable salt thereof, may be administered to the subject at a dose, such as a fixed dose, of about 1 mg to about 2 mg (e.g. about 1.2 mg); wherein said administration is about once every 4, 5, 6, 7, 8, 9, 10, 11, 12, 13, or 14 days. Preferably, the combination therapy is administered about once a week.

By way of further example, the AMYR agonist, or pharmaceutically acceptable salt thereof, may be administered to the subject at a dose, such as a fixed dose, of about 1 mg to about 2 mg (for example, about 1.50 mg or about 1.60 mg), and the GLP-1R agonist (e.g., GLP-1R/GCGR dual agonist), or pharmaceutically acceptable salt thereof, may be administered to the subject at a dose, such as a fixed dose, of about 8 mg to about 10 mg (e.g. about 9.4 mg); wherein said administration is about once every 4, 5, 6, 7, 8, 9, 10, 11, 12, 13, or 14 days. Preferably, the combination therapy is administered about once a week.

By way of further example, the AMYR agonist, or pharmaceutically acceptable salt thereof, may be administered to the subject at a dose, such as a fixed dose, of about 1 mg to about 2 mg (for example, about 1.50 mg or about 1.60 mg), and the GLP-1R agonist (e.g., GLP-1R/GCGR dual agonist), or pharmaceutically acceptable salt thereof, may be administered to the subject at a dose, such as a fixed dose, of about 5 mg to about 10 mg (e.g. about 7 mg); wherein said administration is about once every 4, 5, 6, 7, 8, 9, 10, 11, 12, 13, or 14 days. Preferably, the combination therapy is administered about once a week.

By way of further example, the AMYR agonist, or pharmaceutically acceptable salt thereof, may be administered to the subject at a dose, such as a fixed dose, of about 1 mg to about 2 mg (for example, about 1.50 mg or about 1.60 mg), and the GLP-1R agonist (e.g., GLP-1R/GCGR dual agonist), or pharmaceutically acceptable salt thereof, may be administered to the subject at a dose, such as a fixed dose, of about 3 mg to about 5 mg (e.g. about 4.6 mg); wherein said administration is about once every 4, 5, 6, 7, 8, 9, 10, 11, 12, 13, or 14 days. Preferably, the combination therapy is administered about once a week.

By way of further example, the AMYR agonist, or pharmaceutically acceptable salt thereof, may be administered to the subject at a dose, such as a fixed dose, of about 1 mg to about 2 mg (for example, about 1.50 mg or about 1.60 mg), and the GLP-1R agonist (e.g., GLP-1R/GCGR dual agonist), or pharmaceutically acceptable salt thereof, may be administered to the subject at a dose, such as a fixed dose, of about 2 mg to about 4 mg (e.g. about 2.3 mg); wherein said administration is about once every 4, 5, 6, 7, 8, 9, 10, 11, 12, 13, or 14 days. Preferably, the combination therapy is administered about once a week.

By way of further example, the AMYR agonist, or pharmaceutically acceptable salt thereof, may be administered to the subject at a dose, such as a fixed dose, of about 1 mg to about 2 mg (for example, about 1.50 mg or about 1.60 mg), and the GLP-1R agonist (e.g., GLP-1R/GCGR dual agonist), or pharmaceutically acceptable salt thereof, may be administered to the subject at a dose, such as a fixed dose, of about 1 mg to about 2 mg (e.g. about 1.2 mg); wherein said administration is about once every 4, 5, 6, 7, 8, 9, 10, 11, 12, 13, or 14 days. Preferably, the combination therapy is administered about once a week.

By way of further example, the AMYR agonist, or pharmaceutically acceptable salt thereof, may be administered to the subject at a dose, such as a fixed dose, of about 9 mg, and the GLP-1R agonist (e.g., GLP-1R/GCGR dual agonist), or pharmaceutically acceptable salt thereof, may be administered to the subject at a dose, such as a fixed dose, of about 9.4 mg; wherein said administration is about once every 4, 5, 6, 7, 8, 9, 10, 11, 12, 13, or 14 days. Preferably, the combination therapy is administered about once a week.

By way of further example, the AMYR agonist, or pharmaceutically acceptable salt thereof, may be administered to the subject at a dose, such as a fixed dose, of about 9 mg, and the GLP-1R agonist (e.g., GLP-1R/GCGR dual agonist), or pharmaceutically acceptable salt thereof, may be administered to the subject at a dose, such as a fixed dose, of about 7 mg; wherein said administration is about once every 4, 5, 6, 7, 8, 9, 10, 11, 12, 13, or 14 days. Preferably, the combination therapy is administered about once a week.

By way of further example, the AMYR agonist, or pharmaceutically acceptable salt thereof, may be administered to the subject at a dose, such as a fixed dose, of about 9 mg, and the GLP-1R agonist (e.g., GLP-1R/GCGR dual agonist), or pharmaceutically acceptable salt thereof, may be administered to the subject at a dose, such as a fixed dose, of about 4.6 mg; wherein said administration is about once every 4, 5, 6, 7, 8, 9, 10, 11, 12, 13, or 14 days. Preferably, the combination therapy is administered about once a week.

By way of further example, the AMYR agonist, or pharmaceutically acceptable salt thereof, may be administered to the subject at a dose, such as a fixed dose, of about 9 mg, and the GLP-1R agonist (e.g., GLP-1R/GCGR dual agonist), or pharmaceutically acceptable salt thereof, may be administered to the subject at a dose, such as a fixed dose, of about 2.3 mg; wherein said administration is about once every 4, 5, 6, 7, 8, 9, 10, 11, 12, 13, or 14 days. Preferably, the combination therapy is administered about once a week.

By way of further example, the AMYR agonist, or pharmaceutically acceptable salt thereof, may be administered to the subject at a dose, such as a fixed dose, of about 9 mg, and the GLP-1R agonist (e.g., GLP-1R/GCGR dual agonist), or pharmaceutically acceptable salt thereof, may be administered to the subject at a dose, such as a fixed dose, of about 1.2 mg; wherein said administration is about once every 4, 5, 6, 7, 8, 9, 10, 11, 12, 13, or 14 days. Preferably, the combination therapy is administered about once a week.

By way of further example, the AMYR agonist, or pharmaceutically acceptable salt thereof, may be administered to the subject at a dose, such as a fixed dose, of about 6 mg, and the GLP-1R agonist (e.g., GLP-1R/GCGR dual agonist), or pharmaceutically acceptable salt thereof, may be administered to the subject at a dose, such as a fixed dose, of about 9.4 mg; wherein said administration is about once every 4, 5, 6, 7, 8, 9, 10, 11, 12, 13, or 14 days. Preferably, the combination therapy is administered about once a week.

By way of further example, the AMYR agonist, or pharmaceutically acceptable salt thereof, may be administered to the subject at a dose, such as a fixed dose, of about 6 mg, and the GLP-1R agonist (e.g., GLP-1R/GCGR dual agonist), or pharmaceutically acceptable salt thereof, may be administered to the subject at a dose, such as a fixed dose, of about 7 mg; wherein said administration is about once every 4, 5, 6, 7, 8, 9, 10, 11, 12, 13, or 14 days. Preferably, the combination therapy is administered about once a week.

By way of further example, the AMYR agonist, or pharmaceutically acceptable salt thereof, may be administered to the subject at a dose, such as a fixed dose, of about 6 mg, and the GLP-1R agonist (e.g., GLP-1R/GCGR dual agonist), or pharmaceutically acceptable salt thereof, may be administered to the subject at a dose, such as a fixed dose, of about 4.6 mg; wherein said administration is about once every 4, 5, 6, 7, 8, 9, 10, 11, 12, 13, or 14 days. Preferably, the combination therapy is administered about once a week.

By way of further example, the AMYR agonist, or pharmaceutically acceptable salt thereof, may be administered to the subject at a dose, such as a fixed dose, of about 6 mg, and the GLP-1R agonist (e.g., GLP-1R/GCGR dual agonist), or pharmaceutically acceptable salt thereof, may be administered to the subject at a dose, such as a fixed dose, of about 2.3 mg; wherein said administration is about once every 4, 5, 6, 7, 8, 9, 10, 11, 12, 13, or 14 days. Preferably, the combination therapy is administered about once a week.

By way of further example, the AMYR agonist, or pharmaceutically acceptable salt thereof, may be administered to the subject at a dose, such as a fixed dose, of about 6 mg, and the GLP-1R agonist (e.g., GLP-1R/GCGR dual agonist), or pharmaceutically acceptable salt thereof, may be administered to the subject at a dose, such as a fixed dose, of about 1.2 mg; wherein said administration is about once every 4, 5, 6, 7, 8, 9, 10, 11, 12, 13, or 14 days. Preferably, the combination therapy is administered about once a week.

By way of further example, the AMYR agonist, or pharmaceutically acceptable salt thereof, may be administered to the subject at a dose, such as a fixed dose, of about 4.5 mg, and the GLP-1R agonist (e.g., GLP-1R/GCGR dual agonist), or pharmaceutically acceptable salt thereof, may be administered to the subject at a dose, such as a fixed dose, of about 9.4 mg; wherein said administration is about once every 4, 5, 6, 7, 8, 9, 10, 11, 12, 13, or 14 days. Preferably, the combination therapy is administered about once a week.

By way of further example, the AMYR agonist, or pharmaceutically acceptable salt thereof, may be administered to the subject at a dose, such as a fixed dose, of about 4.5 mg, and the GLP-1R agonist (e.g., GLP-1R/GCGR dual agonist), or pharmaceutically acceptable salt thereof, may be administered to the subject at a dose, such as a fixed dose, of about 7 mg; wherein said administration is about once every 4, 5, 6, 7, 8, 9, 10, 11, 12, 13, or 14 days. Preferably, the combination therapy is administered about once a week.

By way of further example, the AMYR agonist, or pharmaceutically acceptable salt thereof, may be administered to the subject at a dose, such as a fixed dose, of about 4.5 mg, and the GLP-1R agonist (e.g., GLP-1R/GCGR dual agonist), or pharmaceutically acceptable salt thereof, may be administered to the subject at a dose, such as a fixed dose, of about 4.6 mg; wherein said administration is about once every 4, 5, 6, 7, 8, 9, 10, 11, 12, 13, or 14 days. Preferably, the combination therapy is administered about once a week.

By way of further example, the way of example, the AMYR agonist, or pharmaceutically acceptable salt thereof, may be administered to the subject at a dose, such as a fixed dose, of about 4.5 mg, and the GLP-1R agonist (e.g., GLP-1R/GCGR dual agonist), or pharmaceutically acceptable salt thereof, may be administered to the subject at a dose, such as a fixed dose, of about 2.3 mg; wherein said administration is about once every 4, 5, 6, 7, 8, 9, 10, 11, 12, 13, or 14 days. Preferably, the combination therapy is administered about once a week.

By way of further example, the AMYR agonist, or pharmaceutically acceptable salt thereof, may be administered to the subject at a dose, such as a fixed dose, of about 4.5 mg, and the GLP-1R agonist (e.g., GLP-1R/GCGR dual agonist), or pharmaceutically acceptable salt thereof, may be administered to the subject at a dose, such as a fixed dose, of about 1.2 mg; wherein said administration is about once every 4, 5, 6, 7, 8, 9, 10, 11, 12, 13, or 14 days. Preferably, the combination therapy is administered about once a week.

By way of further example, the AMYR agonist, or pharmaceutically acceptable salt thereof, may be administered to the subject at a dose, such as a fixed dose, of about 1.5 mg (for example, about 1.60 mg), and the GLP-1R agonist (e.g., GLP-1R/GCGR dual agonist), or pharmaceutically acceptable salt thereof, may be administered to the subject at a dose, such as a fixed dose, of about 9.4 mg; wherein said administration is about once every 4, 5, 6, 7, 8, 9, 10, 11, 12, 13, or 14 days. Preferably, the combination therapy is administered about once a week.

By way of further example, the AMYR agonist, or pharmaceutically acceptable salt thereof, may be administered to the subject at a dose, such as a fixed dose, of about 1.5 mg (for example, about 1.60 mg), and the GLP-1R agonist (e.g., GLP-1R/GCGR dual agonist), or pharmaceutically acceptable salt thereof, may be administered to the subject at a dose, such as a fixed dose, of about 7 mg; wherein said administration is about once every 4, 5, 6, 7, 8, 9, 10, 11, 12, 13, or 14 days. Preferably, the combination therapy is administered about once a week.

By way of further example, the AMYR agonist, or pharmaceutically acceptable salt thereof, may be administered to the subject at a dose, such as a fixed dose, of about 1.5 mg (for example, about 1.60 mg), and the GLP-1R agonist (e.g., GLP-1R/GCGR dual agonist), or pharmaceutically acceptable salt thereof, may be administered to the subject at a dose, such as a fixed dose, of about 4.6 mg; wherein said administration is about once every 4, 5, 6, 7, 8, 9, 10, 11, 12, 13, or 14 days. Preferably, the combination therapy is administered about once a week.

By way of further example, the AMYR agonist, or pharmaceutically acceptable salt thereof, may be administered to the subject at a dose, such as a fixed dose, of about 1.5 mg (for example, about 1.60 mg), and the GLP-1R agonist (e.g., GLP-1R/GCGR dual agonist), or pharmaceutically acceptable salt thereof, may be administered to the subject at a dose, such as a fixed dose, of about 2.3 mg; wherein said administration is about once every 4, 5, 6, 7, 8, 9, 10, 11, 12, 13, or 14 days. Preferably, the combination therapy is administered about once a week.

By way of further example, the AMYR agonist, or pharmaceutically acceptable salt thereof, may be administered to the subject at a dose, such as a fixed dose, of about 1.5 mg (for example, about 1.60 mg), and the GLP-1R agonist (e.g., GLP-1R/GCGR dual agonist), or pharmaceutically acceptable salt thereof, may be administered to the subject at a dose, such as a fixed dose, of about 1.2 mg; wherein said administration is about once every 4, 5, 6, 7, 8, 9, 10, 11, 12, 13, or 14 days. Preferably, the combination therapy is administered about once a week.

When the combination therapy is administered in multiple administrations, such as repeated administrations, each of said administrations of the AMYR agonist and/or the GLP-1R agonist (e.g. GLP-1R/GCGR dual agonist) may be at a fixed dose as described herein. For example, each administration in a method or use of the invention may comprise a fixed dose of about 1 mg to about 15 mg of the AMYR agonist and about 0.1 mg to about 10 mg of the GLP-1R agonist (e.g. GLP-1R/GCGR dual agonist) as disclosed herein. In such instances, the number of administrations or the frequency of administrations may be any of the options disclosed herein. The multiple administrations, such as repeated administrations of the combination therapy, may be each at the same dose. Two or more administrations of the combination therapy may be each at the same dose. The multiple administrations, such as repeated administrations of the combination therapy, may be each at a dose within a range of doses disclosed herein. For example, the combination therapy may be administered to the subject at a dose, such as a fixed dose, of:
about 7 mg to about 10 mg (e.g., about 9 mg) of the AMYR agonist, or pharmaceutically acceptable salt thereof, and about 8 mg to about 10 mg (e.g. about 9.4 mg) of the GLP-1R agonist (e.g., GLP-1R/GCGR dual agonist), or pharmaceutically acceptable salt thereof;
about 7 mg to about 10 mg (e.g., about 9 mg) of the AMYR agonist, or pharmaceutically acceptable salt thereof, and about 5 mg to about 10 mg (e.g. about 7 mg) of the GLP-1R agonist (e.g., GLP-1R/GCGR dual agonist), or pharmaceutically acceptable salt thereof;
about 7 mg to about 10 mg (e.g., about 9 mg) of the AMYR agonist, or pharmaceutically acceptable salt thereof, and about 3 mg to about 5 mg (e.g. about 4.6 mg) of the GLP-1R agonist (e.g., GLP-1R/GCGR dual agonist), or pharmaceutically acceptable salt thereof;
about 7 mg to about 10 mg (e.g., about 9 mg) of the AMYR agonist, or pharmaceutically acceptable salt thereof, and about 2 mg to about 4 mg (e.g. about 2.3 mg) of the GLP-1R agonist (e.g., GLP-1R/GCGR dual agonist), or pharmaceutically acceptable salt thereof;
about 7 mg to about 10 mg (e.g., about 9 mg) of the AMYR agonist, or pharmaceutically acceptable salt thereof, and about 1 mg to about 2 mg (e.g. about 1.2 mg) of the GLP-1R agonist (e.g., GLP-1R/GCGR dual agonist), or pharmaceutically acceptable salt thereof;
about 4 mg to about 7 mg (e.g. about 6 mg) of the AMYR agonist, or pharmaceutically acceptable salt thereof, and about 8 mg to about 10 mg (e.g. about 9.4 mg) of the GLP-1R agonist (e.g., GLP-1R/GCGR dual agonist), or pharmaceutically acceptable salt thereof;
about 4 mg to about 7 mg (e.g. about 6 mg) of the AMYR agonist, or pharmaceutically acceptable salt thereof, and about 5 mg to about 10 mg (e.g. about 7 mg) of the GLP-1R agonist (e.g., GLP-1R/GCGR dual agonist), or pharmaceutically acceptable salt thereof;
about 4 mg to about 7 mg (e.g. about 6 mg) of the AMYR agonist, or pharmaceutically acceptable salt thereof, and about 3 mg to about 5 mg (e.g. about 4.6 mg) of the GLP-1R agonist (e.g., GLP-1R/GCGR dual agonist), or pharmaceutically acceptable salt thereof;
about 4 mg to about 7 mg (e.g. about 6 mg) of the AMYR agonist, or pharmaceutically acceptable salt thereof, and about 2 mg to about 4 mg (e.g. about 2.3 mg) of the GLP-1R agonist (e.g., GLP-1R/GCGR dual agonist), or pharmaceutically acceptable salt thereof;
about 4 mg to about 7 mg (e.g. about 6 mg) of the AMYR agonist, or pharmaceutically acceptable salt thereof, and about 1 mg to about 2 mg (e.g. about 1.2 mg) of the GLP-1R agonist (e.g., GLP-1R/GCGR dual agonist), or pharmaceutically acceptable salt thereof;
about 4 mg to about 5 mg (e.g. about 4.5 mg) of the AMYR agonist, or pharmaceutically acceptable salt thereof, and about 8 mg to about 10 mg (e.g. about 9.4 mg) of the GLP-1R agonist (e.g., GLP-1R/GCGR dual agonist), or pharmaceutically acceptable salt thereof;
about 4 mg to about 5 mg (e.g. about 4.5 mg) of the AMYR agonist, or pharmaceutically acceptable salt thereof, and about 5 mg to about 10 mg (e.g. about 7 mg) of the GLP-1R agonist (e.g., GLP-1R/GCGR dual agonist), or pharmaceutically acceptable salt thereof;
about 4 mg to about 5 mg (e.g. about 4.5 mg) of the AMYR agonist, or pharmaceutically acceptable salt thereof, and about 3 mg to about 5 mg (e.g. about 4.6 mg) of the GLP-1R agonist (e.g., GLP-1R/GCGR dual agonist), or pharmaceutically acceptable salt thereof;
about 4 mg to about 5 mg (e.g. about 4.5 mg) of the AMYR agonist, or pharmaceutically acceptable salt thereof, and about 2 mg to about 4 mg (e.g. about 2.3 mg) of the GLP-1R agonist (e.g., GLP-1R/GCGR dual agonist), or pharmaceutically acceptable salt thereof;
about 4 mg to about 5 mg (e.g. about 4.5 mg) of the AMYR agonist, or pharmaceutically acceptable salt thereof, and about 1 mg to about 2 mg (e.g. about 1.2 mg) of the GLP-1R agonist (e.g., GLP-1R/GCGR dual agonist), or pharmaceutically acceptable salt thereof;
about 1 mg to about 2 mg (for example, about 1.50 mg or about 1.60 mg) of the AMYR agonist, or pharmaceutically acceptable salt thereof, and about 8 mg to about 10 mg (e.g. about 9.4 mg) of the GLP-1R agonist (e.g., GLP-1R/GCGR dual agonist), or pharmaceutically acceptable salt thereof;
about 1 mg to about 2 mg (for example, about 1.50 mg or about 1.60 mg) of the AMYR agonist, or pharmaceutically acceptable salt thereof, and about 5 mg to about 10 mg (e.g. about 7 mg) of the GLP-1R agonist (e.g., GLP-1R/GCGR dual agonist), or pharmaceutically acceptable salt thereof;
about 1 mg to about 2 mg (for example, about 1.50 mg or about 1.60 mg) of the AMYR agonist, or pharmaceutically acceptable salt thereof, and about 3 mg to about 5 mg (e.g. about 4.6 mg) of the GLP-1R agonist (e.g., GLP-1R/GCGR dual agonist), or pharmaceutically acceptable salt thereof;
about 1 mg to about 2 mg (for example, about 1.50 mg or about 1.60 mg) of the AMYR agonist, or pharmaceutically acceptable salt thereof, and about 2 mg to about 4 mg (e.g. about 2.3 mg) of the GLP-1R agonist (e.g., GLP-1R/GCGR dual agonist), or pharmaceutically acceptable salt thereof; or
about 1 mg to about 2 mg (for example, about 1.50 mg or about 1.60 mg) of the AMYR agonist, or pharmaceutically acceptable salt thereof, and about 1 mg to about 2 mg (e.g. about 1.2 mg) of the GLP-1R agonist (e.g., GLP-1R/GCGR dual agonist), or pharmaceutically acceptable salt thereof.

By way of example, the method may comprise administering to the subject multiple doses of:
about 9 mg of the AMYR agonist, or pharmaceutically acceptable salt thereof and about 9.4 mg of the GLP-1R agonist (e.g., GLP-1R/GCGR dual agonist), or pharmaceutically acceptable salt thereof;
about 9 mg of the AMYR agonist, or pharmaceutically acceptable salt thereof and about 7 mg of the GLP-1R agonist (e.g., GLP-1R/GCGR dual agonist), or pharmaceutically acceptable salt thereof;
about 9 mg of the AMYR agonist, or pharmaceutically acceptable salt thereof and about 4.6 mg of the GLP-1R agonist (e.g., GLP-1R/GCGR dual agonist), or pharmaceutically acceptable salt thereof;
about 9 mg of the AMYR agonist, or pharmaceutically acceptable salt thereof and about 2.3 mg of the GLP-1R agonist (e.g., GLP-1R/GCGR dual agonist), or pharmaceutically acceptable salt thereof;
about 9 mg of the AMYR agonist, or pharmaceutically acceptable salt thereof and about 1.2 mg of the GLP-1R agonist (e.g., GLP-1R/GCGR dual agonist), or pharmaceutically acceptable salt thereof;
about 6 mg of the AMYR agonist, or pharmaceutically acceptable salt thereof and about 9.4 mg of the GLP-1R agonist (e.g., GLP-1R/GCGR dual agonist), or pharmaceutically acceptable salt thereof;
about 6 mg of the AMYR agonist, or pharmaceutically acceptable salt thereof and about 7 mg of the GLP-1R agonist (e.g., GLP-1R/GCGR dual agonist), or pharmaceutically acceptable salt thereof;
about 6 mg of the AMYR agonist, or pharmaceutically acceptable salt thereof and about 4.6 mg of the GLP-1R agonist (e.g., GLP-1R/GCGR dual agonist), or pharmaceutically acceptable salt thereof;
about 6 mg of the AMYR agonist, or pharmaceutically acceptable salt thereof and about 2.3 mg of the GLP-1R agonist (e.g., GLP-1R/GCGR dual agonist), or pharmaceutically acceptable salt thereof;
about 6 mg of the AMYR agonist, or pharmaceutically acceptable salt thereof and about 1.2 mg of the GLP-1R agonist (e.g., GLP-1R/GCGR dual agonist), or pharmaceutically acceptable salt thereof;
about 4.5 mg of the AMYR agonist, or pharmaceutically acceptable salt thereof and about 9.4 mg of the GLP-1R agonist (e.g., GLP-1R/GCGR dual agonist), or pharmaceutically acceptable salt thereof;
about 4.5 mg of the AMYR agonist, or pharmaceutically acceptable salt thereof and about 7 mg of the GLP-1R agonist (e.g., GLP-1R/GCGR dual agonist), or pharmaceutically acceptable salt thereof;
about 4.5 mg of the AMYR agonist, or pharmaceutically acceptable salt thereof and about 4.6 mg of the GLP-1R agonist (e.g., GLP-1R/GCGR dual agonist), or pharmaceutically acceptable salt thereof;
about 4.5 mg of the AMYR agonist, or pharmaceutically acceptable salt thereof and about 2.3 mg of the GLP-1R agonist (e.g., GLP-1R/GCGR dual agonist), or pharmaceutically acceptable salt thereof;
about 4.5 mg of the AMYR agonist, or pharmaceutically acceptable salt thereof and about 1.2 mg of the GLP-1R agonist (e.g., GLP-1R/GCGR dual agonist), or pharmaceutically acceptable salt thereof;
about 1.5 mg or 1.6 mg of the AMYR agonist, or pharmaceutically acceptable salt thereof and about 9.4 mg of the GLP-1R agonist (e.g., GLP-1R/GCGR dual agonist), or pharmaceutically acceptable salt thereof;
about 1.5 mg or 1.6 mg of the AMYR agonist, or pharmaceutically acceptable salt thereof and about 7 mg of the GLP-1R agonist (e.g., GLP-1R/GCGR dual agonist), or pharmaceutically acceptable salt thereof;
about 1.5 mg or 1.6 mg of the AMYR agonist, or pharmaceutically acceptable salt thereof and about 4.6 mg of the GLP-1R agonist (e.g., GLP-1R/GCGR dual agonist), or pharmaceutically acceptable salt thereof;
about 1.5 mg or 1.6 mg of the AMYR agonist, or pharmaceutically acceptable salt thereof and about 2.3 mg of the GLP-1R agonist (e.g., GLP-1R/GCGR dual agonist), or pharmaceutically acceptable salt thereof; or
about 1.5 mg or 1.6 mg of the AMYR agonist, or pharmaceutically acceptable salt thereof and about 1.2 mg of the GLP-1R agonist (e.g., GLP-1R/GCGR dual agonist), or pharmaceutically acceptable salt thereof.

In accordance with the invention, the combination therapy may be administered to a subject multiple times over a period of at least 1 month, 2 months, 3 months, 6 months, 9 months, 1 year, 2 years or 5 years. The combination therapy may be administered to a subject about once a week over a period of at least 1 month, 2 months, 3 months, 6 months, 9 months, 1 year, 2 years or 5 years. The subject may have body weight reduction following administration of multiple doses after 3 months. The subject may have body weight reduction following administration of multiple doses after 6 months. The subject may have body weight reduction following administration of multiple doses after 9 months. The subject may have body weight reduction following administration of multiple doses between a period of about 3 months and about 9 months, such as a period of about 3 months and about 6 months. The combination therapy may be administered at a dosing interval of about once a week over these periods of time.

For example, the combination therapy may be administered to the subject at a dose, such as a fixed dose, as described herein about once a week over a period of at least 3 months. By way of non-limiting example, the method may comprise administering to the subject a dose of:
about 7 mg to about 10 mg (e.g., about 9 mg) of the AMYR agonist, or pharmaceutically acceptable salt thereof, and about 8 mg to about 10 mg (e.g. about 9.4 mg) of the GLP-1R agonist (e.g., GLP-1R/GCGR dual agonist), or pharmaceutically acceptable salt thereof;
about 7 mg to about 10 mg (e.g., about 9 mg) of the AMYR agonist, or pharmaceutically acceptable salt thereof, and about 5 mg to about 10 mg (e.g. about 7 mg) of the GLP-1R agonist (e.g., GLP-1R/GCGR dual agonist), or pharmaceutically acceptable salt thereof;
about 7 mg to about 10 mg (e.g., about 9 mg) of the AMYR agonist, or pharmaceutically acceptable salt thereof, and about 3 mg to about 5 mg (e.g. about 4.6 mg) of the GLP-1R agonist (e.g., GLP-1R/GCGR dual agonist), or pharmaceutically acceptable salt thereof;
about 7 mg to about 10 mg (e.g., about 9 mg) of the AMYR agonist, or pharmaceutically acceptable salt thereof, and about 2 mg to about 4 mg (e.g. about 2.3 mg) of the GLP-1R agonist (e.g., GLP-1R/GCGR dual agonist), or pharmaceutically acceptable salt thereof;
about 7 mg to about 10 mg (e.g., about 9 mg) of the AMYR agonist, or pharmaceutically acceptable salt thereof, and about 1 mg to about 2 mg (e.g. about 1.2 mg) of the GLP-1R agonist (e.g., GLP-1R/GCGR dual agonist), or pharmaceutically acceptable salt thereof;
about 4 mg to about 7 mg (e.g. about 6 mg) of the AMYR agonist, or pharmaceutically acceptable salt thereof, and about 8 mg to about 10 mg (e.g. about 9.4 mg) of the GLP-1R agonist (e.g., GLP-1R/GCGR dual agonist), or pharmaceutically acceptable salt thereof;
about 4 mg to about 7 mg (e.g. about 6 mg) of the AMYR agonist, or pharmaceutically acceptable salt thereof, and about 5 mg to about 10 mg (e.g. about 7 mg) of the GLP-1R agonist (e.g., GLP-1R/GCGR dual agonist), or pharmaceutically acceptable salt thereof;
about 4 mg to about 7 mg (e.g. about 6 mg) of the AMYR agonist, or pharmaceutically acceptable salt thereof, and about 3 mg to about 5 mg (e.g. about 4.6 mg) of the GLP-1R agonist (e.g., GLP-1R/GCGR dual agonist), or pharmaceutically acceptable salt thereof;
about 4 mg to about 7 mg (e.g. about 6 mg) of the AMYR agonist, or pharmaceutically acceptable salt thereof, and about 2 mg to about 4 mg (e.g. about 2.3 mg) of the GLP-1R agonist (e.g., GLP-1R/GCGR dual agonist), or pharmaceutically acceptable salt thereof;
about 4 mg to about 7 mg (e.g. about 6 mg) of the AMYR agonist, or pharmaceutically acceptable salt thereof, and about 1 mg to about 2 mg (e.g. about 1.2 mg) of the GLP-1R agonist (e.g., GLP-1R/GCGR dual agonist), or pharmaceutically acceptable salt thereof;
about 4 mg to about 5 mg (e.g. about 4.5 mg) of the AMYR agonist, or pharmaceutically acceptable salt thereof, and about 8 mg to about 10 mg (e.g. about 9.4 mg) of the GLP-1R agonist (e.g., GLP-1R/GCGR dual agonist), or pharmaceutically acceptable salt thereof;
about 4 mg to about 5 mg (e.g. about 4.5 mg) of the AMYR agonist, or pharmaceutically acceptable salt thereof, and about 5 mg to about 10 mg (e.g. about 7 mg) of the GLP-1R agonist (e.g., GLP-1R/GCGR dual agonist), or pharmaceutically acceptable salt thereof;
about 4 mg to about 5 mg (e.g. about 4.5 mg) of the AMYR agonist, or pharmaceutically acceptable salt thereof, and about 3 mg to about 5 mg (e.g. about 4.6 mg) of the GLP-1R agonist (e.g., GLP-1R/GCGR dual agonist), or pharmaceutically acceptable salt thereof;
about 4 mg to about 5 mg (e.g. about 4.5 mg) of the AMYR agonist, or pharmaceutically acceptable salt thereof, and about 2 mg to about 4 mg (e.g. about 2.3 mg) of the GLP-1R agonist (e.g., GLP-1R/GCGR dual agonist), or pharmaceutically acceptable salt thereof;
about 4 mg to about 5 mg (e.g. about 4.5 mg) of the AMYR agonist, or pharmaceutically acceptable salt thereof, and about 1 mg to about 2 mg (e.g. about 1.2 mg) of the GLP-1R agonist (e.g., GLP-1R/GCGR dual agonist), or pharmaceutically acceptable salt thereof;
about 1 mg to about 2 mg (for example, about 1.50 mg or about 1.60 mg) of the AMYR agonist, or pharmaceutically acceptable salt thereof, and about 8 mg to about 10 mg (e.g. about 9.4 mg) of the GLP-1R agonist (e.g., GLP-1R/GCGR dual agonist), or pharmaceutically acceptable salt thereof;
about 1 mg to about 2 mg (for example, about 1.50 mg or about 1.60 mg) of the AMYR agonist, or pharmaceutically acceptable salt thereof, and about 5 mg to about 10 mg (e.g. about 7 mg) of the GLP-1R agonist (e.g., GLP-1R/GCGR dual agonist), or pharmaceutically acceptable salt thereof;
about 1 mg to about 2 mg (for example, about 1.50 mg or about 1.60 mg) of the AMYR agonist, or pharmaceutically acceptable salt thereof, and about 3 mg to about 5 mg (e.g. about 4.6 mg) of the GLP-1R agonist (e.g., GLP-1R/GCGR dual agonist), or pharmaceutically acceptable salt thereof;
about 1 mg to about 2 mg (for example, about 1.50 mg or about 1.60 mg) of the AMYR agonist, or pharmaceutically acceptable salt thereof, and about 2 mg to about 4 mg (e.g. about 2.3 mg) of the GLP-1R agonist (e.g., GLP-1R/GCGR dual agonist), or pharmaceutically acceptable salt thereof; or
about 1 mg to about 2 mg (for example, about 1.50 mg or about 1.60 mg) of the AMYR agonist, or pharmaceutically acceptable salt thereof, and about 1 mg to about 2 mg (e.g. about 1.2 mg) of the GLP-1R agonist (e.g., GLP-1R/GCGR dual agonist), or pharmaceutically acceptable salt thereof;
   about once a week over a period of at least 3 months.

The AMYR agonist, or pharmaceutically acceptable salt thereof, may be administered to the subject at a dose, such as a fixed dose as described herein about once a week over a period of at least 6 months. By way of non-limiting example, the method may comprise administering to the subject a dose of:
about 7 mg to about 10 mg (e.g., about 9 mg) of the AMYR agonist, or pharmaceutically acceptable salt thereof, and about 8 mg to about 10 mg (e.g. about 9.4 mg) of the GLP-1R agonist (e.g., GLP-1R/GCGR dual agonist), or pharmaceutically acceptable salt thereof;
about 7 mg to about 10 mg (e.g., about 9 mg) of the AMYR agonist, or pharmaceutically acceptable salt thereof, and about 5 mg to about 10 mg (e.g. about 7 mg) of the GLP-1R agonist (e.g., GLP-1R/GCGR dual agonist), or pharmaceutically acceptable salt thereof;
about 7 mg to about 10 mg (e.g., about 9 mg) of the AMYR agonist, or pharmaceutically acceptable salt thereof, and about 3 mg to about 5 mg (e.g. about 4.6 mg) of the GLP-1R agonist (e.g., GLP-1R/GCGR dual agonist), or pharmaceutically acceptable salt thereof;
about 7 mg to about 10 mg (e.g., about 9 mg) of the AMYR agonist, or pharmaceutically acceptable salt thereof, and about 2 mg to about 4 mg (e.g. about 2.3 mg) of the GLP-1R agonist (e.g., GLP-1R/GCGR dual agonist), or pharmaceutically acceptable salt thereof;
about 7 mg to about 10 mg (e.g., about 9 mg) of the AMYR agonist, or pharmaceutically acceptable salt thereof, and about 1 mg to about 2 mg (e.g. about 1.2 mg) of the GLP-1R agonist (e.g., GLP-1R/GCGR dual agonist), or pharmaceutically acceptable salt thereof;
about 4 mg to about 7 mg (e.g. about 6 mg) of the AMYR agonist, or pharmaceutically acceptable salt thereof, and about 8 mg to about 10 mg (e.g. about 9.4 mg) of the GLP-1R agonist (e.g., GLP-1R/GCGR dual agonist), or pharmaceutically acceptable salt thereof;
about 4 mg to about 7 mg (e.g. about 6 mg) of the AMYR agonist, or pharmaceutically acceptable salt thereof, and about 5 mg to about 10 mg (e.g. about 7 mg) of the GLP-1R agonist (e.g., GLP-1R/GCGR dual agonist), or pharmaceutically acceptable salt thereof;
about 4 mg to about 7 mg (e.g. about 6 mg) of the AMYR agonist, or pharmaceutically acceptable salt thereof, and about 3 mg to about 5 mg (e.g. about 4.6 mg) of the GLP-1R agonist (e.g., GLP-1R/GCGR dual agonist), or pharmaceutically acceptable salt thereof;
about 4 mg to about 7 mg (e.g. about 6 mg) of the AMYR agonist, or pharmaceutically acceptable salt thereof, and about 2 mg to about 4 mg (e.g. about 2.3 mg) of the GLP-1R agonist (e.g., GLP-1R/GCGR dual agonist), or pharmaceutically acceptable salt thereof;
about 4 mg to about 7 mg (e.g. about 6 mg) of the AMYR agonist, or pharmaceutically acceptable salt thereof, and about 1 mg to about 2 mg (e.g. about 1.2 mg) of the GLP-1R agonist (e.g., GLP-1R/GCGR dual agonist), or pharmaceutically acceptable salt thereof;
about 4 mg to about 5 mg (e.g. about 4.5 mg) of the AMYR agonist, or pharmaceutically acceptable salt thereof, and about 8 mg to about 10 mg (e.g. about 9.4 mg) of the GLP-1R agonist (e.g., GLP-1R/GCGR dual agonist), or pharmaceutically acceptable salt thereof;
about 4 mg to about 5 mg (e.g. about 4.5 mg) of the AMYR agonist, or pharmaceutically acceptable salt thereof, and about 5 mg to about 10 mg (e.g. about 7 mg) of the GLP-1R agonist (e.g., GLP-1R/GCGR dual agonist), or pharmaceutically acceptable salt thereof;
about 4 mg to about 5 mg (e.g. about 4.5 mg) of the AMYR agonist, or pharmaceutically acceptable salt thereof, and about 3 mg to about 5 mg (e.g. about 4.6 mg) of the GLP-1R agonist (e.g., GLP-1R/GCGR dual agonist), or pharmaceutically acceptable salt thereof;
about 4 mg to about 5 mg (e.g. about 4.5 mg) of the AMYR agonist, or pharmaceutically acceptable salt thereof, and about 2 mg to about 4 mg (e.g. about 2.3 mg) of the GLP-1R agonist (e.g., GLP-1R/GCGR dual agonist), or pharmaceutically acceptable salt thereof;
about 4 mg to about 5 mg (e.g. about 4.5 mg) of the AMYR agonist, or pharmaceutically acceptable salt thereof, and about 1 mg to about 2 mg (e.g. about 1.2 mg) of the GLP-1R agonist (e.g., GLP-1R/GCGR dual agonist), or pharmaceutically acceptable salt thereof;
about 1 mg to about 2 mg (for example, about 1.50 mg or about 1.60 mg) of the AMYR agonist, or pharmaceutically acceptable salt thereof, and about 8 mg to about 10 mg (e.g. about 9.4 mg) of the GLP-1R agonist (e.g., GLP-1R/GCGR dual agonist), or pharmaceutically acceptable salt thereof;
about 1 mg to about 2 mg (for example, about 1.50 mg or about 1.60 mg) of the AMYR agonist, or pharmaceutically acceptable salt thereof, and about 5 mg to about 10 mg (e.g. about 7 mg) of the GLP-1R agonist (e.g., GLP-1R/GCGR dual agonist), or pharmaceutically acceptable salt thereof;
about 1 mg to about 2 mg (for example, about 1.50 mg or about 1.60 mg) of the AMYR agonist, or pharmaceutically acceptable salt thereof, and about 3 mg to about 5 mg (e.g. about 4.6 mg) of the GLP-1R agonist (e.g., GLP-1R/GCGR dual agonist), or pharmaceutically acceptable salt thereof;
about 1 mg to about 2 mg (for example, about 1.50 mg or about 1.60 mg) of the AMYR agonist, or pharmaceutically acceptable salt thereof, and about 2 mg to about 4 mg (e.g. about 2.3 mg) of the GLP-1R agonist (e.g., GLP-1R/GCGR dual agonist), or pharmaceutically acceptable salt thereof; or
about 1 mg to about 2 mg (for example, about 1.50 mg or about 1.60 mg) of the AMYR agonist, or pharmaceutically acceptable salt thereof, and about 1 mg to about 2 mg (e.g. about 1.2 mg) of the GLP-1R agonist (e.g., GLP-1R/GCGR dual agonist), or pharmaceutically acceptable salt thereof;
   about once a week over a period of at least 6 months.

For example, the combination therapy may be administered to the subject at a dose, such as a fixed dose, as described herein about once a week over a period of at least 9 months. By way of non-limiting example, the method may comprise administering to the subject a dose of:
about 7 mg to about 10 mg (e.g., about 9 mg) of the AMYR agonist, or pharmaceutically acceptable salt thereof, and about 8 mg to about 10 mg (e.g. about 9.4 mg) of the GLP-1R agonist (e.g., GLP-1R/GCGR dual agonist), or pharmaceutically acceptable salt thereof;
about 7 mg to about 10 mg (e.g., about 9 mg) of the AMYR agonist, or pharmaceutically acceptable salt thereof, and about 5 mg to about 10 mg (e.g. about 7 mg) of the GLP-1R agonist (e.g., GLP-1R/GCGR dual agonist), or pharmaceutically acceptable salt thereof;
about 7 mg to about 10 mg (e.g., about 9 mg) of the AMYR agonist, or pharmaceutically acceptable salt thereof, and about 3 mg to about 5 mg (e.g. about 4.6 mg) of the GLP-1R agonist (e.g., GLP-1R/GCGR dual agonist), or pharmaceutically acceptable salt thereof;
about 7 mg to about 10 mg (e.g., about 9 mg) of the AMYR agonist, or pharmaceutically acceptable salt thereof, and about 2 mg to about 4 mg (e.g. about 2.3 mg) of the GLP-1R agonist (e.g., GLP-1R/GCGR dual agonist), or pharmaceutically acceptable salt thereof;
about 7 mg to about 10 mg (e.g., about 9 mg) of the AMYR agonist, or pharmaceutically acceptable salt thereof, and about 1 mg to about 2 mg (e.g. about 1.2 mg) of the GLP-1R agonist (e.g., GLP-1R/GCGR dual agonist), or pharmaceutically acceptable salt thereof;
about 4 mg to about 7 mg (e.g. about 6 mg) of the AMYR agonist, or pharmaceutically acceptable salt thereof, and about 8 mg to about 10 mg (e.g. about 9.4 mg) of the GLP-1R agonist (e.g., GLP-1R/GCGR dual agonist), or pharmaceutically acceptable salt thereof;
about 4 mg to about 7 mg (e.g. about 6 mg) of the AMYR agonist, or pharmaceutically acceptable salt thereof, and about 5 mg to about 10 mg (e.g. about 7 mg) of the GLP-1R agonist (e.g., GLP-1R/GCGR dual agonist), or pharmaceutically acceptable salt thereof;
about 4 mg to about 7 mg (e.g. about 6 mg) of the AMYR agonist, or pharmaceutically acceptable salt thereof, and about 3 mg to about 5 mg (e.g. about 4.6 mg) of the GLP-1R agonist (e.g., GLP-1R/GCGR dual agonist), or pharmaceutically acceptable salt thereof;
about 4 mg to about 7 mg (e.g. about 6 mg) of the AMYR agonist, or pharmaceutically acceptable salt thereof, and about 2 mg to about 4 mg (e.g. about 2.3 mg) of the GLP-1R agonist (e.g., GLP-1R/GCGR dual agonist), or pharmaceutically acceptable salt thereof;
about 4 mg to about 7 mg (e.g. about 6 mg) of the AMYR agonist, or pharmaceutically acceptable salt thereof, and about 1 mg to about 2 mg (e.g. about 1.2 mg) of the GLP-1R agonist (e.g., GLP-1R/GCGR dual agonist), or pharmaceutically acceptable salt thereof;
about 4 mg to about 5 mg (e.g. about 4.5 mg) of the AMYR agonist, or pharmaceutically acceptable salt thereof, and about 8 mg to about 10 mg (e.g. about 9.4 mg) of the GLP-1R agonist (e.g., GLP-1R/GCGR dual agonist), or pharmaceutically acceptable salt thereof;
about 4 mg to about 5 mg (e.g. about 4.5 mg) of the AMYR agonist, or pharmaceutically acceptable salt thereof, and about 5 mg to about 10 mg (e.g. about 7 mg) of the GLP-1R agonist (e.g., GLP-1R/GCGR dual agonist), or pharmaceutically acceptable salt thereof;
about 4 mg to about 5 mg (e.g. about 4.5 mg) of the AMYR agonist, or pharmaceutically acceptable salt thereof, and about 3 mg to about 5 mg (e.g. about 4.6 mg) of the GLP-1R agonist (e.g., GLP-1R/GCGR dual agonist), or pharmaceutically acceptable salt thereof;
about 4 mg to about 5 mg (e.g. about 4.5 mg) of the AMYR agonist, or pharmaceutically acceptable salt thereof, and about 2 mg to about 4 mg (e.g. about 2.3 mg) of the GLP-1R agonist (e.g., GLP-1R/GCGR dual agonist), or pharmaceutically acceptable salt thereof;
about 4 mg to about 5 mg (e.g. about 4.5 mg) of the AMYR agonist, or pharmaceutically acceptable salt thereof, and about 1 mg to about 2 mg (e.g. about 1.2 mg) of the GLP-1R agonist (e.g., GLP-1R/GCGR dual agonist), or pharmaceutically acceptable salt thereof;
about 1 mg to about 2 mg (for example, about 1.50 mg or about 1.60 mg) of the AMYR agonist, or pharmaceutically acceptable salt thereof, and about 8 mg to about 10 mg (e.g. about 9.4 mg) of the GLP-1R agonist (e.g., GLP-1R/GCGR dual agonist), or pharmaceutically acceptable salt thereof;
about 1 mg to about 2 mg (for example, about 1.50 mg or about 1.60 mg) of the AMYR agonist, or pharmaceutically acceptable salt thereof, and about 5 mg to about 10 mg (e.g. about 7 mg) of the GLP-1R agonist (e.g., GLP-1R/GCGR dual agonist), or pharmaceutically acceptable salt thereof;
about 1 mg to about 2 mg (for example, about 1.50 mg or about 1.60 mg) of the AMYR agonist, or pharmaceutically acceptable salt thereof, and about 3 mg to about 5 mg (e.g. about 4.6 mg) of the GLP-1R agonist (e.g., GLP-1R/GCGR dual agonist), or pharmaceutically acceptable salt thereof;
about 1 mg to about 2 mg (for example, about 1.50 mg or about 1.60 mg) of the AMYR agonist, or pharmaceutically acceptable salt thereof, and about 2 mg to about 4 mg (e.g. about 2.3 mg) of the GLP-1R agonist (e.g., GLP-1R/GCGR dual agonist), or pharmaceutically acceptable salt thereof; or
about 1 mg to about 2 mg (for example, about 1.50 mg or about 1.60 mg) of the AMYR agonist, or pharmaceutically acceptable salt thereof, and about 1 mg to about 2 mg (e.g. about 1.2 mg) of the GLP-1R agonist (e.g., GLP-1R/GCGR dual agonist), or pharmaceutically acceptable salt thereof;
   about once a week over a period of at least 9 months.

The AMYR agonist, or pharmaceutically acceptable salt thereof, may be administered to the subject at a dose, such as a fixed dose as described herein about once a week over a period of between about 3 months to about 9 months, such as about 3 months to about 6 months. By way of non-limiting example, the method may comprise administering to the subject a dose of:
about 7 mg to about 10 mg (e.g., about 9 mg) of the AMYR agonist, or pharmaceutically acceptable salt thereof, and about 8 mg to about 10 mg (e.g. about 9.4 mg) of the GLP-1R agonist (e.g., GLP-1R/GCGR dual agonist), or pharmaceutically acceptable salt thereof;
about 7 mg to about 10 mg (e.g., about 9 mg) of the AMYR agonist, or pharmaceutically acceptable salt thereof, and about 5 mg to about 10 mg (e.g. about 7 mg) of the GLP-1R agonist (e.g., GLP-1R/GCGR dual agonist), or pharmaceutically acceptable salt thereof;
about 7 mg to about 10 mg (e.g., about 9 mg) of the AMYR agonist, or pharmaceutically acceptable salt thereof, and about 3 mg to about 5 mg (e.g. about 4.6 mg) of the GLP-1R agonist (e.g., GLP-1R/GCGR dual agonist), or pharmaceutically acceptable salt thereof;
about 7 mg to about 10 mg (e.g., about 9 mg) of the AMYR agonist, or pharmaceutically acceptable salt thereof, and about 2 mg to about 4 mg (e.g. about 2.3 mg) of the GLP-1R agonist (e.g., GLP-1R/GCGR dual agonist), or pharmaceutically acceptable salt thereof;
about 7 mg to about 10 mg (e.g., about 9 mg) of the AMYR agonist, or pharmaceutically acceptable salt thereof, and about 1 mg to about 2 mg (e.g. about 1.2 mg) of the GLP-1R agonist (e.g., GLP-1R/GCGR dual agonist), or pharmaceutically acceptable salt thereof;
about 4 mg to about 7 mg (e.g. about 6 mg) of the AMYR agonist, or pharmaceutically acceptable salt thereof, and about 8 mg to about 10 mg (e.g. about 9.4 mg) of the GLP-1R agonist (e.g., GLP-1R/GCGR dual agonist), or pharmaceutically acceptable salt thereof;
about 4 mg to about 7 mg (e.g. about 6 mg) of the AMYR agonist, or pharmaceutically acceptable salt thereof, and about 5 mg to about 10 mg (e.g. about 7 mg) of the GLP-1R agonist (e.g., GLP-1R/GCGR dual agonist), or pharmaceutically acceptable salt thereof;
about 4 mg to about 7 mg (e.g. about 6 mg) of the AMYR agonist, or pharmaceutically acceptable salt thereof, and about 3 mg to about 5 mg (e.g. about 4.6 mg) of the GLP-1R agonist (e.g., GLP-1R/GCGR dual agonist), or pharmaceutically acceptable salt thereof;
about 4 mg to about 7 mg (e.g. about 6 mg) of the AMYR agonist, or pharmaceutically acceptable salt thereof, and about 2 mg to about 4 mg (e.g. about 2.3 mg) of the GLP-1R agonist (e.g., GLP-1R/GCGR dual agonist), or pharmaceutically acceptable salt thereof;
about 4 mg to about 7 mg (e.g. about 6 mg) of the AMYR agonist, or pharmaceutically acceptable salt thereof, and about 1 mg to about 2 mg (e.g. about 1.2 mg) of the GLP-1R agonist (e.g., GLP-1R/GCGR dual agonist), or pharmaceutically acceptable salt thereof;
about 4 mg to about 5 mg (e.g. about 4.5 mg) of the AMYR agonist, or pharmaceutically acceptable salt thereof, and about 8 mg to about 10 mg (e.g. about 9.4 mg) of the GLP-1R agonist (e.g., GLP-1R/GCGR dual agonist), or pharmaceutically acceptable salt thereof;
about 4 mg to about 5 mg (e.g. about 4.5 mg) of the AMYR agonist, or pharmaceutically acceptable salt thereof, and about 5 mg to about 10 mg (e.g. about 7 mg) of the GLP-1R agonist (e.g., GLP-1R/GCGR dual agonist), or pharmaceutically acceptable salt thereof;
about 4 mg to about 5 mg (e.g. about 4.5 mg) of the AMYR agonist, or pharmaceutically acceptable salt thereof, and about 3 mg to about 5 mg (e.g. about 4.6 mg) of the GLP-1R agonist (e.g., GLP-1R/GCGR dual agonist), or pharmaceutically acceptable salt thereof;
about 4 mg to about 5 mg (e.g. about 4.5 mg) of the AMYR agonist, or pharmaceutically acceptable salt thereof, and about 2 mg to about 4 mg (e.g. about 2.3 mg) of the GLP-1R agonist (e.g., GLP-1R/GCGR dual agonist), or pharmaceutically acceptable salt thereof;
about 4 mg to about 5 mg (e.g. about 4.5 mg) of the AMYR agonist, or pharmaceutically acceptable salt thereof, and about 1 mg to about 2 mg (e.g. about 1.2 mg) of the GLP-1R agonist (e.g., GLP-1R/GCGR dual agonist), or pharmaceutically acceptable salt thereof;
about 1 mg to about 2 mg (for example, about 1.50 mg or about 1.60 mg) of the AMYR agonist, or pharmaceutically acceptable salt thereof, and about 8 mg to about 10 mg (e.g. about 9.4 mg) of the GLP-1R agonist (e.g., GLP-1R/GCGR dual agonist), or pharmaceutically acceptable salt thereof;
about 1 mg to about 2 mg (for example, about 1.50 mg or about 1.60 mg) of the AMYR agonist, or pharmaceutically acceptable salt thereof, and about 5 mg to about 10 mg (e.g. about 7 mg) of the GLP-1R agonist (e.g., GLP-1R/GCGR dual agonist), or pharmaceutically acceptable salt thereof;
about 1 mg to about 2 mg (for example, about 1.50 mg or about 1.60 mg) of the AMYR agonist, or pharmaceutically acceptable salt thereof, and about 3 mg to about 5 mg (e.g. about 4.6 mg) of the GLP-1R agonist (e.g., GLP-1R/GCGR dual agonist), or pharmaceutically acceptable salt thereof;
about 1 mg to about 2 mg (for example, about 1.50 mg or about 1.60 mg) of the AMYR agonist, or pharmaceutically acceptable salt thereof, and about 2 mg to about 4 mg (e.g. about 2.3 mg) of the GLP-1R agonist (e.g., GLP-1R/GCGR dual agonist), or pharmaceutically acceptable salt thereof; or
about 1 mg to about 2 mg (for example, about 1.50 mg or about 1.60 mg) of the AMYR agonist, or pharmaceutically acceptable salt thereof, and about 1 mg to about 2 mg (e.g. about 1.2 mg) of the GLP-1R agonist (e.g., GLP-1R/GCGR dual agonist), or pharmaceutically acceptable salt thereof;
   about once a week over a period of about 3 months to about 9 months, such as about 3 months to about 6 months (e.g. about 3 months, about 4 months, about 5 months, about 6 months, about 7 months, about 8 months, or about 9 months).

The combination therapy may be titrated to a final dose over a period of time. The combination therapy may be titrated to a final dose over a period between about 1 week to about 36 weeks. The combination therapy may be titrated to a final dose over a period between about 1 week to about 21 weeks, such as up to about 20 weeks. The combination therapy may be titrated to a final dose over a period between about 1 week to about 17 weeks, such as up to about 16 weeks. The combination therapy may be titrated to a final dose over a period between about 1 week to about 8 weeks. For example, the combination therapy may be titrated to a final dose over a period of about 20 weeks.

The combination therapy may be titrated to a final dose over a period between about 2 weeks to about 36 weeks. The combination therapy may be titrated to a final dose over a period between about 2 weeks to about 21 weeks (e.g. up to about 20 weeks). The combination therapy may be titrated to a final dose over a period between about 2 weeks to about 17 weeks (e.g. up to about 16 weeks). The combination therapy may be titrated to a final dose over a period between about 2 weeks to about 8 weeks. The combination therapy may be titrated to a final dose over a period between about 2 weeks to about 6 weeks.

The combination therapy may be titrated to a final dose over a period between about 4 weeks to about 8 weeks.

The combination therapy may be titrated to a final dose over about 2 weeks. The combination therapy may be titrated to a final dose over about 4 weeks. The combination therapy may be titrated to a final dose over about 6 weeks. The combination therapy may be titrated to a final dose over about 8 weeks. The combination therapy may be titrated to a final dose over about 17 weeks. The combination therapy may be titrated to a final dose over about 21 weeks. The combination therapy may be titrated to a final dose over about 36 weeks.

Where the combination therapy comprises a dose of about 9 mg AMYR agonist, the AMYR agonist may be titrated to a dose of about 9 mg over about 21 weeks. Where the combination therapy comprises a dose of about 9 mg AMYR agonist, the AMYR agonist may be titrated to a dose of about 9 mg over about 20 weeks. Where the combination therapy comprises a dose of about 9 mg AMYR agonist, the AMYR agonist may be titrated to a dose of about 9 mg over about 17 weeks. Where the AMYR agonist is a dose of about 9 mg, the AMYR agonist may be titrated to a dose of about 9 mg over about 8 weeks. Where the AMYR agonist is a dose of about 9 mg, the AMYR agonist may be titrated to a dose of about 9 mg over about 6 weeks. Where the combination therapy comprises a dose of about 6.0 mg AMYR agonist, the AMYR agonist may be titrated to a dose of about 6 mg over about 17 weeks. Where the AMYR agonist is a dose of about 6 mg, the AMYR agonist may be titrated to a dose of about 9 mg over about 8 weeks. Where the AMYR agonist is a dose of about 6 mg, the AMYR agonist may be titrated to a dose of about 9 mg over about 4 weeks. Where the combination therapy comprises a dose of about 1.5 mg AMYR agonist (e.g. about 1.6 mg), the AMYR agonist may be titrated to a dose of about 1.5 mg (e.g. about 1.6 mg) over about 5 weeks. Where the combination therapy comprises a dose of about 1.5 mg AMYR agonist (e.g. about 1.6 mg), the AMYR agonist may be titrated to a dose of about 1.5 mg (e.g. about 1.6 mg) over about 4 weeks.

Where the combination therapy comprises a dose of about 9.4 mg GLP-1R agonist (e.g. GLP-1R/GCGR dual agonist), the GLP-1R agonist may be titrated to a dose of about 9.4 mg over about 21 weeks. Where the combination therapy comprises a dose of about 9.4 mg GLP-1R agonist (e.g. GLP-1R/GCGR dual agonist), the GLP-1R agonist may be titrated to a dose of about 9.4 mg over about 20 weeks. Where the combination therapy comprises a dose of about 9.4 mg GLP-1R agonist (e.g. GLP-1R/GCGR dual agonist), the GLP-1R agonist may be titrated to a dose of about 9.4 mg over about 17 weeks. Where the combination therapy comprises a dose of about 9.4 mg GLP-1R agonist (e.g. GLP-1R/GCGR dual agonist), the GLP-1R agonist may be titrated to a dose of about 9.4 mg over about 8 weeks. Where the combination therapy comprises a dose of about 9.4 mg GLP-1R agonist (e.g. GLP-1R/GCGR dual agonist), the GLP-1R agonist may be titrated to a dose of about 9.4 mg over about 6 weeks. Where the combination therapy comprises a dose of about 7 mg GLP-1R agonist (e.g. GLP-1R/GCGR dual agonist), the GLP-1R agonist may be titrated to a dose of about 7 mg over about 21 weeks. Where the combination therapy comprises a dose of about 7 mg GLP-1R agonist (e.g. GLP-1R/GCGR dual agonist), the GLP-1R agonist may be titrated to a dose of about 7 mg over about 20 weeks. Where the combination therapy comprises a dose of about 7 mg GLP-1R agonist (e.g. GLP-1R/GCGR dual agonist), the GLP-1R agonist may be titrated to a dose of about 7 mg over about 17 weeks. Where the combination therapy comprises a dose of about 7 mg GLP-1R agonist (e.g. GLP-1R/GCGR dual agonist), the GLP-1R agonist may be titrated to a dose of about 7 mg over about 8 weeks. Where the combination therapy comprises a dose of about 7 mg GLP-1R agonist (e.g. GLP-1R/GCGR dual agonist), the GLP-1R agonist may be titrated to a dose of about 7 mg over about 6 weeks. Where the combination therapy comprises a dose of about 4.6 mg GLP-1R agonist (e.g. GLP-1R/GCGR dual agonist), the GLP-1R agonist may be titrated to a dose of about 4.6 mg over about 17 weeks. Where the combination therapy comprises a dose of about 4.6 mg GLP-1R agonist (e.g. GLP-1R/GCGR dual agonist), the GLP-1R agonist may be titrated to a dose of about 4.6 mg over about 8 weeks. Where the combination therapy comprises a dose of about 4.6 mg GLP-1R agonist (e.g. GLP-1R/GCGR dual agonist), the GLP-1R agonist may be titrated to a dose of about 4.6 mg over about 4 weeks. Where the combination therapy comprises a dose of about 1.2 mg GLP-1R agonist (e.g. GLP-1R/GCGR dual agonist), the GLP-1R agonist may be titrated to a dose of about 1.2 mg over about 5 weeks. Where the combination therapy comprises a dose of about 1.2 mg GLP-1R agonist (e.g. GLP-1R/GCGR dual agonist), the GLP-1R agonist may be titrated to a dose of about 1.2 mg over about 4 weeks.

Titration may involve a single step-change in the dose of the AMYR agonist and/or the GLP-1R agonist (e.g. GLP-1R/GCGR dual agonist). Each step-change may comprise one more administrations of a dose (e.g. 1, 2, 3, or 4 doses) at a given titration level.

Alternatively, titration may involve multiple step-changes (e.g. 2, 3 or more step-changes) in the dose of the combination therapy. Each step-change may comprise one more administrations of a dose (e.g. 1, 2, 3, or 4 doses) at a given titration level. Thus, the combination therapy may be titrated at a first dose over a first period of time, increased to one or more intermediate doses over a second or subsequent (e.g., a third, fourth, or fifth, etc.) period of time, and then increased to a final dose. The first, second, and subsequent periods of time may be the same or different lengths of time. The first period of time may be between about 1 week to about 8 weeks. The first period of time may be between about 2 weeks to about 8 weeks. The first period of time may be between about 2 weeks to about 6 weeks. The first period of time may be between about 4 weeks to about 8 weeks. The first period of time may be about 1 week. The first period of time may be about 2 weeks. The first period of time may be about 4 weeks. The first period of time may be about 6 weeks. The first period of time may be about 8 weeks. The second and/or subsequent period of time may be between about 2 weeks to about 8 weeks. The second and/or subsequent period of time may be between about 2 weeks to about 6 weeks. The second and/or subsequent period of time may be between about 4 weeks to about 8 weeks. The second and/or subsequent period of time may be about 2 weeks. The second and/or subsequent period of time may be about 4 weeks. The second and/or subsequent period of time may be about 6 weeks. The second and/or subsequent period of time may be about 8 weeks.

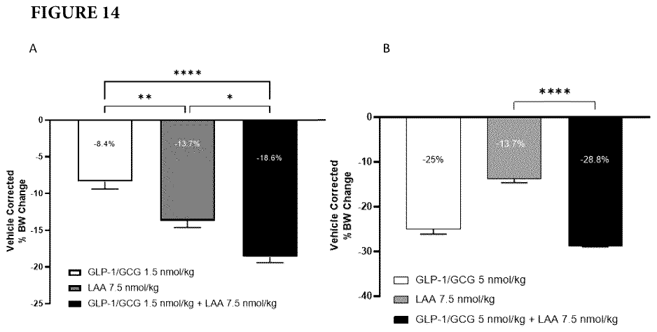

By way of non-limiting example, titration to a final dose of 6 mg AMYR agonist may comprise one or more dose (e.g. 1, 2, 3, or 4 doses) of 0.4 mg, followed by one or more dose (e.g. 1, 2, 3 or 4 doses) at 0.8 mg, followed by one or more dose (e.g. 1, 2, 3 or 4 doses) at 1.6 mg, followed by one or more dose (e.g. 1, 2, 3 or 4 doses) at 3 mg, followed by one or more dose (e.g. 1, 2, 3 or 4 doses) at 4.5 mg, followed by the remaining doses at the final dose of 6 mg. By way of non-limiting example, titration to a final dose of 4.6 mg GLP-1R agonist (e.g. GLP-1R/GCGR dual agonist) may comprise one or more dose (e.g. 1, 2, 3, or 4 doses) of 0.3 mg, followed by one or more dose (e.g. 1, 2, 3 or 4 doses) at 0.6 mg, followed by one or more dose (e.g. 1, 2, 3 or 4 doses) at 1.2 mg, followed by one or more dose (e.g. 1, 2, 3 or 4 doses) at 2.3 mg, followed by one or more dose (e.g. 1, 2, 3 or 4 doses) at 3.5 mg, followed by the remaining doses at the final dose of 4.6 mg. Further exemplary titration schedules are set out in FIGS. 14-17.

In some particularly preferred embodiments, the combination therapy comprises or consists of (a) an AMYR agonist polypeptide, consisting of the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, at any dose disclosed herein; and (b) a GLP-1R agonist (e.g. a GLP-1R/GCGR dual-agonist) polypeptide consisting of the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, at any dose disclosed herein.

### Amylin Receptor Agonists

### Efficacy

Typically the AMYR is human AYMR (hAMYR). Human AMYR is formed of the human calcitonin receptor hCTR complexed with at least one of the human receptor activity modifying proteins designated hRAMP1, hRAMP2 and hRAMP3. Thus, hAMY1R is a complex of hCTR and hRAMP1; hAMY2R is a complex of hCTR and hRAMP2; and hAMY3R is a complex of hCTR and hRAMP3. An exemplary amino acid sequence for hRAMP1 is given in SEQ ID NO: 1. An exemplary amino acid sequence for hRAMP2 is given in SEQ ID NO: 2. An exemplary amino acid sequence for hRAMP3 is given in SEQ ID NO: 3. An exemplary amino acid sequence for hCTR is given in SEQ ID NO: 4.

An AMYR agonist of the invention may activate one or more of hAMY1R, hAMY2R and/or hAMY3R. Thus, an AMYR agonist of the invention may activate hAMY1R; hAMY2R; hAMY3R; hhAMY1R and AMY2R; hAMY1R and AhMY3R; hAMY2R and hAMY3R; or hAMY1R, hAMY2R and hAMY3R.

An AMYR agonist is not native amylin but exhibits activity at the AMYR of about at least 1% or more relative to native amylin. In some embodiments, an AMYR agonist exhibits activity at the AMYR of at least 5%, 10%, 20%, 30%, 40%, 50%, 60%, 70%, 80%, 90%, 95%, or more relative to native amylin. This may be measured using a cAMP assay, and quantified by Effective Concentration (EC) values, such as asymptotic maximum attainable response, Eₘₐₓ, or EC₅₀, as described further below.

The ability to induce cAMP formation as a result of binding to the relevant receptor or receptor complex is typically regarded as indicative of agonist activity. Other intracellular signaling pathways or events may also be used as readouts for amylin receptor agonist activity. These may include calcium release, arrestin recruitment, receptor internalization, kinase activation or inactivation, lipase activation, inositol phosphate release, diacylglycerol release or nuclear transcription factor translocation.

EC₅₀ values may be used as a measure of agonist potency at a given receptor. An EC₅₀ value is a measure of the concentration of a compound required to achieve half of that compound's maximal activity in a particular assay, for example a cAMP assay as described in Example 2 of WO 2022/129254, which is herein incorporated by reference in its entirety.

In particular, an AMYR agonist of the invention has selectivity to AMYR (preferably hAMYR) over a CTR (preferably hCTR). Such amylin agonists are described as selective amylin receptor agonists (SARAs). Thus, in some embodiments, the AMYR agonist of the invention is a SARA. In some embodiments, the AMYR agonist of the invention is not a dual amylin and calcitonin receptor agonist (DACRA). A SARA may have selectivity for AMYR over a CTR as described further below in the context of AMYR agonists of the invention.

An AMYR agonist of the invention may exhibit greater or similar selectivity to hAMYR over hCTR as pramlintide, optionally as measured using cAMP release from binding to hAMYR and hCTR. Pramlintide exhibits at least 10-fold selectivity to hAMYR as compared to hCTR.

An AMYR agonist of the invention may exhibit a lower or similar selectivity to hAMYR over hCTR as pramlintide, optionally as measured using cAMP release from binding to hAMYR and hCTR. Where an AMYR agonist of the invention exhibits a lower selectivity to hAMYR over hCTR compared with pramlintide, the AMYR agonist of the invention is still selective for hAMYR over hCTR. Pramlintide exhibits at least 10-fold selectivity to hAMYR as compared to hCTR.

In some embodiments, the AMYR agonist of the invention is not pramlintide. Thus, in some embodiments, the AMYR agonist of the invention is not a DACRA and is not pramlintide. In some embodiments, the AMYR agonist of the invention is a SARA and is not pramlintide.

An AMYR agonist of the invention may have at least a 2-fold, at least 3-fold, at least 4-fold, at least 5-fold, at least 6-fold, at least 7-fold, at least 8-fold, at least 9-fold, at least 10-fold, at least 12-fold, at least 15-fold, at least 17-fold, at least 20-fold, or at least 25-fold, at least about 50-fold, at least about 75-fold, or at least about 100-fold selectivity to hAMYR over hCTR. In preferred embodiments, the AMYR agonist has at least a 5-fold selectivity to AMYR (e.g. hAMYR) over CTR (e.g. hCTR). In other preferred embodiments, the AMYR agonist has at least a 10-fold selectivity to AMYR (e.g. hAMYR) over CTR (e.g. hCTR).

In some embodiments, an AMYR agonist of the invention has around 12-20-fold, around 14-18-fold, optionally around 16-fold selectivity to AMYR (e.g. hAMYR) over CTR (e.g. hCTR).

In some embodiments, the AMYR agonist of the invention has an EC₅₀ measured under the conditions described in Example 2 of WO 2022/129254 (i.e. containing 0.1% bovine serum albumin (BSA)) of below about 1.4 nM, below about 1.2 nM, below about 1 nM, below about 0.8 nM, below about 0.6 nM, below about 0.4 nM, below about 0.3 nM, or below about 0.2 nM for an AMYR, particularly AMY3R.

In contrast, a DACRA may have less than 2-fold selectivity, less than 1.5-fold selectivity, or less than 1.2-fold selectivity for hAMYR over hCTR. A DACRA may have approximately equal selectivity for hAMYR and hCTR. A DACRA may have at least a 1.2-fold, at least a 1.5-fold, or at least a 2-fold selectivity to hCTR over hAMYR.

### Classification

The AMYR agonist of the invention may be a polypeptide, a small molecule drug, an antibody, an antibody-drug conjugate (ADC), or an aptamer; or a pharmaceutically acceptable salt thereof.

The AMYR agonist of the invention may be a small molecule drug. As used herein a “small molecule drug” refers to a low molecular weight compound, typically an organic compound. Typically, a small molecule has a maximum molecule weight of 900 Da, allowing for rapid diffusion across cell membranes. In some embodiments, the maximum molecular weight of a small molecule is 500 Da. Typically a small molecule has a size in the order of 1 nm.

The AMYR agonist of the invention may be an aptamer. Aptamers are generally nucleic acid molecules that bind a specific target molecule. Aptamers can be engineered completely in vitro, are readily produced by chemical synthesis, possess desirable storage properties, and elicit little or no immunogenicity in therapeutic applications. These characteristics make them particularly useful in pharmaceutical and therapeutic utilities.

As used herein, “aptamer” refers in general to a single or double stranded oligonucleotide or a mixture of such oligonucleotides, wherein the oligonucleotide or mixture is capable of binding specifically to a target. Oligonucleotide aptamers will be discussed here, but the skilled reader will appreciate that other aptamers having equivalent binding characteristics can also be used, such as peptide aptamers. In general, aptamers may comprise oligonucleotides that are at least 5, at least 10 or at least 15 nucleotides in length. Aptamers may comprise sequences that are up to 40, up to 60 or up to 100 or more nucleotides in length. For example, aptamers may be from 5 to 100 nucleotides, from 10 to 40 nucleotides, or from 15 to 40 nucleotides in length. Where possible, aptamers of shorter length are preferred as these will often lead to less interference by other molecules or materials.

The AMYR agonist of the invention may be an antibody. As used herein, the term antibody encompasses the use of a monoclonal antibody or polyclonal antibody, as well as the antigen-binding fragments of a monoclonal or polyclonal antibody, or a peptide which binds to REV-ERB with specificity. The antibody may be a Fab, F(ab′)2, Fv, scFv, Fd, Fc or dAb.

The AMYR agonist of the invention may be an ADC, in which a pharmaceutically active moiety is conjugated, directly or indirectly (e.g. via a linker) to an antibody or fragment thereof. For example, an AMYR agonist may be an amylin analogue conjugated, directly or indirectly, to an Fc fragment. The linker may be cleavable or non-cleavable.

Preferably, an AMYR agonist of the invention is a polypeptide. An AMYR agonist polypeptide may be of between about 15 to about 40 amino acids in length, preferably between about 30 to about 40 amino acids in length, most preferably about 37 amino acids in length. For the avoidance of doubt, any and all disclosure herein in relation to AMYR agonists of the invention applies particularly to AMYR agonist polypeptides unless expressly stated to the contrary.

### AMYR Agonist Polypeptides

Throughout this specification, amino acid positions of an AMYR agonist polypeptide (e.g. lipidated AMYR agonist polypeptides) are numbered according to the corresponding position in pramlintide having the sequence set forth in SEQ ID NO: 5.

Throughout this specification, amino acids are referred to by their conventional three-letter or single-letter abbreviations (e.g. Ala or A for alanine, Arg or R for arginine, etc.). In the case of certain less common or non-naturally occurring amino acids (i.e. amino acids other than the 20 encoded by the standard mammalian genetic code), unless they are referred to by their full name, frequently employed three- or four-character codes are employed for residues thereof, including αMeSer ((S)-2-amino-3-hydroxy-2-methylphenylpropanoic acid), αMePhe ((S)-2-amino-2-methyl-3-phenylpropanoic acid), Aib (2-amino-2-methylpropanoic acid), Dab (2,4-diaminobutanoic acid) and γ-Glu (γ-glutamic acid, γE).

An AMYR agonist polypeptide of the invention may comprise or consist of an amino acid sequence having at least 70%, at least 80%, at least 90%, at least 95%, at least 96%, at least 97%, at least 98%, at least 99% or more, up to 100% identity to pramlintide (KCNTATCATQRLANFLVHSSNNFGPILPPTNVGSNTY-amide, SEQ ID NO: 5). Preferably, an AMYR agonist polypeptide of the invention may comprise or consist of an amino acid sequence having at least 90%, at least 95%, at least 96%, at least 97%, at least 98%, at least 99% or more, up to 100% identity to pramlintide (KCNTATCATQRLANFLVHSSNNFGPILPPTNVGSNTY-amide, SEQ ID NO: 5).

AMYR agonist polypeptides disclosed here can be formulated in or chemically conjugated to e.g. a protein, polymeric drug carrier or advance drug delivery system that further enhance the chemical stability and or physical stability and or the circulatory exposure of the AMYR agonist polypeptide.

In embodiments of any aspect of the invention, the polypeptides (e.g. lipidated polypeptides) of the invention are isolated polypeptides (e.g. isolated lipidated polypeptides).

### Modified Amino Acids

An AMYR agonist polypeptide (e.g. lipidated polypeptides) of the invention may comprise one or more amino acid modifications or substitutions compared to the pramlintide sequence (SEQ ID NO: 5). The AMYR agonist polypeptide may comprise one or more non-proteinogenic amino acids, particularly 2,4-diaminobutanoic acid (Dab). Alternatively or in addition, the AMYR agonist polypeptide comprises one or more alpha methyl amino acids, particularly selected from 2-amino-2-methylpropanoic acid (Aib) and/or alpha methyl phenylalanine (αMePhe or aMeF). The reference to αMePhe and aMeF herein refers to (S)-2-amino-2-methyl-3-phenylpropanoic acid.

### Lipidation and Linkers

An AMYR agonist polypeptide of the invention may be lipidated. Preferably, lipidated polypeptides may have extended half-life compared to pramlintide but without the fibril-forming tendency of lipidated pramlintide analogues described in the art. Without being bound by theory, it is thought that the lipid acts as an albumin binding moiety and protects the AMYR agonist polypeptide against clearance and degradation, thereby extending the half-life of the AMYR agonist polypeptide.

Accordingly, in some embodiments, the lipid may comprise a hydrocarbon chain having from 10 to 26 C atoms, e.g. from 14 to 24 C atoms, e.g. from 16 to 22 C atoms. For example, the hydrocarbon chain may contain 14, 15, 16, 17, 18, 19, 20, 21, 22, 23 or 24 C atoms. In preferred embodiments, the lipid has 18 to 20 C atoms. In particular, the lipid may have 18 C atoms or 20 C atoms. The hydrocarbon chain may be linear or branched, and may be saturated or unsaturated. Furthermore, it can include a functional group at the end of the lipophilic chain, e.g. a carboxylic acid group which may or may not be protected during synthesis.

In some preferred embodiments, the AMYR agonist polypeptide of the invention is lipidated with a lipid is selected from the group consisting of C12diacid, C14diacid, C16diacid, C17diacid, C18diacid, C19diacid or C20diacid. In some particularly preferred embodiments, the lipid is C18diacid or C20diacid.

The AMYR agonist polypeptide may comprise at least one lipidated amino acid residue. In some embodiments, the AMYR agonist polypeptide comprises at least two lipidated amino acid residues. In preferred embodiments, the AMYR agonist polypeptide contains only one lipidated amino acid residue. The lipid may be attached to an amino acid residue of the polypeptide.

In some embodiments, the lipid is attached to the amino acid residue through a linker (referred to herein as “linker-lipid”). In alternative embodiments, the lipid is directly attached to the amino acid residue without an intervening linker. The lipid may be attached to the amino acid residue via an ester, a sulfonyl ester, a thioester, an amide, an amine or a sulphonamide. Accordingly, it will be understood that the lipid or the linker includes an acyl group, a sulphonyl group, an N atom, an O atom or an S atom which forms part of the ester, sulphonyl ester, thioester, amide, amine or sulphonamide. Optionally, an acyl group in the lipid or linker forms part of an amide or ester with the amino acid residue. Accordingly, in preferred embodiments the lipid is attached to an acylation site on the amino acid residue.

The linker may be attached to any residue of the AMYR agonist polypeptide. In some embodiments, the linker is attached to the side chain of an amino acid residue in the polypeptide, for example to the ε-N of a lysine residue. Preferably, the linker is attached to the N-terminus of the polypeptide, (e.g. to a lysine at the N-terminus of the polypeptide).

The linker may comprise one or more residues of any naturally occurring or non-naturally occurring amino acid. The linker may comprise a combination of residues, as single or repeating units. For example, the linker may comprise multiple combinations of residues, as single or repeating units, each of which may independently be a residue of Glu (E), y-Glu (γE), Lys, ε-Lys, Asp, β-Asp, Gaba, β-Ala (3-aminopropanoyl), O2Oc (2-(2-(2-aminoethoxy)ethoxy)acetic acid), PEG2 (3-(2-(2-aminoethoxy)ethoxy)propanoic acid), PEG4 (1-amino-3,6,9,12-tetraoxapentadecan-15-oic acid), PEGS (1-amino-3,6,9,12,15,18,21,24-octaoxaheptacosan-27-oic acid, PEG12 (1-amino-3,6,9,12,15,18,21,24,27,30,33,36-dodecaoxanonatriacontan-39-oic acid). y-Glu and β-Asp refer to amino acids where the alpha-amino group and the side chain carboxyl group participate in peptide bond formation. ε-Lys refers to an amino acid where the epsilon-amino and carboxyl group of lysine participate in peptide bond formation.

In some embodiments, an AMYR agonist polypeptide comprises a lipid is attached to an amino acid residue in the AMYR agonist polypeptide by a linker, wherein the linker comprises y-glutamic acid (γE), γE-γE, ((O2Oc)-(O2Oc)-γE), ((O2Oc)-(O2Oc)-γEγE), or ((PEG2)-(PEG2)-γE). In some embodiments, an AMYR agonist polypeptide comprises a lipid is attached to an amino acid residue in the AMYR agonist polypeptide by a linker, wherein the linker consists of γE, γE-γE, ((O2Oc)-(O2Oc)-γE), ((O2Oc)-(O2Oc)-γEγE), or ((PEG2)-(PEG2)-γE).

Combinations of linker, lipid and polypeptide acylation sites are described in WO 2022/129254.

In some preferred embodiments, the lysine at position 1 of the AMYR agonist polypeptide, or pharmaceutically acceptable salt thereof, is lipidated. In some particularly preferred embodiments, wherein the lysine at position 1 of the AMYR agonist polypeptide is lipidated, the lipid is linked to the lysine via a γE-γE linker, and the lipid is octadecanedioic acid (C18diacid).

### Exemplary AMYR Agonist Polypeptides

In some preferred embodiments, the lipidated polypeptides of WO 2022/129254 may be used in the combination therapies of the present invention. Thus, an AMYR agonist polypeptide, or pharmaceutically acceptable salt thereof, may comprise or consist of an amino acid sequence having at least 90%, at least 95 at least 97%, at least 98%, at least 99% or more, up to 100% identity to an amino acid sequence selected from the group consisting of:

(SEQ ID NO: 6)
C18diacid-γE-[CNTATC]ATQRLAEFLRHSSNN(αMePhe)
GPILPPTNVGSNTY-amide;
(SEQ ID NO: 7)
K(γE-γE-C18diacid)[CNTATC]ATQRLAEFLRHSSNN(αMePhe)
GPILPPTNVGSNTY-amide;
(SEQ ID NO: 8)
K(γE-C18diacid)K[CNTATC]ATQRLAEFLRHSSNN(αMePhe)
GPILPPTNVGSNTY-amide;
(SEQ ID NO: 9)
K(γE-C18diacid)K[CNTATC]ATQRLANFLRHSSNN(αMePhe)
GPILPPTEVGSNTY-amide;
(SEQ ID NO: 10)
K(O20c-O2Oc-γE-18diacid)[CNTATC]ATQRLANFLRHSSNN
(αMePhe)GPILPPTEVGSNTY-amide;
(SEQ ID NO: 11)
K(γE-γE-C18diacid)[CNTATC]ATQRLANFLRHSSNN(αMePhe)
GPILPPTEVGSNTY-amide;
(SEQ ID NO: 12)
K(γE-C18diacid)[CNTATC]ATQRLANFLRHSSNN(αMePhe)
GPILPPTNVGSNTY-amide;
(SEQ ID NO: 13)
K(γE-γE-C18diacid)[CNTATC]ATQRLANFLRHSSNN(αMePhe)
GPILPPTNVGSNTY-amide;
(SEQ ID NO: 14)
K(γE-γE-C18diacid)[CNTATC]ATQRLANFLVHSSNN(αMePhe)
GPILPPTNVGSRTY-amide;
(SEQ ID NO: 15)
K(γE-γE-C18diacid)[CNTATC]ATQRLANFLVHSSNN(αMePhe)
GPILPPTRVGSNTY-amide;
(SEQ ID NO: 16)
K(γE-C18diacid)[CNTATC]ATQRLANFLVHSSNN(αMePhe)
GPILPPTRVGSNTY-amide;
(SEQ ID NO: 17)
K(γE-C18diacid)K[CNTATC]ATQRLA(Dab)
FLVHSSNNFGPILPPTNVGSNTY-amide;
(SEQ ID NO: 18)
K(γE-C18diacid)K[CNTATC]ATQRLANFLVHSS(Aib)
NFGPILPPTHVGSNTY-amide;
(SEQ ID NO: 19)
K(γE-C18diacid)[CNTATC]ATQRLANFLRHSS(Aib)
NFGPILPPTEVGSNTY-amide;
(SEQ ID NO: 20)
K(γE-C18diacid)[CNTATC]ATQRLANFLRHSS(Aib)
NFGPILPPTNVGSNTY-amide;
(SEQ ID NO: 21)
K(γE-γE-C18diacid)K[CNTATC]ATQRLANFLRHSS(Aib)
NFGPILPPTNVGSNTY-amide;
(SEQ ID NO: 22)
K(γE-γE-C18diacid)K[CNTATC]ATQRLANFLVHSS(Aib)
NFGPILPPTRVGSNTY-amide;
(SEQ ID NO: 23)
K(γE-C18diacid)K[CNTATC]ATQRLANFLVHSS(Aib)
NFGPILPPT(Aib)VGSNTY-amide;
(SEQ ID NO: 24)
K(γE-C18diacid)K[CNTATC]ATQRLANFL(Aib)
HSSNNFGPILPPTNVGSNTY-amide;
and
(SEQ ID NO: 25)
K(γE-C18diacid)K[CNTATC]ATQRLA(Dab)FL(Aib)
HSSNNFGPILPPTEVGSNTY-amide.

In some preferred embodiments, an AMYR agonist polypeptide comprises an amino acid sequence having at least 70%, at least 80%, at least 90%, at least 95%, at least 96%, at least 97%, at least 98%, at least 99% or more, up to 100% identity to the amino acid sequence

(SEQ ID NO: 26)
K[CNTATC]ATQRLANFLRHSSNN(αMePhe)
GPILPPTEVGSNTY-amide

In some preferred embodiments, an AMYR agonist polypeptide consists of an amino acid sequence having at least 70%, at least 80%, at least 90%, at least 95%, at least 97%, at least 98%, at least 99% or more, up to 100% identity to the amino acid sequence

(SEQ ID NO: 26)
K[CNTATC]ATQRLANFLRHSSNN(αMePhe)
GPILPPTEVGSNTY-amide

In some particularly preferred embodiments, an AMYR agonist polypeptide comprises an amino acid sequence having at least 90%, at least 95%, at least 97%, at least 98%, at least 99% or more, up to 100% identity to the amino acid sequence

(SEQ ID NO: 26)
K[CNTATC]ATQRLANFLRHSSNN(αMePhe)
GPILPPTEVGSNTY-amide

In some particularly preferred embodiments, an AMYR agonist polypeptide consists of an amino acid sequence having at least 90%, at least 95%, at least 97%, at least 98%, at least 99% or more, up to 100% identity to the amino acid sequence

(SEQ ID NO: 26)
K[CNTATC]ATQRLANFLRHSSNN(αMePhe)
GPILPPTEVGSNTY-amide

More preferably, the AMYR agonist polypeptide, or pharmaceutically acceptable salt thereof, comprises the amino acid sequence K(γE-γE-C18diacid)[CNTATC]ATQRLANFLRHSSNN(αMePhe)GPILPPTEVGSNTY-amide (SEQ ID NO: 11).

Most preferably, the AMYR agonist polypeptide, or pharmaceutically acceptable salt thereof, consists of the amino acid sequence K(γE-γE-C18diacid)[CNTATC]ATQRLANFLRHSSNN(αMePhe)GPILPPTEVGSNTY-amide (SEQ ID NO: 11).

In any one of the methods disclosed herein, the AMYR agonist may be a polypeptide, or a pharmaceutically acceptable salt thereof, comprising the amino acid sequence K(γE-γE-C18diacid)[CNTATC]ATQRLANFLRHSSNN(αMePhe)GPILPPTEVGSNTY-amide (SEQ ID NO: 11) in an amount of about 1.5 mg (e.g. 1.6 mg). The subject may have obesity or an obesity-related condition.

In any one of the methods disclosed herein, the AMYR agonist may be a polypeptide, or a pharmaceutically acceptable salt thereof, comprising the amino acid sequence K(γE-γE-C18diacid)[CNTATC]ATQRLANFLRHSSNN(αMePhe)GPILPPTEVGSNTY-amide (SEQ ID NO: 11) in an amount of about 4.5 mg. The subject may have obesity or an obesity-related condition.

In any one of the methods disclosed herein, the AMYR agonist may be a polypeptide, or a pharmaceutically acceptable salt thereof, comprising the amino acid sequence K(γE-γE-C18diacid)[CNTATC]ATQRLANFLRHSSNN(αMePhe)GPILPPTEVGSNTY-amide (SEQ ID NO: 11) in an amount of about 6 mg. The subject may have obesity or an obesity-related condition.

In any one of the methods disclosed herein, the AMYR agonist may be a polypeptide, or a pharmaceutically acceptable salt thereof, comprising the amino acid sequence K(γE-γE-C18diacid)[CNTATC]ATQRLANFLRHSSNN(αMePhe)GPILPPTEVGSNTY-amide (SEQ ID NO: 11) in an amount of about 9 mg. The subject may have obesity or an obesity-related condition.

In any one of the methods disclosed herein, the AMYR agonist may be a polypeptide, or a pharmaceutically acceptable salt thereof, comprising the amino acid sequence K(γE-γE-C18diacid)[CNTATC]ATQRLANFLRHSSNN(αMePhe)GPILPPTEVGSNTY-amide (SEQ ID NO: 11) in an amount of about 1.5 mg (e.g. 1.6 mg). The subject may have a metabolic disease.

In any one of the methods disclosed herein, the AMYR agonist may be a polypeptide, or a pharmaceutically acceptable salt thereof, comprising the amino acid sequence K(γE-γE-C18diacid)[CNTATC]ATQRLANFLRHSSNN(αMePhe)GPILPPTEVGSNTY-amide (SEQ ID NO: 11) in an amount of about 4.5 mg. The subject may have a metabolic disease.

In any one of the methods disclosed herein, the AMYR agonist may be a polypeptide, or a pharmaceutically acceptable salt thereof, comprising the amino acid sequence K(γE-γE-C18diacid)[CNTATC]ATQRLANFLRHSSNN(αMePhe)GPILPPTEVGSNTY-amide (SEQ ID NO: 11) in an amount of about 6 mg. The subject may have a metabolic disease.

In any one of the methods disclosed herein, the AMYR agonist may be a polypeptide, or a pharmaceutically acceptable salt thereof, comprising the amino acid sequence K(γE-γE-C18diacid)[CNTATC]ATQRLANFLRHSSNN(αMePhe)GPILPPTEVGSNTY-amide (SEQ ID NO: 11) in an amount of about 9 mg. The subject may have a metabolic disease.

### Pharmacokinetics

The AMYR agonist polypeptides used in the invention may exhibit favourable pharmacokinetic properties as compared to pramlintide. For example, the AMYR agonist polypeptides may have an extended half-life as compared to pramlintide.

As used herein, the term “half-life” is used to refer to the time taken for the concentration of isolated polypeptide in plasma to decline to 50% of its original level. Methods to determine the half-life of proteins are known in the art and are described in Example 4 of WO 2022/129254. It will be recognised that an extended half-life is advantageous, as it permits the therapeutic proteins to be administered according to a safe and convenient dosing schedule, e.g. lower doses that can be administered less frequently. Moreover, the achievement of lower doses may provide further advantages such as the provision of an improved safety profile. To the contrary, pramlintide requires frequent and inconvenient administration.

The AMYR agonist polypeptides may have a half-life of at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours or at least 14 hours in rat models. In preferred embodiments, the AMYR agonist polypeptides have a half-life of at least 14 hours.

### Reduced Fibrillation

The AMYR agonist polypeptides may exhibit reduced tendency to undergo fibrillation in pharmaceutically relevant aqueous media, especially at pH values in the range from 4 to 7, as compared to lipidated pramlintide. In some embodiments, the AMYR agonist polypeptides exhibit reduced tendency to undergo fibrillation in pharmaceutically relevant aqueous media, especially at pH values in the range from 4 to 7, as compared to pramlintide which is lipidated in a similar manner e.g. the same lipid is attached, the lipid is attached through the same linker and/or the lipid is attached at the same position. Exemplary lipidated AMYR agonist polypeptides, for example SEQ ID NOs: 6-25.

Accordingly, the AMYR agonist polypeptides may be suited for formulation in acidic media (e.g. pH 4) and in neutral or near-neutral media (e.g. pH 7 or 7.4). Such AMYR agonist polypeptides may be well suited for coformulation with, for example, insulin, various insulin analogues and/or other therapeutic (e.g. anti-diabetic or anti-obesity) agents that require a neutral or near-neutral formulation pH.

In some embodiments, the AMYR agonist polypeptides show no detectable fibrillation after about 5 hours, after about 7 hours, after about 9 hours, after about 11 hours, after about 13 hours, after about 15 hours, after about 17 hours or after about 20 hours, after about 48 hours, after about 72 hours, after about 96 hours, after about 108 hours, after about 120 hours, after 132 about hours or after about 144 hours at pH 4 and 37° C. Suitable assays for quantifying fibrillation are described in Example 3 of WO 2022/129254 (which describes a Thioflavin T fibrillation assay).

In preferred embodiments, the AMYR agonist polypeptides are soluble at concentrations required for therapeutic efficacy.

### GLP-1 Receptor Agonists

### Efficacy

Native GLP-1 refers to naturally-occurring GLP-1, typically human GLP-1, and is a generic term that encompasses, e.g., GLP-1(7-36) amide (SEQ ID NO: 27), GLP-1(7-37) acid (SEQ ID NO: 28), or a mixture of those two compounds. Typically the GLP-1 receptor (GLP-1R) is human GLP-1R (hGLP-1R). An exemplary hGLP-1R sequence is given in SEQ ID NO: 29.

As used herein, a general reference “GLP-1” in the absence of any further designation is intended to mean native human GLP-1. Unless otherwise indicated, “GLP-1” refers to human GLP-1. Similarly, a general reference “GLP-1R” in the absence of any further designation is intended to mean native human GLP-1R. Unless otherwise indicated, “GLP-1R” refers to human GLP-1R.

A GLP-1 receptor agonist of the invention activates GLP-1R. As used herein, a GLP-1R agonist is not native GLP-1 but exhibits activity at the GLP-1R of about at least 1% or more relative to native GLP-1. In some aspects, a GLP-1R agonist exhibits activity at the GLP-1 receptor of at least 5%, 10%, 20%, 30%, 40%, 50%, 60%, 70%, 80%, 90%, 95%, or more relative to native GLP-1. This may be measured using a cAMP assay, as described herein.

EC₅₀ values may be used as a measure of agonist potency at a given receptor, such as GLP-1R agonist potency. An EC₅₀ value is a measure of the concentration of a compound required to achieve half of that compound's maximal activity in a particular assay, for example a cAMP assay as described in Example 2 of WO 2023/148366, which is herein incorporated by reference in its entirety.

### GLP-1/GCG Dual-Agonists

In some preferred embodiments, a GLP-1R agonist of the invention may also be a glucagon receptor (GCGR) agonist. For the avoidance of doubt, all references herein to GLP-1R agonists of the invention apply equally and without reservation to GLP-1R/GCGR dual-agonists.

As used herein the term “native glucagon” refers to naturally-occurring glucagon, e.g., human glucagon, comprising the sequence of SEQ ID NO: 30. Typically the GCG receptor (GCGR) is human GCGR (hGCGR). An exemplary hGCGR sequence is given in SEQ ID NO: 31.

As used herein, a general reference to “glucagon” in the absence of any further designation is intended to mean native human glucagon 1. Unless otherwise indicated, “glucagon” refers to human glucagon. Similarly, a general reference “GCGR” in the absence of any further designation is intended to mean native human GCGR. Unless otherwise indicated, “GCGR” refers to human GCGR.

A GCGR agonist is not native GCG but exhibits activity at the GCGR of about at least 1% or more relative to native GCG. In some aspects, a GCGR agonist exhibits activity at the GCGR of at least 5%, 10%, 20%, 30%, 40%, 50%, 60%, 70%, 80%, 90%, 95%, or more relative to native GCG. Again, this may be measured using a cAMP assay, as described further herein.

An agonist with both GLP-1R and GCGR agonist activity may be referred to interchangeably as a “GLP-1R/GCGR agonist,” “GLP-1R/GCGR co-agonist,” “GLP-1R and GCGR dual agonist” or “GLP-1R and GCGR dual co-agonist”. Thus, a “GLP-1R/GCGR agonist,” “GLP-1R/GCGR co-agonist,” “GLP-1R and GCGR dual agonist” or “GLP-1R and GCGR dual co-agonist” is not native GLP-1 and is not native GCG, and exhibits activity at the GCGR of at least 1% or more relative to native GCG and also exhibits activity at the GLP-1 receptor of about at least 1% or more relative to native GLP-1. In some aspects, a “GLP-1R/GCGR agonist” or a “GLP-1 and GCG dual agonist” exhibits activity at the GCGR of at least 5%, 10%, 20%, 30%, 40%, 50%, 60%, 70%, 80%, 90%, 95%, or more relative to native GCG and also exhibits activity at the GLP-1R of about at least 5%, 10%, 20%, 30%, 40%, 50%, 60%, 70%, 80%, 90%, 95%, or more relative to native GLP-1. This may be measured using a cAMP assay, as described herein.

In some embodiments, the GLP-1R/GCGR dual-agonists the invention exhibit in vitro potencies at the GLP-1R as shown by an EC₅₀ measured under the conditions described in Example 2 of WO 2023/148366 of less than 10,000 pM, less than 5000 pM, less than 2500 pM, less than 1000 pM, less than 900 pM, less than 800 pM, less than 700 pM, less than 600 pM, less than 500 pM, less than 400 pM, less than 300 pM, less than 200 pM, less than 100 pM, less than 50 pM, less than 25 pM, less than 20 pM, less than 15 pM, less than 10 pM, less than 5 pM, less than 4 pM, less than 3 pM, or less than 2 pM.

In some embodiments, the GLP-1R/GCGR dual-agonists the invention exhibit in vitro potencies at the GCGR as shown by an EC₅₀ measured under the conditions described in Example 2 of WO 2023/148366 of less than 10,000 pM, less than 5000 pM, less than 2500 pM, less than 1000 pM, less than 900 pM, less than 800 pM, less than 700 pM, less than 600 pM, less than 500 pM, less than 400 pM, less than 300 pM, less than 200 pM, less than 100 pM, less than 50 pM, less than 25 pM, less than 20 pM, less than 15 pM, less than 10 pM, less than 5 pM, less than 4 pM, less than 3 pM, or less than 2 pM.

In embodiments wherein a GLP-1R agonist activates both GLP-1R and GCGR, i.e. wherein the GLP-1R agonist is a GLP-1R/GCGR dual-agonist, said GLP-1R/GCGR dual-agonist may activate GLP-1R and GCGR equally.

Preferably, in embodiments wherein a GLP-1R agonist agonises activates both GLP-1R and GCGR, i.e. wherein the GLP-1R agonist is a GLP-1R/GCGR dual-agonist, said GLP-1R/GCGR dual-agonist may have selectivity for GLP-1R as compared to GCGR.

A GLP-1R/GCGR dual-agonist of the invention may have has at least a 1.5-fold, at least 2-fold, at least 3-fold, at least 4-fold, at least 5-fold, at least 6-fold, at least 7-fold, at least 8-fold, at least 9-fold, at least 10-fold, at least 12-fold, at least 15-fold, at least 17-fold, at least 20-fold, or at least 25-fold selectivity to GLP-1R as compared to GCGR. In preferred embodiments, the GLP-1R/GCGR dual-agonist has at least a 1.5-fold selectivity to GCGR as compared to GLP-1R.

The selectivity ratio of a GLP-1R/GCGR dual-agonist of the invention to GLP-1R vs GCGR is defined as: Relative Potency Ratio (RPR)=% GLP-1R activity relative to GLP-1/% GCGR activity relative to glucagon. In other words, the higher the RPR, the greater the selectivity for GLP-1R.

A GLP-1R/GCGR dual-agonist of the invention may have a hGLP-1R/hGCGR relative potency ratio (RPR) of about 1 to about 25 (with an RPR of 1 meaning the GLP-1R/GCGR dual-agonist activates GLP-1R and GCGR equally, and an RPR of 25 meaning that a GLP-1R/GCGR dual-agonist has 25-fold selectivity for GLP-1R compared with GCGR). In certain aspects, a GLP-1R/GCGR dual-agonist may have a hGLP-1R/hGCGR relative potency ratio of about 1 to about 20. In certain aspects, a GLP-1R/GCGR dual-agonist may have a hGLP1R/hGCGR relative potency ratio of about 1 to about 15. In certain aspects, a GLP-1R/GCGR dual-agonist may have a hGLP-1R/hGCGR relative potency ratio of about 1 to about 10.

A GLP-1R/GCGR dual-agonist may have a hGLP-1R/hGCGR relative potency ratio of about 2 to about 25. In certain aspects, a GLP-1R/GCGR dual-agonist may have a hGLP-1R/hGCGR relative potency ratio of about 2 to about 20. In certain aspects, a GLP-1R/GCGR dual-agonist may have a hGLP1R/hGCGR relative potency ratio of about 2 to about 15. In certain aspects, a GLP-1R/GCGR dual-agonist may have a hGLP-1R/hGCGR relative potency ratio of about 2 to about 10.

### Classification

A GLP-1R agonist of the invention (e.g. a GLP-1R/GCGR dual-agonist) may be a polypeptide, a small molecule drug, an antibody, an antibody-drug conjugate (ADC), or an aptamer; or a pharmaceutically acceptable salt thereof.

A GLP-1R agonist of the invention (e.g. a GLP-1R/GCGR dual-agonist) may be a small molecule drug. As used herein a “small molecule drug” refers to a low molecular weight compound, typically an organic compound. Typically, a small molecule has a maximum molecule weight of 900 Da, allowing for rapid diffusion across cell membranes. In some embodiments, the maximum molecular weight of a small molecule is 500 Da. Typically a small molecule has a size in the order of 1 nm.

A GLP-1R agonist of the invention (e.g. a GLP-1R/GCGR dual-agonist) may be an aptamer. Aptamers are generally nucleic acid molecules that bind a specific target molecule. Aptamers can be engineered completely in vitro, are readily produced by chemical synthesis, possess desirable storage properties, and elicit little or no immunogenicity in therapeutic applications. These characteristics make them particularly useful in pharmaceutical and therapeutic utilities.

As used herein, “aptamer” refers in general to a single or double stranded oligonucleotide or a mixture of such oligonucleotides, wherein the oligonucleotide or mixture is capable of binding specifically to a target. Oligonucleotide aptamers will be discussed here, but the skilled reader will appreciate that other aptamers having equivalent binding characteristics can also be used, such as peptide aptamers.

In general, aptamers may comprise oligonucleotides that are at least 5, at least 10 or at least 15 nucleotides in length. Aptamers may comprise sequences that are up to 40, up to 60 or up to 100 or more nucleotides in length. For example, aptamers may be from 5 to 100 nucleotides, from 10 to 40 nucleotides, or from 15 to 40 nucleotides in length. Where possible, aptamers of shorter length are preferred as these will often lead to less interference by other molecules or materials.

A GLP-1R agonist of the invention (e.g. a GLP-1R/GCGR dual-agonist) may be an antibody. As used herein, the term antibody encompasses the use of a monoclonal antibody or polyclonal antibody, as well as the antigen-binding fragments of a monoclonal or polyclonal antibody, or a peptide which binds to REV-ERB with specificity. The antibody may be a Fab, F(ab′)2, Fv, scFv, Fd, Fc or dAb.

A GLP-1R agonist of the invention (e.g. a GLP-1R/GCGR dual-agonist) may be an ADC, in which a pharmaceutically active moiety is conjugated, directly or indirectly (e.g. via a linker) to an antibody. For example, a GLP-1R agonist may be an GLP-1 analogue conjugated, directly or indirectly, to an Fc fragment. The linker may be cleavable or non-cleavable.

Preferably, a GLP-1R agonist of the invention (e.g. a GLP-1R/GCGR dual-agonist) is a polypeptide. A GLP-1R agonist polypeptide of the invention (e.g. a GLP-1R/GCGR dual-agonist polypeptide) may be of between about 15 to about 40 amino acids in length, preferably between about 25 to about 35 amino acids in length, most preferably about 30 or 31 amino acids in length. For the avoidance of doubt, any and all disclosure herein in relation to GLP-1R agonists of the invention (e.g. GLP-1R/GCGR dual-agonists) of the invention applies particularly to GLP-1R agonist polypeptides (e.g. GLP-1R/GCGR dual-agonist polypeptides) unless expressly stated to the contrary.

### GLP-1R Agonist Polypeptides

Throughout this specification, amino acid positions of a GLP-1R agonist polypeptide of the invention (e.g. a GLP-1R/GCGR dual-agonist polypeptide) are numbered according to the corresponding position in cotadutide having the sequence set forth in SEQ ID NO: 32.

A GLP-1R agonist polypeptide of the invention (e.g. a GLP-1R/GCGR dual-agonist polypeptide) may comprise or consist of an amino acid sequence having at least 60%, at least 65%, at least 70%, at least 75%, at least 80%, at least 85%, at least 90%, at least 95%, at least 97%, at least 98%, at least 99% or more, up to 100% identity to cotadutide (HSQGTFTSD-X10-SEYLDSERARDFVAWLEAGG-acid, wherein X10=Lys[ε-γE-Palmitoyl], SEQ ID NO: 32). In some preferred embodiments, a GLP-1R agonist polypeptide of the invention (e.g. a GLP-1R/GCGR dual-agonist polypeptide) may comprise or consist of an amino acid sequence having at least 65% identity to cotadutide (SEQ ID NO: 32).

A GLP-1R agonist polypeptide of the invention (e.g. a GLP-1R/GCGR dual-agonist polypeptide) may comprise an amino acid sequence having at least 60%, at least 65%, at least 70%, at least 75%, at least 80%, at least 85%, at least 90%, at least 95%, at least 97%, at least 98%, at least 99% or more, up to 100% identity to

(SEQ ID NO: 33)
H(Aib)QGTFTSDVSK(αMePhe)LDTKRARDFVQWLLE(Aib)
G-acid
or
(SEQ ID NO: 34)
H(Aib)HGS(αMePhe)TSDVSK(αMePhe)LDSRAAKD(αMePhe)
VQ(Aib)IANT-amide.

A GLP-1R agonist polypeptide of the invention (e.g. a GLP-1R/GCGR dual-agonist polypeptide) may consist of an amino acid sequence having at least 60%, at least 65%, at least 70%, at least 75%, at least 80%, at least 85%, at least 90%, at least 95%, at least 97%, at least 98%, at least 99% or more, up to 100% identity to

(SEQ ID NO: 33)
H(Aib)QGTFTSDVSK(αMePhe)LDTKRARDFVQWLLE(Aib)
G-acid
or
(SEQ ID NO: 34)
H(Aib)HGS(αMePhe)TSDVSK(αMePhe)LDSRAAKD(αMePhe)
VQ(Aib)IANT-amide.

A GLP-1R agonist polypeptide of the invention (e.g. a GLP-1R/GCGR dual-agonist polypeptide) may comprise an amino acid sequence having at least 90%, at least 95%, at least 97%, at least 98%, at least 99% or more, up to 100% identity to

(SEQ ID NO: 33)
H(Aib)QGTFTSDVSK(αMePhe)LDTKRARDFVQWLLE(Aib)
G-acid
or
(SEQ ID NO: 34)
H(Aib)HGS(αMePhe)TSDVSK(αMePhe)LDSRAAKD(αMePhe)
VQ(Aib)IANT-amide.

A GLP-1R agonist polypeptide of the invention (e.g. a GLP-1R/GCGR dual-agonist polypeptide) may consist of an amino acid sequence having at least 90%, at least 95%, at least 97%, at least 98%, at least 99% or more, up to 100% identity to

(SEQ ID NO: 33)
H(Aib)QGTFTSDVSK(αMePhe)LDTKRARDFVQWLLE(Aib)G-
acid
or
(SEQ ID NO: 34)
H(Aib)HGS(αMePhe)TSDVSK(αMePhe)LDSRAAKD(αMePhe)
VQ(Aib)IANT-amide.

A GLP-1R agonist polypeptide of the invention (e.g. a GLP-1R/GCGR dual-agonist polypeptide) may comprise an amino acid sequence having at least 60%, at least 65%, at least 70%, at least 75%, at least 80%, at least 85%, at least 90%, at least 95%, at least 97%, at least 98%, at least 99% or more, up to 100% identity to

(SEQ ID NO: 33)
H(Aib)QGTFTSDVSK(αMePhe)LDTKRARDFVQWLLE(Aib)G-
acid

A GLP-1R agonist polypeptide of the invention (e.g. a GLP-1R/GCGR dual-agonist polypeptide) may consist of an amino acid sequence having at least 60%, at least 65%, at least 70%, at least 75%, at least 80%, at least 85%, at least 90%, at least 95%, at least 97%, at least 98%, at least 99% or more, up to 100% identity to

(SEQ ID NO: 33)
H(Aib)QGTFTSDVSK(αMePhe)LDTKRARDFVQWLLE(Aib)G-
acid

A GLP-1R agonist polypeptide of the invention (e.g. a GLP-1R/GCGR dual-agonist polypeptide) may comprise an amino acid sequence having at least 90%, at least 95%, at least 97%, at least 98%, at least 99% or more, up to 100% identity to

(SEQ ID NO: 33)
H(Aib)QGTFTSDVSK(αMePhe)LDTKRARDFVQWLLE(Aib)G-
acid

A GLP-1R agonist polypeptide of the invention (e.g. a GLP-1R/GCGR dual-agonist polypeptide) may consist of an amino acid sequence having at least 90%, at least 95%, at least 97%, at least 98%, at least 99% or more, up to 100% identity to

(SEQ ID NO: 33)
H(Aib)QGTFTSDVSK(αMePhe)LDTKRARDFVQWLLE(Aib)G-acid

GLP-1R agonist polypeptides of the invention (e.g. GLP-1R/GCGR dual-agonist polypeptides) disclosed here can be formulated in or chemically conjugated to e.g. a protein, polymeric drug carrier or advance drug delivery system that further enhance the chemical stability and or physical stability and or the circulatory exposure of the GLP-1R agonist polypeptides of the invention (e.g. GLP-1R/GCGR dual-agonist polypeptides).

In embodiments of any aspect of the invention, the GLP-1R agonist polypeptides of the invention (e.g. GLP-1R/GCGR dual-agonist polypeptides) of the invention are isolated polypeptides.

### Modified Amino Acids

A GLP-1R agonist polypeptide of the invention (e.g. a GLP-1R/GCGR dual-agonist polypeptide) may comprise one or more amino acid modifications or substitutions compared to the cotadutide sequence (SEQ ID NO: 32). In particular, a GLP-1R agonist polypeptide of the invention (e.g. a GLP-1R/GCGR dual-agonist polypeptide) may comprise one or more alpha methyl amino acids, particularly selected from 2-amino-2-methylpropanoic acid (Aib) and/or alpha methyl phenylalanine (aMePhe or aMeF).

### Lipidation and Linkers

A GLP-1R agonist polypeptide of the invention (e.g. a GLP-1R/GCGR dual-agonist polypeptide) may be lipidated. Without being bound by theory, it is thought that the lipid acts as an albumin binding moiety and protects the GLP-1R agonist polypeptide of the invention (e.g. a GLP-1R/GCGR dual-agonist polypeptide) against clearance and degradation, thereby extending the half-life of the GLP-1R agonist polypeptide of the invention (e.g. a GLP-1R/GCGR dual-agonist polypeptide).

Accordingly, in some embodiments, the lipid may comprise a hydrocarbon chain having from 10 to 26 C atoms. In preferred embodiments, the lipid has 18 to 20 C atoms. In particular, the lipid may have 18 C atoms or 20 C atoms. The hydrocarbon chain may be linear or branched, and may be saturated or unsaturated. Furthermore, it can include a functional group at the end of the lipophilic chain, e.g. a carboxylic acid group which may or may not be protected during synthesis.

The GLP-1R agonist polypeptide of the invention (e.g. a GLP-1R/GCGR dual-agonist polypeptide), or pharmaceutically acceptable salt thereof, may be lipidated with a lipid selected from the group consisting of C18diacid, or C20diacid. The lipid may be octadecanedioic acid (C18diacid). The lipid may be icosanedioic acid (C20diacid).

The GLP-1R agonist polypeptide of the invention (e.g. a GLP-1R/GCGR dual-agonist polypeptide) may comprise at least one lipidated amino acid residue. In some embodiments, the GLP-1R agonist polypeptide of the invention (e.g. a GLP-1R/GCGR dual-agonist polypeptide) comprises at least two lipidated amino acid residues. In preferred embodiments, the GLP-1R agonist polypeptide of the invention (e.g. a GLP-1R/GCGR dual-agonist polypeptide) contains only one lipidated amino acid residue. The lipid may be attached to an amino acid residue of the polypeptide.

In some embodiments, the lipid is attached to the amino acid residue through a linker (referred to herein as “linker-lipid”). In alternative embodiments, the lipid is directly attached to the amino acid residue without an intervening linker. The lipid may be attached to the amino acid residue via an ester, a sulfonyl ester, a thioester, an amide, an amine or a sulphonamide. Accordingly, it will be understood that the lipid or the linker includes an acyl group, a sulphonyl group, an N atom, an O atom or an S atom which forms part of the ester, sulphonyl ester, thioester, amide, amine or sulphonamide. Optionally, an acyl group in the lipid or linker forms part of an amide or ester with the amino acid residue. Accordingly, in preferred embodiments the lipid is attached to an acylation site on the amino acid residue.

In some aspects, one or more lysine residues of the GLP-1R agonist polypeptide of the invention (e.g. a GLP-1R/GCGR dual-agonist polypeptide) are acylated. In some aspects, the lysine at position 17 is acylated. In some aspects, the lysine at position 20 is acylated.

In some aspects, one or more lysine resides of the GLP-1R agonist polypeptide of the invention (e.g. a GLP-1R/GCGR dual-agonist polypeptide) are lipidated. In some aspects, the lysine at position 17 is lipidated. In some aspects, the lysine at position 20 is lipidated. In some aspects, the linker is linked to the epsilon amino group of the residue at position 17 or 20.

The linker may comprise one or more residues of any naturally occurring or non-naturally occurring amino acid, such as those described herein in relation to the AMYR agonist polypeptides. The disclosure of linkers in the context of AMYR agonist polypeptides applies equally and without reservation to linkers suitable for use with a GLP-1R agonist polypeptide of the invention (e.g. a GLP-1R/GCGR dual-agonist polypeptide).

In some embodiments, a GLP-1R agonist polypeptide of the invention (e.g. a GLP-1R/GCGR dual-agonist polypeptide) comprises a lipid is attached to an amino acid residue in the GLP-1R agonist polypeptide of the invention (e.g. a GLP-1R/GCGR dual-agonist polypeptide) by a linker, wherein the linker comprises ((O2Oc)-(O2Oc)-γE) or ((O2Oc)-(O2Oc)-γEγE) in the C- to N-terminal orientation. In some embodiments, a GLP-1R agonist polypeptide of the invention (e.g. a GLP-1R/GCGR dual-agonist polypeptide) comprises a lipid is attached to an amino acid residue in the GLP-1R agonist polypeptide of the invention (e.g. a GLP-1R/GCGR dual-agonist polypeptide) by a linker, wherein the linker consists of ((O2Oc)-(O2Oc)-γE) or ((O2Oc)-(O2Oc)-γEγE) in the C- to N-terminal orientation. In some embodiments, a GLP-1R agonist polypeptide of the invention (e.g. a GLP-1R/GCGR dual-agonist polypeptide) comprises a lipid is attached to an amino acid residue in the GLP-1R agonist polypeptide of the invention (e.g. a GLP-1R/GCGR dual-agonist polypeptide) by a linker, wherein the linker comprises or consists of ((O2Oc)-(O2Oc)-γE) in the C- to N-terminal orientation. In some embodiments, a GLP-1R agonist polypeptide of the invention (e.g. a GLP-1R/GCGR dual-agonist polypeptide) comprises a lipid is attached to an amino acid residue in the GLP-1R agonist polypeptide of the invention (e.g. a GLP-1R/GCGR dual-agonist polypeptide) by a linker, wherein the linker comprises or consists of ((O2Oc)-(O2Oc)-γEγE) in the C- to N-terminal orientation.

Combinations of linker, lipid and polypeptide acylation sites are described in WO 2023/148366.

In some preferred embodiments, the amino acid residue at position 17 or 20 of the GLP-1R agonist polypeptide of the invention (e.g. the GLP-1R/GCGR dual-agonist polypeptide), or pharmaceutically acceptable salt thereof, is lipidated. In some preferred embodiments, wherein the amino acid residue at position 17 of the GLP-1R agonist polypeptide of the invention (e.g. the GLP-1R/GCGR dual-agonist polypeptide) is lipidated, the lipid is linked to the residue via a ((O2Oc)-(O2Oc)-γE) linker, and the lipid is octadecanedioic acid (C18diacid) or icosanedioic acid (C20diacid).

In some particularly preferred embodiments, wherein the amino acid residue at position 17 of the GLP-1R agonist polypeptide of the invention (e.g. the GLP-1R/GCGR dual-agonist polypeptide) is lipidated, the lipid is linked to the residue via a ((O2Oc)-(O2Oc)-γE) linker, and the lipid is octadecanedioic acid (C18diacid).

In particular, in preferred embodiments wherein the lysine at position 17 of the GLP-1R agonist polypeptide (e.g. the GLP-1R/GCGR dual-agonist polypeptide) is lipidated and/or acylated the lipid is linked to the epsilon amino group of lysine at position 17 of the GLP-1R agonist polypeptide (e.g. the GLP-1R/GCGR dual-agonist polypeptide) via a ((O2Oc)-(O2Oc)-γE) linker in the C- to N-terminal orientation, and the lipid is octadecanedioic acid (C18diacid) or icosanedioic acid (C20diacid).

In some particularly preferred embodiments wherein the lysine at position 17 of the GLP-1R agonist polypeptide (e.g. the GLP-1R/GCGR dual-agonist polypeptide) is lipidated and/or acylated the lipid is linked to the epsilon amino group of lysine at position 17 of the GLP-1R agonist polypeptide (e.g. the GLP-1R/GCGR dual-agonist polypeptide) via a ((O2Oc)-(O2Oc)-γE) linker in the C- to N-terminal orientation, and the lipid is octadecanedioic acid (C18diacid).

Combinations of linker, lipid and polypeptide acylation sites for GLP-1R agonist polypeptide of the invention (e.g. a GLP-1R/GCGR dual-agonist polypeptide) are described in WO 2023/148366, which is herein incorporated by reference in its entirety.

Exemplary GLP-1R Agonist Polypeptides (e.g. GLP-1R/GCGR Dual-Agonist Polypeptides)

In some preferred embodiments, the lipidated polypeptides of WO 2023/148366 may be used in the combination therapies of the present invention. Thus, a GLP-1R agonist polypeptide (e.g. a GLP-1R/GCGR dual-agonist polypeptide), or pharmaceutically acceptable salt thereof, may comprise or consist of an amino acid sequence having at least 60%, at least 65%, at least 70%, at least 75%, at least 80%, at least 85%, at least 90%, at least 95%, at least 97%, at least 98%, at least 99% or more, up to 100% identity to

(SEQ ID NO: 33)
H(Aib)QGTFTSDVSK(αMePhe)LDTKRARDFVQWLLE(Aib)G-
acid
or
(SEQ ID NO: 34)
H(Aib)HGS(αMePhe)TSDVSK(αMePhe)LDSRAAKD(αMePhe)
VQ(Aib)IANT-amide.

In some preferred embodiments, a GLP-1R agonist polypeptide of the invention (e.g. a GLP-1R/GCGR dual-agonist polypeptide) may comprise or consist of an amino acid sequence having at least 90%, at least 95%, at least 97%, at least 98%, at least 99% or more, up to 100% identity to H(Aib)QGTFTSDVSK(αMePhe)LDTKRARDFVQWLLE(Aib)G-acid (SEQ ID NO: 33) or H(Aib)HGS(αMePhe)TSDVSK(αMePhe)LDSRAAKD(αMePhe)VQ(Aib)IANT-amide (SEQ ID NO: 34).

In some particularly preferred embodiments, a GLP-1R agonist polypeptide of the invention (e.g. a GLP-1R/GCGR dual-agonist polypeptide) may comprise or consist of an amino acid sequence having at least 60%, at least 65%, at least 70%, at least 75%, at least 80%, at least 85%, at least 90%, at least 95%, at least 97%, at least 98%, at least 99% or more, up to 100% identity to H(Aib)QGTFTSDVSK(αMePhe)LDTKRARDFVQWLLE(Aib)G-acid (SEQ ID NO: 33).

In some particularly preferred embodiments, a GLP-1R agonist polypeptide of the invention (e.g. a GLP-1R/GCGR dual-agonist polypeptide) may comprise an amino acid sequence having at least 90%, at least 95%, at least 97%, at least 98%, at least 99% or more, up to 100% identity to H(Aib)QGTFTSDVSK(αMePhe)LDTKRARDFVQWLLE(Aib)G-acid (SEQ ID NO: 33).

In some particularly preferred embodiments, a GLP-1R agonist polypeptide of the invention (e.g. a GLP-1R/GCGR dual-agonist polypeptide) may consist of an amino acid sequence having at least 90%, at least 95%, at least 97%, at least 98%, at least 99% or more, up to 100% identity to H(Aib)QGTFTSDVSK(αMePhe)LDTKRARDFVQWLLE(Aib)G-acid (SEQ ID NO: 33).

More preferably, the GLP-1R agonist polypeptide of the invention (e.g. the GLP-1R/GCGR dual-agonist polypeptide, or pharmaceutically acceptable salt thereof, comprises the amino acid sequence H(Aib)QGTFTSDVSK(αMePhe)LDTK(O2Oc-O2Oc-γE-C18diacid)RARDFVQWLLE(Aib)G-acid (SEQ ID NO: 35).

Most preferably, the GLP-1R agonist polypeptide of the invention (e.g. the GLP-1R/GCGR dual-agonist polypeptide, or pharmaceutically acceptable salt thereof, consists of the amino acid sequence H(Aib)QGTFTSDVSK(αMePhe)LDTK(O2Oc-O2Oc-γE-C18diacid)RARDFVQWLLE(Aib)G-acid (SEQ ID NO: 35).

In some preferred embodiments, the GLP-1R agonist polypeptide of the invention (e.g. the GLP-1R/GCGR dual-agonist polypeptide, or pharmaceutically acceptable salt thereof, comprises the amino acid sequence H(Aib)QGTFTSDVSK(αMePhe)LDTK(O2Oc-O2Oc-γE-C20diacid)RARDFVQWLLE(Aib)G-acid (SEQ ID NO: 37).

Most preferably, the GLP-1R agonist polypeptide of the invention (e.g. the GLP-1R/GCGR dual-agonist polypeptide, or pharmaceutically acceptable salt thereof, consists of the amino acid sequence H(Aib)QGTFTSDVSK(αMePhe)LDTK(O2Oc-O2Oc-γE-C20diacid)RARDFVQWLLE(Aib)G-acid (SEQ ID NO: 37).

In some embodiments, a GLP-1R agonist polypeptide of the invention (e.g. a GLP-1R/GCGR dual-agonist polypeptide) may comprise or consist of an amino acid sequence having at least 60%, at least 65%, at least 70%, at least 75%, at least 80%, at least 85%, at least 90%, at least 95%, at least 97%, at least 98%, at least 99% or more, up to 100% identity to

(SEQ ID NO: 36)
H(Aib)HGS(αMePhe)TSDVSK(αMePhe)LDSRAAK(ε-(O20c)-
(O20c)-γE-C18diacid)D(αMePhe)VQ(Aib)IANT-amide

In some embodiments, a GLP-1R agonist polypeptide of the invention (e.g. a GLP-1R/GCGR dual-agonist polypeptide) may comprise or consist of an amino acid sequence having at least 90%, at least 95%, at least 97%, at least 98%, at least 99% or more, up to 100% identity to

(SEQ ID NO: 36)
H(Aib)HGS(αMePhe)TSDVSK(αMePhe)LDSRAAK(ε-(O20c)-
(O20c)-γE-C18diacid)D(αMePhe)VQ(Aib)IANT-amide

In some embodiments, a GLP-1R agonist polypeptide of the invention (e.g. a GLP-1R/GCGR dual-agonist polypeptide) may comprise or consist of an amino acid sequence having at least 60%, at least 65%, at least 70%, at least 75%, at least 80%, at least 85%, at least 90%, at least 95%, at least 97%, at least 98%, at least 99% or more, up to 100% identity to

(SEQ ID NO: 38)
H(Aib)HGS(αMePhe)TSDVSK(αMePhe)LDSRAAK(ε-(O20c)-
(O20c)-γE-γE-C20diacid)D(αMePhe)VQ(Aib)IANT-amide

In some embodiments, a GLP-1R agonist polypeptide of the invention (e.g. a GLP-1R/GCGR dual-agonist polypeptide) may comprise or consist of an amino acid sequence having at least 90%, at least 95%, at least 97%, at least 98%, at least 99% or more, up to 100% identity to

(SEQ ID NO: 38)
H(Aib)HGS(αMePhe)TSDVSK(αMePhe)LDSRAAK(ε-(O20c)-
(O20c)-γE-γE-C20diacid)D(αMePhe)VQ(Aib)IANT-amide

In any one of the methods disclosed herein, the GLP-1R agonist polypeptide of the invention (e.g. the GLP-1R/GCGR dual-agonist polypeptide, or pharmaceutically acceptable salt thereof, comprises the amino acid sequence H(Aib)QGTFTSDVSK(αMePhe)LDTK(O2Oc-O2Oc-γE-C18diacid)RARDFVQWLLE(Aib)G-acid (SEQ ID NO: 35) in an amount of about 1.2 mg. The subject may have obesity or an obesity-related condition.

In any one of the methods disclosed herein, the GLP-1R agonist polypeptide of the invention (e.g. the GLP-1R/GCGR dual-agonist polypeptide, or pharmaceutically acceptable salt thereof, comprises the amino acid sequence H(Aib)QGTFTSDVSK(αMePhe)LDTK(O2Oc-O2Oc-γE-C18diacid)RARDFVQWLLE(Aib)G-acid (SEQ ID NO: 35) in an amount of about 2.3 mg. The subject may have obesity or an obesity-related condition.

In any one of the methods disclosed herein, the GLP-1R agonist polypeptide of the invention (e.g. the GLP-1R/GCGR dual-agonist polypeptide, or pharmaceutically acceptable salt thereof, comprises the amino acid sequence H(Aib)QGTFTSDVSK(αMePhe)LDTK(O2Oc-O2Oc-γE-C18diacid)RARDFVQWLLE(Aib)G-acid (SEQ ID NO: 35) in an amount of about 4.6 mg. The subject may have obesity or an obesity-related condition.

In any one of the methods disclosed herein, the GLP-1R agonist polypeptide of the invention (e.g. the GLP-1R/GCGR dual-agonist polypeptide, or pharmaceutically acceptable salt thereof, comprises the amino acid sequence H(Aib)QGTFTSDVSK(αMePhe)LDTK(O2Oc-O2Oc-γE-C18diacid)RARDFVQWLLE(Aib)G-acid (SEQ ID NO: 35) in an amount of about 7 mg. The subject may have obesity or an obesity-related condition.

In any one of the methods disclosed herein, the GLP-1R agonist polypeptide of the invention (e.g. the GLP-1R/GCGR dual-agonist polypeptide, or pharmaceutically acceptable salt thereof, comprises the amino acid sequence H(Aib)QGTFTSDVSK(αMePhe)LDTK(O2Oc-O2Oc-γE-C18diacid)RARDFVQWLLE(Aib)G-acid (SEQ ID NO: 35) in an amount of about 9.4 mg. The subject may have obesity or an obesity-related condition.

In any one of the methods disclosed herein, the GLP-1R agonist polypeptide of the invention (e.g. the GLP-1R/GCGR dual-agonist polypeptide, or pharmaceutically acceptable salt thereof, comprises the amino acid sequence H(Aib)QGTFTSDVSK(αMePhe)LDTK(O2Oc-O2Oc-γE-C18diacid)RARDFVQWLLE(Aib)G-acid (SEQ ID NO: 35) in an amount of about 1.2 mg. The subject may have a metabolic disease.

In any one of the methods disclosed herein, the GLP-1R agonist polypeptide of the invention (e.g. the GLP-1R/GCGR dual-agonist polypeptide, or pharmaceutically acceptable salt thereof, comprises the amino acid sequence H(Aib)QGTFTSDVSK(αMePhe)LDTK(O2Oc-O2Oc-γE-C18diacid)RARDFVQWLLE(Aib)G-acid (SEQ ID NO: 35) in an amount of about 2.3 mg. The subject may have a metabolic disease

In any one of the methods disclosed herein, the GLP-1R agonist polypeptide of the invention (e.g. the GLP-1R/GCGR dual-agonist polypeptide, or pharmaceutically acceptable salt thereof, comprises the amino acid sequence H(Aib)QGTFTSDVSK(αMePhe)LDTK(O2Oc-O2Oc-γE-C18diacid)RARDFVQWLLE(Aib)G-acid (SEQ ID NO: 35) in an amount of about 4.6 mg. The subject may have a metabolic disease.

In any one of the methods disclosed herein, the GLP-1R agonist polypeptide of the invention (e.g. the GLP-1R/GCGR dual-agonist polypeptide, or pharmaceutically acceptable salt thereof, comprises the amino acid sequence H(Aib)QGTFTSDVSK(αMePhe)LDTK(O2Oc-O2Oc-γE-C18diacid)RARDFVQWLLE(Aib)G-acid (SEQ ID NO: 35) in an amount of about 7 mg. The subject may have a metabolic disease.

In any one of the methods disclosed herein, the GLP-1R agonist polypeptide of the invention (e.g. the GLP-1R/GCGR dual-agonist polypeptide, or pharmaceutically acceptable salt thereof, comprises the amino acid sequence H(Aib)QGTFTSDVSK(αMePhe)LDTK(O2Oc-O2Oc-γE-C18diacid)RARDFVQWLLE(Aib)G-acid (SEQ ID NO: 35) in an amount of about 9.4 mg. The subject may have a metabolic disease.

### Pharmacokinetics

A GLP-1R agonist polypeptide (e.g. a GLP-1R/GCGR dual-agonist polypeptide) used in the invention may exhibit favourable pharmacokinetic properties as compared to cotadutide. For example, the GLP-1R agonist polypeptides (e.g. the GLP-1R/GCGR dual-agonist polypeptides) may have an extended half-life as compared to cotadutide.

In some aspects, a GLP-1R agonist polypeptide (e.g. a GLP-1R/GCGR dual-agonist polypeptides) has at least 50%, at least 60%, at least 70%, at least 75%, at least 80%, at least 85%, at least 90%, at least 95, or 100% of intact GLP-1R agonist polypeptide (e.g. GLP-1R/GCGR dual-agonist polypeptide) remaining after incubation with a protease at 37° C. for 5 min, 10 min, 15 min, 30 min, 2 hr, 4 hr or 24 hr. In some aspects, the protease is selected from the group consisting of neprilysin, pepsin, pancreatin, simulated gastric fluid with pepsin, and simulated intestinal fluid with pancreatin.

In some aspects, the GLP-1R agonist polypeptide (e.g. the GLP-1R/GCGR dual-agonist polypeptide) has a half-life in cynomolgus monkeys after intravenous administration of at least 45 hours, at least 50 hours, at least 60 hours, at least 70 hours, at least 80 hours, at least 90 hours, at least 100 hours, at least 110 hours, at least 120 hours, or about 130 hours.

In some aspects, the GLP-1R agonist polypeptide (e.g. the GLP-1R/GCGR dual-agonist polypeptide) has an s.c. bioavailability in cynomolgus monkeys of at least 75%, at least 80%, at least 90%, or about 95%.

In preferred embodiments, the GLP-1R agonist polypeptides (e.g. the GLP-1R/GCGR dual-agonist polypeptides) are soluble at concentrations required for therapeutic efficacy.

### Combination Therapy

As exemplified herein, the present inventors have demonstrated that combining a particular class of AMYR agonists with GLP-1R agonists provides advantageous properties. In particular, as exemplified herein, combining AMYR agonists which has selectivity for AMYR as compared with CTR with GLP-1R agonists results in fat-mass specific weight loss and reduced aversion in pre-clinical rat models. As further demonstrated herein, GLP1-R agonists of the invention are well-tolerated in humans whilst driving significant body weight loss. Accordingly, the present inventors provide for the first time doses suitable for humans for a combination AMYR agonist and GLP1-R agonist treatment which promotes fat-mass loss and preserves lean-mass without compromising tolerability, making this a clinically attractive treatment paradigm. Thus, this combination treatment has the potential to provide synergistic effects beyond those observed using AMYR agonists and GLP1-R agonists as monotherapies. The present invention is based, at least in part on these findings.

Accordingly, provided herein is a combination therapy comprising about 1 mg to about 15 mg of an amylin receptor (AMYR) agonist; and about 0.1 mg to about 10 mg of a GLP-1 receptor (GLP-1R) agonist.

Examples of AMYR agonists with selectivity for AMYR as compared with CTR suitable for use in a combination therapy of the invention are described herein. Similarly, examples of GLP-1R agonists suitable for use in a combination therapy of the invention are described herein. Any AMYR agonists with selectivity for AMYR as compared with CTR as described herein may be combined with any GLP-1R agonist as described herein.

In some preferred embodiments, the AMYR agonist with selectivity for AMYR as compared with CTR is combined with a GLP-1R/GCGR dual-agonist.

In some preferred embodiments, the AMYR agonist with selectivity for AMYR as compared with CTR is an AMYR agonist polypeptide and GLP-1R agonist is a GLP-1R agonist polypeptide, as described herein.

In some particularly preferred embodiments, the AMYR agonist with selectivity for AMYR as compared with CTR is an AMYR agonist polypeptide and GLP-1R agonist is a GLP-1R/GCGR dual-agonist polypeptide, as described herein.

Typically the AMYR agonist polypeptide and/or the GLP-1R agonist polypeptide (e.g. the GLP-1R/GCGR dual-agonist polypeptide) are lipidated. Preferably, both the AMYR agonist polypeptide and the GLP-1R agonist polypeptide (e.g. the GLP-1R/GCGR dual-agonist polypeptide) are lipidated.

By way of non-limiting example, in some preferred embodiments, a combination therapy comprises or consists of (a) an AMYR agonist polypeptide, or pharmaceutically acceptable salt thereof comprising an amino acid sequence having at least 70%, at least 80%, at least 90%, at least 95%, at least 96%, at least 97%, at least 98%, at least 99% or more, up to 100% identity to the amino acid sequence K[CNTATC]ATQRLANFLRHSSNN(αMePhe)GPILPPTEVGSNTY-amide (SEQ ID NO: 26); and (b) a GLP-1R agonist polypeptide, or pharmaceutically acceptable salt thereof comprising an amino acid sequence having at least 60%, at least 65%, at least 70%, at least 75%, at least 80%, at least 85%, at least 90%, at least 95%, at least 97%, at least 98%, at least 99% or more, up to 100% identity to H(Aib)QGTFTSDVSK(αMePhe)LDTKRARDFVQWLLE(Aib)G-acid (SEQ ID NO: 33).

By way of further non-limiting example, in some preferred embodiments, a combination therapy comprises or consists of (a) an AMYR agonist polypeptide consisting of an amino acid sequence having at least 70%, at least 80%, at least 90%, at least 95%, at least 96%, at least 97%, at least 98%, at least 99% or more, up to 100% identity to the amino acid sequence K[CNTATC]ATQRLANFLRHSSNN(αMePhe)GPILPPTEVGSNTY-amide (SEQ ID NO: 26), or pharmaceutically acceptable salt thereof; and (b) a GLP-1R agonist polypeptide consisting of an amino acid sequence having at least 60%, at least 65%, at least 70%, at least 75%, at least 80%, at least 85%, at least 90%, at least 95%, at least 97%, at least 98%, at least 99% or more, up to 100% identity to H(Aib)QGTFTSDVSK(αMePhe)LDTKRARDFVQWLLE(Aib)G-acid (SEQ ID NO: 33), or pharmaceutically acceptable salt thereof.

By way of further non-limiting example, in some preferred embodiments, a combination therapy comprises or consists of (a) an AMYR agonist polypeptide, or pharmaceutically acceptable salt thereof comprising an amino acid sequence having at least 90%, at least 95%, at least 96%, at least 97%, at least 98%, at least 99% or more, up to 100% identity to the amino acid sequence K[CNTATC]ATQRLANFLRHSSNN(αMePhe)GPILPPTEVGSNTY-amide (SEQ ID NO: 26); and (b) a GLP-1R agonist polypeptide, or pharmaceutically acceptable salt thereof comprising an amino acid sequence having at least 90%, at least 95%, at least 97%, at least 98%, at least 99% or more, up to 100% identity to

(SEQ ID NO: 33)
H(Aib)QGTFTSDVSK(αMePhe)LDTKRARDFVQWLLE(Aib)G-
acid

By way of further non-limiting example, in some preferred embodiments, a combination therapy comprises or consists of (a) an AMYR agonist polypeptide consisting of an amino acid sequence having at least 90%, at least 95%, at least 96%, at least 97%, at least 98%, at least 99% or more, up to 100% identity to the amino acid sequence K[CNTATC]ATQRLANFLRHSSNN(αMePhe)GPILPPTEVGSNTY-amide (SEQ ID NO: 26), or pharmaceutically acceptable salt thereof; and (b) a GLP-1R agonist polypeptide consisting of an amino acid sequence having at least 90%, at least 95%, at least 97%, at least 98%, at least 99% or more, up to 100% identity to H(Aib)QGTFTSDVSK(αMePhe)LDTKRARDFVQWLLE(Aib)G-acid (SEQ ID NO: 33), or pharmaceutically acceptable salt thereof.

By way of further non-limiting example, in some particularly preferred embodiments, a combination therapy comprises or consists of (a) an AMYR agonist polypeptide, or pharmaceutically acceptable salt thereof, comprising the amino acid sequence K(γE-γE-C18diacid)[CNTATC]ATQRLANFLRHSSNN(αMePhe)GPILPPTEVGSNTY-amide (SEQ ID NO: 11); and (b) a GLP-1R agonist polypeptide, or pharmaceutically acceptable salt thereof, comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35).

By way of further non-limiting example, in some particularly preferred embodiments, a combination therapy comprises or consists of (a) an AMYR agonist polypeptide, consisting of the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof; and (b) a GLP-1R agonist polypeptide consisting of the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof.

By way of further non-limiting example, in some particularly preferred embodiments, a combination therapy comprises or consists of (a) between about 1 mg to about 15 mg of an AMYR agonist polypeptide, consisting of the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof; and (b) about 0.1 mg to about 10 mg of a GLP-1R agonist (e.g. a GLP-1R/GCGR dual-agonist) polypeptide consisting of the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 7 mg to about 10 mg (e.g., about 9 mg), and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 8 mg to about 10 mg (e.g. about 9.4 mg). Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 7 mg to about 10 mg (e.g., about 9 mg), and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 5 mg to about 10 mg (e.g. about 7 mg). Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 7 mg to about 10 mg (e.g., about 9 mg), and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 3 mg to about 5 mg (e.g. about 4.6 mg). Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 7 mg to about 10 mg (e.g., about 9 mg), and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 2 mg to about 4 mg (e.g. about 2.3 mg). Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 7 mg to about 10 mg (e.g., about 9 mg), and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 1 mg to about 2 mg (e.g. about 1.2 mg). Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 4 mg to about 7 mg (e.g. about 6 mg), and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 8 mg to about 10 mg (e.g. about 9.4 mg). Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 4 mg to about 7 mg (e.g. about 6 mg), and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 5 mg to about 10 mg (e.g. about 7 mg). Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 4 mg to about 7 mg (e.g. about 6 mg), and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 3 mg to about 5 mg (e.g. about 4.6 mg). Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 4 mg to about 7 mg (e.g. about 6 mg), and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about about 2 mg to about 4 mg (e.g. about 2.3 mg). Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 4 mg to about 7 mg (e.g. about 6 mg), and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 1 mg to about 2 mg (e.g. about 1.2 mg). Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 4 mg to about 5 mg (e.g. about 4.5 mg), and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 8 mg to about 10 mg (e.g. about 9.4 mg). Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 4 mg to about 5 mg (e.g. about 4.5 mg), and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 5 mg to about 10 mg (e.g. about 7 mg). Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 4 mg to about 5 mg (e.g. about 4.5 mg), and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 3 mg to about 5 mg (e.g. about 4.6 mg). Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 4 mg to about 5 mg (e.g. about 4.5 mg), and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 2 mg to about 4 mg (e.g. about 2.3 mg). Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 4 mg to about 5 mg (e.g. about 4.5 mg), and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 1 mg to about 2 mg (e.g. about 1.2 mg). Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 1 mg to about 2 mg (for example, about 1.50 mg or about 1.60 mg), and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 8 mg to about 10 mg (e.g. about 9.4 mg). Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 1 mg to about 2 mg (for example, about 1.50 mg or about 1.60 mg), and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 5 mg to about 10 mg (e.g. about 7 mg). Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 1 mg to about 2 mg (for example, about 1.50 mg or about 1.60 mg), and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 3 mg to about 5 mg (e.g. about 4.6 mg). Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 1 mg to about 2 mg (for example, about 1.50 mg or about 1.60 mg), and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 2 mg to about 4 mg (e.g. about 2.3 mg). Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 1 mg to about 2 mg (for example, about 1.50 mg or about 1.60 mg), and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 1 mg to about 2 mg (e.g. about 1.2 mg). Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 9 mg, and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 9.4 mg. Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 9 mg, and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 7 mg. Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 9 mg, and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 4.6 mg. Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 9 mg, and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 2.3 mg. Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 9 mg, and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 1.2 mg. Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 6 mg, and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 9.4 mg. Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 6 mg, and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 7 mg. Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 6 mg, and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 4.6 mg. Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 6 mg, and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 2.3 mg. Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 6 mg, and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 1.2 mg. Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 4.5 mg, and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 9.4 mg. Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 4.5 mg, and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 7 mg. Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 4.5 mg, and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 4.6 mg. Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 4.5 mg, and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 2.3 mg. Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 4.5 mg, and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 1.2 mg. Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 1.5 mg (for example, about 1.60 mg), and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 9.4 mg. Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 1.5 mg (for example, about 1.60 mg), and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 7 mg. Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 1.5 mg (for example, about 1.60 mg), and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 4.6 mg. Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 1.5 mg (for example, about 1.60 mg), and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 2.3 mg. Administration may be about once every 4, 5, 6, 7, 8, 9, 10, 11, 12, 13, or 14 days. Preferably, this combination therapy is administered about once a week.

The AMYR agonist comprising the amino acid sequence K(γE-γE-C18diacid)[CNTATC]ATQRLANFLRHSSNN(αMePhe)GPILPPTEVGSNTY-amide (SEQ ID NO: 11), or pharmaceutically acceptable salt thereof, may be administered to the subject at a dose, such as a fixed dose, of about 1.5 mg (for example, about 1.60 mg), and the GLP-1R agonist (e.g. a GLP-1R/GCGR dual-agonist) polypeptide comprising the amino acid sequence H(Aib)QGTFTSDVSK(αMePhe)LDTK(O2Oc-O2Oc-γE-C18diacid)RARDFVQWLLE(Aib)G-acid (SEQ ID NO: 35), or pharmaceutically acceptable salt thereof, may be administered to the subject at a dose, such as a fixed dose, of about 1.2 mg. Administration may be about once every 4, 5, 6, 7, 8, 9, 10, 11, 12, 13, or 14 days. Preferably, this combination therapy is administered about once a week.

As described herein, the GLP-1R agonist may be a GLP-1R/GCGR dual-agonist. Alternatively, a combination therapy of the invention may comprise a separate GCGR agonist. In other words, a combination therapy of the invention may comprise or consist of (a) an AMYR agonist; (b) a GLP-1R agonist; and (c) GCGR agonist; wherein each of (a)-(c) are separate agents. In such embodiments, each of (a)-(c) may be independently selected a polypeptide, small molecule drug, antibody, antibody-drug conjugate, or aptamer; or a pharmaceutically acceptable salt thereof. In some preferred embodiments, each of (a)-(c) is an agonist polypeptide. Any AMYR agonist and/or GLP-1R agonist described herein, particularly any AMYR agonist polypeptide and/or GLP-1R agonist polypeptide described herein, may be used in such embodiments.

A combination therapy of the invention may comprise one or more additional active agent useful in the treatment of a disease or disorder as described herein. By way of non-limiting example, a combination therapy may further comprise a gastric inhibitory polypeptide (GIP) agonist. In some embodiments, a GLP-1R agonist of the invention (e.g. a GLP-1R/GCGR dual-agonist) may further comprise GIP agonist activity. Alternatively, a separate GIP agonist may be combined with an AMYR agonist and a GLP-1R agonist of the invention (e.g. a GLP-1R/GCGR dual-agonist).

As exemplified herein, a combination therapy of the invention can provide surprising benefits compared with monotherapy using an AMYR agonist or a GLP-1R agonist (e.g. a GLP-1R/GCGR dual-agonist).

A combination therapy of the invention may provide increased body weight loss as described herein. Typically, a combination therapy of the invention provides increased body weight loss (in absolute or % terms) compared with monotherapy using either the AMYR agonist or a GLP-1R agonist (e.g. a GLP-1R/GCGR dual-agonist) used in the combination thereof.

In any method of the invention, the subject's body weight may be reduced by ≥5%, optionally, the subject's body weight may be reduced by ≥10%, ≥15%, or ≥20%.

By way of non-limiting example, a combination therapy of the invention may reduce body weight in humans from baseline (in terms of percent change in body weight and/or absolute change in body weight (in grams)), as exemplified herein. By way of non-limiting example, a combination therapy of the invention may reduce body weight in humans by more than 5% from baseline, and optionally, the subject's body weight may be reduced by ≥10%, ≥15%, or ≥20% from baseline, as exemplified herein. By way of non-limiting example, a combination therapy of the invention may reduce body weight in humans in a greater amount as compared to a monotherapy for weight loss (such as a dual amylin and calcitonin receptor agonist (DACRA) or an incretin mimetic drug, such as a GLP-1R agonist (e.g., semaglutide) (in terms of percent change in body weight and/or absolute change in body weight (in grams)), as exemplified herein. Such comparisons are discussed further herein.

Alternatively or in addition, a combination therapy of the invention may provide fat-mass specific body weight loss as described herein. A combination therapy of the invention may result in increased fat mass specific body weight loss (and/or preservation of lean mass) when compared to matched total body weight loss levels when the same or a different subject are treated with a combination of a DACRA and a GLP-1R agonist (e.g. a GLP-1R/GCGR dual-agonist), and/or compared with monotherapy using either the AMYR agonist or a GLP-1R agonist (e.g. a GLP-1R/GCGR dual-agonist) used in the combination thereof, or a monotherapy using a different anti-obesity drug, such as a DACRA (e.g., cagrilintide).

As used herein, the term “DACRA” refers to an agonist which can equally activate both AMYR and CTR, or which has less than 2-fold selectivity to AMYR as compared to CTR, as described herein.

Alternatively or in addition, a combination therapy of the invention may reduce nausea and/or vomiting. Said reduction in nausea and/or vomiting may be compared with nausea and/or vomiting experienced when the same or a different subject are treated with a combination of a DACRA and a GLP-1R agonist (e.g. a GLP-1R/GCGR dual-agonist), and/or compared with nausea and/or vomiting experienced when the same or a different subject are treated with monotherapy using either the AMYR agonist or a GLP-1R agonist (e.g. a GLP-1R/GCGR dual-agonist) used in the combination thereof, or a monotherapy using a different anti-obesity drug, such as a DACRA (e.g., cagrilintide or petrelintide).

### Add-On Therapy

AMYR agonists, particularly those with selectivity for AMYR as compared with CTR, can drive additional weight loss when added to existing therapy with a GLP-1R agonist in pre-clinical rat models of type 2 diabetes. Accordingly, the present inventors provide an AMYR agonist at a dose of about 1 mg to about 15 mg as an add-on or additional therapy in subjects who are overweight (≥27 kg/m²) or obese (≥30 kg/m²) and/or have type 2 diabetes, and who are on a stable dose of a GLP-1R agonist (e.g., at a dose of about 0.1 mg to about 10 mg). Particularly, the present inventors provide about 1 mg to about 15 mg of an AMYR agonist as an add-on or additional therapy in subjects who are overweight (≥27 kg/m²) or obese (≥30 kg/m²) and have type 2 diabetes, and who are on a stable dose of a GLP-1R agonist (e.g., at a dose of about 0.1 mg to about 10 mg). By a “stable dose of a GLP-1R agonist”, it will be understood that the subject is on a stable diabetes maintenance dose of said GLP-1R agonist for at least two months, preferably at least three months, more preferably at least six months at the time the add-on therapy with an AMYR agonist commences (e.g., at a dose of about 0.1 mg to about 10 mg).

Accordingly, also provided herein is a method of inhibiting or reducing weight gain, promoting weight loss, reducing food intake, increasing satiety, and/or reducing excess body weight in a subject, the method comprising administering about 1 mg to about 15 mg of the AMYR agonist, or pharmaceutically acceptable salts thereof, to the subject, wherein said subject: (i) is overweight or obese and has type 2 diabetes; and (ii) who is being treated with a GLP-1R agonist at the time treatment with the AMYR agonist commences, and optionally who continues treatment with about 0.1 mg to about 10 mg of a GLP-1R agonist after treatment with the AMYR agonist commences.

Said weight loss may be fat-mass specific, as described herein. In addition, said method may improve glycemic and/or metabolic control in a subject, also as described herein.

Examples of AMYR agonists, particularly AMYR agonists with selectivity for AMYR as compared with CTR, suitable for use in such methods are described herein. Similarly, examples of GLP-1R agonists of which a subject may be receiving a stable dose prior to such a method are described herein. Any AMYR agonists with selectivity for AMYR as compared with CTR as described herein may be combined with any GLP-1R agonist as described herein.

The AMYR agonist with selectivity for AMYR as compared with CTR may be that of SEQ ID NO: 11. Alternatively or in addition, the GLP-1R agonist may be selected from the group consisting of Exenatide (Byetta), Liraglutide (Victoza), Dulaglutide (Trulicity), Semaglutide (Ozempic, Rybelsus), and Lixisenatide (Adlyxin).

Also provided is an AMYR agonist, or pharmaceutically acceptable salt thereof, for use in a method of inhibiting or reducing weight gain, promoting weight loss, reducing food intake, increasing satiety, and/or reducing excess body weight in a subject, the method comprising administering about 1 mg to about 15 mg of the AMYR agonist, or pharmaceutically acceptable salt thereof, wherein said subject: (i) is overweight or obese and has type 2 diabetes; and (ii) who is being treated with a GLP-1R agonist at the time treatment with the AMYR agonist commences, and optionally who continues treatment with about 0.1 mg to about 10 mg of a GLP-1R agonist after treatment with the AMYR agonist commences.

### Pharmaceutical Compositions

In aspects of the invention, an AMYR agonist and a GLP-1R agonist (e.g. a GLP-1R/GCGR dual-agonist) of the invention are provided in a pharmaceutical composition. As described herein, said AMYR agonist and/or GLP-1R agonist (e.g. a GLP-1R/GCGR dual-agonist) are typically agonist polypeptides.

Further provided are compositions, e.g., pharmaceutical compositions, that contain an effective amount of an AMYR agonist (e.g. about 1 mg to about 15 mg of AMYR agonist) and a GLP-1R agonist (e.g. a GLP-1R/GCGR dual-agonist) (e.g. about 0.1 mg to about 10 mg of GLP-1R agonist) as provided herein. In some embodiments, the compositions are formulated for the treatment of obesity, an obesity-related condition or a metabolic disease as described herein, e.g., obesity, type 2 diabetes, and/or MASH.

The pharmaceutical compositions of the invention may comprise one or more excipient(s), carrier, buffer, stabilizer or other materials well known to those skilled in the art. Such materials should be non-toxic and should not interfere with the efficacy of the active ingredient. The precise nature of the carrier or other material will depend on the route of administration. Pharmaceutically acceptable excipients are known in the art, see for instance Remington's Pharmaceutical Sciences (by Joseph P. Remington, 18th ed., Mack Publishing Co., Easton, PA), which is incorporated herein in its entirety.

Composition can be in a variety of forms, including, but not limited to an aqueous solution, an emulsion, a gel, a suspension, lyophilized form, or any other form known in the art. In addition, the composition can contain pharmaceutically acceptable additives including, for example, diluents, binders, stabilizers, and preservatives. Once formulated, compositions of the invention can be administered directly to the subject.

For injectable formulations, e.g., for subcutaneous injection, the active ingredient will be in the form of a parenterally acceptable aqueous solution which is pyrogen-free and has suitable pH, isotonicity and stability.

An AMYR agonist and a GLP-1R agonist (e.g. a GLP-1R/GCGR dual-agonist) as described herein may be formulated in liquid, semi-solid or solid forms depending on the physicochemical properties of the molecule and the route of delivery. Formulations may include excipients, or combinations of excipients, for example: sugars, amino acids and surfactants. Liquid formulations may include a wide range of antibody concentrations and pH. Solid formulations may be produced by lyophilisation, spray drying, or drying by supercritical fluid technology, for example. Treatment may be given by injection (for example, subcutaneously, or intra-venously. The treatment may be administered orally. The route of administration can be determined by the physicochemical characteristics of the treatment, by special considerations for the disease or by the requirement to optimize efficacy or to minimize side-effects.

A preferred route of administering pharmaceutical compositions of the present invention is subcutaneous administration.

In some aspects, the pharmaceutical composition is a solid composition. Such solid compositions may be reconstituted prior to administration. In some aspects, the pharmaceutical composition is a liquid composition.

A composition may be administered alone or in combination with other treatments, either simultaneously or sequentially dependent upon the condition to be treated.

An AMYR agonist and a GLP-1R agonist (e.g. a GLP-1R/GCGR dual-agonist) may be used as part of a combination therapy in conjunction with an additional medicinal component. Combination treatments may be used to provide significant synergistic effects, particularly the combination of an AMYR agonist and a GLP-1R agonist (e.g. a GLP-1R/GCGR dual-agonist) with one or more other drugs. An AMYR agonist and a GLP-1R agonist (e.g. a GLP-1R/GCGR dual-agonist) may be administered concurrently or sequentially or as a combined preparation with another therapeutic agent or agents, for the treatment of one or more of the conditions listed herein.

### Methods of Production

The polypeptides of the invention, particularly the AMYR agonist polypeptides and/or the GLP-1R agonist polypeptides may be produced by any method known in the art.

The production of polypeptides such as AMYR agonist polypeptides, and GLP-1/glucagon agonist polypeptides, is well known in the art. The AMYR agonist polypeptides and/or GLP-1/glucagon agonist polypeptides of the invention can thus be produced by chemical synthesis, e.g. solid phase polypeptide synthesis using t-Boc or Fmoc chemistry, or other well-established techniques as described by Merrifield (1963, J. Am. Chem. Soc. 85:2149-2154). Solid phase peptide synthesis can be accomplished, e.g., by using automated synthesizers, using standard reagents, e.g., as explained in Example 1 of WO 2014/091316, which is herein incorporated by reference in its entirety.

Alternatively, AMYR agonist polypeptides and/or GLP-1/glucagon agonist polypeptides provided herein can be produced recombinantly using a convenient vector/host cell combination as would be well known to the person of ordinary skill in the art, e.g., by recombinant expression of a nucleic acid molecule encoding a fusion polypeptide in a host cell. Generally, a polynucleotide sequence encoding the polypeptide is inserted into an appropriate expression vehicle, e.g., a vector which contains the necessary elements for the transcription and translation of the inserted coding sequence. The nucleic acid encoding the polypeptide is inserted into the vector in proper reading frame. The expression vector is then transfected into a suitable host cell which will express the polypeptide. Suitable host cells include without limitation bacteria, yeast, or mammalian cells. A variety of commercially-available host-expression vector systems can be utilized to express the polypeptides described herein.

Following synthesis, the AMYR agonist polypeptides and/or GLP-1/glucagon agonist polypeptides may optionally be isolated or purified.

### Articles of Manufacture and Kits

The present invention provides an article of manufacture comprising AMYR agonist polypeptides and/or GLP-1/glucagon agonist polypeptides or pharmaceutical compositions of the invention. The AMYR agonist polypeptide is typically provided in one or more dose of about 1 mg to about 15 mg. The GLP-1R agonist (e.g., GLP-1R/GCGR dual-agonist) polypeptide is typically provided in one or more dose of about 0.1 mg to about 10 mg.

The present invention further provides a kit comprising AMYR agonist polypeptides and/or GLP-1/glucagon agonist polypeptides or pharmaceutical compositions of the invention. The AMYR agonist polypeptide is typically provided in one or more dose of about 1 mg to about 15 mg. The GLP-1R agonist (e.g., GLP-1R/GCGR dual-agonist) polypeptide is typically provided in one or more dose of about 0.1 mg to about 10 mg. The kit may comprise a package containing the AMYR agonist polypeptides and/or GLP-1/glucagon agonist polypeptides or pharmaceutical composition, optionally with instructions. The instructions may be directed to use of the AMYR agonist polypeptide and GLP-1R agonist polypeptide (e.g., GLP-1R/GCGR dual-agonist) for treating and/or preventing a disease or disorder in a subject at a dose of about 1 mg to about 15 mg AMYR agonist and about 0.1 mg to about 10 mg GLP-1R agonist. The instructions may be directed to use of the AMYR agonist polypeptide and GLP-1R agonist polypeptide (e.g., GLP-1R/GCGR dual-agonist) for treating and/or preventing obesity or an obesity-related condition in a subject at a dose of about 1 mg to about 15 mg AMYR agonist and about 0.1 mg to about 10 mg GLP-1R agonist. The instructions may be directed to use of the AMYR agonist polypeptide and GLP-1R agonist polypeptide (e.g., GLP-1R/GCGR dual-agonist) for treating and/or preventing a metabolic disease in a subject at a dose of about 1 mg to about 15 mg AMYR agonist and about 0.1 mg to about 10 mg GLP-1R agonist.

In some embodiments, in said kit or article of manufacture, the AMYR agonist polypeptides and/or GLP-1/glucagon agonist polypeptides or pharmaceutical compositions of the invention are formulated in single dose vials or a container closure system (e.g. pre-filled syringe). Optionally associated with such container(s) can be a notice in the form prescribed by a governmental agency regulating the manufacture, use or sale of pharmaceuticals or biological products, which notice reflects approval by the agency of manufacture, use or sale for human administration.

In another embodiment, in said kit or article of manufacture, the AMYR agonist polypeptides and/or GLP-1/glucagon agonist polypeptides) or pharmaceutical compositions of the invention are formulated in a dual-chamber device, e.g., a dual-chamber syringe (DCS). Typically, such devices comprise a pre-filled double chamber mechanism for simultaneous administration of two active ingredients, in which a first active ingredient is formulated in a first syringe chamber or cartridge, and a second active ingredient is formulated in a second syringe chamber or cartridge. The chambers may be separated by a middle plunger that on activation allows the two active ingredients to mix in one drug chamber immediately before administration. The main advantages of such pre-filled devices are the increase of dose accuracy, the lower risk of microbial contamination, and the reduction of the procedure time and handling steps required. Some DCSs incorporate a parallel post-injection safety sheath chamber for enclosing a sharpened needle tip. These syringes are developed as a protection of the inadvertent contact with the needles after the syringe has been used, which can be extremely important in the reduction of needle-stick injuries and contact with blood-transmitted diseases such as hepatitis or acquired immune deficiency syndrome.

### Brief Description of Sequence Listing

Where present, a square bracket [ ] between the two cysteine residues (Cys2 and Cys7) indicate the presence of an intramolecular disulphide bridge.
γE or γE=gamma-glutamate
SEQ ID NO: 1 exemplary amino acid sequence for hRAMP1
SEQ ID NO: 2 exemplary amino acid sequence for hRAMP2
SEQ ID NO: 3 exemplary amino acid sequence for hRAMP3
SEQ ID NO: 4 exemplary amino acid sequence for hCTR
SEQ ID NO: 5 pramlintide amino acid sequence

SEQ ID NO: 6
C18diacid-γE-
[CNTATC]ATQRLAEFLRHSSNN(αMePhe)GPILPPTNVGSNTY-amide
SEQ ID NO: 7
K(γE-γE-
C18diacid)[CNTATC]ATQRLAEFLRHSSNN(αMePhe)GPILPPTNVGSNTY-amide
SEQ ID NO: 8
K(γE-
C18diacid)K[CNTATC]ATQRLAEFLRHSSNN(αMePhe)GPILPPTNVGSNTY-amide);
SEQ ID NO: 9
K(γE-
C18diacid)K[CNTATC]ATQRLANFLRHSSNN(αMePhe)GPILPPTEVGSNTY-amide
SEQ ID NO: 10
K(O20c-O20c-γE-
18diacid)[CNTATC]ATQRLANFLRHSSNN(αMePhe)GPILPPTEVGSNTY-amide
SEQ ID NO: 11
K(γE-γE-
C18diacid)[CNTATC]ATQRLANFLRHSSNN(αMePhe)GPILPPTEVGSNTY-amide
SEQ ID NO: 12
K(γE-
C18diacid)[CNTATC]ATQRLANFLRHSSNN(αMePhe)GPILPPTNVGSNTY-amide
SEQ ID NO: 13
K(γE-γE-
C18diacid)[CNTATC]ATQRLANFLRHSSNN(αMePhe)GPILPPTNVGSNTY-amide
SEQ ID NO: 14
K(γE-γE-
C18diacid)[CNTATC]ATQRLANFLVHSSNN(αMePhe)GPILPPTNVGSRTY-amide
SEQ ID NO: 15
K(γE-γE-
C18diacid)[CNTATC]ATQRLANFLVHSSNN(αMePhe)GPILPPTRVGSNTY-amide
SEQ ID NO: 16
K(γE-
C18diacid)[CNTATC]ATQRLANFLVHSSNN(αMePhe)GPILPPTRVGSNTY-amide
SEQ ID NO: 17
K(γE-
C18diacid)K[CNTATC]ATQRLA(Dab)FLVHSSNNFGPILPPTNVGSNTY-amide
SEQ ID NO: 18
K(γE-
C18diacid)K[CNTATC]ATQRLANFLVHSS(Aib)NFGPILPPTHVGSNTY-amide
SEQ ID NO: 19
K(γE-
C18diacid)[CNTATC]ATQRLANFLRHSS(Aib)NFGPILPPTEVGSNTY-amide
SEQ ID NO: 20
K(γE-
C18diacid)[CNTATC]ATQRLANFLRHSS(Aib)NFGPILPPTNVGSNTY-amide
SEQ ID NO: 21
K(γE-γE-
C18diacid)K[CNTATC]ATQRLANFLRHSS(Aib)NFGPILPPTNVGSNTY-amide
SEQ ID NO: 22
K(γE-γE-
C18diacid)K[CNTATC]ATQRLANFLVHSS(Aib)NFGPILPPTRVGSNTY-amide
SEQ ID NO: 23
K(γE-
C18diacid)K[CNTATC]ATQRLANFLVHSS(Aib)NFGPILPPT(Aib)VGSNTY-amide
SEQ ID NO: 24
K(γE-
C18diacid)K[CNTATC]ATQRLANFL(Aib)HSSNNFGPILPPTNVGSNTY-amide
SEQ ID NO: 25
K(γE-
C18diacid)K[CNTATC]ATQRLA(Dab)FL(Aib)HSSNNFGPILPPTEVGSNTY-amide
SEQ ID NO: 26
K[CNTATC]ATQRLANFLRHSSNN(αMePhe)GPILPPTEVGSNTY-
amide

SEQ ID NO: 27 exemplary GLP-1(7-36) amide amino acid sequence
SEQ ID NO: 28 exemplary GLP-1(7-37) acid amino acid sequence
SEQ ID NO: 29 exemplary hGLP-1R amino acid sequence
SEQ ID NO: 30 exemplary hGCG amino acid sequence
SEQ ID NO: 31 exemplary hGCGR amino acid sequence
SEQ ID NO: 32 cotadutide amino acid sequence

SEQ ID NO: 33
H(Aib)QGTFTSDVSK(αMePhe)LDTKRARDFVQWLLE(Aib)G-acid
SEQ ID NO: 34
H(Aib)HGS(αMePhe)TSDVSK(αMePhe)LDSRAAKD(αMePhe)
VQ(Aib)IANT-amide
SEQ ID NO: 35
H(Aib)QGTFTSDVSK(αMePhe)LDTK(O20c-O20c-γE-
C18diacid)RARDFVQWLLE(Aib)G-acid
SEQ ID NO: 36
H(Aib)HGS(αMePhe)TSDVSK(αMePhe)LDSRAAK(ε-(O20c)-
(O20c)-γE-C18diacid)D(αMePhe)VQ(Aib)IANT-amide
SEQ ID NO: 37
H(Aib)QGTFTSDVSK(αMePhe)LDTK(O20c-O20c-γE-
C20diacid)RARDFVQWLLE(Aib)G-acid
SEQ ID NO: 38
H(Aib)HGS(αMePhe)TSDVSK(αMePhe)LDSRAAK(ε-(O20c)-
(O20c)-γE-γE-C20diacid)D(αMePhe)VQ(Aib)IANT-amide

### EXAMPLES

The invention is now described with reference to the Examples below. These are not limiting in terms of the scope of the invention, and a person skilled in the art would appreciate that suitable equivalents could be used within the scope of the present invention. Thus, the Examples may be considered component parts of the invention, and the individual aspects described therein may be considered as disclosed independently, or in any combination.

### Example 1: A Phase I Single Ascending Dose (SAD) Study and a Phase Ib Repeated Dose Study of Administering an AMYR Agonist to Healthy Subjects Who are Overweight or Obese

Pre-clinical studies have demonstrated that an AMYR agonist promotes, maintains and enhances weight loss without compromising tolerability in animal models. In particular, 20 nmol/kg of an AMYR agonist results in a reduction in overall body weight loss (in grams) in diet-induced obese (DIO) rats in a 15 day treatment as compared to vehicle (data not shown). In particular, the AMYR agonist was found to drive good quality weight loss by inducing more fat mass specific body weight loss and preserving more lean muscle mass as compared to a GLP-1 receptor agonist drug, semaglutide (10 nmol/kg), and rats pair-fed to the AMYR agonist (i.e., rats having restricted food intake to match the treatment group diet). In a separate experiment, a AMYR agonist (30 nmol/kg) was found to be highly tolerable as measured by rodent aversion studies (an experiment in which a rodent's aversion to saccharin following administration of a drug is a correlate for nausea), in which the AMYR agonist demonstrated significantly less aversion in comparison to vehicle as compared to a DACRA (cagrilintide 10 nmol/kg, p<0.05), a GLP-1 receptor agonist (semaglutide 2.4 nmol/kg, p<0.01), and a combination thereof (p<0.0001).

Following the pre-clinical studies, a Phase I randomised, single-blind, placebo-controlled study was performed in order to assess the safety, tolerability, and pharmacokinetics of an AMYR agonist following single ascending dose (SAD) administration to healthy subjects who are overweight or obese (trial identifier: D8750000001; ClinicalTrials.gov identifier: NCT05511025). The inclusion criteria for the study was healthy male and female participants aged 18 to 55 years and a body mass index (BMI) of 25 to 35 kg/m² inclusive (at the time of screening) and weighing at least 50 kg. A Japanese cohort was also designed to include natives of Japan (defined as having both parents and four grandparents who are Japanese, including second and third generation subjects of Japanese descent whose parents or grandparents are living in a country other than Japan) and having a BMI of 23 to 35 kg/m² inclusive (at the time of screening) and weighing at least 50 kg.

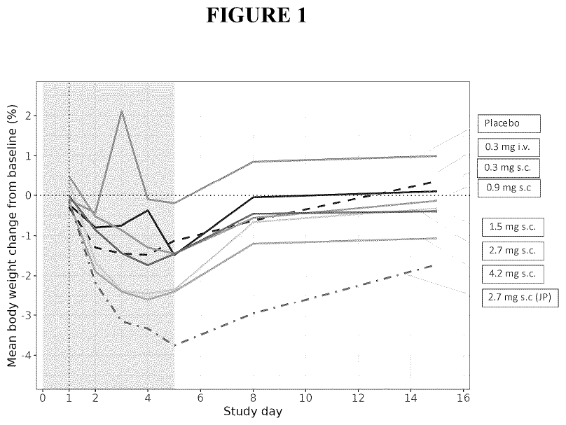

In the study, 54 participants were dosed with a single dose of between 0.3 mg to 4.2 mg of an AMYR agonist or placebo by subcutaneous administration, a single dose of 0.3 mg by intravenous administration, or a single dose of 2.7 mg by subcutaneous administration in the Japanese cohort. Subjects were monitored for a period of 15 days. The AMYR agonist was found to demonstrate good efficacy with competitive tolerability. In particular, the AMYR agonist demonstrated dose dependent weight loss (approximately 1.5%-3% mean body weight change from baseline) at doses of 0.3 mg, 0.9 mg, 1.5 mg, 2.7 mg, and 4.2 mg. Peak weight loss was demonstrated at 4-5 days in all groups. Strong weight loss profiles were seen in the 1.5 mg and 4.2 mg cohorts in particular. Following a single 2.7 mg dose of AMYR agonist, a mean weight loss of 1.7% was seen in the global cohort (s.c.), and 3.8% in the Japanese cohort (s.c.; see FIG. 1).

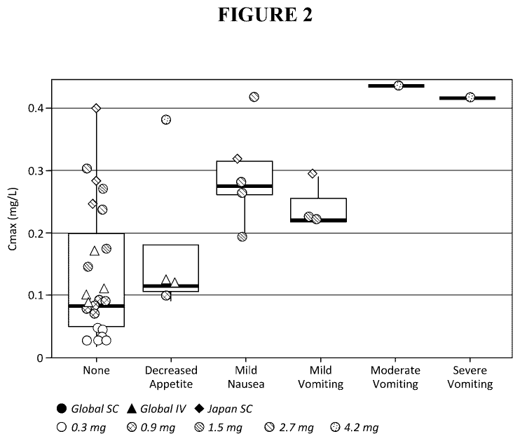

The AMYR agonist also demonstrated a good tolerability profile, in which no safety concerns were identified at single doses up to 4.2 mg. Moderate or severe vomiting were only demonstrated for some subjects in the 4.2 mg group (s.c.), while some incidences of mild nausea and/or mild vomiting occurred in the 1.5 mg and 2.7 mg (global and Japanese cohorts) groups (see FIG. 2).

A Phase Ib study designed to assess the safety, tolerability, pharmacokinetics, and the impact on body weight of repeated doses of an AMYR agonist in healthy participants with overweight or obesity (trial identifier: D8750000002; ClinicalTrials.gov identifier: NCT06132841) is currently ongoing. The inclusion criteria for the study is healthy male and female participants aged 18 to 55 years and a body mass index (EMI) of 25 to 40 kg/m² inclusive (at the time of screening) and weighing at least 60 kg.

The study involves a treatment period of 6 weeks (cohort 1) and 12 weeks (cohort 2 and 3) during which the participants receive once-weekly repeated doses of the drug at doses between 1.5 mg to 9 mg during residency at a clinical unit. Cohort 1 (n=15+5) receive repeated doses of 2.7 mg of the drug, or placebo, over six weeks. Cohorts 2 (n=15+5) and 3 (n=9+3) receive repeated doses gradually titrated from an initial 1.5 mg to 9 mg by the end of the study over a 12 week period, or placebo. Eligible participants were randomized to AMYR agonist and placebo in a 3:1 ratio.

Based on interim data from a subset of the participants following s.c. weekly administration of between 1.5 mg to 9 mg AMYR agonist, the AMYR agonist was well tolerated and no safety concerns were identified.

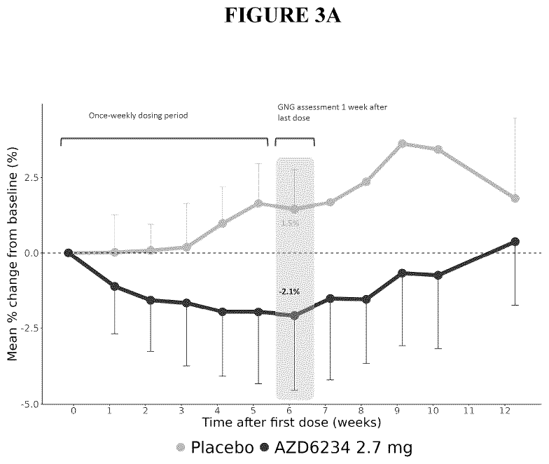

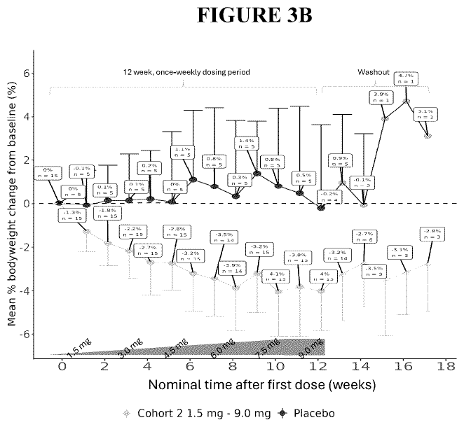

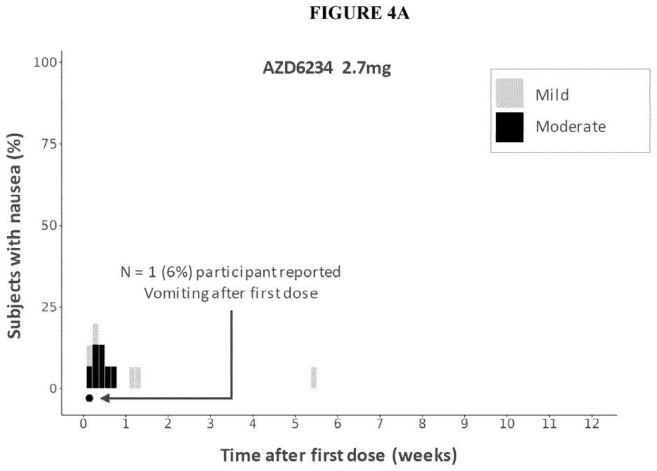

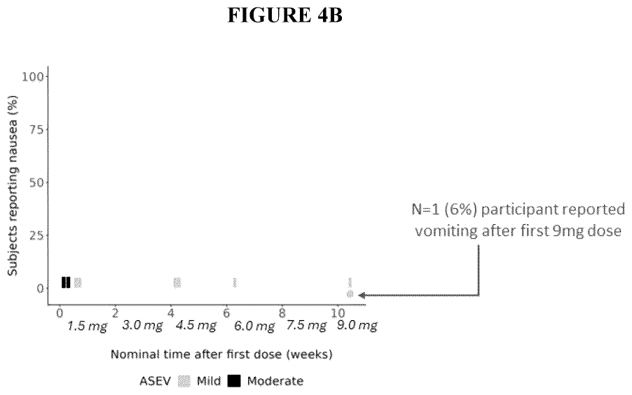

In particular, following 6 weeks of weekly repeat dosing with 2.7 mg AMYR agonist (n=14; Cohort 1), 2.1% weight loss (3.5% placebo-corrected) was measured (see FIG. 3A). Further, four (27%) and one (6%) participants on active treatment of 2.7 mg reported mild to moderate nausea and vomiting, respectively. In particular, the sole report of vomiting occurred within a week of the first dose, all reports of moderate nausea occurred within a week of the first dose, with only one report of mild nausea beyond the second dose. Thus, the vast majority of these incidents occurred in the first week after the first dose (see FIG. 4A).

Following 12 weeks of treatment gradually titrated from 1.5 mg to 9 mg (Cohort 2), 4% weight loss (3.8% placebo-corrected) was measured (see FIG. 3B). Three participants reported mild to moderate nausea (20%) and one reported mild vomiting (6%) following 12 weeks of weekly repeat dosing. The sole report of vomiting occurred shortly after the escalation to a 9 mg dose (see FIG. 4B).

These studies demonstrate a dose-dependent reduction in bodyweight across the dose range of 1.5 to 9 mg in humans, which based on preclinical models is expected to be almost exclusively due to fat mass loss. Further, it was observed that even relatively high doses of AMYR agonist therapy are surprisingly well tolerated in human subjects.

In these studies, the AMYR agonist was AZD6234 (SEQ ID NO: 11). AZD6234 is a potent agonist of human, cynomolgus monkey, and rat amylin receptors, displaying in vitro selectivity over calcitonin receptor activation.

### Example 2: A Phase I Single Ascending Dose (SAD) Study Evaluating Administration of a GLP-1R/GCGR Dual Agonist Peptide to Healthy Subjects and a Phase I/II Study to Evaluate the Safety, Tolerability, Pharmacokinetics, and Pharmacodynamics of a GLP-1R/GCGR Dual Agonist Peptide in Overweight and Obese Participants with or without Type 2 Diabetes Mellitus

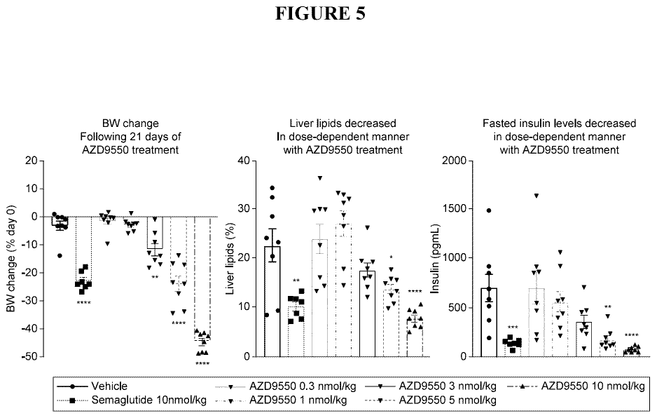

Pre-clinical studies in obese mice have demonstrated that a GLP-1R/GCGR dual agonist results in dose-dependent reductions in body weight (BW) in obese mice. In particular, diet-induced obese mice (n=7-8/group) were dosed once daily (QD) subcutaneously (SC) for 21 days with vehicle, GLP-1 RA semaglutide (10 nmol/kg) or a GLP-1/GCG dual agonist (0.3, 1, 3, 5 or 10 nmol/kg). BW was measured daily; liver lipid levels were measured at the end of the study via nuclear magnetic resonance (Bruker Corporation); plasma insulin levels were measured following a 6 h fast on study day 20 using the Meso Scale Discovery assay (Meso Scale Diagnostics, K152BZC). Weight reduction (±standard error of the mean) was 1.5±1.2%, 2.9±0.8%, 11.7±2.1%, 23.8±2.7% and 44.4±1.2% at 0.3, 1, 3, 5 and 10 nmol/kg, respectively, versus 3.1±1.6% decrease with vehicle treatment. Further, liver lipids and plasma insulin levels decreased in a dose-dependent manner following GLP-1/GCG dual agonist administration (see FIG. 5; One-way ANOVA, Dunnett post hoc; *p<0.05, **p<0.01, ***p<0.001, ****p<0.0001, vs vehicle).

Following the pre-clinical studies, a Phase I randomised, single-blind, placebo-controlled study was performed (trial identifier: D8460000001) in order to assess the safety, tolerability, and pharmacokinetics of an GLP-1R/GCGR dual agonist following single ascending dose (SAD) administration to healthy participants (N=40; age 18-55 years; body mass index between 18 and 30 kg/m² inclusive), randomized to GLP-1R/GCGR dual agonist or placebo.

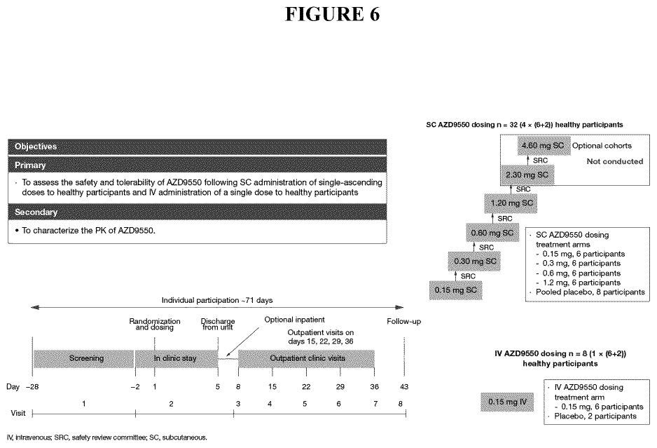

Single ascending doses of 0.15, 0.3, 0.6 or 1.2 mg subcutaneous (SC) and a single 0.15 mg intravenous (IV) dose were administered across five cohorts (“Part A”), each with six participants receiving an GLP-1R/GCGR dual agonist and two receiving placebo (see FIG. 6). A further 8 Japanese healthy volunteers received a single 0.6 mg SC dose of the GLP-1R/GCGR dual agonist or placebo (“Part B”). The primary objective was to assess the safety and tolerability of a GLP-1R/GCGR dual agonist following SC administration of single-ascending doses to healthy participants and IV administration of a single dose to healthy participants. The secondary objective was to characterize the PK of the GLP-1R/GCGR dual agonist.

The study included 54 human participants: 46 male and 8 female; mean body mass index was 28.8 kg/m² (SD 2.7) and mean baseline body weight was 87.7 kg. Median age was 42.5 years.

No deaths or other serious adverse events were reported and there were no safety concerns with the SC or IV doses studied. Most frequently reported adverse events (AEs) following SC dosing in Part A were decreased appetite (33.3%), nausea (29.2%), vomiting (25.0%), and headache (25.0%) (Table 1). These were mainly mild-to-moderate, with one case of severe vomiting in the AZD9550 1.2 mg dose cohort. There were no reports of nausea, vomiting or diarrhoea for the 8 participants in Part B.

TABLE 1
Summary of treatment-emergent adverse events (SC cohorts) in Part
A: Number (n) and percent (%) of participants with any reported
adverse event (AE), any possibly related AE and most frequent
AEs sorted by decreasing frequency on preferred term level.

| | AZD9550 | | | | | Placebo |
|---|---|---|---|---|---|---|
| Variables, n | 0.15 mg | 0.30 mg | 0.60 mg | 1.20 mg | Pooled | Pooled |
| (%) | n = 6 | n = 6 | n = 6 | n = 6 | n = 24 | n = 8 |
| Any AE | 2 (33.3) | 5 (83.3) | 4 (66.7) | 6 (100) | 17 (70.8) | 3 (37.5) |
| Any possibly | 1 (16.7) | 1 (16.7) | 3 (50.0) | 6 (100) | 11 (45.8) | 0 (0.0) |
| related AE | | | | | | |
| Decreased | 0 (0.0) | 0 (0.0) | 2 (33.3) | 6 (100) | 8 (33.3) | 0 (0.0) |
| appetite | | | | | | |
| Nausea | 0 (0.0) | 0 (0.0) | 2 (33.3) | 5 (83.3) | 7 (29.2) | 0 (0.0) |
| Vomiting | 0 (0.0) | 0 (0.0) | 1 (16.7) | 5 (83.3) | 6 (25.0) | 0 (0.0) |
| Headache | 0 (0.0) | 0 (0.0) | 2 (33.3) | 4 (66.7) | 6 (25.0) | 0 (0.0) |

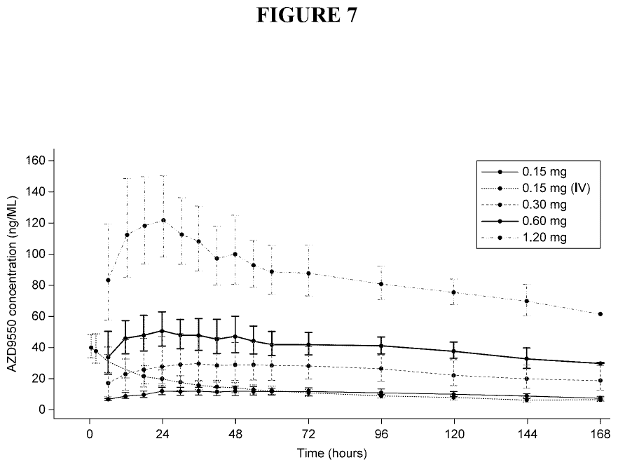

Dose-proportional pharmacokinetics were observed across the dose range, 0.15 mg to 1.20 mg, following single SC dose (FIG. 7; data are presented as geometric mean with SD). Peak plasma concentrations (Cmax) occurred 24 h to 49 h post-dose at SC dosing. Terminal half-life was around 5-6 days. The GLP-1R/GCGR dual agonist had approximately 100% absolute bioavailability based on 0.15 mg SC and IV dosing.

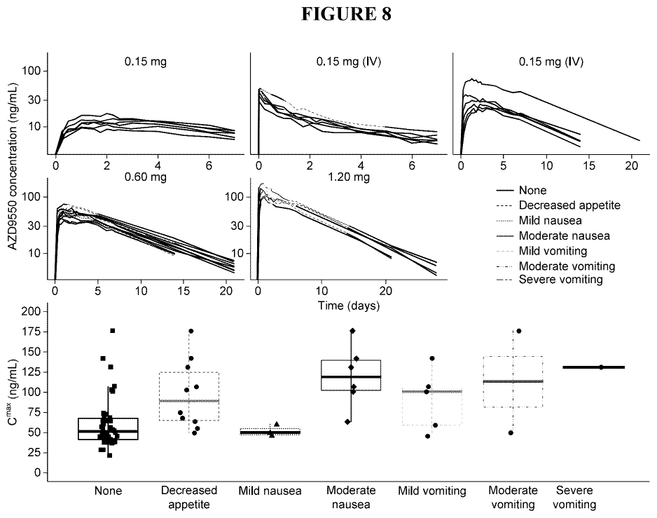

The rate and severity of nausea and vomiting were associated with increased GLP-1R/GCGR dual agonist dose and Cmax (FIG. 8). 0.6 mg SC was considered a tolerable starting dose for further studies.

A Phase I/II, Randomised, Single-blind, Placebo-controlled, Multiple-ascending-dose Study to Evaluate the Safety, Tolerability, Pharmacokinetics, and Pharmacodynamics of a GLP-1R/GCGR dual agonist peptide in Overweight and Obese Participants with or without Type 2 Diabetes Mellitus (T2DM; trial identifier: D8460000002; ‘CONTEMPO’) is currently ongoing. This Phase I/II, randomised, single-blind, placebo-controlled, MAD study is assessing the safety and tolerability of a GLP-1R/GCGR dual agonist peptide and characterising the PK and PD of a GLP-1R/GCGR dual agonist peptide following SC administration to overweight and obese participants living with or without T2DM, including men and women of non-childbearing potential.

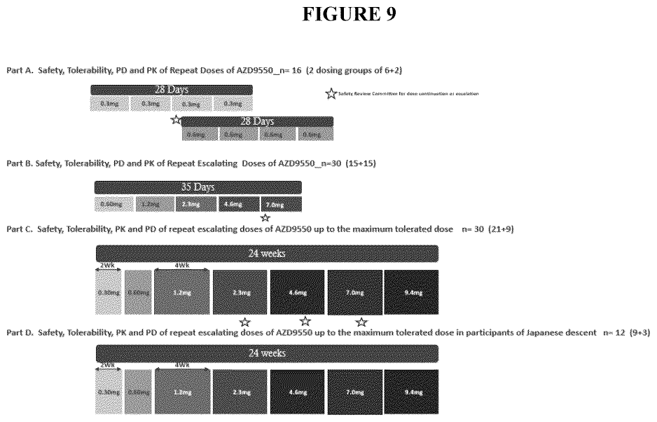

The study consists of four parts (Parts A-D), each involving an assessment following administration of repeat doses of a GLP-1R/GCGR dual agonist peptide (the titration schedule is set out in FIG. 9). Specifically, the study involves:
Repeat administration of 0.3 mg (n=6) or 0.6 mg (n=6) over a treatment period of 28 days (Part A),
Repeat escalating doses titrated up to 7 mg over a treatment period of 35 days (Part B, n=30 (15+15))
Repeat escalating doses titrated up to 9.4 mg over a treatment period of 24 weeks (Part C, n=30 (21+9)) and
Repeat escalating doses titrated up to 9.4 mg over a treatment period of 24 weeks in participants of Japanese descent (Part D, n=12 (9+3)).

The inclusion criteria for the study is:
Males or females of non-childbearing potential age 18 through 65 years at the time of screening.
Parts A, B, C only: Participants with or without T2DM. If participants have a diagnosis of T2DM, the glucose control managed with diabetes diet and in addition to metformin treatment no more than two treatment options (with a stable dose 3 months prior to screening).
Part D only: Participants who are diagnosed with T2DM, have inadequate glycaemic control with diet and exercise. Participants who are prescribed an oral anti-diabetic agent such as metformin, a DPP IV inhibitor, sulphonylurea, glinides, alphaglucosidase inhibitors, and an SGLT2 inhibitor may be eligible to enter the study following a washout of 4-weeks or 5-half lives (whichever is longer) washout period.
Participants with a screening HbAlc value within the target range of
   ≥42 to ≤75 mmol/mol (6% to 9%) for T2DM patients
   <48 mmol/mol (<6.5%) for participants without T2DM
Body mass index from ≥27 (≥25 in Part D) to ≤39.9 kg/m2 (inclusive).
Contraceptive use by males or females should be consistent with local regulations regarding the methods of contraception for those participating in clinical studies.
Written informed consent and any locally required authorization (eg, European Union Data Privacy Directive) obtained from the participant prior to performing any protocol-related procedures, including screening evaluations
Ability to complete and meet all eligibility requirements for randomisation within 60 days after signing the ICF.
Venous access suitable for multiple cannulations.
Willing and able to self-administer weekly SC injections (Parts C and D only).

Based on interim data, the half-life of the GLP-1R/GCGR dual agonist was −7 days, supporting once-weekly dosing. The GLP-1R/GCG dual agonist was well tolerated and no safety concerns were identified. Low incidences of nausea and vomiting were recorded (data not shown).

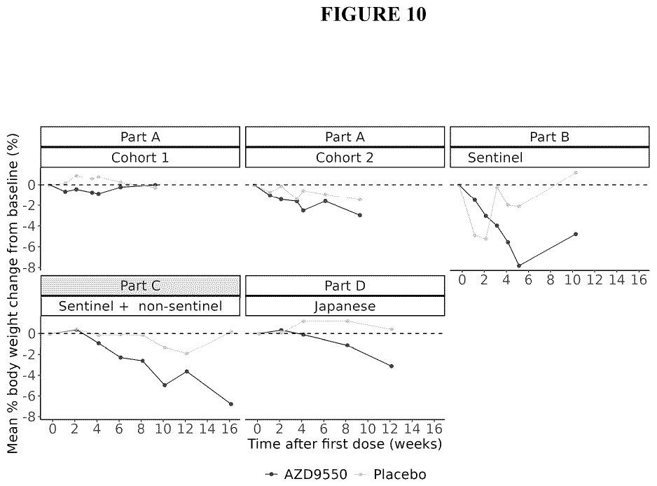

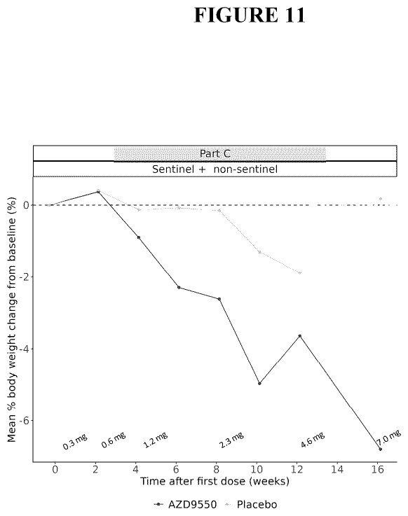

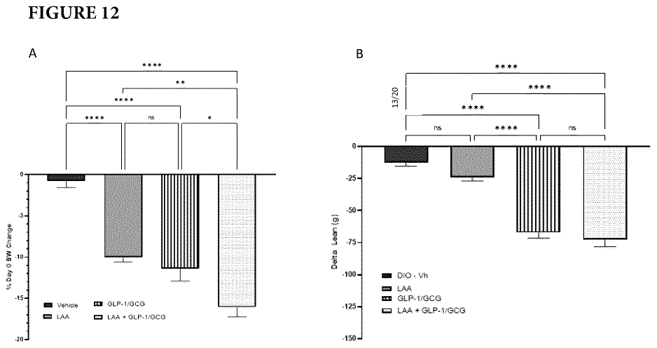

Based on interim body weight loss data, administration of multiple-once weekly ascending doses of GLP-1R/GCGR dual agonist (s.c.) achieved dose dependent body weight loss across all parts of the study (FIG. 10). In particular, based on interim body weight loss data from a subset of the participants (Part C), approximately 6.8% (6.6% placebo-corrected) weight loss was achieved following multiple-once weekly ascending doses of GLP-1R/GCGR dual agonist (s.c.) for 16 weeks treatment titrated up to a (non-final) dose of 7 mg (n=6 active, n=1 placebo) (FIG. 11).

In these studies, the GLP-1/GCG dual agonist was AZD9550 (SEQ ID NO: 35). AZD9550 is a synthetic GLP-1R/GCGR dual peptide agonist designed with a GLP-1R:GCGR potency ratio similar to cotadutide, but with a longer half-life to optimize exposure and dosing frequency in the clinic.

In summary, in healthy participants, there were no safety or tolerability concerns with a pharmacokinetic profile supporting once-weekly dosing of a GLP-1/GCG dual agonist.

### Example 3: Pre-Clinical Studies Confirm that Co-Administration of an AMYR Agonist and a GLP-1/GCG Dual Agonist Synergistically Drives Additional Body Weight Loss Compared with Monotherapy in DIO Rats

Pre-clinical studies in obese rodents treated with either AZD9550 or AZD6234 monotherapy demonstrate body weight loss (data not shown). In addition, a 3-month toxicology study in rats showed that the combination of AZD9550 and AZD6234 is well tolerated and provided the expected pharmacological effects, but with greater body weight reductions for the combination as compared to either agent alone. No unexpected toxicology findings were observed for the combination as compared to either agent alone (data not shown).

Pre-clinical studies also demonstrate that the combination of AZD9550 and AZD6234 drives additional body weight loss compared with monotherapy in DIO rats. In particular, Male diet-induced obese (DIO) Sprague-Dawley rats were fed a high fat diet (45% fat, D12451; Research Diets) for 34 weeks prior to study start. Additionally, age-matched lean rats were fed a control diet (10% fat, D12450K; Research Diets). DIO rats were then dosed (n=8/group) with Vehicle, GLP-1/GCG (5.4 nmol/kg), LAA (10 nmol/kg) or combination of a GLP-1/GCG dual agonist and a selective AMYR agonist (referred to as long-acting amylin analogue, LAA) subcutaneously (SC) once daily (QD) for 28 days. Average body weight for all DIO rats at start of study was 722.4±10.1 grams. Body composition was measured by EchoMRI on day −7 and day 26. In this experiment, the LAA was that of SEQ ID NO: 11, and the GLP-1/GCG dual-agonist was cotadutide.

Data reported as mean±standard error of the mean (SEM); data analysis performed using GraphPad Prism—statistical analysis performed using One-way ANOVA with Tukey ad hoc analysis. (*p<0.05, **p<0.01, ***0<0.001, ****p<0.0001).

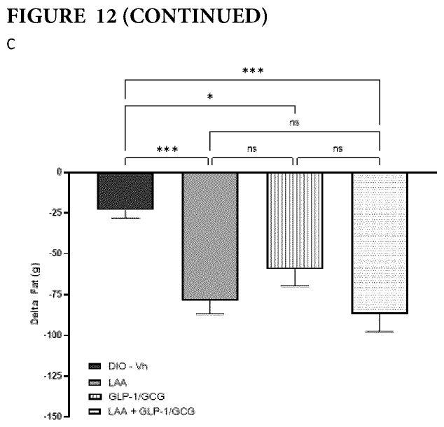

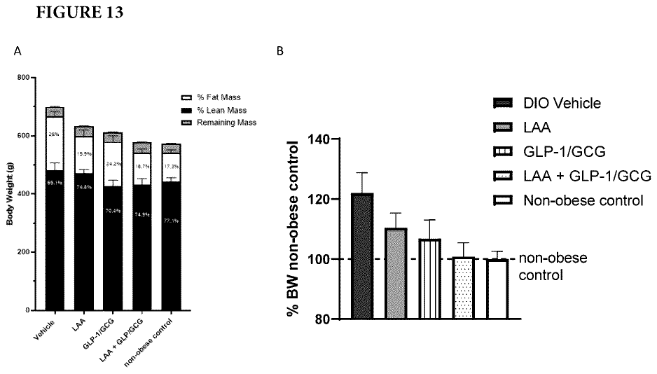

DIO rats treated with LAA, GLP-1R/GCGR or a combination of both lost significantly more body weight than Vehicle treated rats. Additionally, animals treated with LAA+GLP1/GCG lost significantly more weight than either LAA or GLP-1/GCG mono treatments (FIG. 12A).

Further, as also shown with data from study day 26, animals treated with LAA+GLP1/GCG reached a body weight similar to non-obese control animals (FIG. 2B). Body composition as measured by EchoMRI (FIGS. 12B, 12C, 13A) shows that rats treated with LAA monotherapy lost a significant amount of fat mass compared to Vehicle treatment, with little effect on lean mass. Rats treated with GLP-1/GCG monotherapy lost both fat and lean mass compared to Vehicle treated animals. Rats receiving the combination of LAA and GLP-1/GCG lost both fat and lean mass. However, the effect on lean mass loss was not different from than of the GLP-1/GCG mono-treatment, suggesting the additional weight driven by combination with LAA is fat mass specific.

In a further study, male DIO Sprague-Dawley rats were fed a high-fat diet (32.5% kcal fat, D12266B; Research Diets) for approximately 8 weeks prior to study start. Rats were dosed once daily SC for 28 days with Vehicle, GLP-1/GCG (1.5 nmol/kg or 5 nmol/kg), LAA (7.5 nmol/kg) or a combination of GLP-1/GCG and LAA (1.5 and 7.5 nmol/kg, or 5 nmol/kg and 7.5 nmol/kg, respectively) with an n=6/group. Body weight was monitored daily throughout the study. Data reported as mean±standard error of the mean (SEM); data analysis performed using GraphPad Prism—statistical analysis performed using One-way ANOVA with Tukey ad hoc analysis (*p<0.05, **p<0.01, ***0<0.001, ****p<0.0001). In this experiment, the LAA was that of SEQ ID NO: 11, and the GLP-1/GCG dual-agonist was cotadutide.

At end of study (day 28) animals treated with GLP-1/GCG at 1.5 nmol/kg had a mean change in body weight of −8.4% and rats treated with LAA at 7.5 nmol/kg had a change of −13.7% when normalized to Vehicle. Rats that received both LAA and GLP-1/GCG treatment had a body weight change of −18.6% on day 28 when normalized to Vehicle (FIG. 14A). When rats were treated with GLP-1/GCG at 5 nmol/kg they had a mean body weight change of −25%, and when combined with LAA at 7.5 nmol/kg a mean body weight Vehicle corrected change of −28.8% was observed (FIG. 14B). Bliss independence modelling suggests this effect is additive for the LAA and GLP-1/GCG combination at 7.5 and 1.5 nmol/kg and trends towards synergy for the LAA and GLP-1/GCG combination at 7.5 and 5 nmol/kg, assuming that each compound is acting independently of one another and a maximum body-weight reduction of 30% in this preclinical model.

An additional study provides evidence that the combination of a selective amylin receptor agonist and a GLP-1R/GCGR dual agonist synergistically delayed gastric emptying as compared to monotherapy in DIO rats.

Specifically, DIO male pair-housed rats fed a 32.5% high-fat diet (D12266B) for 7 weeks reached a mean body weight (BW) of −595 g. Rats were dosed SC daily for 28 days with vehicle (Vh), GLP-1/GCG dual agonist (1.5 or 5 nmol/kg), LAA (2.5 or 7.5 nmol/kg), or combination (1.5+2.5 nmol/kg, 1.5+7.5 nmol/kg, 5+2.5 nmol/kg, or 5+7.5 nmol/kg, respectively). In chow fed lean rats, gastric emptying (GE) was measured via X-ray fluoroscopic quantification following SC injections of Vh, GLP-1/GCG dual agonist at 1, 3, or 10 nmol/kg or LAA at 1, 3, or 10 nmol/kg. In a subsequent acute GE study, lean rats received a single dose of Vh, GLP-1/GCG dual agonist (2 nmol/kg), LAA (2 nmol/kg) or GLP-1/GCG dual agonist+LAA combination (2+2 nmol/kg). In this experiment, the LAA was that of SEQ ID NO: 11, and the GLP-1/GCG dual-agonist was cotadutide.

In a GE study in lean rats, acute treatment with LAA or GLP-1/GCG dual agonist dose-dependently decreased GE represented as GE AUC as % of Vehicle (101%, 34% and 8% with LAA at 1, 3, and 10 nmol/kg and 85%, 49% and 24% with GLP-1/GCG dual agonist at 1, 3, and 10 nmol/kg respectively). In a subsequent GE study, combination of sub-maximal doses of 2 nmol/kg of LAA and GLP-1/GCG dual agonist showed a greater effect on GE compared to monotherapy arms.

Overall, treatment with GLP-1/GCG dual agonist or LAA showed an expected decrease in GE in dose-dependent manner, and an additional effect on GE was observed with combination treatment at sub-maximal doses as compared to monotherapy, indicating a synergistic effect on gastric emptying in the combination group.

### Example 4: A 3-Month Pre-Clinical Study Confirms that Co-Administration of an AMYR Agonist and a GLP-1/GCG Dual Agonist Synergistically Drives Additional Body Weight Loss Compared with Monotherapy in DIO Rats

Male (9-11 weeks of age) and female (10-11 weeks of age) Wistar Han rats were SC administered with Vehicle/Vehicle (group 1), LAA and GLP-1/GCG (group 2-500 μg/kg and 5 μg/kg, respectively; group 3-2000 μg/kg and 5 μg/kg, respectively), LAA and Vehicle (group 4-2000 μg/kg and 0 μg/kg, respectively), or Vehicle and GLP-1/GCG (group 5-0 and 5 μg/kg respectively) daily for 91 days. Dosing for LAA was dose escalated with group 2 receiving 250 μg/kg from day 1-5 and 500 μg/kg for duration of study, and groups 3 and 4 receiving 250 μg/kg from day 1-5, 500 μg/kg from days 7-13 and 2000 μg/kg for duration of study. In the study, the LAA was AZD6234 (SEQ ID NO: 11) and GLP-1/GCG was AZD9550 (SEQ ID NO: 35); n=10/group per gender.

Body weight comparison for rats (both male and female) at study day 92 were significantly lower in all groups receiving for LAA or GLP-1/GCG mono treatment or in combination (groups 2-5) than animals receiving only Vehicle administration. As shown in Table 2, data for male rats indicate a synergistic body-weight effect for the combination in Group 3 (according to Bliss independence model), while data for female rats indicate an additive effect for the combination in Group 3 (Bliss independence model), assuming that each compound is acting independently of one another and a maximum body-weight reduction of 30% in this preclinical model.

TABLE 2
Effect on mean body weight for different treatments with the LAA of SEQ
ID NO: 11 or GLP-1/GCG of SEQ ID NO: 35 in mono- or combination therapy

| | Dose levels | | | | |
|---|---|---|---|---|---|
| | (g/kg/dose: | Mean Body | Mean Body | | Body Weight |
| | LAA of SEQ ID | Weight at | Weight at | Body Weight | Comparison, |
| | NO: 11 and GLP- | Day −1 | Day 92 | Gain (Day 92 | Day 92 vs. |
| Test Material | 1/GCG of SEQ | (grams, | (grams, | vs Day −1, | Control Group 1 |
| (Group No.) | ID NO: 35 | M/F) | M/F) | M/F) | Day 92 (M/F)^{a} |
| Group 1 | 0/0 | 282.8/208.5 | 405.3/250.3 | 143%/120% | 100%/100% |
| Group 2 | 500/5 | 276/201.3 | 279.8/197.1 | 100%/98% | 69%**/78%** |
| Group 3 | 2000/5 | 269.6/210.2 | 258.9/207 | 96%/98% | 64%**/83%** |
| Group 4 | 2000/0 | 268.6/209.8 | 319.2/222.1 | 119%/110% | 79%**/89%** |
| Group 5 | 0/5 | 274/201.6 | 325.5/225.4 | 119%/112% | 80%**/90%** |

^{a}Comparison of control group means (Group 1) to corresponding treated groups. Asterisks indicate statistically significant lower weights compared to control (**= p ≤ 0.01 at Day 92). Body weights were statistically significantly lower than control body weights beginning on Day 4 though Day 92 for males in Groups 2 to 5 and Group 2 females, beginning Day 18 through Day 92 for Groups 3 and 4 females, and beginning Day 77 through Day 92 for Group 5 females.

In a further study, male DIO Sprague-Dawley rats were fed a high-fat diet (32.5% kcal fat, D12266B; Research Diets) for approximately 6 weeks prior to study start. Rats were dosed every other day (Q2D) SC for 19 days with Vehicle, GLP1-Fc (0.1 or 1 mg/kg), Fc-LAA (0.1 or 2 mg/kg) or a combination of GLP1-Fc and Fc-LAA (0.1+0.1 mg/kg, 0.1+2 mg/kg, 1+0.1 mg/kg or 1+2 mg/kg respectively) with an n=7/group. Body weight was monitored daily through-out study and shown below as % change of vehicle treatment. Data reported as mean±standard error of the mean (SEM). In this experiment, the LAA was an Fc-Amylin, and the GLP-1 agonist was dulaglutide (a GLP-1 agonist covalently linked to an Fc fragment of IgG4).

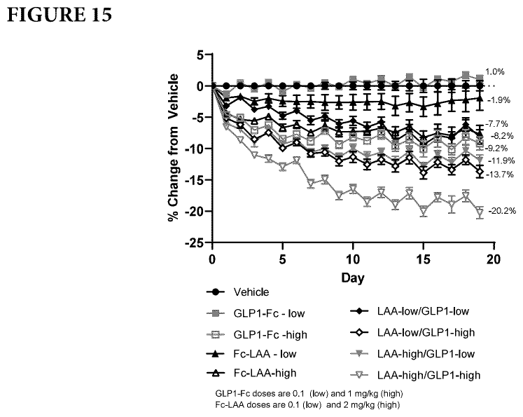

As shown in FIG. 15, DIO rats treated with GLP1-Fc gained 1.0% or lost −9.2% body weight normalized to vehicle (0.1 and 1 mg/kg respectively). Rats treated with Fc-LAA mono-treatment lost weight in dose dependent manner with −1.9% or −7.7% (0.1 and 2 mg/kg respectively). In combination, there was further body weight loss observed. GLP1-Fc (0.1 mg/kg) and Fc-LAA (0.1 mg/kg) saw a change of −8.2%, GLP1-Fc (1 mg/kg) and Fc-LAA (0.1 mg/kg) observed a decrease of −13.7%, GLP-1-Fc (0.1 mg/kg) and Fc-LAA (2 mg/kg) treatment led to a decrease of −11.9%, and GLP-1-Fc (1 mg/kg) and Fc-LAA (2 mg/kg) had an observed decrease of −20.2% body weight normalized to vehicle treatment. All GLP1-Fc and Fc-LAA combinations were predicted to be synergistic according to the Bliss independence model, assuming that each drug acts independently of the other, and a maximum body-weight reduction of 30% in this preclinical model.

### Example 5: Administering a Selective Amylin Receptor (AMYR) Agonist Polypeptide as an Adjunct Therapy while Maintaining an Existing GLP-1 Agonist Polypeptide Therapy in DIO Rats Synergistically Drives Additional Body Weight Loss

Male DIO Sprague-Dawley rats were fed a high-fat diet (32.5% kcal fat, D12266B; Research Diets) for approximately 8 weeks prior to study start. Rats were dosed SC for 15 days with Vehicle (n=16) or GLP-1RA (n=32) (5 nmol/kg) once-daily (QD). Beginning on day 16 rats initially treated with Vehicle either continued with Vehicle (n=8) or were switched to LAA (7.5 nmol/kg, n=8) administration. Rats initially receiving GLP-1 RA treatment either continued with GLP-1RA, were switched to LAA (7.5 nmol/kg), were switched to Vehicle, or continued with GLP-1RA with addition of LAA treatment (7.5 nmol/kg) for duration of study (day 30), with n=8/group. Body weight was measured daily through-out study. Data reported as mean±standard error of the mean (SEM); data analysis performed using GraphPad Prism In this experiment, the LAA was that of SEQ ID NO: 11, and the GLP-1R agonist was semaglutide.

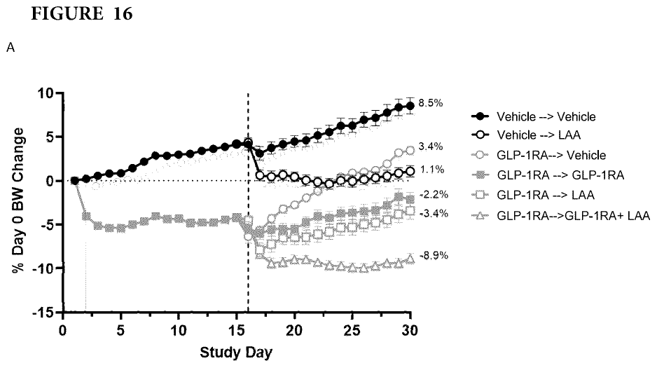

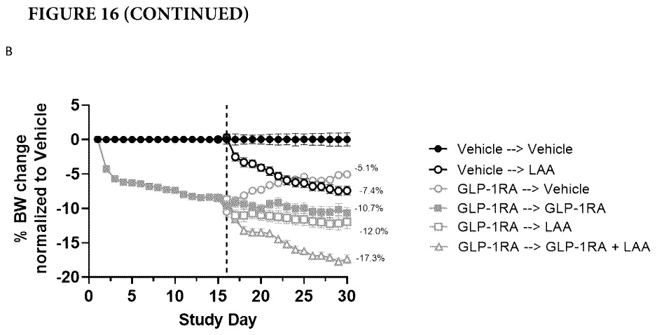

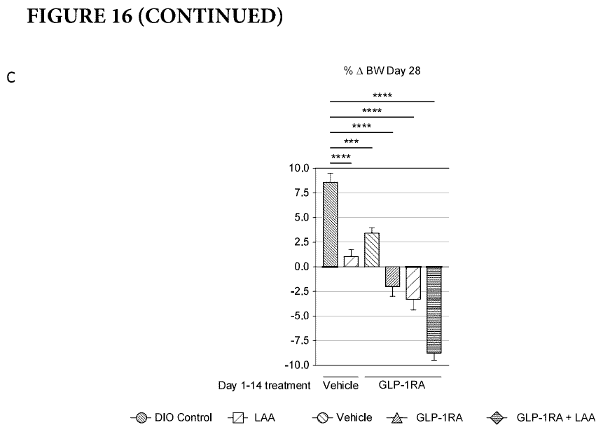

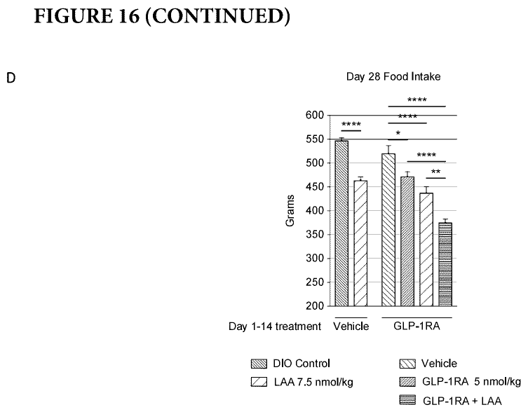

As shown in FIG. 16A, DIO rats treated with a GLP-1RA lost weight following 15 days of treatment, mean of −4.2% of day 0 body weight, while animals treated with Vehicle gained an average of +4.2% of day 0 body weight. Following treatment switch, rats maintained on Vehicle treatment continued to gain weight (+8.5% of day 0 body weight) through end of study (day 30), whereas rats that were switched from Vehicle to LAA lost weight from day 16 to day 30, with an overall mean of +1.1% day 0 body weight. Rats that started with GLP-1RA treatment and continued to receive only GLP-1 RA maintained body weight effect with an average BW loss of −2.2% at day 30; and animals that were switched from GLP-1 RA to LAA treatment had a BW loss mean of −3.4%. Rats switched from GLP-1RA to Vehicle gained weight from day 16 to day 30 with a mean of +3.4% from their Day 0 body weight. Rats that started with GLP-1RA treatment and continued with GLP-1RA plus LAA administration lost more weight than mono-treatment groups with a mean of −8.9% weight loss at day 30 (as shown in FIG. 16C). When body weight was normalized to the vehicle treatment through duration of study (as shown in FIG. 16B), rats that were switched from VH to LAA had a −7.1% change in body weight. Animals treated with GLP-1RA for the duration of the study had a −10.7% change in body weight, while animals that were switched from GLP-1 RA to LAA had a −12% change, animals switched from GLP-1 RA to Vehicle weighed −5.1% less, and animals that remained on GLP-1RA plus LAA for second half of study had a −17.3% change in body weight when normalized to Vehicle treatment—this data suggests that with a lead in phase of GLP-1RA treatment, the addition of LAA may lead to further weight loss than mono-treatment. Bliss independence model indicates an additive effect (with a trend towards synergy) for combinations of LAA and GLP-1RA during Day 16-30, after pretreatment with GLP-1RA on Day 1-15, assuming that each compound is acting independently of one another and a maximum body-weight reduction of 30% in this preclinical model.

As shown in FIG. 16D, the greatest reduction in food intake at the end of the study was observed in the animals that maintained GLP-1RA therapy combined with the LAA adjunct therapy for the second half of the study. This reduction was significantly greater than in animals either continuing GLP-1 monotherapy throughout or switching from GLP-1 therapy to LAA therapy at day 15.

Following the pre-clinical studies, a Phase II, randomised, parallel-group, double-blind, placebo-controlled, multi-centre study was designed to assess the efficacy, safety, and tolerability of a LAA compared with placebo, given once weekly as a subcutaneous (SC) injection, in adults living with overweight or obesity and type 2 diabetes who are on a stable dose of GLP-1 RA (semaglutide). In this study, the LAA is that of SEQ ID NO: 11. The primary objectives are in the context of determining whether treatment with LAA is superior to placebo for weight loss in participants with BMI≥27 kg/m² and type 2 diabetes, who are on a stable treatment dose of GLP-1 RA, for type 2 diabetes, after 26 weeks of treatment.

### Example 6: Combination Therapy with Selective Amylin Receptor (AMYR) Agonist and a GLP-1/GIP Dual Agonist Polypeptide Resulted in Synergistically Greater Body Weight Loss in DIO Rats Compared with Monotherapies

Male rats were fed a high-fat diet (45% kcal fat—D12451; Research Diets) or chow control matched diet (10% kcal fat—D12450K; Research Diets) for 17-21 weeks prior to study start. DIO rats were dosed once daily SC for 28 days (n=8/group) with either Vehicle, LAA (10 nmol/kg), GLP/GIP dual agonist (2, 10 or 30 nmol/kg), or a combination of LAA (10 nmol/kg for all groups) and GLP/GIP dual agonist (2, 10 or 30 nmol/kg). Control rats were dosed SC with Vehicle. Body weight was monitored daily through-out study and body composition measured by EchoMRI at baseline (week −1) and end of study (week 4). In this experiment, the LAA was that of SEQ ID NO: 11, and the GLP-1/GCG dual-agonist was tirzepatide.

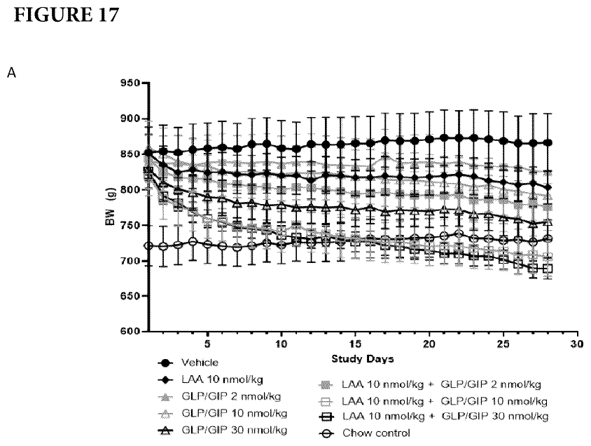

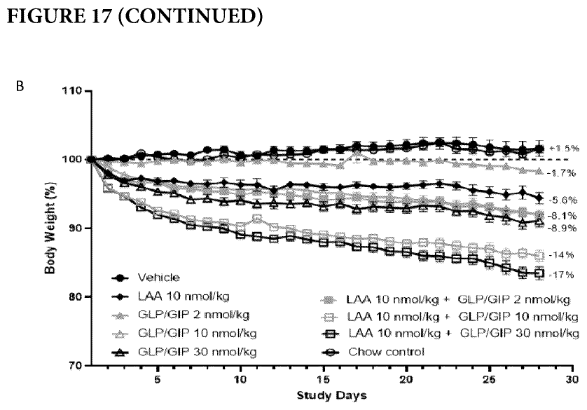

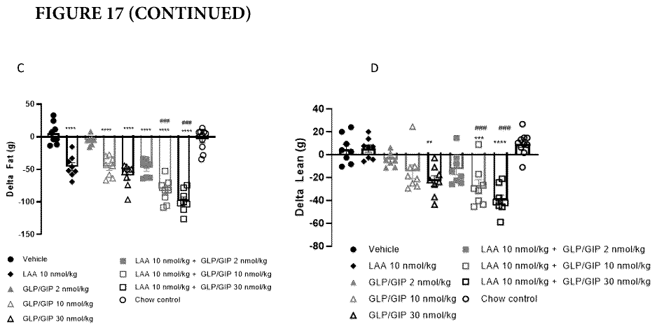

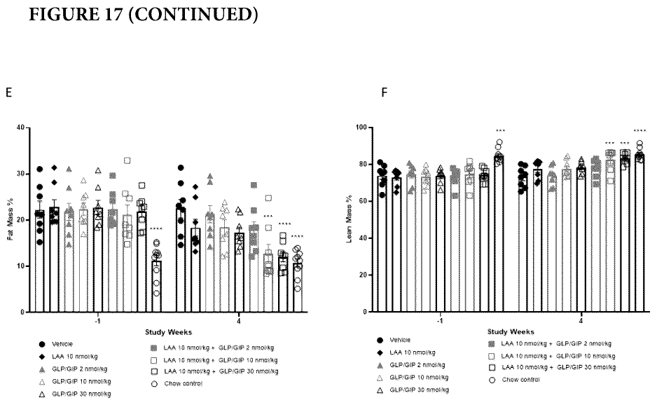

As shown in FIGS. 17A and 17B, rats that were treated with Vehicle gained 1.5% body weight over duration of study (both control and HFD groups). Rats treated with LAA mono-treatment saw a body weight change of −5.6% on day 28, and rats treated with GLP/GIP mono-treatment lost weight in a dose-dependent manner at −1.7, −8.1 and −8.9% at 2, 10 and 30 nmol/kg respectively. Rats that were treated with LAA and GLP/GIP in combination had a further decrease in BW. LAA was fixed at 10 nmol/kg in all combination groups with GLP/GIP and a dose-dependent BW decrease of −8.1, −14 and −17% was observed when LAA combined with GLP/GIP at 2, 10 and 30 nmol/kg respectively. Bliss independence modelling suggests this is an additive effect with LAA and combination of GLP/GIP at 2 or 10 nmol/kg. LAA and GLP/GIP at 30 nmol/kg suggests a synergistic effect on body weight assuming a max BW reduction of 30% in this preclinical model.

Body composition measured at end of study showed a significant decrease in fat mass (g) (FIG. 17C) with LAA mono-treatment, and a dose-dependent decrease in fat mass in GLP/GIP mono-treatment groups. The combination of LAA and GLP/GIP saw a further decrease in fat mass in a dose-dependent manner. As shown in FIG. 17D, LAA mono-treatment in rats saw no effect on lean mass (g) change, where GLP/GIP mono-treatment had a dose-dependent decrease of lean mass—no effect at 2 nmol/kg but a significant reduction vs. vehicle at 30 nmol/kg. When LAA and GLP/GIP were treated in combination there was a significant reduction in lean mass in groups receiving GLP/GIP at either 10 or 30 nmol/kg. When body composition was normalized to body weight, rats treated with LAA and GLP/GIP at either 10 or 30 nmol/kg had a significant decrease of % fat mass compared to Vehicle, and similar % fat mass to chow control animals (FIG. 17E). Lean mass normalized to body weight showed a significant increase in % lean mass in animals receiving LAA in combination with GLP/GIP at 10 or 30 nmol/kg (FIG. 17F).

### Example 7: Selective Amylin Receptor (AMYR) Agonist Monotherapy Reduces Body Weight and Food Intake in a Dose-Dependent Manner and Drives Fat-Mass Specific Body Weight Loss and Preserves Lean-Mass and Reduced Aversion as Compared to a Dual Amylin and Calcitonin Receptor Agonist (DACRA) Monotherapy in DIO Rats

Study 1 Design: High-fat diet (DIO) fed rats were used to profile SARA or DACRA monotherapy to evaluate food intake, body weight and body composition effects in a 4-week study. In this experiment, the SARA was that of SEQ ID NO: 11 and the DACRA was cagrilintide.

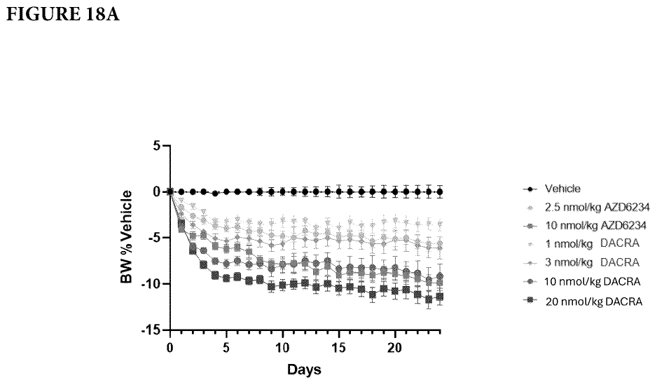

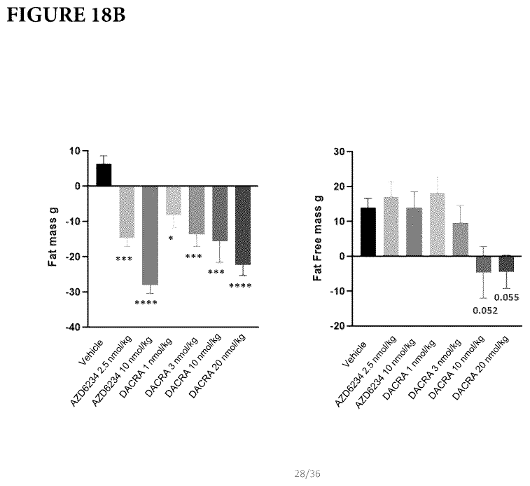

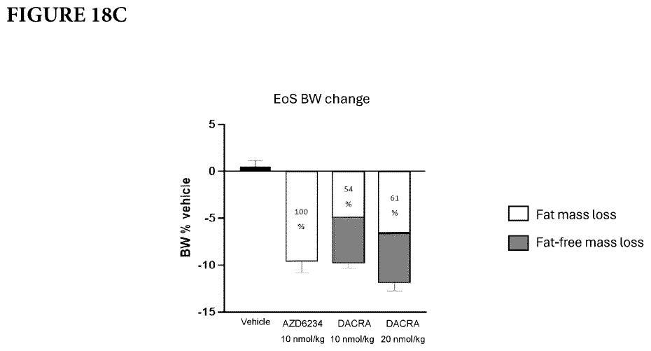

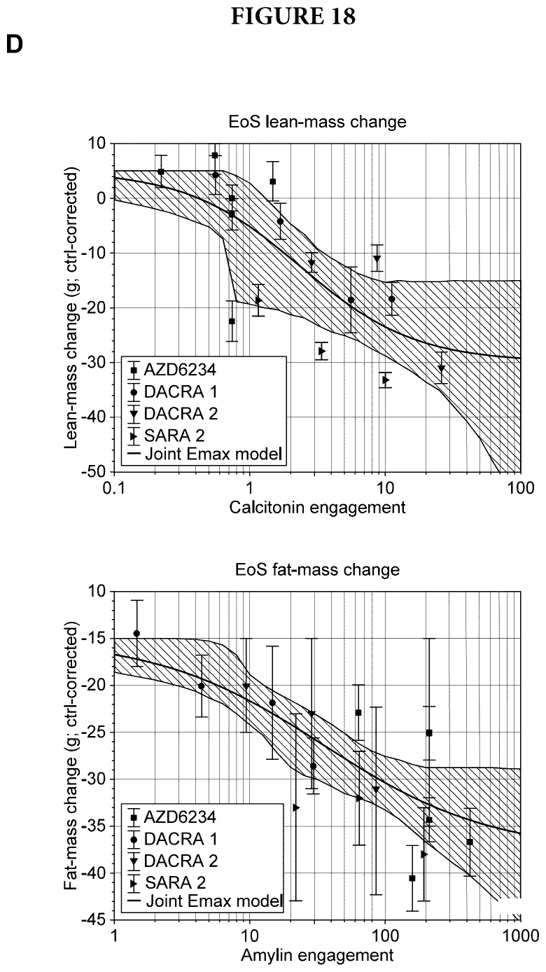

Study 1 Data: AMYR agonist (2.5 or 10 nmol/kg) or DACRA (1, 3, 10, or 20 nmol/kg) over a 4-week study in DIO rats resulted in dose-dependent reduction in body weight compared to vehicle treated rats. BW reduction was −5.2±0.6 and −9.7±0.7% for AMYR agonist (2.5 or 10 nmol/kg), and −3.0±0.6, −5.3±1.1, −8.8±1.4 and −11.6±0.9% for DACRA (1, 3, 10, or 20 nmol/kg), respectively (see FIG. 18A). A reduction of cumulative food intake of −14.3±1.2 and −20.9±0.7% of vehicle for AMYR agonist (2.5 or 10 nmol/kg), and −7.1±2.1, −16.0±2.5, −23.9±3.5 and −23.7±1.1% of vehicle for DACRA (1, 3, 10, or 20 nmol/kg), respectively, was observed. Body-composition measurements showed that AMYR agonist, unlike DACRA, promoted fat-mass-specific body weight loss while preserving lean-mass. The greatest fat-mass loss observed for AMYR agonist 10 nmol/kg was −28.0±2.3 g, which was nearly double that observed for matched body weight loss DACRA 10 nmol/kg of −15.5±6.0 g fat. Lean mass was preserved in the AMYR agonist groups, unlike in the DACRA groups (see FIG. 18B). Overall, AMYR agonist resulted in more fat mass loss and preservation of lean mass as compared to DACRA at matched body weight loss levels. In particular, 100% of body weight loss was fat-mass specific in the AMYR agonist 10 nmol/kg group, as compared to 54% and 61% for DACRA 10 and 20 nmol/kg groups, respectively (see FIG. 18C).

Study 2 Design: Target engagement was estimated in the following way for each compound. The average steady-state concentration (Cavg) after repeated subcutaneous injection was estimated in rats based on measured rat clearance in separate rat pharmacokinetic studies, dose level, and dosing interval. Plasma protein binding was determined in vitro using 3B Pharmaceutical's Escalate Equilibrium Shift Assay or an in-house Surface Plasmon Resonance (SPR) assay. Peptide in vitro potency was determined via HTRF cAMP accumulation assay using HEK293 cells recombinantly expressing the rat calcitonin receptor or rat amylin receptor (calcitonin receptor plus RAMP3) in saline buffer containing 0.1% bovine serum albumin. The in vitro potencies (IC50s) for amylin and calcitonin were adjusted to unbound values by taking into account the albumin concentration in the assay and fraction unbound data, according to an established method (Wan H and Bergstrom F (2007), High Throughput Screening of Drug-Protein Binding in Drug Discovery, Journal of Liquid Chromatography & Related Technologies, 30:5, 681-700; doi:10.1080/10826070701190989). Finally, the target engagement was calculated as the unbound Cavg divided by the unbound IC50, both for amylin and calcitonin. The Emax model was defined as Lean_mass=E0−Emax×(ENG\hat{ }h/(ENG\hat{ }h+ED50\hat{ }h)), where E0 is the baseline mass, Emax is the maximum induced mass change, ENG is the receptor engagement, and h is the slope (Hill) parameter. Parameter estimation was performed according to a maximum likelihood approach with an additive error model. Uncertainty of parameter estimates was determined by bootstrapping, sampling single measurements randomly with replacement within each experiment (N=500). Numeric analyses were performed in MATLAB (R2023b; The MathWorks, Natick, MA). The lean and fat mass changes are given as mean and standard error (N=8 per group).

Study 2 Data: Lean-mass loss correlated with the degree of calcitonin receptor engagement for DACRA, while fat-mass loss correlated with the degree of amylin receptor engagement (see FIG. 18D). Thus, the AMYR agonist reduces body weight and food intake in a dose-dependent manner in DIO rats and elicits fat-mass specific weight loss with preservation of lean mass.

Study 3 Design: In a separate study, male Wistar Han rats (9 weeks old) on a chow diet had overnight water restriction on day −1. On study day 1 they were given saccharin water for 4 hours and during this time were dosed once SC. After a 4-hour exposure to saccharin, it was removed and rats had access to water as usual. A preference test was performed 72 hours after compound administration. During this time, rats are given a choice of both saccharin and water, and the intake of both are measured over a 24-hour period. Rats generally prefer the sweet saccharin water, and if they associate the saccharin water with feeling malaise or nausea they will not drink as much of the saccharin and prefer water. The saccharin preference is calculated a percent of saccharin intake (g)/total intake (saccharin and water). Data reported as mean±standard error of the mean (SEM); data analysis performed using GraphPad Prism—statistical analysis performed using One-way ANOVA with Tukey ad hoc analysis. (*p<0.05, **p<0.01, ***0<0.001, ****p<0.0001 vs. Vehicle). In this experiment, the AMYR agonist was that of SEQ ID NO: 11, and the DACRA was cagrilintide.

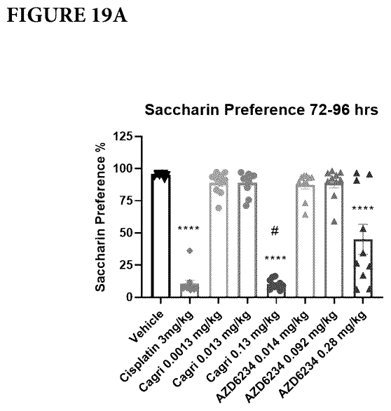

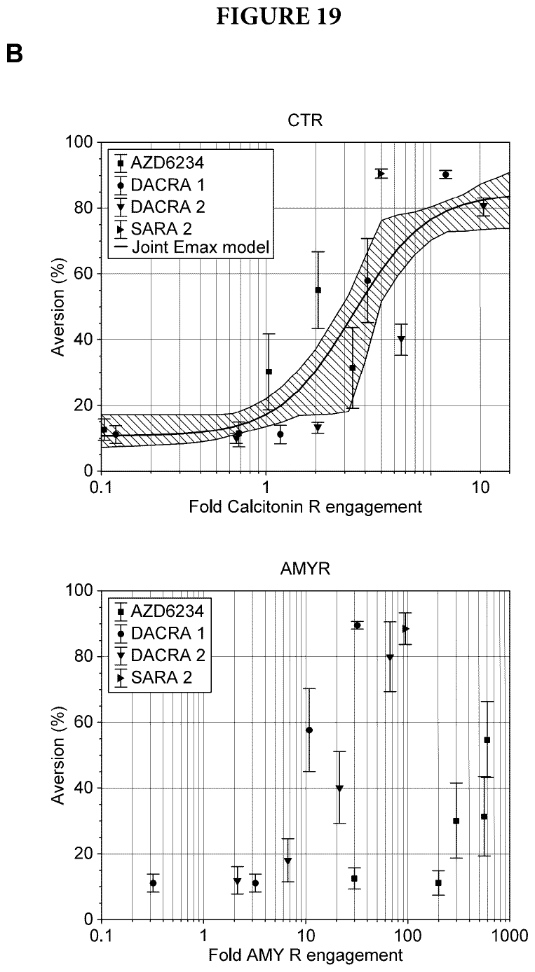

Study 3 Data: As shown in FIG. 19A, rats treated with vehicle had a saccharin preference of >90%, while animals treated with 0.13 mg/kg DACRA had a significantly lower saccharin preference, at a similar level to 3 mg/kg cisplatin (positive control). Rats treated with 0.28 mg/kg AMYR agonist on average had a lower saccharin preference than Vehicle, albeit with a wide spread, and the average tending to lower aversion (higher saccharine preference) than the highest dose DACRA group.

The AMYR agonist exhibits ~28 fold more amylin receptor engagement than the DACRA molecule, and the DACRA exhibits ~2-3 fold more calcitonin engagement than AMYR agonist.

Study 4: The inventors discovered that saccharine aversion in lean rats correlates to calcitonin receptor engagement but not to amylin receptor engagement (see FIG. 19B). Target engagement was estimated in the following way for each compound. The maximum concentration (Cmax) after subcutaneous injection was measured in separate rat pharmacokinetic studies and was dose-adjusted to the tested dose levels when appropriate, assuming dose linearity. Plasma protein binding was determined in vitro using 3B Pharmaceutical's Escalate Equilibrium Shift Assay or an in-house Surface Plasmon Resonance (SPR) assay. Peptide in vitro potency was determined via HTRF cAMP accumulation assay using HEK293 cells recombinantly expressing the rat calcitonin receptor or rat amylin receptor (calcitonin receptor plus RAMP3) in saline buffer containing 0.1% bovine serum albumin. The in vitro potencies (IC50s) for amylin and calcitonin were adjusted to unbound values by taking into account the albumin concentration in the assay and fraction unbound data, according to an established method (Wan H and Bergstrom F (2007), High Throughput Screening of Drug-Protein Binding in Drug Discovery, Journal of Liquid Chromatography & Related Technologies, 30:5, 681-700; doi:10.1080/10826070701190989). Finally, the target engagement was calculated as the unbound Cmax divided by the unbound IC50, both for amylin and calcitonin. The Emax model was defined as aversion=E0+Emax×(ENG\hat{ }h/(ENG\hat{ }h+ED50\hat{ }h)), where E0 is the baseline aversion, Emax is the maximum induced aversion, ENG is the receptor engagement, and h is the slope (Hill) parameter. Parameter estimation was performed according to a maximum likelihood approach with an additive error model, incorporating data from all compounds simultaneously. Uncertainty of parameter estimates was determined by bootstrapping, sampling single measurements randomly with replacement within each experiment (N=500). Numeric analyses were performed in MATLAB (R2023b; The MathWorks, Natick, MA). The aversion data are given as mean and standard error (N=9-11 per group). In this experiment, the AMYR agonist was that of SEQ ID NO: 11, SARA2 was eloralintide, DACRA1 was cagrilintide, and DACRA2 was petrelintide.

TABLE 3
Relative potency ratios

Relative potency ratio
(AMY3R/CTR), ^{ab}

| Molecule | | Rat | Human |
|---|---|---|---|
| hAmylin | SARA | 0.9 | 1.8 |
| Pramlintide | | 1.0 | 1.0 |
| AMYR agonist/SARA 1 | SARA | 1.6 | 0.9 |
| SARA 2 | SARA | 0.14 | 0.24 |
| DACRA 1 | DACRA | 0.01 | 0.08 |
| DACRA 2 | DACRA | 0.03 | 0.05 |

^{a} Assays in the presence of 0.1% bovine serum albumin, n ≥ 5.
^{b} Relative potency ratio = (amylin assay potency relative to pramlintide 100%)/(calcitonin assay potency relative to pramlintide 100%).
AMY, amylin; CTR, calcitonin, all except hAmylin and Pramlintide are lipidated peptides.

### Example 8: A Phase IIb Randomised, Double-Blind, Placebo-Controlled Study to Evaluate the Efficacy, Safety and Tolerability of Co-Administration of an AMYR Agonist and a GLP-1/GCG Dual Agonist in Participants Living with Obesity or Overweight with Co-Morbidity

Following the pre-clinical studies, a Phase IIb, global, randomised, parallel group, double-blind, placebo-controlled, multi-centre, reduced factorial study (‘ASCEND’ study) was designed to evaluate the efficacy, safety and tolerability of treatment with AZD9550 and AZD6234 in combination compared with placebo and with monotherapy in adults who are living with obesity or overweight with at least one weight-related co-morbidity.

The study was designed to be conducted in approximately 70 sites in approximately 7 countries globally. The study was designed for adult human subjects (aged between 18 and 75 years, inclusive) living with obesity (defined as a BMI 30 kg/m²) or overweight (defined as BMI≥27 kg/m²) with at least one of hypertension (without clinically manifested end-organ damage (as judged by the investigator)), dyslipidaemia or obstructive sleep apnoea. Individuals with known diabetes, diabetes symptoms or laboratory results suggestive of diabetes at screening were not eligible to participate in the study.

An eligibility screening protocol was designed. The eligibility screening period is up to 35 days prior to randomisation. The screening protocol was designed to include eligible consenting participants according to age (18 to 75 years of age, inclusive, at the time of signing the informed consent, with suitable veins for cannulation or repeated venipuncture), disease characteristics (at screening, have a BMI of (a) ≥30 kg/m² inclusive, or (b) ≥27 kg/m² and at least one of the following comorbidities (with documented medical history or currently being treated for): (i) Hypertension (without clinically manifested end-organ damage (as judged by the investigator)), (ii) Dyslipidaemia, and/or (iii) Obstructive sleep apnoea), weight (a stable body weight for 3 months prior to screening (±5% body weight change)), contraceptive requirements, and a negative pregnancy test (at screening and randomisation) and non-breastfeeding with respect to female participants of childbearing potential. Exclusion criteria included weight criteria, including subjects having obesity induced by other endocrine disorders (such as Cushing's syndrome, insulinoma, or Prader-Willi syndrome), subjects receiving prescription or non-prescription medication for weight loss or diabetes within the 3 months prior to screening, and/or previous or planned (within study period) bariatric surgery or fitting of a weight loss device (e.g. gastric balloon or duodenal barrier); diabetes status, including a history of type 1 or type 2 diabetes mellitus or symptoms indicative of insulinopenia or poor glucose control or at least one laboratory value suggestive of diabetes at screening (including one or more of HbA1c≥6.5% (48 mmol/mol), fasting serum glucose ≥126 mg/dL (7.0 mmol/L) or random glucose ≥200 mg/dL (11.1 mmol/L) at screening); gastrointestinal health status (e.g., gastroparesis, gastric outlet obstruction, IBD, and/or pancreatitis); cardiovascular/renal health status (e.g., impaired renal function, cardiovascular disease, and/or severely uncontrolled hypertension); mental health status; other disease or diagnostic status (e.g., HIV and/or hepatitis B or hepatitis C antibody); other medical history of a clinically important disease or disorder which may put the participant at risk (e.g., history of conditions relating to absorption, distribution, metabolism or excretion of drugs, history of neoplastic disease, history of drug or alcohol abuse, and recent drug history); prior or concomitant therapy associated with weight gain or that deemed clinically relevant within 3 months to screening; and prior or concomitant clinical study experience.

Approximately 600 participants are screened/enrolled in the study with approximately 360 eligible participants randomised to receive study intervention. Eligible participants are randomised on Study Day 1 (or Study Day −1) to receive both AZD9550 and AZD6234 in combination, AZD9550 alone or AZD6234 alone in a 4:1 ratio of active treatment to volume-matched placebo in each of the following 9 treatment arms:

TABLE 4
Phase II treatment schedule

| Treatment arm | AZD6234 (mg) | AZD9550 (mg) |
|---|---|---|
| High-high | 9.0 | 7.0 |
| Med-Med | 6.0 | 4.6 |
| Low-Low | 1.6 | 1.2 |
| Med-High | 6.0 | 7.0 |
| High-Med | 9.0 | 4.6 |
| Med-Low | 6.0 | 1.2 |
| Low-Med | 1.6 | 4.6 |
| PBO-High | Placebo | 7.0 |
| High-PBO | 9.0 | Placebo |

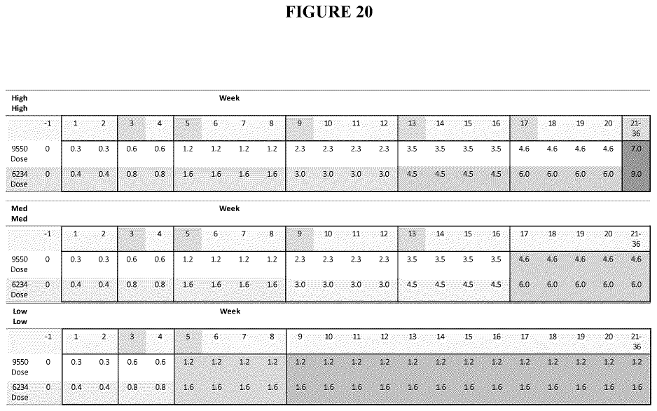

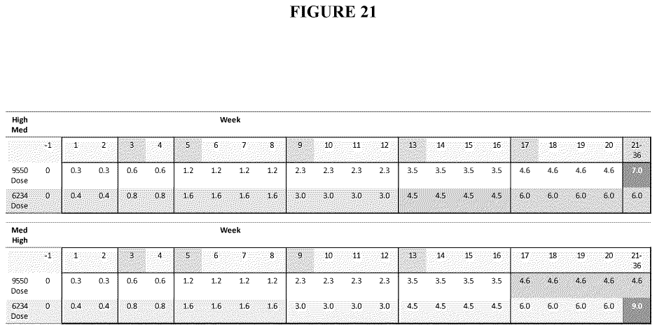

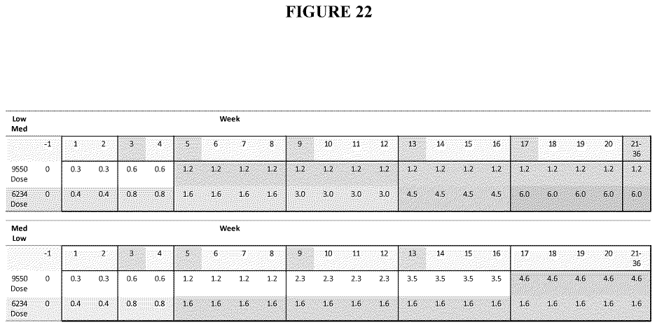

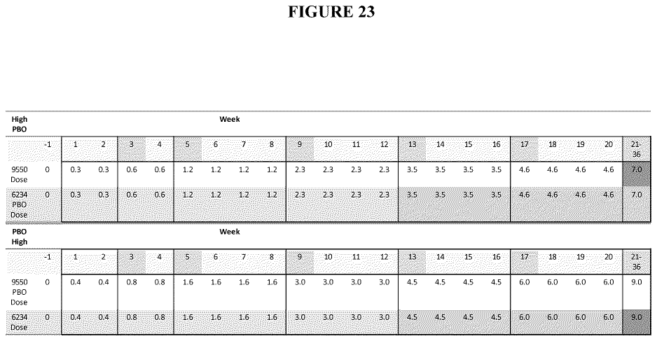

Based on the well-characterised class safety profiles of GLP-1, GCG and amylin agonists, and the available non-clinical and Phase I clinical safety data to date, GI effects such as nausea and vomiting are potential risks and will be monitored throughout the study. To achieve higher doses with tolerable GI AEs, GLP-1 RAs and amylin analogues may be up-titrated from a lower starting dose to gradually reach therapeutic doses. An exemplary titration schedule is set out in FIGS. 20-23.

Following the screening period, a 36-week Treatment Period commences during which participants receive study intervention once weekly from Study Day 1/Week 1 until Study Day 246/Week 36. Each once-weekly treatment comprises 2 SC injections. The first SC injection of study intervention on Study Day 1/Week 1 is administered in the clinic by a medical professional. Subject to completion of training, the second SC injection of study intervention on Study Day 1/Week 1 is administered in the clinic by the participant (self-administration) under medical supervision. Thereafter, study intervention may be administered in the clinic by the participant under medical supervision. After Study Day 29/Week 5, subsequent doses of study intervention are administered by the participant, either in the clinic during scheduled study visits or at home (home-dosing). Up-titration from the starting dose to the target dose takes place over a period of up to 20 weeks (e.g., up to 16 weeks), depending on the allocated treatment arm. The final dose of study intervention is administered on Study Day 246/Week 36. Following the Baseline Visit (Study Day 1/Week 1), participants attend weekly site visits until Study Day 29/Week 5. Thereafter, site visits are every 4 weeks until Study Day 253/Week 37 (EOEA Visit).

The starting doses of AZD9550 (0.3 mg) and AZD6234 (0.4 mg) for all treatment arms in this study were selected since they were shown to be well tolerated based on observations from the respective SAD/MAD studies. To reach higher doses, the dose of each study intervention will be gradually titrated to reach the 3 planned target doses for AZD9550 (1.2, 4.6 and 7.0 mg) and AZD6234 (1.6, 6.0 and 9.0 mg). The highest doses of AZD9550 (7.0 mg) and AZD6234 (9.0 mg) in this study were selected based on the combined considerations of safety, tolerability, PK and PD (weight loss) data and emerging clinical data. At the highest doses tested, 7.0 mg AZD9550 and 9.0 mg AZD6234, both study interventions were considered tolerated with no major safety concerns identified following up-titration.

A total treatment duration of 36 weeks has been selected to accommodate an up-titration period of 20 weeks for the highest target dose of AZD9550 (7.0 mg) and of AZD6234 (9.0 mg) followed by a maintenance treatment period at those doses of 16 weeks. For the low and medium target doses, the total duration of treatment will also be 36 weeks but up-titration to the target doses will be completed in less than 20 weeks. The total treatment duration of 36 weeks for participants in this study is expected to result in at least 80% of the maximally projected weight loss reported for similar patient populations (Wegovy US Prescribing Information, Zepbound US Prescribing Information). The maximum increment between successive up-titrations will be 2-fold and, to minimise the burden on sites and participants, the schedule of up-titration will be the same for both AZD9550 and AZD6234.

The co-primary endpoints, AZD9550 and AZD6234 in combination vs placebo, are percent change in body weight from baseline evaluated after 36 weeks of study intervention and weight loss ≥5% from baseline after 36 weeks of treatment. All study interventions are administered as separate SC injections. Administration is either carried out at the clinic, or by self-administration. Determination of subjects permitted to self-administer is determined by the medical team, who provide instructions and training for self-administration, including injection site selection and rotation.

Based on modelling and simulation, AZD9550/AZD6234 dose combinations of 1.2 mg/1.6 mg, 4.6 mg/6.0 mg and 7.0 mg/9.0 mg are predicted to reduce body weight from baseline at Week 36 by approximately 26%, 29% and 29%, respectively.

The dose range of AZD9550/AZD6234 evaluated in this study will provide adequate information on body weight loss to establish the dose- and/or exposure-relationship for weight loss. It will also delineate the effects of AZD9550 and AZD6234 monotherapy on body weight loss and selected tolerability endpoints from those of the AZD9550/AZD6234 combination to support dose selection for Phase Ill.

Synthesis of AMYR agonists, such as lipidated pramlintide analogue polypeptides, and GLP-1/GCG dual agonist peptides, have been described elsewhere (see WO 2022/129254 and WO 2023/148366, respectively, the contents of which are hereby incorporated by reference).

In this clinical trial, the AMYR agonist is AZD6234 (SEQ ID NO: 11) and the GLP-1/GCG dual agonist is AZD9550 (SEQ ID NO: 35). AZD6234 is a potent agonist of human, cynomolgus monkey, and rat amylin receptors, displaying in vitro selectivity over calcitonin receptor activation. AZD9550 is a synthetic GLP-1R/GCGR dual peptide agonist designed with a GLP-1R:GCGR potency ratio similar to cotadutide, but with a longer half-life to optimize exposure and dosing frequency in the clinic. In vitro studies and in vivo toxicology studies demonstrated acceptable safety profiles.

### Primary Objectives

The co-primary objectives of this study are:

| Objectives | End-points |
|---|---|
| To determine whether treatment with | Percent change in body weight from |
| AZD9550 and AZD6234 in combination is | baseline after 36 weeks of treatment |
| superior to placebo for weight loss | |
| To assess the effect of treatment with | Weight loss ≥5% from baseline after 36 |
| AZD9550 and AZD6234 in combination vs | weeks of treatment |
| placebo on the proportion of participants | |
| with weight loss ≥5% | |

### Secondary Objectives:

The secondary objectives of this study are:

| Objectives | End-points |
|---|---|
| To determine whether treatment with | Absolute change in body weight from |
| AZD9550 and AZD6234 in combination is | baseline after 36 weeks of treatment |
| superior to placebo for weight loss | |
| To determine whether treatment with | Percent and absolute change in body |
| AZD9550 and AZD6234 in combination is | weight from baseline after 36 weeks of |
| superior to AZD9550 and AZD6234 | treatment |
| monotherapy for weight loss | |
| To assess the effect of treatment with | Weight loss ≥5% from baseline after 36 |
| AZD9550 and AZD6234 in combination vs | weeks of treatment |
| monotherapy on the proportion of | |
| participants with weight loss ≥5% | |
| To assess the effect of treatment with | Weight loss ≥10% and ≥15% from |
| AZD9550 and AZD6234 in combination vs | baseline after 36 weeks of treatment |
| placebo and monotherapy on the proportion | |
| of participants with weight loss ≥10% | |
| and ≥15% | |
| To assess the immunogenicity profile of | Prevalence, incidence and titres of ADAs to |
| treatment with AZD9550 and AZD6234 in | AZD9550 and AZD6234 in combination and |
| combination and as monotherapies | as monotherapies after 36 weeks of |
| | treatment |

### Safety Objectives

The safety and tolerability of AZD9550 and AZD6234 in combination compared with placebo and with monotherapy is to be assessed by monitoring incidence of adverse events (AE), vital signs, and clinical and laboratory assessments. In particular, occurrence and time to first occurrence of AE, Serious AEs (SAE), SAE with outcome of death, AE leading to discontinuation of study intervention (DAE), and AE possibly related to study intervention as assessed by the investigator; observed values, change from baseline and time to first treatment-emergent abnormality in vital signs (systolic and diastolic BP, pulse rate, respiration rate, body temperature) and 12-lead ECG parameters; observed values, change from baseline and time to first treatment-emergent abnormality in specified safety laboratory parameters; occurrence and time to first occurrence of Adverse event(s) of special interest (AESIs): acute pancreatitis; cholelithiasis, cholecystitis and biliary disease; thyroid cancer; suicidal ideation and behaviour; MACE; acute renal impairment; severe GI events; hypoglycaemia; hypocalcaemia; and injection site reactions (ISRs).

### Exploratory Endpoints

Further exploratory endpoints relating to AZD9550 and AZD6234 in combination as compared to placebo and/or monotherapies are to be assessed by monitoring the following parameters.

### Pharmacokinetics

Plasma concentrations of AZD9550 and AZD6234
   Glucose Homeostasis, β-Cell Function, and Insulin Sensitivity (Vs. Placebo or Monotherapy):
Change from baseline in HOMA scores (HOMA-IR, HOMA-B, and HOMA-S) after Week 36
Change from screening in HbAlc after Week 36
Change from baseline in fasting glucose, fasting insulin, fasting c-peptide, proinsulin and pro-insulin/c-peptide ratio and adiponectin levels after Week 36
   Lipid Profile (Vs. Placebo or Monotherapy)
Change from baseline after 36 weeks treatment on total cholesterol, HDL-cholesterol, LDL-cholesterol, non-HDL-cholesterol (calculated), VLDL-cholesterol (calculated), Triglycerides, and non-esterified fatty acids
   End-Organ Protection (Vs. Placebo or Monotherapy):
Change over time on:
   CV endpoints (hs Troponin T and hs Troponin I)
   Renal biomarkers: eGFR (creatinine and cystatin C), UACR (urine albumin and creatinine ratio)
   Liver biomarkers: FIB-4 (calculated AST, ALT, age, platelet count), ELF, Pro C3
   Biomarkers of chronic inflammation: hs CRP, hs IL-6
   Sarcopenia Index (based on serum creatinine/cystatin C)
   Thyroid function (based on TSH, total T3, fT4)
      Alcohol Consumption (Vs. Placebo or Monotherapy)
Change over time in Phosphatidylethanol (Peth) value
   Physical Function and/or Mood or Cognitive Function (Vs. Placebo or Monotherapy)
Change from baseline in C-SSRS, PHQ-9, FACIT-GP5 and PGI of limitation of physical function after 36 weeks of treatment
   Weight Loss Quality (Vs. Placebo or Monotherapy):
Change from baseline in visceral adiposity distribution after 36 weeks of treatment:
   Waist circumference
   Hip circumference
   Waist-to-hip ratio

### Blood Pressure (BP) and Heart Rate (HR)

Change from baseline in mean systolic and diastolic BP, and HR over 24 hours and during the day and night after 36 weeks treatment.
   Immunogenicity Profile (Vs. Placebo or Monotherapy):
Incidence and titres of ADAs to AZD9550 and AZD6234 in combination and as monotherapies for up to 12 months after the ET Visit or the FU Visit.

1. A method for treating and/or preventing a disease or disorder in a subject, the method comprising administering to the subject
(a) about 1 mg to about 15 mg of an amylin receptor (AMYR) agonist; and
(b) about 0.1 mg to about 10 mg of a GLP-1 receptor (GLP-1R) agonist.

2. The method according to claim 1, wherein the GLP-1R agonist activates both a GLP-1R and a glucagon receptor (GCGR).

3. The method according to claim 1, wherein the method further comprises administering to the subject a glucagon receptor (GCGR) agonist.

4. The method of claim 1, wherein the amount of GLP-1R agonist is from about 0.1 mg to about 7 mg.

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. The method of claim 1, wherein the amount of AMYR agonist is from about 1 mg to about 10 mg.

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. The method of claim 1, wherein the AMYR agonist, GLP-1R agonist, and/or GCGR agonist is administered by subcutaneous injection.

64. The method of claim 1, wherein the AMYR agonist, GLP-1R agonist, and/or GCGR agonist is administered to the subject by self-administration.

65. The method of claim 1, wherein the AMYR agonist, GLP-1R agonist, and/or GCGR agonist is administered about once every 4, 5, 6, 7, 8, 9, 10, 11, 12, 13, or 14 days; optionally wherein the AMYR agonist, GLP-1R agonist, and/or GCGR agonist is administered about once a week.

66. The method of claim 1, wherein the AMYR agonist, GLP-1R agonist, and/or GCGR agonist is administered to the subject multiple times over a period of at least 3 months, 6 months, 9 months, 1 year, 2 years, or 5 years.

67. The method of claim 1, wherein the AMYR agonist, GLP-1R agonist, and/or GCGR agonist is titrated to a final dose over a period between about 4 weeks and about 36 weeks, optionally over a period of about 5 weeks, about 17 weeks or about 21 weeks.

68. (canceled)

69. The method of claim 1, wherein the subject's body weight is reduced by ≥5%, optionally, wherein the subject's body weight is reduced by ≥10%, ≥15%, or ≥20%.

70. The method of claim 1, wherein the disease or disorder is obesity.

71. The method of claim 1, wherein the disease or disorder is an obesity-related condition; optionally wherein the obesity-related condition is overweight, morbid obesity, obesity prior to surgery, obesity-linked inflammation, obesity-linked gallbladder disease, sleep apnoea and respiratory problems, hyperlipidaemia, degeneration of cartilage, osteoarthritis, or reproductive health complications of obesity or overweight such as infertility.

72. (canceled)

73. (canceled)

74. The method of claim 1, wherein the method further comprises treating, improving, and/or protecting liver and/or kidney function in the subject.

75. (canceled)

76. The method of claim 1, wherein the method of treatment reduces the body weight of a subject, optionally wherein the subject's reduction in body weight is fat-specific weight loss.

77. (canceled)

78. The method of claim 1, wherein the subject experiences reduced nausea as compared to a therapeutically effective dose of a dual amylin-calcitonin receptor agonist (DACRA), a GLP-1R agonist, a GLP-1R/GCGR dual agonist; a GLP1/GCG/GIP triple agonist; and/or a DACRA-GLP-1Ra combination.

79. The method of claim 1, wherein the AMYR agonist, GLP-1R agonist, and/or GCGR agonist is a polypeptide, small molecule drug, antibody, antibody-drug conjugate, or aptamer; or a pharmaceutically acceptable salt thereof.

80. (canceled)

81. (canceled)

82. The method of claim 1, wherein the AMYR agonist has selectivity to AMYR as compared to a calcitonin receptor (CTR).

83. (canceled)

84. The method of claim 1, wherein the AMYR agonist is an AMYR agonist polypeptide, or a pharmaceutically acceptable salt thereof, which comprises an amino acid sequence having at least 90% identity to pramlintide (KCNTATCATQRLANFLVHSSNNFGPILPPTNVGSNTY-amide, SEQ ID NO: 5).

85. (canceled)

86. (canceled)

87. (canceled)

88. (canceled)

89. (canceled)

90. The method of claim 1, wherein the GLP-1R agonist polypeptide, or pharmaceutically acceptable salt thereof, comprises an amino acid sequence having at least 90%, at least 95%, at least 97%, at least 99%, or 100% identity to the amino acid sequence
(SEQ ID NO: 33)
H(Aib)QGTFTSDVSK(αMePhe)LDTKRARDFVQWLLE(Aib)
G-acid.

91.-115. (canceled)
